(19) United States
(12) Patent Application Publication
(10) Pub. No.: US 2024/0043386 A1
(43) Pub. Date: Feb. 8, 2024

(54) Modulators of the Cystic Fibrosis Transmembrane Conductance Regulator Protein and Methods of Use

(71) Applicant: AbbVie Global Enterprises Ltd., Hamilton (BM)

(72) Inventors: Andrew Bogdan, Evanston, IL (US); David J. Hardee, Vernon Hills, IL (US); Timothy R. Hodges, Milwaukee, WI (US); Henry Korman, Vernon Hills, IL (US); Huan-Qiu Li, Wilmette, IL (US); Michael R. Schrimpf, Grayslake, IL (US)

(21) Appl. No.: 18/378,518

(22) Filed: Oct. 10, 2023

Related U.S. Application Data
(63) Continuation of application No. 17/554,086, filed on Dec. 17, 2021, now abandoned.
(60) Provisional application No. 63/134,392, filed on Jan. 6, 2021.

(51) Int. Cl. C07D 215/36; C07D 401/12

(52) U.S. Cl. CPC C07D 215/36; C07D 401/12

(57) ABSTRACT

The present invention provides for compounds of Formula (I)

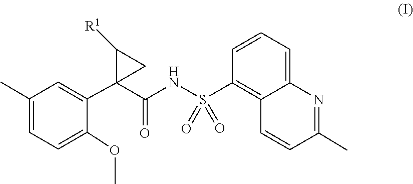

wherein R¹ has any of the values defined in the specification, and pharmaceutically acceptable salts thereof, pharmaceutical compositions comprising the same, and methods of treating cystic fibrosis by administering a compound of the invention.

### RELATED APPLICATION

This application is a continuation of U.S. patent application Ser. No. 17/554,086, filed Dec. 17, 2021, which claims the benefit of U.S. Provisional Application No. 63/134,392, filed Jan. 6, 2021, the contents of which are each herein incorporated in its entirety.

### FIELD OF THE INVENTION

This invention pertains to substituted cyclopropyl compounds which are modulators of the Cystic Fibrosis Transmembrane Conductance Regulator (CFTR) protein, useful in treating diseases and conditions mediated and modulated by CFTR. The invention also relates to compositions containing compounds of the invention.

### BACKGROUND

Cystic fibrosis is the most common fatal genetic disease in humans (Bobadilla, J. L., Macek, M., Jr, Fine, J. P., Farrell, P. M., 2002. Cystic fibrosis: a worldwide analysis of CFTR mutations—correlation with incidence data and application to screening. Hum. Mutat. 19, 575-606. doi:10.1002/humu.10041). It is caused by mutations in the gene for CFTR, an anion channel that regulates mucus secretions in epithelial cells of the lungs. In the United States, about one in every 2,500 infants is affected, and up to 10 million individuals carry a single copy of the defective gene without apparent ill effects. In contrast, individuals with two copies of the mutated CFTR gene suffer from the debilitating and fatal effects of CF, including chronic lung infections.

Pulmonary exacerbations resulting in hospitalization are common in CF patients. Over time, progressive damage to the lungs from chronic infection can result in a need for lung transplantation. Median age of death in the United States is approximately 31 years (Marshall, B.; Faro, A. et al. Cystic Fibrosis Foundation Patient Registry 2017 Annual Data Report, Cystic Fibrosis Foundation, 2018).

Standard treatment protocols for CF include daily airway clearance regimens, digestive enzyme supplements and the liberal use of antibiotics to control infection. The extensive treatment burden has a substantial effect on quality of life for CF patients and caregivers (Sawicki, G. S.; Sellers, D. E.; Robinson, W. M.; 2009. High Treatment Burden in Adults with Cystic Fibrosis: Challenges to Disease Self-Management. J. Cyst. Fibr. 8, 91-96. https://doi.org/10.1016/j.jcf.2008.09.007). New modulator therapies are available for certain genotypes, including the G55ID and F508del populations, but these are not universally effective and are not approved for many other CFTR mutations. Accordingly, there is a need for novel compounds able to modulate CFTR.

### SUMMARY

In certain embodiments, the invention provides for compounds of Formula (I), or a pharmaceutically acceptable salt thereof,

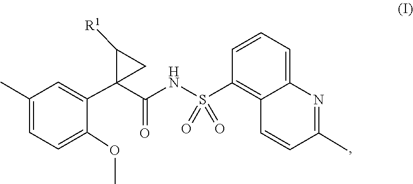

wherein
R¹ is selected from the group consisting of phenyl and 6-membered heteroaryl; wherein R¹ is optionally substituted with one or more R²
R² is selected from the group consisting of fluoro, chloro, bromo, C₁-C₄ alkyl, C₁-C₄ haloalkyl, C₂-C₆ alkoxyalkyl, —OR^{2a}, and —NR^{2b}R^{2c};
R^{2a} is C₁-C₄ alkyl; and
R^{2b} and R^{2c} are each independently selected from the group consisting of hydrogen and C₁-C₄ alkyl.

In certain embodiments of compounds of Formula (I), or a pharmaceutically acceptable salt thereof, R¹ is selected from the group consisting of phenyl, pyridyl, pyrazinyl, pyridazinyl, and pyrimidinyl; wherein R¹ is optionally substituted with one or more R².

In certain embodiments of compounds of Formula (I), or a pharmaceutically acceptable salt thereof, R¹ is selected from the group consisting of

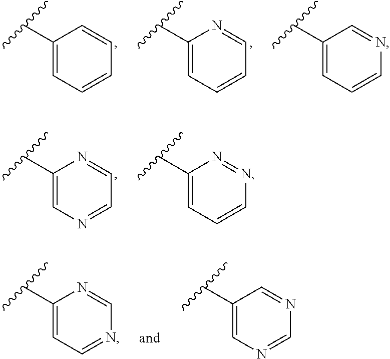

wherein R¹ is optionally substituted with one or more R².

In certain embodiments of compounds of Formula (I), or a pharmaceutically acceptable salt thereof, R¹ is pyridyl; wherein R¹ is optionally substituted with one or more R². In certain embodiments of compounds of Formula (I), or a pharmaceutically acceptable salt thereof, R² is selected from the group consisting of C₁-C₄ alkyl, C₁-C₄ haloalkyl, C₂-C₆ alkoxyalkyl, and —OR^{2a}. In certain embodiments of compounds of Formula (I), or a pharmaceutically acceptable salt thereof, R² is selected from the group consisting of C₁-C₄ alkyl and —OR^{2a}. In certain embodiments of compounds of Formula (I), or a pharmaceutically acceptable salt thereof, R² is —OR^{2a}.

In certain embodiments of compounds of Formula (I), or a pharmaceutically acceptable salt thereof, R¹ is

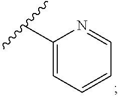

wherein R¹ is optionally substituted with one or more R². In certain embodiments of compounds of Formula (I), or a pharmaceutically acceptable salt thereof, additionally, R² is selected from the group consisting of C₁-C₄ alkyl and —OR^{2a}.

In certain embodiments of compounds of Formula (I), or a pharmaceutically acceptable salt thereof, R¹ is phenyl; wherein R¹ is optionally substituted with one or more R²; and R² is selected from the group consisting of fluoro, C₁-C₄ alkyl, C₁-C₄ haloalkyl, and —OR^{2a}.

In certain embodiments of compounds of Formula (I), or a pharmaceutically acceptable salt thereof, R¹ is pyrazinyl; wherein R¹ is optionally substituted with one or more R²; and R² is selected from the group consisting of C₁-C₄ alkyl and —OR^{2a}.

In certain embodiments of compounds of Formula (I), or a pharmaceutically acceptable salt thereof, R¹ is pyrimidinyl; wherein R¹ is optionally substituted with one or more R².

In certain embodiments, a compound is provided which is

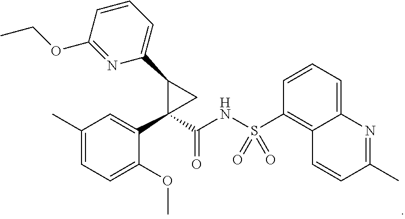 [SMILES: CCOc1cccc([C@H]2C[C@@]2(C(=O)NS(=O)(=O)c2cccc3nc(C)ccc23)c2cc(C)ccc2OC)n1]

In certain embodiments, a compound, or a pharmaceutically acceptable salt thereof, is provided.

In certain embodiments, a compound is provided which is (1S,2S)-2-(6-ethoxypyridin-2-yl)-1-(2-methoxy-5-methylphenyl)-N-(2-methylquinoline-5-sulfonyl)cyclopropane-1-carboxamide. In certain embodiments, the pharmaceutically acceptable salt of (1S,2S)-2-(6-ethoxypyridin-2-yl)-1-(2-methoxy-5-methylphenyl)-N-(2-methylquinoline-5-sulfonyl)cyclopropane-1-carboxamide is provided. In certain embodiments, a compound, or a pharmaceutically acceptable salt thereof, is provided.

Certain embodiments of the invention relate to a pharmaceutical composition comprising a therapeutically effective amount of a compound of Formula (I) according to claim 1, or a pharmaceutically acceptable salt thereof, in combination with a pharmaceutically acceptable carrier. Certain embodiments, relate to a pharmaceutical composition comprising a compound of claim 1, or a pharmaceutically acceptable salt thereof, one or more potentiator, and one or more additional correctors.

Certain embodiments of the invention, relate to a method for treating cystic fibrosis in a subject comprising administering a therapeutically effective amount of a compound of claim 1, or a pharmaceutically acceptable salt thereof, to a subject in need thereof. Certain embodiments of the invention, relate to a method for treating cystic fibrosis in a subject comprising administering a therapeutically effective amount of a compound of claim 1, or a pharmaceutically acceptable salt thereof, one or more potentiator, and one or more additional correctors, to a subject in need thereof.

### DETAILED DESCRIPTION

The present invention describes compounds which inhibit the activity of

Disclosed herein are compounds of Formula (I)

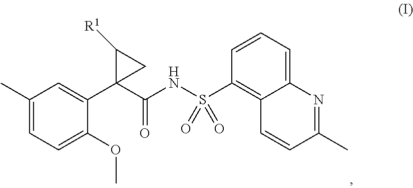

wherein R¹ is defined above in the Summary and below in the Detailed Description. Further, compositions comprising such compounds and methods for treating conditions and disorders using such compounds are also disclosed.

Compounds disclosed herein may contain one or more variable(s) that occur more than one time in any substituent or in the Formulae herein. Definition of a variable on each occurrence is independent of its definition at another occurrence. Further, combinations of substituents are permissible only if such combinations result in stable compounds. Stable compounds are compounds, which can be isolated from a reaction mixture.

### Definitions

Certain terms as used in the specification are intended to refer to the following definitions, as detailed below.

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds. Reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “alkoxy,” as used herein, refers to an alkyl group, as defined herein, appended to the parent molecular moiety through an oxygen atom. The alkoxy group may have one, two, three, four, or five carbons unless otherwise specified. Representative examples of alkoxy include, but are not limited to, methoxy, ethoxy, propoxy, 2-propoxy, butoxy, tert-butoxy, and pentyloxy, and the like.

The term “alkoxyalkyl,” as used herein, refers to an alkoxy group, as defined herein, appended to the parent molecular moiety through an alkyl group, as defined herein. The alkoxyalkoxy group may have two, three, four, five, or six carbons unless otherwise specified Representative examples of alkoxyalkyl include, but are not limited to, tert-butoxymethyl, 2-ethoxyethyl, 2-methoxyethyl, and methoxymethyl, and the like.

The term “alkyl,” as used herein, refers to a saturated, straight or branched hydrocarbon chain radical having one, two, three, or four carbons unless otherwise specified. Representative examples of alkyl include, but are not limited to, methyl, ethyl, n-propyl, isopropyl, n-butyl, sec-butyl, isobutyl, tert-butyl, and the like.

The term “halo” or “halogen,” as used herein, means Cl, Br, I, and F.

The term “haloalkyl,” as used herein, refers to an alkyl group, as defined herein, in which one or more hydrogen atoms are replaced by halogen having one, two, three, or four carbons unless otherwise specified Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, trifluorobutyl, trifluoropropyl, and the like.

The term “heteroaryl,” as used herein, refers to an aromatic ring radical containing one or more heteroatoms or a ring system. The monocyclic heteroaryl is a five- or six-membered ring. The five-membered ring contains two double bonds and one or more heteroatoms selected from O, S, and N. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl, and the like.

The term “heteroatom,” as used herein, means a nitrogen, oxygen, or sulfur atom.

In some instances, the number of carbon atoms in a moiety is indicated by the prefix “Cx-Cy”, wherein x is the minimum and y is the maximum number of carbon atoms in the substituent. Thus, for example, “C1-C6 alkyl” means an alkyl substituent containing from 1 to 6 carbon atoms and “C1-C3 alkyl” means an alkyl substituent containing from 1 to 3 carbon atoms.

In some instances, the number of ring atoms in a moiety is indicated by the prefix “x-y membered”, wherein x is the minimum and y is the maximum number of ring atoms in the substituent. Thus, for example, the term “5- to 6-membered heteroaryl” means a heteroaryl containing 5 to 6 ring atoms.

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below.

The phrase “pharmaceutical composition” refers to a composition suitable for administration in medical or veterinary use.

The phrase “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio.

The terms “prevent,” “preventing,” and “prevention” refer to a method of preventing the onset of a disease and/or its attendant symptoms or barring a subject from acquiring a disease. As used herein, “prevent,” “preventing,” and “prevention” also include delaying the onset of a disease and/or its attendant symptoms and reducing a subject's risk of acquiring or developing a disease or disorder.

The term “stable” refers to compounds that possess stability sufficient to allow manufacture and that maintain the integrity of the compound for a sufficient period of time to be useful for the purpose detailed herein.

If a moiety is described as “substituted,” a non-hydrogen radical is in the place of hydrogen radical of any substitutable atom of the moiety. Thus, for example, a substituted heteroaryl moiety is a heteroaryl moiety in which at least one non-hydrogen radical is in the place of a hydrogen radical on the heteroaryl. It should be recognized that if there are more than one substitution on a moiety, each non-hydrogen radical may be identical or different (unless otherwise stated).

The phrase “therapeutically effective amount” refers to an amount of a compound, or a pharmaceutically acceptable salt thereof, sufficient to prevent the development of or to alleviate to some extent one or more of the symptoms of the condition or disorder being treated when administered for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The terms “treat,” “treating,” and “treatment,” as used herein, refer to a method of alleviating or abrogating a disease and/or its attendant symptoms.

The term “one or more” refers to one to five. In certain embodiments, it refers to one or four. In certain embodiments, it refers to one to four. In certain embodiments, it refers to one or three. In certain embodiments, it refers to one to three. In certain embodiments, it refers to one to two. In certain embodiments, it refers to two. In yet other further embodiment, it refers to one.

### Compounds

Compounds of the invention have the general Formula (I) as described above.

In some embodiments, the invention provides compounds of Formula (I), or a pharmaceutically acceptable salt thereof,

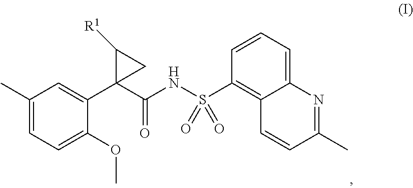

wherein
R¹ is selected from the group consisting of phenyl and 6-membered heteroaryl; wherein R¹ is optionally substituted with one or more R²
R² is selected from the group consisting of fluoro, chloro, bromo, C₁-C₄ alkyl, C₁-C₄ haloalkyl, C₂-C₆ alkoxyalkyl, —OR^{2a}, and —NR^{2b}R^{2c};
R^{2a} is C₁-C₄ alkyl; and
R^{2b} and R^{2c} are each independently selected from the group consisting of hydrogen and C₁-C₄ alkyl.

In certain embodiments of formula (I), or a pharmaceutically acceptable salt thereof, R¹ is 6-membered heteroaryl; and the remaining variables are as defined for formula (I).

In certain embodiments of formula (I), or a pharmaceutically acceptable salt thereof, R¹ is selected from the group consisting of phenyl, pyridyl, pyrazinyl, pyridazinyl, and pyrimidinyl; wherein R¹ is optionally substituted with one or more R²; and the remaining variables are as defined for formula (I).

In certain embodiments of formula (I), or a pharmaceutically acceptable salt thereof, R¹ is selected from the group consisting of

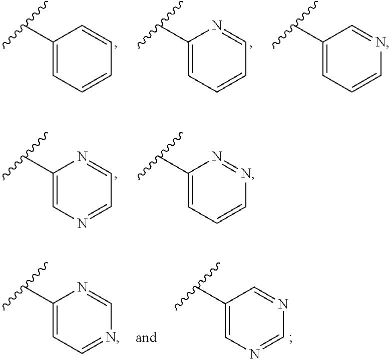

wherein R¹ is optionally substituted with one or more R²; and the remaining variables are as defined for formula (I).

In certain embodiments of formula (I), or a pharmaceutically acceptable salt thereof, R¹ is pyridyl; wherein R¹ is optionally substituted with one or more R²; and the remaining variables are as defined for formula (I).

In certain embodiments of formula (I), or a pharmaceutically acceptable salt thereof, R¹ is pyridyl; wherein R¹ is optionally substituted with one or more R²; R² is selected from the group consisting of C₁-C₄ alkyl, C₁-C₄ haloalkyl, C₂-C₆ alkoxyalkyl, and —OR^{2a}; and the remaining variables are as defined for formula (I).

In certain embodiments of formula (I), or a pharmaceutically acceptable salt thereof, R¹ is pyridyl; wherein R¹ is optionally substituted with one or more R²; R² is selected from the group consisting of C₁-C₄ alkyl and —OR^{2a}; and the remaining variables are as defined for formula (I).

In certain embodiments of formula (I), or a pharmaceutically acceptable salt thereof, R¹ is pyridyl; wherein R¹ is optionally substituted with one or more R²; R² is —OR^{2a}; and the remaining variables are as defined for formula (I).

In certain embodiments of formula (I), or a pharmaceutically acceptable salt thereof, the compound is

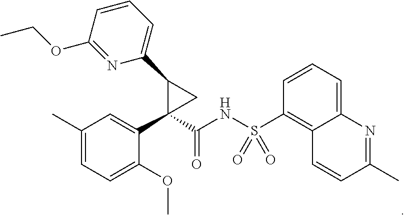 [SMILES: CCOc1cccc([C@H]2C[C@@]2(C(=O)NS(=O)(=O)c2cccc3nc(C)ccc23)c2cc(C)ccc2OC)n1]

In certain embodiments of formula (I), the compound is

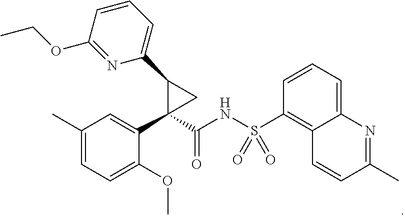 [SMILES: CCOc1cccc([C@H]2C[C@@]2(C(=O)NS(=O)(=O)c2cccc3nc(C)ccc23)c2cc(C)ccc2OC)n1]

In certain embodiments of formula (I), or a pharmaceutically acceptable salt thereof, R¹ is

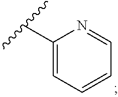

wherein R¹ is optionally substituted with one or more R²; and the remaining variables are as defined for formula (I).

In certain embodiments of formula (I), or a pharmaceutically acceptable salt thereof, R¹ is

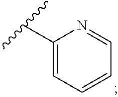

wherein R¹ is optionally substituted with one or more R²; R² is selected from the group consisting of C₁-C₄ alkyl and —OR^{2a}; and the remaining variables are as defined for formula (I).

In certain embodiments of formula (I), or a pharmaceutically acceptable salt thereof, R¹ is phenyl; and the remaining variables are as defined for formula (I).

In certain embodiments of formula (I), or a pharmaceutically acceptable salt thereof, R¹ is pyrazinyl; wherein R¹ is optionally substituted with one or more R²; and the remaining variables are as defined for formula (I).

In certain embodiments of formula (I), or a pharmaceutically acceptable salt thereof, R¹ is pyrazinyl; wherein R¹ is optionally substituted with one or more R²; R² is selected from the group consisting of C₁-C₄ alkyl and —OR^{2a}; and the remaining variables are as defined for formula (I).

In certain embodiments of formula (I), or a pharmaceutically acceptable salt thereof, R¹ is pyridazinyl; wherein R¹ is optionally substituted with one or more R²; and the remaining variables are as defined for formula (I).

In certain embodiments of formula (I), or a pharmaceutically acceptable salt thereof, R¹ is pyrimidinyl; wherein R¹ is optionally substituted with one or more R²; and the remaining variables are as defined for formula (I).

Compound names are assigned by using Name 2019 by Advanced Chemical Development (ACD)/ChemSketch 2019.1.1 naming algorithm, or for some intermediates, using Struct=Name naming algorithm as part of CHEMIDRAW® Professional v. 15.0.0.106.

In certain embodiments a compound, or a pharmaceutically acceptable salt thereof, is provided selected from the group consisting of
rac-(1r,2s)-1-(2-methoxy-5-methylphenyl)-N-(2-methylquinoline-5-sulfonyl)-2-phenylcyclopropane-1-carboxamide;
(1R,2S)-1-(2-methoxy-5-methylphenyl)-N-(2-methylquinoline-5-sulfonyl)-2-phenylcyclopropane-1-carboxamide;
(1S,2R)-1-(2-methoxy-5-methylphenyl)-N-(2-methylquinoline-5-sulfonyl)-2-phenylcyclopropane-1-carboxamide;
rac-(1r,2s)-1-(2-methoxy-5-methylphenyl)-2-(4-methylphenyl)-N-(2-methylquinoline-5-sulfonyl)cyclopropane-1-carboxamide;
rac-(1r,2s)-1-(2-methoxy-5-methylphenyl)-N-(2-methylquinoline-5-sulfonyl)-2-[4-(trifluoromethyl)phenyl]cyclopropane-1-carboxamide;
(1R,2S)-1-(2-methoxy-5-methylphenyl)-2-(4-methylphenyl)-N-(2-methylquinoline-5-sulfonyl)cyclopropane-1-carboxamide;
(1S,2R)-1-(2-methoxy-5-methylphenyl)-2-(4-methylphenyl)-N-(2-methylquinoline-5-sulfonyl)cyclopropane-1-carboxamide;
rac-(1r,2s)-2-(3-fluorophenyl)-1-(2-methoxy-5-methylphenyl)-N-(2-methylquinoline-5-sulfonyl)cyclopropane-1-carboxamide;
rac-(1r,2s)-1-(2-methoxy-5-methylphenyl)-2-(4-methoxyphenyl)-N-(2-methylquinoline-5-sulfonyl)cyclopropane-1-carboxamide;
(1R,2S)-1-(2-methoxy-5-methylphenyl)-2-(6-methylpyridin-3-yl)-N-(2-methylquinoline-5-sulfonyl)cyclopropane-1-carboxamide;
(1S,2R)-1-(2-methoxy-5-methylphenyl)-2-(6-methylpyridin-3-yl)-N-(2-methylquinoline-5-sulfonyl)cyclopropane-1-carboxamide;
rac-(1r,2s)-1-(2-methoxy-5-methylphenyl)-N-(2-methylquinoline-5-sulfonyl)-2-[3-(trifluoromethyl)phenyl]cyclopropane-1-carboxamide;
(1R,2R)-1-(2-methoxy-5-methylphenyl)-N-(2-methylquinoline-5-sulfonyl)-2-(pyridin-2-yl)cyclopropane-1-carboxamide;
(1S,2S)-1-(2-methoxy-5-methylphenyl)-N-(2-methylquinoline-5-sulfonyl)-2-(pyridin-2-yl)cyclopropane-1-carboxamide;
rac-(1r,2s)-1-(2-methoxy-5-methylphenyl)-2-(3-methoxyphenyl)-N-(2-methylquinoline-5-sulfonyl)cyclopropane-1-carboxamide;
(1R,2S)-1-(2-methoxy-5-methylphenyl)-2-(4-methoxyphenyl)-N-(2-methylquinoline-5-sulfonyl)cyclopropane-1-carboxamide;
(1S,2R)-1-(2-methoxy-5-methylphenyl)-2-(4-methoxyphenyl)-N-(2-methylquinoline-5-sulfonyl)cyclopropane-1-carboxamide;
(1S,2R)-1-(2-methoxy-5-methylphenyl)-N-(2-methylquinoline-5-sulfonyl)-2-[4-(trifluoromethyl)phenyl]cyclopropane-1-carboxamide;
(1R,2S)-1-(2-methoxy-5-methylphenyl)-N-(2-methylquinoline-5-sulfonyl)-2-[4-(trifluoromethyl)phenyl]cyclopropane-1-carboxamide;
(1R,2S)-2-(3-fluorophenyl)-1-(2-methoxy-5-methylphenyl)-N-(2-methylquinoline-5-sulfonyl)cyclopropane-1-carboxamide;
(1S,2R)-2-(3-fluorophenyl)-1-(2-methoxy-5-methylphenyl)-N-(2-methylquinoline-5-sulfonyl)cyclopropane-1-carboxamide;
rac-(1r,2r)-2-(2-fluorophenyl)-1-(2-methoxy-5-methylphenyl)-N-(2-methylquinoline-5-sulfonyl)cyclopropane-1-carboxamide;
(1S,2R)-1-(2-methoxy-5-methylphenyl)-N-(2-methylquinoline-5-sulfonyl)-2-[3-(trifluoromethyl)phenyl]cyclopropane-1-carboxamide;
(1R,2S)-1-(2-methoxy-5-methylphenyl)-N-(2-methylquinoline-5-sulfonyl)-2-[3-(trifluoromethyl)phenyl]cyclopropane-1-carboxamide;
(1S,2S)-2-(2-fluorophenyl)-1-(2-methoxy-5-methylphenyl)-N-(2-methylquinoline-5-sulfonyl)cyclopropane-1-carboxamide;
(1R,2R)-2-(2-fluorophenyl)-1-(2-methoxy-5-methylphenyl)-N-(2-methylquinoline-5-sulfonyl)cyclopropane-1-carboxamide;
(1R,2S)-1-(2-methoxy-5-methylphenyl)-2-(3-methoxyphenyl)-N-(2-methylquinoline-5-sulfonyl)cyclopropane-1-carboxamide;
(1S,2R)-1-(2-methoxy-5-methylphenyl)-2-(3-methoxyphenyl)-N-(2-methylquinoline-5-sulfonyl)cyclopropane-1-carboxamide;
rac-(1r,2s)-1-(2-methoxy-5-methylphenyl)-N-(2-methylquinoline-5-sulfonyl)-2-[2-(trifluoromethyl)phenyl]cyclopropane-1-carboxamide;
(1S,2R)-1-(2-methoxy-5-methylphenyl)-N-(2-methylquinoline-5-sulfonyl)-2-[2-(trifluoromethyl)phenyl]cyclopropane-1-carboxamide;
(1R,2S)-1-(2-methoxy-5-methylphenyl)-N-(2-methylquinoline-5-sulfonyl)-2-[2-(trifluoromethyl)phenyl]cyclopropane-1-carboxamide;
(1S,2R)-1-(2-methoxy-5-methylphenyl)-2-(6-methoxypyridin-3-yl)-N-(2-methylquinoline-5-sulfonyl)cyclopropane-1-carboxamide;
(1R,2S)-1-(2-methoxy-5-methylphenyl)-2-(6-methoxypyridin-3-yl)-N-(2-methylquinoline-5-sulfonyl)cyclopropane-1-carboxamide;
(1S,2R)-1-(2-methoxy-5-methylphenyl)-2-(2-methylphenyl)-N-(2-methylquinoline-5-sulfonyl)cyclopropane-1-carboxamide;
rac-(1r,2r)-1-(2-methoxy-5-methylphenyl)-2-(6-methylpyrazin-2-yl)-N-(2-methylquinoline-5-sulfonyl)cyclopropane-1-carboxamide;
rac-(1r,2s)-1-(2-methoxy-5-methylphenyl)-2-(2-methoxypyrimidin-5-yl)-N-(2-methylquinoline-5-sulfonyl)cyclopropane-1-carboxamide;
(1R,2S)-1-(2-methoxy-5-methylphenyl)-2-(2-methoxypyrimidin-5-yl)-N-(2-methylquinoline-5-sulfonyl)cyclopropane-1-carboxamide;
(1S,2R)-1-(2-methoxy-5-methylphenyl)-2-(2-methoxypyrimidin-5-yl)-N-(2-methylquinoline-5-sulfonyl)cyclopropane-1-carboxamide;
rac-(1r,2r)-1-(2-methoxy-5-methylphenyl)-2-(5-methoxypyrazin-2-yl)-N-(2-methylquinoline-5-sulfonyl)cyclopropane-1-carboxamide;
rac-(1r,2s)-1-(2-methoxy-5-methylphenyl)-2-(2-methoxypyridin-3-yl)-N-(2-methylquinoline-5-sulfonyl)cyclopropane-1-carboxamide;
rac-(1r,2s)-1-(2-methoxy-5-methylphenyl)-2-(5-methoxypyridin-3-yl)-N-(2-methylquinoline-5-sulfonyl)cyclopropane-1-carboxamide;
rac-(1r,2s)-2-(5-fluoropyridin-3-yl)-1-(2-methoxy-5-methylphenyl)-N-(2-methylquinoline-5-sulfonyl)cyclopropane-1-carboxamide;
(1s,2s)-1-(2-methoxy-5-methylphenyl)-2-(6-methoxypyridin-2-yl)-N-(2-methylquinoline-5-sulfonyl)cyclopropane-1-carboxamide;
(1R,2R)-1-(2-methoxy-5-methylphenyl)-2-(6-methoxypyridin-2-yl)-N-(2-methylquinoline-5-sulfonyl)cyclopropane-1-carboxamide;
(1R,2R)-1-(2-methoxy-5-methylphenyl)-2-(6-methylpyrazin-2-yl)-N-(2-methylquinoline-5-sulfonyl)cyclopropane-1-carboxamide;
(1S,2S)-1-(2-methoxy-5-methylphenyl)-2-(6-methylpyrazin-2-yl)-N-(2-methylquinoline-5-sulfonyl)cyclopropane-1-carboxamide;
(1R,2R)-1-(2-methoxy-5-methylphenyl)-2-(5-methoxypyrazin-2-yl)-N-(2-methylquinoline-5-sulfonyl)cyclopropane-1-carboxamide;
(1S,2S)-1-(2-methoxy-5-methylphenyl)-2-(5-methoxypyrazin-2-yl)-N-(2-methylquinoline-5-sulfonyl)cyclopropane-1-carboxamide;
(1R,2S)-1-(2-methoxy-5-methylphenyl)-2-(2-methoxypyridin-3-yl)-N-(2-methylquinoline-5-sulfonyl)cyclopropane-1-carboxamide;
(1S,2R)-1-(2-methoxy-5-methylphenyl)-2-(2-methoxypyridin-3-yl)-N-(2-methylquinoline-5-sulfonyl)cyclopropane-1-carboxamide;
(1R,2S)-1-(2-methoxy-5-methylphenyl)-2-(5-methoxypyridin-3-yl)-N-(2-methylquinoline-5-sulfonyl)cyclopropane-1-carboxamide;
(1S,2R)-1-(2-methoxy-5-methylphenyl)-2-(5-methoxypyridin-3-yl)-N-(2-methylquinoline-5-sulfonyl)cyclopropane-1-carboxamide;
(1R,2S)-2-(5-fluoropyridin-3-yl)-1-(2-methoxy-5-methylphenyl)-N-(2-methylquinoline-5-sulfonyl)cyclopropane-1-carboxamide;
(1S,2R)-2-(5-fluoropyridin-3-yl)-1-(2-methoxy-5-methylphenyl)-N-(2-methylquinoline-5-sulfonyl)cyclopropane-1-carboxamide;
(1S,2S)-2-(6-ethoxypyridin-2-yl)-1-(2-methoxy-5-methylphenyl)-N-(2-methylquinoline-5-sulfonyl)cyclopropane-1-carboxamide;
(1R,2R)-2-(6-ethoxypyridin-2-yl)-1-(2-methoxy-5-methylphenyl)-N-(2-methylquinoline-5-sulfonyl)cyclopropane-1-carboxamide;
(1S,2S)-1-(2-methoxy-5-methylphenyl)-N-(2-methylquinoline-5-sulfonyl)-2-[6-(propan-2-yl)pyridin-2-yl]cyclopropane-1-carboxamide;
(1S,2R)-2-(6-ethoxypyridin-3-yl)-1-(2-methoxy-5-methylphenyl)-N-(2-methylquinoline-5-sulfonyl)cyclopropane-1-carboxamide;
(1R,2S)-2-(6-ethoxypyridin-3-yl)-1-(2-methoxy-5-methylphenyl)-N-(2-methylquinoline-5-sulfonyl)cyclopropane-1-carboxamide;
(1S,2S)-2-[6-(dimethylamino)pyridin-2-yl]-1-(2-methoxy-5-methylphenyl)-N-(2-methylquinoline-5-sulfonyl)cyclopropane-1-carboxamide;
rac-(1r,2r)-1-(2-methoxy-5-methylphenyl)-N-(2-methylquinoline-5-sulfonyl)-2-[5-(propan-2-yl)pyrazin-2-yl]cyclopropane-1-carboxamide;
rac-(1r,2r)-2-(5-ethoxypyrazin-2-yl)-1-(2-methoxy-5-methylphenyl)-N-(2-methylquinoline-5-sulfonyl)cyclopropane-1-carboxamide;
(1R,2R)-1-(2-methoxy-5-methylphenyl)-N-(2-methylquinoline-5-sulfonyl)-2-[5-(propan-2-yl)pyrazin-2-yl]cyclopropane-1-carboxamide;
(1S,2S)-1-(2-methoxy-5-methylphenyl)-N-(2-methylquinoline-5-sulfonyl)-2-[5-(propan-2-yl)pyrazin-2-yl]cyclopropane-1-carboxamide;
(1R,2R)-2-(5-ethoxypyrazin-2-yl)-1-(2-methoxy-5-methylphenyl)-N-(2-methylquinoline-5-sulfonyl)cyclopropane-1-carboxamide;
(1S,2S)-2-(5-ethoxypyrazin-2-yl)-1-(2-methoxy-5-methylphenyl)-N-(2-methylquinoline-5-sulfonyl)cyclopropane-1-carboxamide;
(1S,2R)-1-(2-methoxy-5-methylphenyl)-N-(2-methylquinoline-5-sulfonyl)-2-[6-(propan-2-yl)pyridin-3-yl]cyclopropane-1-carboxamide;
(1S,2R)-2-[6-(dimethylamino)pyridin-3-yl]-1-(2-methoxy-5-methylphenyl)-N-(2-methylquinoline-5-sulfonyl)cyclopropane-1-carboxamide;
(1S,2S)-1-(2-methoxy-5-methylphenyl)-N-(2-methylquinoline-5-sulfonyl)-2-[6-(propan-2-yl)pyridazin-3-yl]cyclopropane-1-carboxamide;
(1S,2R)-2-[6-(difluoromethyl)pyridin-3-yl]-1-(2-methoxy-5-methylphenyl)-N-(2-methylquinoline-5-sulfonyl)cyclopropane-1-carboxamide;
(1S,2R)-2-(2-ethoxypyridin-3-yl)-1-(2-methoxy-5-methylphenyl)-N-(2-methylquinoline-5-sulfonyl)cyclopropane-1-carboxamide;
(1S,2R)-1-(2-methoxy-5-methylphenyl)-2-(6-methoxy-2-methylpyridin-3-yl)-N-(2-methylquinoline-5-sulfonyl)cyclopropane-1-carboxamide;
(1R,2S)-2-(2-ethoxypyridin-3-yl)-1-(2-methoxy-5-methylphenyl)-N-(2-methylquinoline-5-sulfonyl)cyclopropane-1-carboxamide;
(1R,2R)-1-(2-methoxy-5-methylphenyl)-2-(6-methoxypyrazin-2-yl)-N-(2-methylquinoline-5-sulfonyl)cyclopropane-1-carboxamide;
(1S,2S)-1-(2-methoxy-5-methylphenyl)-2-(6-methoxypyrazin-2-yl)-N-(2-methylquinoline-5-sulfonyl)cyclopropane-1-carboxamide;
(1S,2R)-2-(5-fluoro-6-methoxypyridin-3-yl)-1-(2-methoxy-5-methylphenyl)-N-(2-methylquinoline-5-sulfonyl)cyclopropane-1-carboxamide;
(1S,2S)-2-[6-(difluoromethyl)pyridin-2-yl]-1-(2-methoxy-5-methylphenyl)-N-(2-methylquinoline-5-sulfonyl)cyclopropane-1-carboxamide;
rac-(1r,2s)-1-(2-methoxy-5-methylphenyl)-2-[2-methoxy-6-(propan-2-yl)pyridin-3-yl]-N-(2-methylquinoline-5-sulfonyl)cyclopropane-1-carboxamide;
rac-(1r,2s)-2-(2,6-dimethoxypyridin-3-yl)-1-(2-methoxy-5-methylphenyl)-N-(2-methylquinoline-5-sulfonyl)cyclopropane-1-carboxamide;
(1S,2S)-2-(5,6-dimethoxypyrazin-2-yl)-1-(2-methoxy-5-methylphenyl)-N-(2-methylquinoline-5-sulfonyl)cyclopropane-1-carboxamide;
(1S,2R)-2-(2,6-dimethylpyridin-3-yl)-1-(2-methoxy-5-methylphenyl)-N-(2-methylquinoline-5-sulfonyl)cyclopropane-1-carboxamide;
(1S,2S)-2-(3,5-dimethoxypyrazin-2-yl)-1-(2-methoxy-5-methylphenyl)-N-(2-methylquinoline-5-sulfonyl)cyclopropane-1-carboxamide;
(1S,2S)-1-(2-methoxy-5-methylphenyl)-2-(5-methoxy-6-methylpyrazin-2-yl)-N-(2-methylquinoline-5-sulfonyl)cyclopropane-1-carboxamide;
(1S,2R)-2-(5-fluoro-2-methoxypyridin-3-yl)-1-(2-methoxy-5-methylphenyl)-N-(2-methylquinoline-5-sulfonyl)cyclopropane-1-carboxamide;
(1R,2S)-1-(2-methoxy-5-methylphenyl)-2-(2-methoxy-6-methylpyridin-3-yl)-N-(2-methylquinoline-5-sulfonyl)cyclopropane-1-carboxamide;
(1S,2R)-1-(2-methoxy-5-methylphenyl)-2-(2-methoxy-6-methylpyridin-3-yl)-N-(2-methylquinoline-5-sulfonyl)cyclopropane-1-carboxamide;
(1R,2S)-1-(2-methoxy-5-methylphenyl)-2-[2-methoxy-6-(propan-2-yl)pyridin-3-yl]-N-(2-methylquinoline-5-sulfonyl)cyclopropane-1-carboxamide;
(1S,2R)-1-(2-methoxy-5-methylphenyl)-2-[2-methoxy-6-(propan-2-yl)pyridin-3-yl]-N-(2-methylquinoline-5-sulfonyl)cyclopropane-1-carboxamide;
rac-(1r,2r)-1-(2-methoxy-5-methylphenyl)-2-(5-methylpyrazin-2-yl)-N-(2-methylquinoline-5-sulfonyl)cyclopropane-1-carboxamide;
(1S,2S)-1-(2-methoxy-5-methylphenyl)-2-[6-(methoxymethyl)pyridin-2-yl]-N-(2-methylquinoline-5-sulfonyl)cyclopropane-1-carboxamide;
(1R,2R)-1-(2-methoxy-5-methylphenyl)-2-(5-methylpyrazin-2-yl)-N-(2-methylquinoline-5-sulfonyl)cyclopropane-1-carboxamide;
(1S,2S)-1-(2-methoxy-5-methylphenyl)-2-(5-methylpyrazin-2-yl)-N-(2-methylquinoline-5-sulfonyl)cyclopropane-1-carboxamide;
(1R,2S)-2-(2,6-dimethoxypyridin-3-yl)-1-(2-methoxy-5-methylphenyl)-N-(2-methylquinoline-5-sulfonyl)cyclopropane-1-carboxamide;
(1S,2R)-2-(2,6-dimethoxypyridin-3-yl)-1-(2-methoxy-5-methylphenyl)-N-(2-methylquinoline-5-sulfonyl)cyclopropane-1-carboxamide;
rac-(1r,2r)-1-(2-methoxy-5-methylphenyl)-2-(2-methoxypyrimidin-4-yl)-N-(2-methylquinoline-5-sulfonyl)cyclopropane-1-carboxamide;
(1S,2S)-1-(2-methoxy-5-methylphenyl)-2-(6-methylpyridin-2-yl)-N-(2-methylquinoline-5-sulfonyl)cyclopropane-1-carboxamide;
(1S,2S)-1-(2-methoxy-5-methylphenyl)-N-(2-methylquinoline-5-sulfonyl)-2-{6-[(propan-2-yl)oxy]pyridin-2-yl}cyclopropane-1-carboxamide;
(1S,2S)-1-(2-methoxy-5-methylphenyl)-2-(2-methoxypyrimidin-4-yl)-N-(2-methylquinoline-5-sulfonyl)cyclopropane-1-carboxamide;
(1R,2R)-1-(2-methoxy-5-methylphenyl)-2-(2-methoxypyrimidin-4-yl)-N-(2-methylquinoline-5-sulfonyl)cyclopropane-1-carboxamide;
(1S,2R)-2-(5-fluoro-6-methylpyridin-3-yl)-1-(2-methoxy-5-methylphenyl)-N-(2-methylquinoline-5-sulfonyl)cyclopropane-1-carboxamide;
(1R,2S)-2-(5-fluoro-6-methylpyridin-3-yl)-1-(2-methoxy-5-methylphenyl)-N-(2-methylquinoline-5-sulfonyl)cyclopropane-1-carboxamide;
(1S,2R)-2-(5-chloro-6-methoxypyridin-3-yl)-1-(2-methoxy-5-methylphenyl)-N-(2-methylquinoline-5-sulfonyl)cyclopropane-1-carboxamide;
(1S,2R)-1-(2-methoxy-5-methylphenyl)-2-(6-methoxy-5-methylpyridin-3-yl)-N-(2-methylquinoline-5-sulfonyl)cyclopropane-1-carboxamide;
(1S,2R)-2-(5,6-dimethoxypyridin-3-yl)-1-(2-methoxy-5-methylphenyl)-N-(2-methylquinoline-5-sulfonyl)cyclopropane-1-carboxamide;
(1S,2S)-2-(6-chloropyridin-2-yl)-1-(2-methoxy-5-methylphenyl)-N-(2-methylquinoline-5-sulfonyl)cyclopropane-1-carboxamide; and
(1S,2S)-2-(6-ethoxy-5-fluoropyridin-2-yl)-1-(2-methoxy-5-methylphenyl)-N-(2-methylquinoline-5-sulfonyl)cyclopropane-1-carboxamide.

In certain embodiments the compound, or a pharmaceutically acceptable salt thereof, is provided which is (1S,2S)-2-(6-ethoxypyridin-2-yl)-1-(2-methoxy-5-methylphenyl)-N-(2-methylquinoline-5-sulfonyl)cyclopropane-1-carboxamide.

In certain embodiments the compound is provided which is (1S,2S)-2-(6-ethoxypyridin-2-yl)-1-(2-methoxy-5-methylphenyl)-N-(2-methylquinoline-5-sulfonyl)cyclopropane-1-carboxamide.

Exemplary compounds of Formula (I) include, but are not limited to, the compounds shown in Table 1 below, and pharmaceutically acceptable salts thereof. It is to be understood that when there is a discrepancy between the name of the compound found herein and the structure found in Table 1, the structure in Table 1 shall prevail.

TABLE 1
Exemplary Compounds

| Ex | Structure |
|---|---|
| 1 | 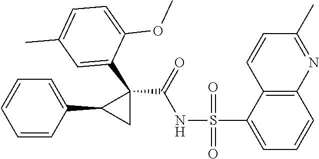 [SMILES: COc1ccc(C)cc1[C@@]1(C(=O)NS(=O)(=O)c2cccc3nc(C)ccc23)C[C@H]1c1ccccc1] |
| 2 | 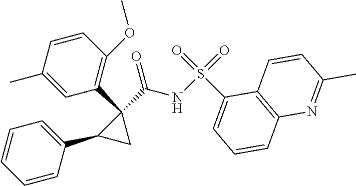 [SMILES: COc1ccc(C)cc1[C@@]1(C(=O)NS(=O)(=O)c2cccc3nc(C)ccc23)C[C@H]1c1ccccc1] |
| 3 | 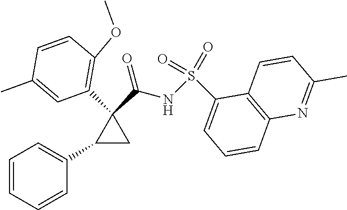 |
| 4 | 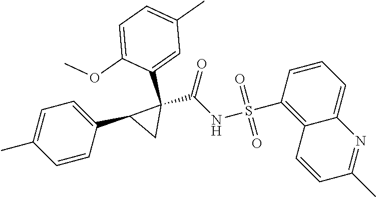 |
| 5 | 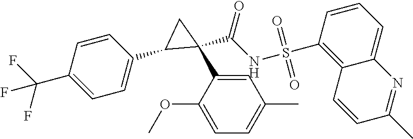 [SMILES: COc1ccc(C)cc1[C@]1(C(=O)NS(=O)(=O)c2cccc3nc(C)ccc23)C[C@H]1c1ccc(C(F)(F)F)cc1] |
| 6 | 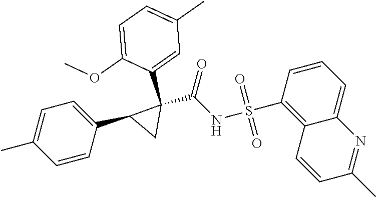 |
| 7 | 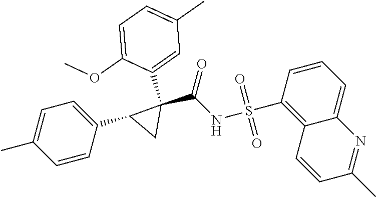 |
| 8 | 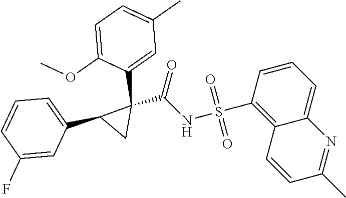 [SMILES: COc1ccc(C)cc1[C@@]1(C(=O)NS(=O)(=O)c2cccc3nc(C)ccc23)C[C@H]1c1cccc(F)c1] |
| 9 | 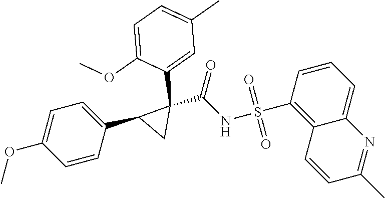 |
| 10 | 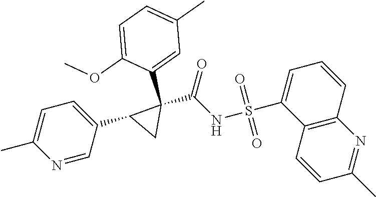 |
| 11 | 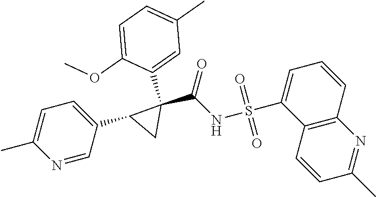 |
| 12 | 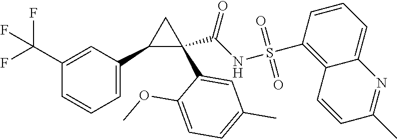 [SMILES: COc1ccc(C)cc1[C@]1(C(=O)NS(=O)(=O)c2cccc3nc(C)ccc23)C[C@@H]1c1cccc(C(F)(F)F)c1] |
| 13 | 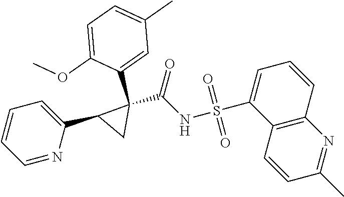 [SMILES: COc1ccc(C)cc1[C@@]1(C(=O)NS(=O)(=O)c2cccc3nc(C)ccc23)C[C@H]1c1ccccn1] |
| 14 | 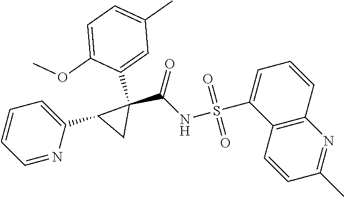 [SMILES: COc1ccc(C)cc1[C@]1(C(=O)NS(=O)(=O)c2cccc3nc(C)ccc23)C[C@@H]1c1ccccn1] |
| 15 | 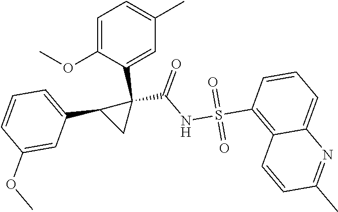 [SMILES: COc1cccc([C@@H]2C[C@]2(C(=O)NS(=O)(=O)c2cccc3nc(C)ccc23)c2cc(C)ccc2OC)c1] |
| 16 | 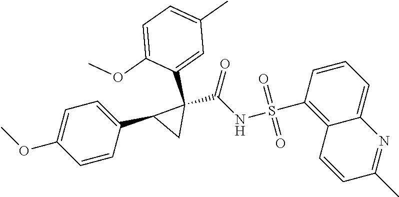 |
| 17 | 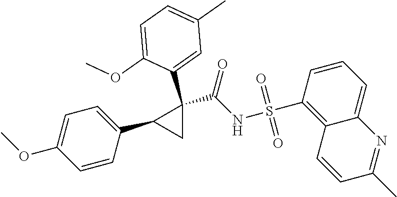 |
| 18 | 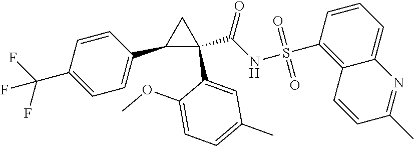 |
| 19 | 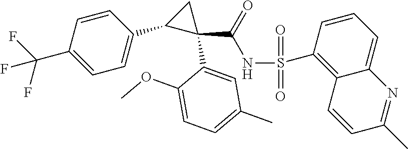 |
| 20 | 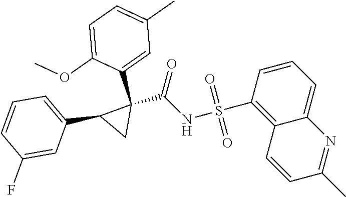 [SMILES: COc1ccc(C)cc1[C@@]1(C(=O)NS(=O)(=O)c2cccc3nc(C)ccc23)C[C@H]1c1cccc(F)c1] |
| 21 | 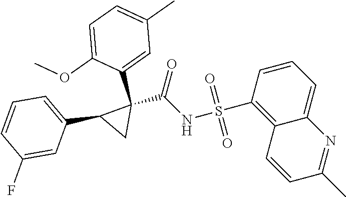 [SMILES: COc1ccc(C)cc1[C@@]1(C(=O)NS(=O)(=O)c2cccc3nc(C)ccc23)C[C@H]1c1cccc(F)c1] |
| 22 | 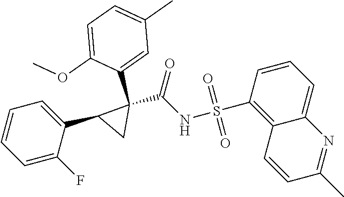 [SMILES: COc1ccc(C)cc1[C@@]1(C(=O)NS(=O)(=O)c2cccc3nc(C)ccc23)C[C@H]1c1ccccc1F] |
| 23 | 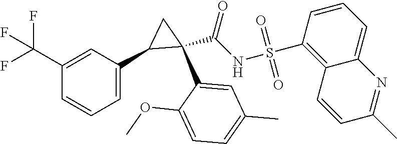 [SMILES: COc1ccc(C)cc1[C@]1(C(=O)NS(=O)(=O)c2cccc3nc(C)ccc23)C[C@@H]1c1cccc(C(F)(F)F)c1] |
| 24 | 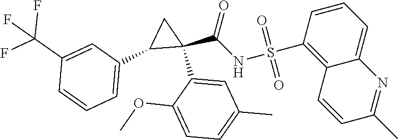 [SMILES: COc1ccc(C)cc1[C@@]1(C(=O)NS(=O)(=O)c2cccc3nc(C)ccc23)C[C@H]1c1cccc(C(F)(F)F)c1] |
| 25 | 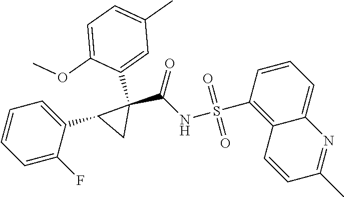 [SMILES: COc1ccc(C)cc1[C@]1(C(=O)NS(=O)(=O)c2cccc3nc(C)ccc23)C[C@@H]1c1ccccc1F] |
| 26 | 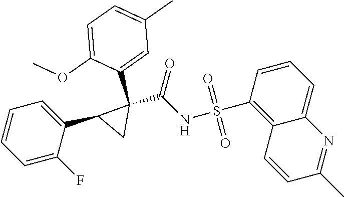 [SMILES: COc1ccc(C)cc1[C@@]1(C(=O)NS(=O)(=O)c2cccc3nc(C)ccc23)C[C@H]1c1ccccc1F] |
| 27 | 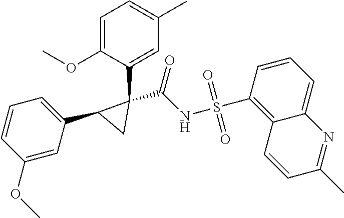 [SMILES: COc1cccc([C@@H]2C[C@]2(C(=O)NS(=O)(=O)c2cccc3nc(C)ccc23)c2cc(C)ccc2OC)c1] |
| 28 | 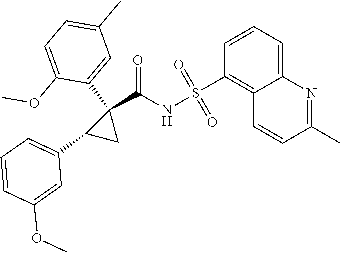 [SMILES: COc1cccc([C@H]2C[C@@]2(C(=O)NS(=O)(=O)c2cccc3nc(C)ccc23)c2cc(C)ccc2OC)c1] |
| 29 | 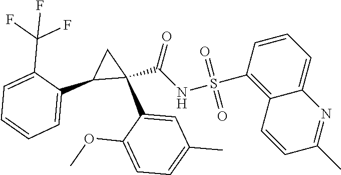 [SMILES: COc1ccc(C)cc1[C@]1(C(=O)NS(=O)(=O)c2cccc3nc(C)ccc23)C[C@@H]1c1ccccc1C(F)(F)F] |
| 30 | 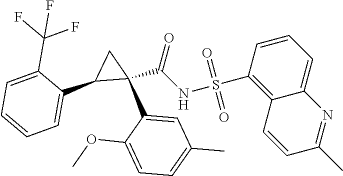 [SMILES: COc1ccc(C)cc1[C@]1(C(=O)NS(=O)(=O)c2cccc3nc(C)ccc23)C[C@@H]1c1ccccc1C(F)(F)F] |
| 31 | 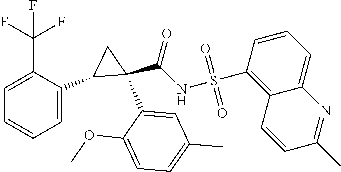 [SMILES: COc1ccc(C)cc1[C@@]1(C(=O)NS(=O)(=O)c2cccc3nc(C)ccc23)C[C@H]1c1ccccc1C(F)(F)F] |
| 32 | 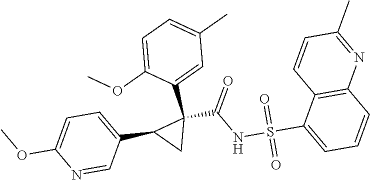 [SMILES: COc1ccc([C@@H]2C[C@]2(C(=O)NS(=O)(=O)c2cccc3nc(C)ccc23)c2cc(C)ccc2OC)cn1] |
| 33 | 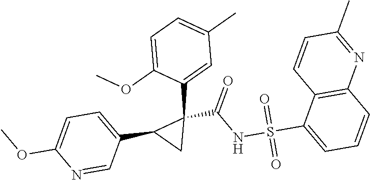 [SMILES: COc1ccc([C@@H]2C[C@]2(C(=O)NS(=O)(=O)c2cccc3nc(C)ccc23)c2cc(C)ccc2OC)cn1] |
| 34 | 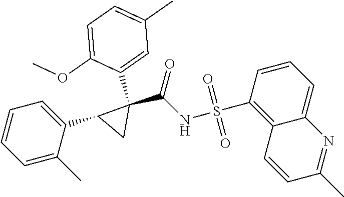 [SMILES: COc1ccc(C)cc1[C@]1(C(=O)NS(=O)(=O)c2cccc3nc(C)ccc23)C[C@@H]1c1ccccc1C] |
| 35 | 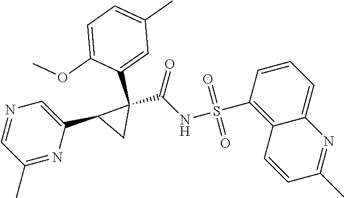 [SMILES: COc1ccc(C)cc1[C@@]1(C(=O)NS(=O)(=O)c2cccc3nc(C)ccc23)C[C@H]1c1cncc(C)n1] |
| 36 | 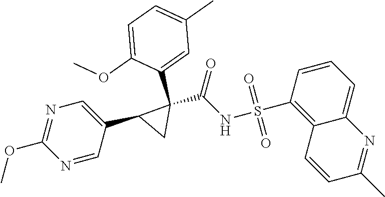 |
| 3 7 | 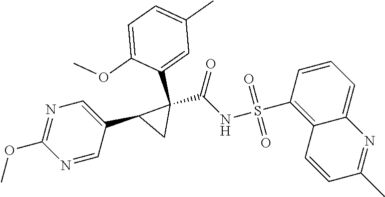 |
| 38 | 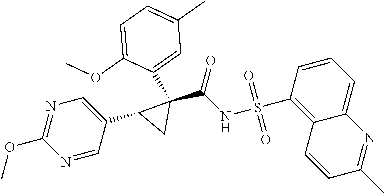 |
| 39 | 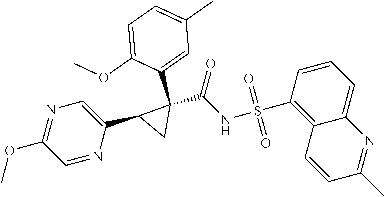 |
| 40 | 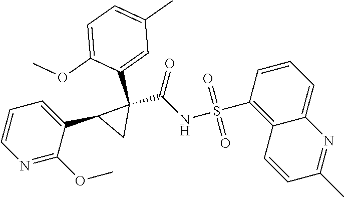 [SMILES: COc1ccc(C)cc1[C@@]1(C(=O)NS(=O)(=O)c2cccc3nc(C)ccc23)C[C@H]1c1cccnc1OC] |
| 41 | 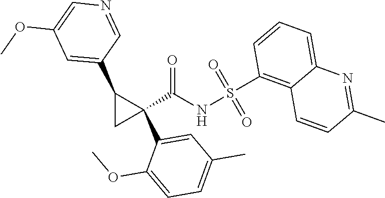 |
| 42 | 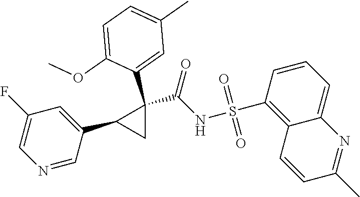 [SMILES: COc1ccc(C)cc1[C@@]1(C(=O)NS(=O)(=O)c2cccc3nc(C)ccc23)C[C@H]1c1cncc(F)c1] |
| 43 | 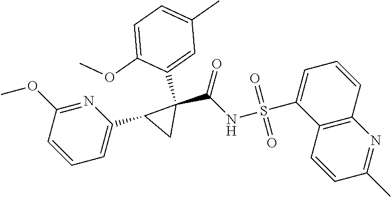 |
| 44 | 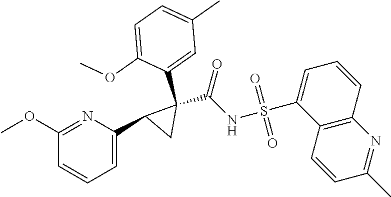 |
| 45 | 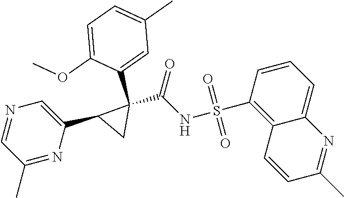 [SMILES: COc1ccc(C)cc1[C@@]1(C(=O)NS(=O)(=O)c2cccc3nc(C)ccc23)C[C@H]1c1cncc(C)n1] |
| 46 | 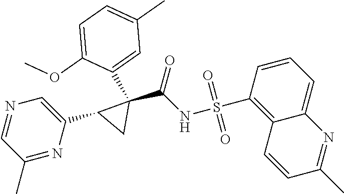 [SMILES: COc1ccc(C)cc1[C@]1(C(=O)NS(=O)(=O)c2cccc3nc(C)ccc23)C[C@@H]1c1cncc(C)n1] |
| 47 | 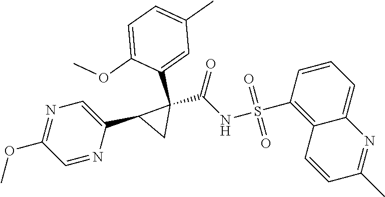 |
| 48 | 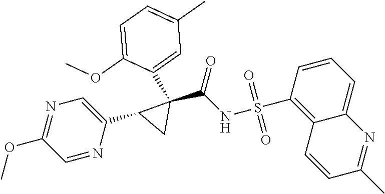 |
| 49 | 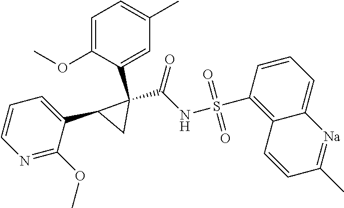 |
| 50 | 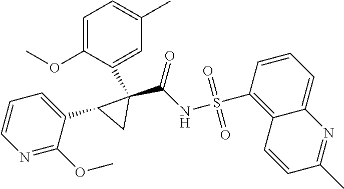 [SMILES: COc1ccc(C)cc1[C@]1(C(=O)NS(=O)(=O)c2cccc3nc(C)ccc23)C[C@@H]1c1cccnc1OC] |
| 51 | 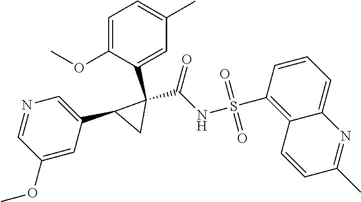 |
| 52 | 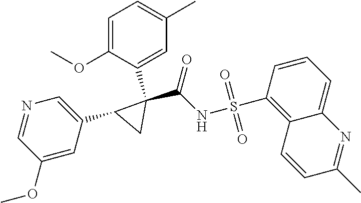 |
| 53 | 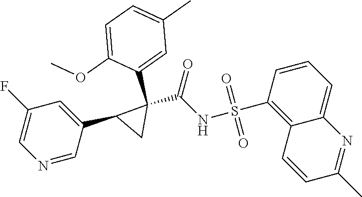 [SMILES: COc1ccc(C)cc1[C@@]1(C(=O)NS(=O)(=O)c2cccc3nc(C)ccc23)C[C@H]1c1cncc(F)c1] |
| 54 | 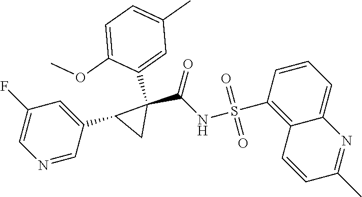 [SMILES: COc1ccc(C)cc1[C@]1(C(=O)NS(=O)(=O)c2cccc3nc(C)ccc23)C[C@@H]1c1cncc(F)c1] |
| 55 | 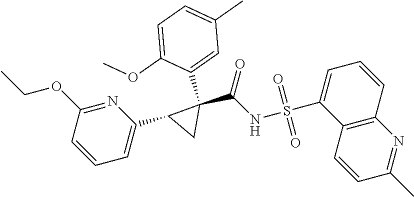 [SMILES: CCOc1cccc([C@H]2C[C@@]2(C(=O)NS(=O)(=O)c2cccc3nc(C)ccc23)c2cc(C)ccc2OC)n1] |
| 56 | 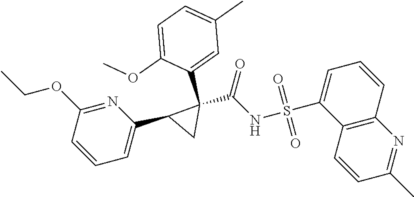 [SMILES: CCOc1cccc([C@@H]2C[C@]2(C(=O)NS(=O)(=O)c2cccc3nc(C)ccc23)c2cc(C)ccc2OC)n1] |
| 57 | 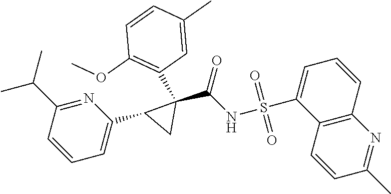 [SMILES: COc1ccc(C)cc1[C@]1(C(=O)NS(=O)(=O)c2cccc3nc(C)ccc23)C[C@@H]1c1cccc(C(C)C)n1] |
| 58 | 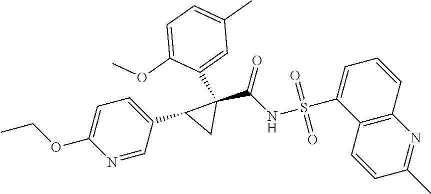 [SMILES: CCOc1ccc([C@H]2C[C@@]2(C(=O)NS(=O)(=O)c2cccc3nc(C)ccc23)c2cc(C)ccc2OC)cn1] |
| 59 | 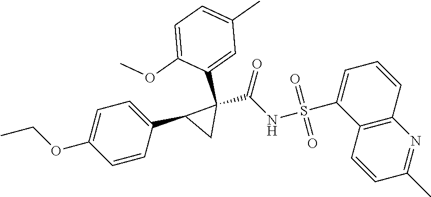 [SMILES: CCOc1ccc([C@@H]2C[C@]2(C(=O)NS(=O)(=O)c2cccc3nc(C)ccc23)c2cc(C)ccc2OC)cc1] |
| 60 | 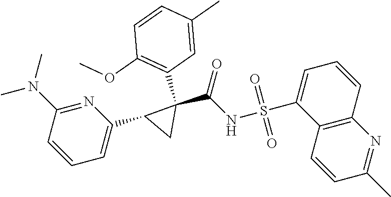 |
| 61 | 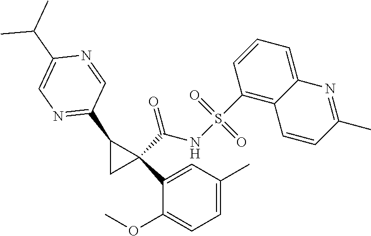 [SMILES: COc1ccc(C)cc1[C@@]1(C(=O)NS(=O)(=O)c2cccc3nc(C)ccc23)C[C@H]1c1cnc(C(C)C)cn1] |
| 62 | 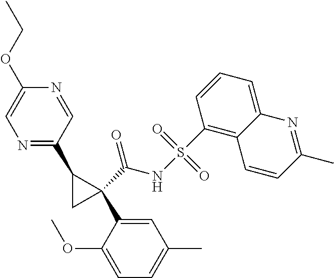 [SMILES: CCOc1cnc([C@@H]2C[C@]2(C(=O)NS(=O)(=O)c2cccc3nc(C)ccc23)c2cc(C)ccc2OC)cn1] |
| 63 | 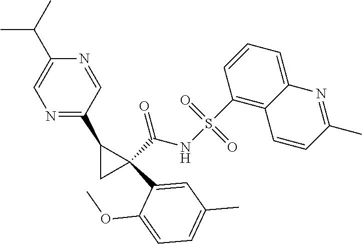 [SMILES: COc1ccc(C)cc1[C@@]1(C(=O)NS(=O)(=O)c2cccc3nc(C)ccc23)C[C@H]1c1cnc(C(C)C)cn1] |
| 64 | 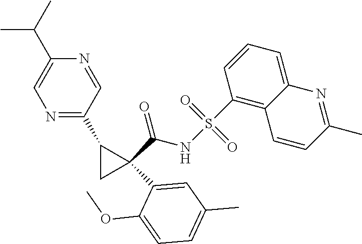 [SMILES: COc1ccc(C)cc1[C@]1(C(=O)NS(=O)(=O)c2cccc3nc(C)ccc23)C[C@@H]1c1cnc(C(C)C)cn1] |
| 65 | 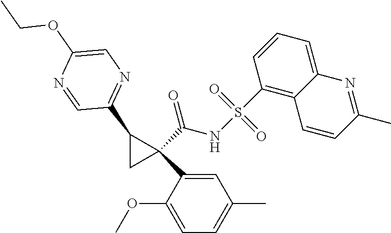 [SMILES: CCOc1cnc([C@@H]2C[C@]2(C(=O)NS(=O)(=O)c2cccc3nc(C)ccc23)c2cc(C)ccc2OC)cn1] |
| 66 | 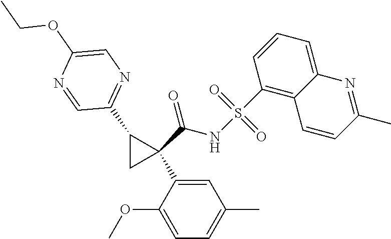 [SMILES: CCOc1cnc([C@H]2C[C@@]2(C(=O)NS(=O)(=O)c2cccc3nc(C)ccc23)c2cc(C)ccc2OC)cn1] |
| 67 | 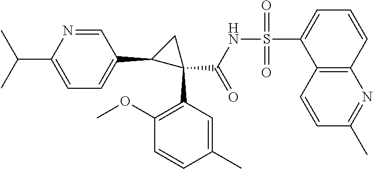 [SMILES: COc1ccc(C)cc1[C@]1(C(=O)NS(=O)(=O)c2cccc3nc(C)ccc23)C[C@@H]1c1ccc(C(C)C)nc1] |
| 68 | 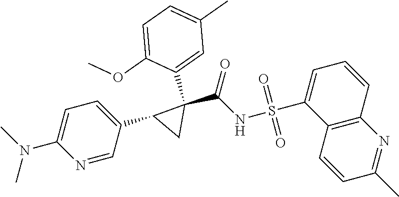 |
| 69 | 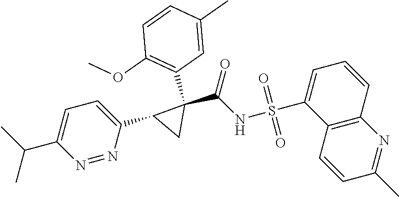 [SMILES: COc1ccc(C)cc1[C@]1(C(=O)NS(=O)(=O)c2cccc3nc(C)ccc23)C[C@@H]1c1ccc(C(C)C)nn1] |
| 70 | 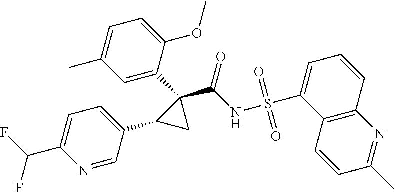 [SMILES: COc1ccc(C)cc1[C@]1(C(=O)NS(=O)(=O)c2cccc3nc(C)ccc23)C[C@@H]1c1ccc(C(F)F)nc1] |
| 71 | 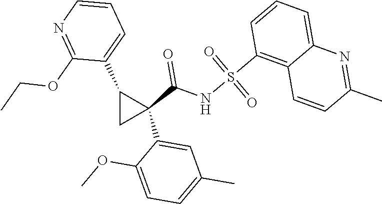 [SMILES: CCOc1ncccc1[C@H]1C[C@@]1(C(=O)NS(=O)(=O)c1cccc2nc(C)ccc12)c1cc(C)ccc1OC] |
| 72 | 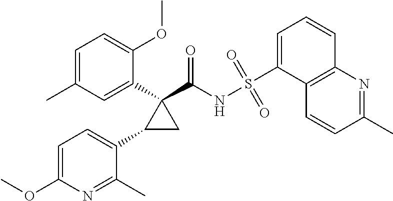 |
| 73 | 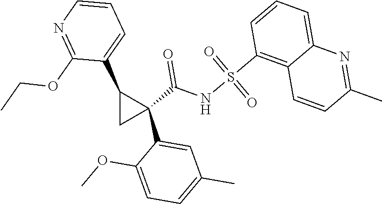 [SMILES: CCOc1ncccc1[C@@H]1C[C@]1(C(=O)NS(=O)(=O)c1cccc2nc(C)ccc12)c1cc(C)ccc1OC] |
| 74 | 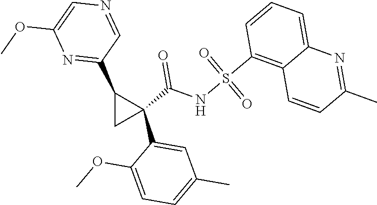 |
| 75 | 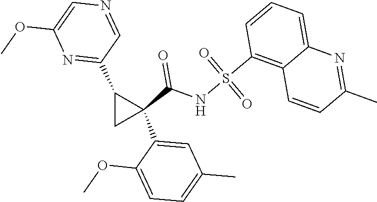 |
| 76 | 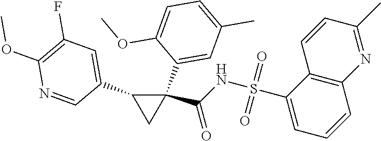 |
| 77 | 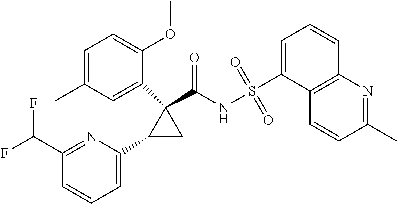 [SMILES: COc1ccc(C)cc1[C@]1(C(=O)NS(=O)(=O)c2cccc3nc(C)ccc23)C[C@@H]1c1cccc(C(F)F)n1] |
| 78 | 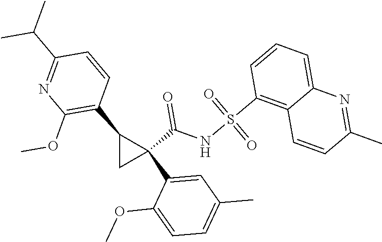 [SMILES: COc1ccc(C)cc1[C@@]1(C(=O)NS(=O)(=O)c2cccc3nc(C)ccc23)C[C@H]1c1ccc(C(C)C)nc1OC] |
| 79 | 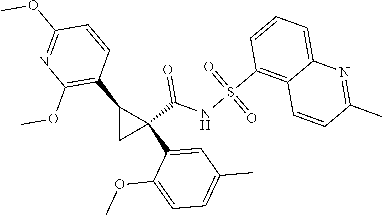 |
| 80 | 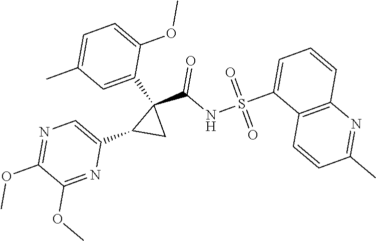 |
| 81 | 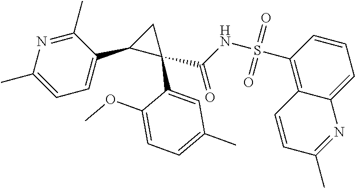 [SMILES: COc1ccc(C)cc1[C@]1(C(=O)NS(=O)(=O)c2cccc3nc(C)ccc23)C[C@@H]1c1ccc(C)nc1C] |
| 82 | 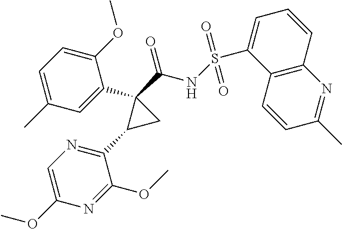 |
| 83 | 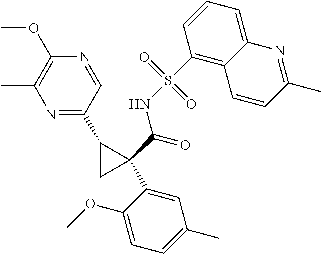 |
| 84 | 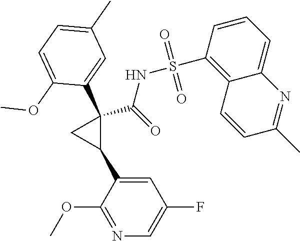 |
| 85 | 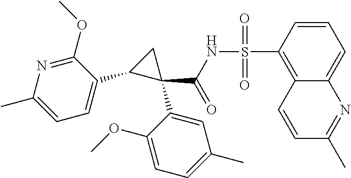 |
| 86 | 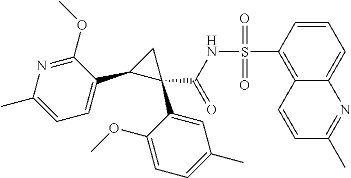 |
| 87 | 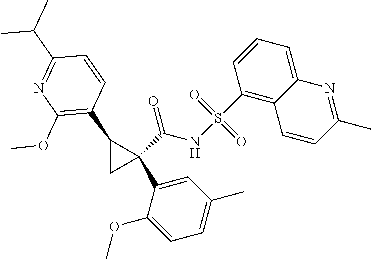 [SMILES: COc1ccc(C)cc1[C@@]1(C(=O)NS(=O)(=O)c2cccc3nc(C)ccc23)C[C@H]1c1ccc(C(C)C)nc1OC] |
| 88 | 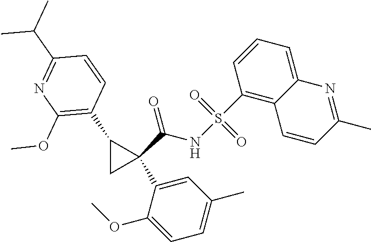 [SMILES: COc1ccc(C)cc1[C@]1(C(=O)NS(=O)(=O)c2cccc3nc(C)ccc23)C[C@@H]1c1ccc(C(C)C)nc1OC] |
| 89 | 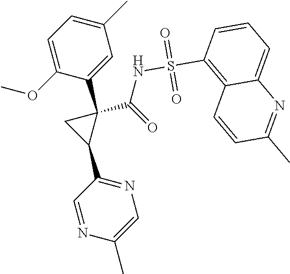 |
| 90 | 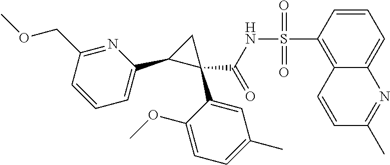 |
| 91 | 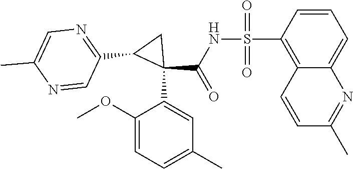 |
| 92 | 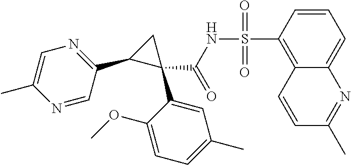 |
| 93 | 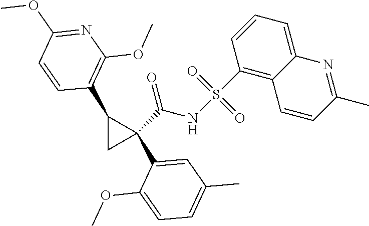 |
| 94 | 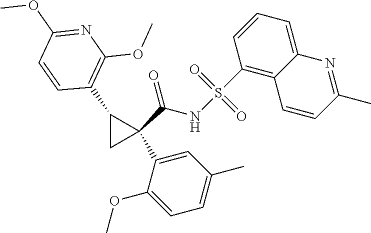 |
| 95 | 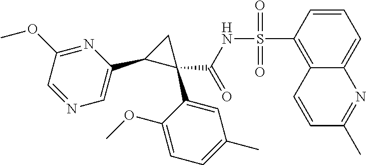 |
| 96 | 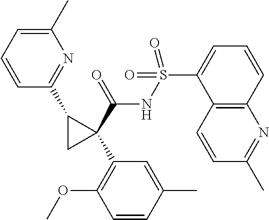 [SMILES: COc1ccc(C)cc1[C@]1(C(=O)NS(=O)(=O)c2cccc3nc(C)ccc23)C[C@@H]1c1cccc(C)n1] |
| 97 | 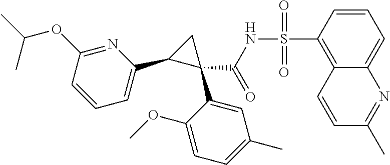 [SMILES: COc1ccc(C)cc1[C@]1(C(=O)NS(=O)(=O)c2cccc3nc(C)ccc23)C[C@@H]1c1cccc(OC(C)C)n1] |
| 98 | 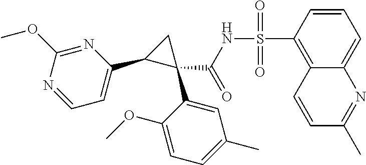 |
| 99 | 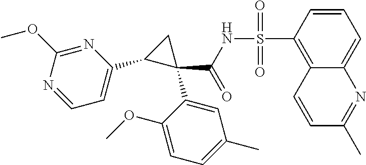 |
| 100 | 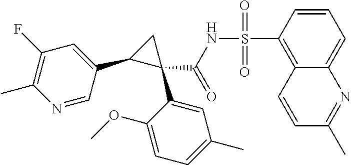 |
| 101 | 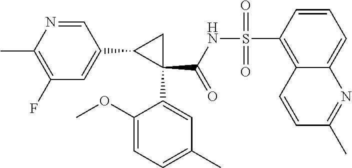 |
| 102 | 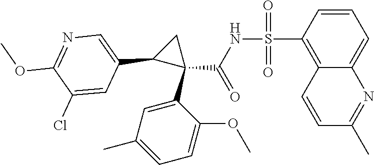 |
| 103 | 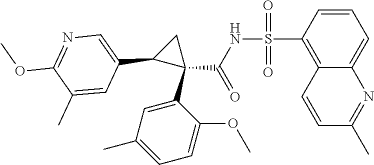 |
| 104 | 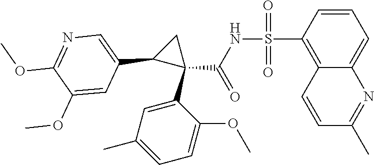 |
| 105 | 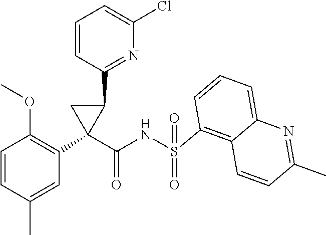 [SMILES: COc1ccc(C)cc1[C@]1(C(=O)NS(=O)(=O)c2cccc3nc(C)ccc23)C[C@H]1c1cccc(Cl)n1] |
| 106 | 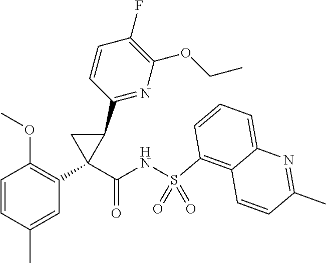 [SMILES: CCOc1nc([C@@H]2C[C@@]2(C(=O)NS(=O)(=O)c2cccc3nc(C)ccc23)c2cc(C)ccc2OC)ccc1F] |

Compounds of Formula (I), Formula (II), or Formula (III) may be used in the form of pharmaceutically acceptable salts.

Compounds of Formula (I), Formula (II), or Formula (III) may contain either a basic or an acidic functionality, or both, and may be converted to a pharmaceutically acceptable salt, when desired, by using a suitable acid or base. The salts may be prepared in situ during the final isolation and purification of the compounds of the invention.

### Methods of Making Exemplary Compounds

The compounds of the invention can be better understood in connection with the following synthetic schemes and methods which illustrate a means by which the compounds can be prepared. The compounds of this invention can be prepared by a variety of synthetic procedures. Representative synthetic procedures are shown in, but not limited to, Scheme 1. The variable R¹ is defined as detailed herein, e.g., in the Summary.

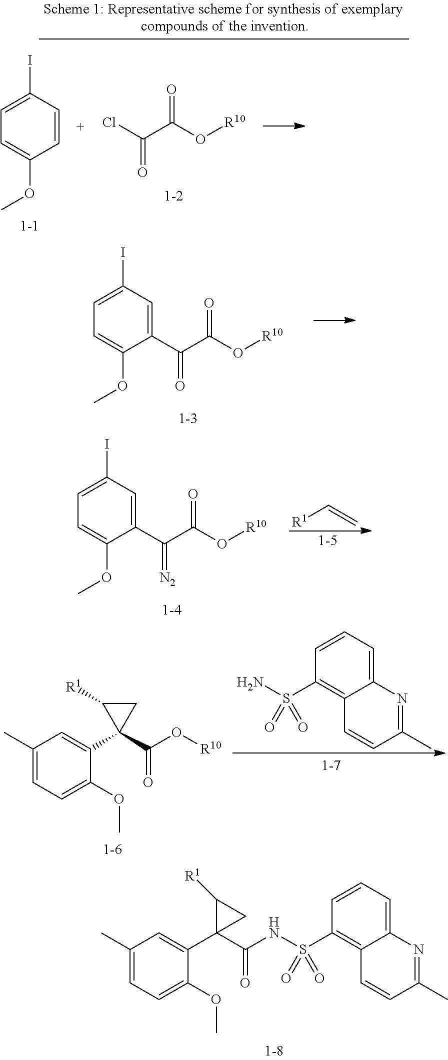

As shown in Scheme 1, compounds 1-8 can be prepared from compounds 1-1. 1-Methoxy-4-methylbenzene 1-1 can be acylated with acyl halides 1-2 where R¹⁰ is alkyl or another suitable carboxylic acid protecting group under Friedel-Crafts conditions using a Lewis acid including for example, AlCl₃ at reduced temperature to afford 2-oxoacetates 1-3. Treatment of 1-3 with 4-methylbenzenesulfonohydrazide at elevated temperature with removal of water affords 2-diazoacetates 1-4. Carbene addition of 1-4 to alkenes 1-5 can be affected by a suitable catalyst including for example, Rh₂(OAc)₄ in a suitable solvent including, for example dichloromethane to afford cyclopropanes 1-6. Ester hydrolysis of 1-6 to the corresponding carboxylic acid 1-6 (where R¹⁰ is hydrogen) by methods known to one skilled in the art including for example treatment with LiOH at elevated temperature followed by coupling with sulfonamides 1-7 affords compounds 1-8. Any suitable coupling conditions known to one skilled in the art can be used to affect the coupling of 1-6 and 1-7, including, for example, treatment with 1-(3-dimethylaminopropyl)-3-ethylcarbodiimide hydrochloride and 4-dimethylaminopyridine in a solvent including, for example, dichloromethane.

It can be appreciated that the synthetic schemes and specific examples as illustrated in the synthetic examples section are illustrative and are not to be read as limiting the scope of the invention as it is defined in the appended claims. All alternatives, modifications, and equivalents of the synthetic methods and specific examples are included within the scope of the claims.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that is incompatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in T. Greene and P. Wuts, Protecting Groups in Organic Synthesis (3^{rd} ed.), John Wiley & Sons, NY (1999), which is incorporated herein by reference in its entirety. Synthesis of the compounds of the invention can be accomplished by methods analogous to those described in the synthetic schemes described hereinabove and in specific examples.

When an optically active form of a compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be prepared by carrying out one of the above procedures using a pure geometric isomer as a starting material, or by resolution of a mixture of the geometric isomers of the compound or intermediates using a standard procedure such as chromatographic separation.

### Pharmaceutical Compositions

When employed as a pharmaceutical, a compound of the invention is typically administered in the form of a pharmaceutical composition. Such composition may be prepared in a manner known in the pharmaceutical art and comprise a therapeutically effective amount of a compound of Formula (I), or a pharmaceutically acceptable salt thereof, together with a pharmaceutically acceptable carrier. The phrase “pharmaceutical composition” refers to a composition suitable for administration in medical or veterinary use.

The term “pharmaceutically acceptable carrier” as used herein, means a non-toxic, inert solid, semi-solid or liquid filler, diluent, encapsulating material or formulation auxiliary of any type.

In certain embodiments, a pharmaceutical composition is provided comprising a therapeutically effective amount of a compound of Formula (I), or a pharmaceutically acceptable salt thereof, in combination with a pharmaceutically acceptable carrier.

In certain embodiments, a pharmaceutical composition is provided comprising a therapeutically effective amount of a compound of Formula (I), or a pharmaceutically acceptable salt thereof, one or more potentiator, and one or more additional correctors.

### Method of Use

The compounds of Formula (I), or pharmaceutically acceptable salts thereof, and pharmaceutical compositions comprising a compound of Formula (I), or a pharmaceutically acceptable salt thereof, using any amount and any route of administration may be administered to a subject for the treatment or prevention of cystic fibrosis.

The term “administering” refers to the method of contacting a compound with a subject.

Compounds of the invention are useful as modulators of CFTR. Thus, the compounds and compositions are particularly useful for treating or lessening the severity or progression of a disease, disorder, or a condition where hyperactivity or inactivity of CFTR is involved. Accordingly, the invention provides a method for treating cystic fibrosis in a subject, wherein the method comprises the step of administering to said subject a therapeutically effective amount of a compound of formula (I) or a preferred embodiment thereof as set forth above, with or without a pharmaceutically acceptable carrier. Particularly, the method is for the treatment or prevention of cystic fibrosis. In a more particular embodiment, the cystic fibrosis is caused by a Class I, II, III, IV, V, and/or VI mutation.

In certain embodiments, the present invention provides compounds of the invention, or pharmaceutical compositions comprising a compound of the invention for use in medicine. In a particular embodiment, the present invention provides compounds of the invention, or a pharmaceutically acceptable salt thereof, or a pharmaceutical composition comprising a compound of the invention, for use in medicine. In a particular embodiment, the present invention provides compounds of the invention or pharmaceutical compositions comprising a compound of the invention, for use in the treatment of cystic fibrosis. In a more particular embodiment, the cystic fibrosis is caused by a Class I, II, III, IV, V, and/or VI mutation.

Certain embodiments are directed to the use of a compound according to formula (I) or a pharmaceutically acceptable salt thereof, in the preparation of a medicament. The medicament optionally can comprise one or more additional therapeutic agents. In some embodiments, the medicament is for use in the treatment of cystic fibrosis. In a more particular embodiment, the cystic fibrosis is caused by a Class I, II, III, IV, V, and/or VI mutation.

This invention also is directed to the use of a compound according to formula (I) or a pharmaceutically acceptable salt thereof, in the manufacture of a medicament for the treatment of cystic fibrosis. The medicament optionally can comprise one or more additional therapeutic agents. In a particular embodiment, the invention is directed to the use of a compound according to formula (I), or a pharmaceutically acceptable salt thereof, in the manufacture of a medicament for the treatment of cystic fibrosis. In a more particular embodiment, the cystic fibrosis is caused by a Class I, II, III, IV, V, and/or VI mutation.

In certain embodiments, the present invention provides pharmaceutical compositions comprising a compound of the invention, or a pharmaceutically acceptable salt thereof, and one or more additional therapeutic agents. In certain embodiments, the present invention provides pharmaceutical compositions comprising a compound of the invention, or a pharmaceutically acceptable salt thereof, and one or more additional therapeutic agents wherein the additional therapeutic agents are selected from the group consisting of CFTR modulators and CFTR amplifiers. In certain embodiments, the present invention provides pharmaceutical compositions comprising a compound of the invention, or a pharmaceutically acceptable salt thereof, and one or more additional therapeutic agents wherein the additional therapeutic agents are CFTR modulators.

The present compounds or pharmaceutically acceptable salts thereof may be administered as the sole active agent or it may be co-administered with other therapeutic agents, including other compounds or a pharmaceutically acceptable salt thereof that demonstrate the same or a similar therapeutic activity and that are determined to be safe and efficacious for such combined administration. The present compounds may be co-administered to a subject. The term “co-administered” means the administration of two or more different therapeutic agents to a subject in a single pharmaceutical composition or in separate pharmaceutical compositions. Thus, co-administration involves administration at the same time of a single pharmaceutical composition comprising two or more therapeutic agents or administration of two or more different compositions to the same subject at the same or different times.

The compounds of the invention or pharmaceutically acceptable salts thereof may be co-administered with a therapeutically effective amount of one or more additional therapeutic agents to treat a CFTR mediated disease, where examples of the therapeutic agents include, but are not limited to antibiotics (for example, aminoglycosides, colistin, aztreonam, ciprofloxacin, and azithromycin), expectorants (for example, hypertonic saline, acetylcysteine, dornase alfa, and denufosol), pancreatic enzyme supplements (for example, pancreatin, and pancrelipase), epithelial sodium channel blocker (ENaC) inhibitors, CFTR modulators (for example, CFTR potentiators, CFTR correctors), and CFTR amplifiers.

In certain embodiments, the compounds of the invention or pharmaceutically acceptable salts thereof may be co-administered with one or two CFTR modulators and one CFTR amplifier. In certain embodiments, the compounds of the invention or pharmaceutically acceptable salts thereof may be co-administered with one potentiator, and one or more correctors. In certain embodiments, the compounds of the invention or pharmaceutically acceptable salts thereof may be co-administered with one potentiator, one or more correctors, and one CFTR amplifier. In certain embodiments, the compounds of the invention or pharmaceutically acceptable salts thereof may be co-administered with one or more CFTR modulators. In certain embodiments, the compounds of the invention or pharmaceutically acceptable salts thereof may be co-administered with one CFTR modulator. In certain embodiments, the compounds of the invention or pharmaceutically acceptable salts thereof may be co-administered with two CFTR modulators. In certain embodiments, the compounds of the invention or pharmaceutically acceptable salts thereof may be co-administered with three CFTR modulators. In certain embodiments, the compounds of the invention or pharmaceutically acceptable salts thereof may be co-administered with one potentiator and one or more correctors. In certain embodiments, the compounds of the invention or pharmaceutically acceptable salts thereof may be co-administered with one potentiator and two correctors. In certain embodiments, the compounds of the invention or pharmaceutically acceptable salts thereof may be co-administered with one potentiator. In certain embodiments, the compounds of the invention or pharmaceutically acceptable salts thereof may be co-administered with one or more correctors. In certain embodiments, the compounds of the invention or pharmaceutically acceptable salts thereof may be co-administered with one corrector. In certain embodiments, the compounds of the invention or pharmaceutically acceptable salts thereof may be co-administered two correctors. In certain embodiments, the compounds of the invention or pharmaceutically acceptable salts thereof may be co-administered with one or more correctors, and one amplifier. In certain embodiments, the compounds of the invention or pharmaceutically acceptable salts thereof may be co-administered with one corrector, and one amplifier. In certain embodiments, the compounds of the invention or pharmaceutically acceptable salts thereof may be co-administered with two correctors, and one amplifier. In certain embodiments, the compounds of the invention or pharmaceutically acceptable salts thereof may be co-administered with one corrector. In certain embodiments, the compounds of the invention or pharmaceutically acceptable salts thereof may be co-administered with two correctors.

Examples of CFTR potentiators include, but are not limited to, Ivacaftor (VX-770), ABBV-2451, 4-amino-7-{[1-(2-fluorophenyl)-1H-pyrazol-4-yl]methyl}-5-[2-(trifluoromethyl)pyrimidin-5-yl]-7H-pyrrolo[2,3-d]pyrimidine-6-carbonitrile, GLPG1837, VX-561, NVS-QBW251, FD1860293, PTI-808, N-(3-carbamoyl-5,5,7,7-tetramethyl-5,7-dihydro-4H-thieno[2,3-c]pyran-2-yl)-1H-pyrazole-5-carboxamide, 3-amino-N-[(2S)-2-hydroxypropyl]-5-{[4-(trifluoromethoxy)phenyl]sulfonyl}pyridine-2-carboxamide and 4-amino-7-{[1-(2-fluorophenyl)-1H-pyrazol-4-yl]methyl}-5-[2-(trifluoromethyl)pyrimidin-5-yl]-7H-pyrrolo[2,3-d]pyrimidine-6-carbonitrile. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014/180562, WO2015018823, WO2016193812 WO2017208115 and WO2018094237.

In certain embodiments, the potentiator is selected from the group consisting of
Ivacaftor (VX-770, N-(2,4-di-tert-butyl-5-hydroxyphenyl)-4-oxo-1,4-dihydroquinoline-3-carboxamide);
ABBV-2451;
GLPG1837;
VX-561;
NVS-QBW251;
FD1860293;
PTI-808;
2-(2-fluorobenzamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-1H-pyrazole-5-carboxamide;
2-(2-hydroxybenzamido)-5,5,7,7-tetramethyl-5,7-dihydro-4H-thieno[2,3-c]pyran-3-carboxamide;
2-(1-hydroxycyclopropanecarboxamido)-5,5,7,7-tetramethyl-5,7-dihydro-4H-thieno[2,3-c]pyran-3-carboxamide;
5,5,7,7-tetramethyl-2-(2-(trifluoromethyl)benzamido)-5,7-dihydro-4H-thieno[2,3-c]pyran-3-carboxamide;
2-(2-hydroxy-2-methylpropanamido)-5,5,7,7-tetramethyl-5,7-dihydro-4H-thieno[2,3-c]pyran-3-carboxamide;
2-(1-(hydroxymethyl)cyclopropanecarboxamido)-5,5,7,7-tetramethyl-5,7-dihydro-4H-thieno[2,3-c]pyran-3-carboxamide;
2-(3-hydroxy-2,2-dimethylpropanamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-5-methyl-1H-pyrazole-3-carboxamide;
N-(3-carbamoyl-5,5,7,7-tetramethyl-5,7-dihydro-4H-thieno[2,3-c]pyran-2-yl)-5-cyclopropyl-1H-pyrazole-3-carboxamide;
N-(3-carbamoyl-5,5,7,7-tetramethyl-5,7-dihydro-4H-thieno[2,3-c]pyran-2-yl)-5-isopropyl-1H-pyrazole-3-carboxamide;
N-(3-carbamoyl-5,5,7,7-tetramethyl-5,7-dihydro-4H-thieno[2,3-c]pyran-2-yl)-5-(trifluoromethyl)-1H-pyrazole-3-carboxamide;
5-tert-butyl-N-(3-carbamoyl-5,5,7,7-tetramethyl-5,7-dihydro-4H-thieno[2,3-c]pyran-2-yl)-1H-pyrazole-3-carboxamide;
N-(3-carbamoyl-5,5,7,7-tetramethyl-5,7-dihydro-4H-thieno[2,3-c]pyran-2-yl)-5-ethyl-1H-pyrazole-3-carboxamide;
N-(3-carbamoyl-5,5,7,7-tetramethyl-5,7-dihydro-4H-thieno[2,3-c]pyran-2-yl)-3-ethyl-4-methyl-1H-pyrazole-5-carboxamide;
2-(2-hydroxypropanamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-4-chloro-1H-pyrazole-3-carboxamide;
N-(3-carbamoyl-5,5,7,7-tetramethyl-5,7-dihydro-4H-thieno[2,3-c]pyran-2-yl)-1,4,6,7-tetrahydropyrano[4,3-c]pyrazole-3-carboxamide;
4-bromo-N-(3-carbamoyl-5,5,7,7-tetramethyl-5,7-dihydro-4H-thieno[2,3-c]pyran-2-yl)-1H-pyrazole-3-carboxamide;
N-(3-carbamoyl-5,5,7,7-tetramethyl-5,7-dihydro-4H-thieno[2,3-c]pyran-2-yl)-4-chloro-5-methyl-1H-pyrazole-3-carboxamide;
N-(3-carbamoyl-5,5,7,7-tetramethyl-5,7-dihydro-4H-thieno[2,3-c]pyran-2-yl)-4-methyl-1H-pyrazole-3-carboxamide;
2-(2-hydroxy-3,3-dimethylbutanamido)-5,5,7,7-tetramethyl-5,7-dihydro-4H-thieno[2,3-c]pyran-3-carboxamide;
2-[(2-hydroxy-4-methyl-pentanoyl)amino]-5,5,7,7-tetramethyl-4H-thieno[2,3-c]pyran-3-carboxamide;
5-(2-methoxy-ethoxy)-1H-pyrazole-3-carboxylic acid (3-carbamoyl-5,5,7,7-tetramethyl-4,7-dihydro-5H-thieno[2,3-c]pyran-2-yl)-amide;
N-(3-carbamoyl-5,5,7,7-tetramethyl-4H-thieno[2,3-c]pyran-2-yl)-4-(3-methoxypropyl)-1H-pyrazole-3-carboxamide;
N-(3-carbamoyl-5,5,7,7-tetramethyl-4H-thieno[2,3-c]pyran-2-yl)-4-(2-ethoxyethyl)-1H-pyrazole-3-carboxamide;
2-[[(2S)-2-hydroxy-3,3-dimethyl-butanoyl]amino]-5,5,7,7-tetramethyl-4H-thieno[2,3-c]pyran-3-carboxamide;
2-[[(2R)-2-hydroxy-3,3-dimethyl-butanoyl]amino]-5,5,7,7-tetramethyl-4H-thieno[2,3-c]pyran-3-carboxamide;
2-[(2-hydroxy-2,3,3-trimethyl-butanoyl)amino]-5,5,7,7-tetramethyl-4H-thieno[2,3-c]pyran-3-carboxamide;
[5-[(3-carbamoyl-5,5,7,7-tetramethyl-4H-thieno[2,3-c]pyran-2-yl)carbamoyl]pyrazol-1-yl]methyl dihydrogen phosphate;
[3-[(3-carbamoyl-5,5,7,7-tetramethyl-4H-thieno[2,3-c]pyran-2-yl)carbamoyl]pyrazol-1-yl]methyl dihydrogen phosphate;
N-(3-carbamoyl-5,5,7,7-tetramethyl-4H-thieno[2,3-c]pyran-2-yl)-4-(1,4-dioxan-2-yl)-1H-pyrazole-3-carboxamide;
5,5,7,7-tetramethyl-2-[[(2S)-3,3,3-trifluoro-2-hydroxy-2-methyl-propanoyl]amino]-4H-thieno[2,3-c]pyran-3-carboxamide;
2-[[(2S)-2-hydroxypropanoyl]amino]-5,5,7,7-tetramethyl-4H-thieno[2,3-c]pyran-3-carboxamide;
3-amino-N-(2-hydroxy-2-methylpropyl)-5-{[4-(trifluoromethoxy)phenyl]sulfonyl}pyridine-2-carboxamide;
3-amino-N-[(4-hydroxy-1-methylpiperidin-4-yl)methyl]-5-{[4-(trifluoromethoxy)phenyl]sulfonyl}pyridine-2-carboxamide;
3-amino-N-(3-hydroxy-2,2-dimethylpropyl)-5-{[4-(trifluoromethoxy)phenyl]sulfonyl}pyridine-2-carboxamide;
3-amino-5-[(4-fluorophenyl)sulfonyl]-N-[(1-hydroxycyclopropyl)methyl]pyridine-2-carboxamide;
3-amino-5-[(4-fluorophenyl)sulfonyl]-N-[(2R)-3,3,3-trifluoro-2-hydroxypropyl]pyridine-2-carboxamide;
3-amino-5-[(3-fluorophenyl)sulfonyl]-N-(2-hydroxy-2-methylpropyl)pyridine-2-carboxamide;
3-amino-N-[2-(cyclopropylamino)-2-oxoethyl]-5-{[4-(trifluoromethoxy)phenyl]sulfonyl}pyridine-2-carboxamide;
(3-amino-5-{[4-(trifluoromethoxy)phenyl]sulfonyl}pyridin-2-yl)(azetidin-1-yl)methanone;
(3-amino-5-{[4-(trifluoromethoxy)phenyl]sulfonyl}pyridin-2-yl)[3-(hydroxymethyl)azetidin-1-yl]methanone;
(3-amino-5-{[4-(trifluoromethoxy)phenyl]sulfonyl}pyridin-2-yl)(3-fluoroazetidin-1-yl)methanone;
3-amino-N-[(2R)-2-hydroxy-3-methoxypropyl]-5-f{[4-(trifluoromethyl)phenyl]sulfonyl}pyridine-2-carboxamide;
(3-amino-5-{[2-fluoro-4-(trifluoromethoxy)phenyl]sulfonyl}pyridin-2-yl)(3-hydroxyazetidin-1-yl)methanone;
(3-amino-5-{[2-(trifluoromethoxy)phenyl]sulfonyl}pyridin-2-yl)(3,3-difluoroazetidin-1-yl)methanone;
rac-3-amino-N-[(3R,4S)-4-hydroxytetrahydro-2H-pyran-3-yl]-5-{[2-(trifluoromethoxy)phenyl]sulfonyl}pyridine-2-carboxamide;
3-amino-5-[(4,4-difluoropiperidin-1-yl)sulfonyl]-N-(3,3,3-trifluoro-2-hydroxypropyl)pyridine-2-carboxamide;
(3-amino-5-{[2-(trifluoromethoxy)phenyl]sulfonyl}pyridin-2-yl)[3-hydroxy-3-(trifluoromethyl)azetidin-1-yl]methanone;
3-amino-N-(2-hydroxy-4-methylpentyl)-5-f{[4-(trifluoromethoxy)phenyl]sulfonyl}pyridine-2-carboxamide;
(3-amino-5-{[4-(trifluoromethyl)phenyl]sulfonyl}pyridin-2-yl)(3-hydroxy-3-methylazetidin-1-yl)methanone;
3-amino-N-(3,3,3-trifluoro-2-hydroxypropyl)-5-{[4-(trifluoromethyl)piperidin-1-yl]sulfonyl}pyridine-2-carboxamide;
3-amino-N-[2-hydroxy-1-(4-methoxyphenyl)ethyl]-5-{[4-(trifluoromethoxy)phenyl]sulfonyl}pyridine-2-carboxamide;
3-amino-5-[(3,3-difluoroazetidin-1-yl)sulfonyl]-N-(3,3,3-trifluoro-2-hydroxypropyl)pyridine-2-carboxamide;
3-amino-5-f{[2-fluoro-4-(trifluoromethyl)phenyl]sulfonyl}-N-[(2S)-2-hydroxypropyl]pyridine-2-carboxamide;
3-amino-5-f{[2-fluoro-4-(trifluoromethyl)phenyl]sulfonyl}-N-[(2R)-2-hydroxy-3-methoxypropyl]pyridine-2-carboxamide;
3-amino-N-[2-oxo-2-(propan-2-ylamino)ethyl]-5-f{[4-(trifluoromethyl)phenyl]sulfonyl}pyridine-2-carboxamide;
(3-amino-5-{[4-(trifluoromethyl)phenyl]sulfonyl}pyridin-2-yl)[3-hydroxy-3-(trifluoromethyl)azetidin-1-yl]methanone;
3-amino-5-f{[2-fluoro-4-(trifluoromethyl)phenyl]sulfonyl}-N-[(3R)-tetrahydrofuran-3-ylmethyl]pyridine-2-carboxamide;
(3-amino-5-f{[2-fluoro-4-(trifluoromethyl)phenyl]sulfonyl}pyridin-2-yl)[3-hydroxy-3-(trifluoromethyl)azetidin-1-yl]methanone;
3-amino-5-f{[2-fluoro-4-(trifluoromethyl)phenyl]sulfonyl}-N-[(3S)-tetrahydrofuran-3-ylmethyl]pyridine-2-carboxamide;
3-amino-5-{[2-fluoro-4-(trifluoromethoxy)phenyl]sulfonyl}-N-[(3S)-tetrahydrofuran-3-ylmethyl]pyridine-2-carboxamide;
3-amino-N-[2-hydroxy-3-(2,2,2-trifluoroethoxy)propyl]-5-{[4-(trifluoromethyl)phenyl]sulfonyl}pyridine-2-carboxamide;
3-amino-N-(3-tert-butoxy-2-hydroxypropyl)-5-{[2-fluoro-4-(trifluoromethyl)phenyl]sulfonyl}pyridine-2-carboxamide;
[3-amino-5-(phenylsulfonyl)pyridin-2-yl][3-hydroxy-3-(trifluoromethyl)azetidin-1-yl]methanone;
{3-amino-5-[(3-fluorophenyl)sulfonyl]pyridin-2-yl}[3-hydroxy-3-(trifluoromethyl)azetidin-1-yl]methanone; and
3-amino-N-[(2S)-2-hydroxypropyl]-5-{[4-(trifluoromethoxy)phenyl]sulfonyl}pyridine-2-carboxamide.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661, tezacaftor), VX-983, ABV-2222, GLPG2665, ABBV-2737, ABBV-2851, ABBV-3221, 1-{5-cyclopropyl-2-[(propan-2-yl)oxy]pyridin-3-yl}-N-(2-methylquinoline-5-sulfonyl)cyclopropane-1-carboxamide, 1-(5-ethyl-2-{[(2R)-1-methoxypropan-2-yl]oxy}phenyl)-N-(2-methylquinoline-5-sulfonyl)cyclopropane-1-carboxamide, 1-{5-ethyl-2-[(propan-2-yl)oxy]pyridin-3-yl}-N-(2-methylquinoline-5-sulfonyl)cyclopropane-1-carboxamide, PTI-801, VX-152, VX-440, VX-659 (bamocaftor), VX-445 (elexacaftor), VX-121, FDL169, FDL304, FD2052160, and FD2035659. Examples of correctors are also disclosed in U.S. Pat. Nos. 9,642,831, 9,567,322, 9,840,513, 10,118,916, 9,796,711, 9,890,158, 10,399,940, and 9,981,910.

In certain embodiments, the corrector(s) can be selected from the group consisting of
Lumacaftor (VX-809);
1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661, tezacaftor);
VX-983;
GLPG2665;
ABBV-2737;
ABBV-3221;
PTI-801;
VX-152;
VX-440;
VX-659;
VX-445 (elexacaftor)
FDL169
FDL304;
FD2052160;
FD2035659;
3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-6-methyl-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-methyl-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-6-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]cyclohexanecarboxylic acid;
3-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-methoxy-3,4-dihydro-2H-chromen-2-yl]cyclohexanecarboxylic acid;
3-[(2R,4R)-4-({[1-(2,2-difluoro-1,3-benzodioxol-5-yl)cyclopropyl]carbonyl}amino)-7-fluoro-3,4-dihydro-2H-chromen-2-yl]benzoic acid;
3-({3-[(2R,4R)-4-({[1-(2,2-difluoro-1,3-benzodioxol-5-yl)cyclopropyl]carbonyl}amino)-7-methyl-3,4-dihydro-2H-chromen-2-yl]benzoyl}amino)-1-methylcyclopentanecarboxylic acid;
3-[(2R,4R)-4-({[1-(2,2-difluoro-1,3-benzodioxol-5-yl)cyclopropyl]carbonyl}amino)-7-methyl-3,4-dihydro-2H-chromen-2-yl]-N-[(2R)-2,3-dihydroxypropyl]benzamide;
3-[(2R,4R)-4-({[1-(2,2-difluoro-1,3-benzodioxol-5-yl)cyclopropyl]carbonyl}amino)-7-(2-methoxyethoxy)-3,4-dihydro-2H-chromen-2-yl]benzoic acid;
3-[(2R,4R)-7-(benzyloxy)-4-({[1-(2,2-difluoro-1,3-benzodioxol-5-yl)cyclopropyl]carbonyl}amino)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(2-fluoroethoxy)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(trifluoromethyl)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(trifluoromethyl)-3,4-dihydro-2H-chromen-2-yl]cyclohexanecarboxylic acid;
4-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-8-fluoro-3,4-dihydro-2H-chromen-2-yl]benzoic acid;
4-[(2R,4R)-4-({[1-(2,2-difluoro-1,3-benzodioxol-5-yl)cyclopropyl]carbonyl}amino)-3,4-dihydro-2H-chromen-2-yl]benzoic acid;
4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid;
rac-3-[(2R,4S)-4-({[1-(2,2-difluoro-1,3-benzodioxol-5-yl)cyclopropyl]carbonyl}amino)tetrahydro-2H-pyran-2-yl]benzoic acid;
rac-4-[(2R,4S)-4-({[1-(2,2-difluoro-1,3-benzodioxol-5-yl)cyclopropyl]carbonyl}amino)tetrahydro-2H-pyran-2-yl]benzoic acid;
3-[(2S,4R)-4-({[1-(2,2-difluoro-1,3-benzodioxol-5-yl)cyclopropyl]carbonyl}amino)tetrahydro-2H-pyran-2-yl]benzoic acid;
3-[(2R,4S)-4-({[1-(2,2-difluoro-1,3-benzodioxol-5-yl)cyclopropyl]carbonyl}amino)tetrahydro-2H-pyran-2-yl]benzoic acid;
rac-3-[(2R,4S,6S)-4-({[1-(2,2-difluoro-1,3-benzodioxol-5-yl)cyclopropyl]carbonyl}amino)-6-phenyltetrahydro-2H-pyran-2-yl]benzoic acid;
3-[(2S,4R,6R)-4-({[1-(2,2-difluoro-1,3-benzodioxol-5-yl)cyclopropyl]carbonyl}amino)-6-phenyltetrahydro-2H-pyran-2-yl]benzoic acid;
3-[(2R,4S,6S)-4-({[1-(2,2-difluoro-1,3-benzodioxol-5-yl)cyclopropyl]carbonyl}amino)-6-phenyltetrahydro-2H-pyran-2-yl]benzoic acid;
4-[(2R,4S)-4-({[1-(2,2-difluoro-1,3-benzodioxol-5-yl)cyclopropyl]carbonyl}amino)tetrahydro-2H-pyran-2-yl]benzoic acid;
4-[6-(4-cyanopiperidin-1-yl)pyridin-3-yl]-3-cyclobutyl-N-(methanesulfonyl)-1-phenyl-1H-pyrazolo[3,4-b]pyridine-6-carboxamide;
3-cyclobutyl-N-(methanesulfonyl)-4-[4-(methoxymethyl)piperidin-1-yl]-1-phenyl-1H-pyrazolo[3,4-b]pyridine-6-carboxamide;
4-[6-(4-cyanopiperidin-1-yl)pyridin-3-yl]-3-cyclobutyl-N-(methanesulfonyl)-1-[2-(morpholin-4-yl)pyridin-4-yl]-1H-pyrazolo[3,4-b]pyridine-6-carboxamide;
N-(methanesulfonyl)-4-[4-(methoxymethyl)piperidin-1-yl]-1-[2-(morpholin-4-yl)pyridin-4-yl]-3-(propan-2-yl)-1H-pyrazolo[3,4-b]pyridine-6-carboxamide;
3-cyclobutyl-4-[4-(methoxymethyl)piperidin-1-yl]-N-[2-(morpholin-4-yl)ethanesulfonyl]-1-phenyl-1H-pyrazolo[3,4-b]pyridine-6-carboxamide;
3-cyclobutyl-N-[2-(dimethylamino)ethanesulfonyl]-4-[4-(methoxymethyl)piperidin-1-yl]-1-phenyl-1H-pyrazolo[3,4-b]pyridine-6-carboxamide;
1-(4-fluorophenyl)-N-(methanesulfonyl)-4-(1′-methyl[4,4′-bipiperidin]-1-yl)-3-(propan-2-yl)-1H-pyrazolo[3,4-b]pyridine-6-carboxamide;
3-cyclobutyl-N-(methanesulfonyl)-4-{4-[2-(morpholin-4-yl)ethyl]piperidin-1-yl}-1-phenyl-1H-pyrazolo[3,4-b]pyridine-6-carboxamide;
3-cyclobutyl-4-[4-(methoxymethyl)piperidin-1-yl]-N-(oxolane-3-sulfonyl)-1-phenyl-1H-pyrazolo[3,4-b]pyridine-6-carboxamide;
3-cyclobutyl-N-(dimethylsulfamoyl)-1-(4-fluorophenyl)-4-(4-methoxy[1,4′-bipiperidin]-1′-yl)-1H-pyrazolo[3,4-b]pyridine-6-carboxamide;
3-cyclobutyl-N-(morpholine-4-sulfonyl)-4-[4-(morpholin-4-yl)piperidin-1-yl]-1-phenyl-1H-pyrazolo[3,4-b]pyridine-6-carboxamide;
3-cyclobutyl-N-(morpholine-4-sulfonyl)-1-phenyl-4-{4-[(pyrrolidin-1-yl)methyl]piperidin-1-yl}-1H-pyrazolo[3,4-b]pyridine-6-carboxamide;
3-cyclobutyl-N-(methanesulfonyl)-4-[4-(morpholin-4-yl)piperidin-1-yl]-1-phenyl-1H-pyrazolo[3,4-b]pyridine-6-carboxamide;
3-cyclobutyl-4-[4-(morpholin-4-yl)piperidin-1-yl]-1-phenyl-1H-pyrazolo[3,4-b]pyridine-6-carboxylic acid;
3-cyclobutyl-1-phenyl-4-{4-[(pyrrolidin-1-yl)methyl]piperidin-1-yl}-1H-pyrazolo[3,4-b]pyridine-6-carboxylic acid;
5-[(2R,4R)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]pyrazine-2-carboxylic acid;
6-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]pyridine-3-carboxylic acid;
trans-4-[(2S,4S)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclohexane-1-carboxylic acid;
6-[(2R,4R)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]pyridine-3-carboxylic acid;
trans-4-[(2S,4S)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]cyclohexane-1-carboxylic acid;
ethyl trans-4-[(2S,4S)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylate;
cis-4-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclohexane-1-carboxylic acid;
trans-4-[(2S,4S)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylic acid;
1-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclopropane-1-carboxylic acid;
trans-4-[(2R,4R)-4-{[(5S)-2,2-difluoro-5-methyl-6,7-dihydro-2H,5H-indeno[5,6-d][1,3]dioxole-5-carbonyl]amino}-7-(trifluoromethoxy)-3,4-dihydro-2H-1-benzopyran-2-yl]cyclohexane-1-carboxylic acid;
trans-4-[(2R,4R)-4-{[(5S)-2,2-difluoro-5-methyl-6,7-dihydro-2H,5H-indeno[5,6-d][1,3]dioxole-5-carbonyl]amino}-7-methoxy-3,4-dihydro-2H-1-benzopyran-2-yl]cyclohexane-1-carboxylic acid;
trans-4-[(2R,4R)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]cyclohexane-1-carboxylic acid;
trans-4-[(2R,4R)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylic acid;
trans-4-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclohexane-1-carboxylic acid;
4-{(2R,4R)-4-[2-(2,2-difluoro-2H-1,3-benzodioxol-5-yl)-2-methylpropanamido]-7-methoxy-3,4-dihydro-2H-1-benzopyran-2-yl}benzoic acid;
4-[(2R,4R)-4-{[1-(3,4-dichlorophenyl)cyclopropane-1-carbonyl]amino}-7-methoxy-3,4-dihydro-2H-1-benzopyran-2-yl]benzoic acid;
4-[(2R,4R)-4-{[1-(4-bromophenyl)cyclopropane-1-carbonyl]amino}-7-methoxy-3,4-dihydro-2H-1-benzopyran-2-yl]benzoic acid;
4-[(2R,4R)-7-methoxy-4-({1-[4-(trifluoromethyl)phenyl]cyclopropane-1-carbonyl}amino)-3,4-dihydro-2H-1-benzopyran-2-yl]benzoic acid;
4-[(2R,4R)-7-methoxy-4-{[1-(4-methylphenyl)cyclopropane-1-carbonyl]amino}-3,4-dihydro-2H-1-benzopyran-2-yl]benzoic acid;
4-{(2R,4R)-4-[(1,5-dimethyl-2,3-dihydro-1H-indene-1-carbonyl)amino]-7-methoxy-3,4-dihydro-2H-1-benzopyran-2-yl}benzoic acid;
3-[(2R,4R)-4-{[(1S)-1,5-dimethyl-2,3-dihydro-1H-indene-1-carbonyl]amino}-7-methoxy-3,4-dihydro-2H-1-benzopyran-2-yl]benzoic acid;
4-[(2R,4R)-4-{[(1S)-1,5-dimethyl-2,3-dihydro-1H-indene-1-carbonyl]amino}-7-methoxy-3,4-dihydro-2H-1-benzopyran-2-yl]benzoic acid;
trans-4-[(2R,4R)-4-{[1-(2,2-difluoro-2H-1,3-benzodioxol-5-yl)cyclopropane-1-carbonyl]amino}-7-methoxy-3,4-dihydro-2H-1-benzopyran-2-yl]cyclohexane-1-carboxylic acid;
trans-4-[(2R,4R)-4-{[1-(2,2-difluoro-2H-1,3-benzodioxol-5-yl)cyclopropane-1-carbonyl]amino}-7-(trifluoromethoxy)-3,4-dihydro-2H-1-benzopyran-2-yl]cyclohexane-1-carboxylic acid; and
4-[(2R,4R)-4-{[1-(2,2-difluoro-2H-1,3-benzodioxol-5-yl)cyclopropane-1-carbonyl]amino}-7-(difluoromethoxy)-3,4-dihydro-2H-1-benzopyran-2-yl]cyclohexane-1-carboxylic acid.

In certain embodiments, the additional therapeutic agent is a CFTR amplifier. CFTR amplifiers enhance the effect of known CFTR modulators, such as potentiators and correctors. Examples of CFTR amplifiers include PTI130 and PTI-428. Examples of amplifiers are also disclosed in International Patent Publication Nos.: WO2015138909 and WO2015138934.

In certain embodiments, the additional therapeutic agent is a CFTR stabilizer. CFTR stabilizers enhance the stability of corrected CFTR that has been treated with a corrector, corrector/potentiator or other CFTR modulator combination(s). An example of a CFTR stabilizer is cavosonstat (N91115). Examples of stabilizers are also disclosed in International Patent Publication No.: WO2012048181.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, VX-371 and ETD001. Additional agents that reduce the activity of the epithelial sodium channel blocker (ENaC) can be found, for example, in International Patent Publication Nos.: WO2009074575 and WO2013043720; and U.S. Pat. No. 8,999,976.

In certain embodiments, the ENaC inhibitor is VX-371.

In certain embodiments, the ENaC inhibitor is SPX-101 (S18).

In certain embodiments, the ENac inhibitor is ETD001.

In certain embodiments, the additional therapeutic agent is a Transmembrane membrane 16A (TMEM16A) potentiator. TMEM16A potentiators enhance the flow of chloride across the lung cell membrane via calcium-activated TMEM16A channels present on the apical membrane of the epithelial cells. The increased chloride flow would result in increased mucus hydration. Examples of TMEME16A potentiators include ETD002. Examples of TMEM16A potentiators are also disclosed in International Patent Publication No.: WO2019145726.

In certain embodiments, a method for treating cystic fibrosis in a subject is provided, the method comprising administering a therapeutically effective amount of a compound of formula (I), or a pharmaceutically acceptable salt thereof, to a subject in need thereof.

This invention also is directed to kits that comprise one or more compounds and/or salts of the invention, and, optionally, one or more additional therapeutic agents.

This invention also is directed to methods of use of the compounds, salts, compositions, and/or kits of the invention to, for example, modulate the Cystic Fibrosis Transmembrane Conductance Regulator (CFTR) protein, and treat a disease treatable by modulating the Cystic Fibrosis Transmembrane Conductance Regulator (CFTR) protein (including cystic fibrosis).

In certain embodiments, the present invention provides compounds of the invention, or pharmaceutical compositions comprising a compound of the invention, for use in medicine. In a particular embodiment, the present invention provides compounds of the invention, or pharmaceutical compositions comprising a compound of the invention, for use in the treatment of diseases or disorders as described herein above.

Certain embodiments are directed to the use of a compound according to Formula (I), or a pharmaceutically acceptable salt thereof in the preparation of a medicament. In some embodiments the medicament is for use in the treatment of diseases and disorders as described herein above.

This invention is also directed to the use of a compound according to Formula (I), or a pharmaceutically acceptable salt thereof in the manufacture of a medicament for the treatment of the diseases and disorders as described herein above.

### EXAMPLES

The following Examples may be used for illustrative purposes and should not be deemed to narrow the scope of the invention.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference.

The following abbreviations have the indicated meaning unless otherwise specified: NMR for nuclear magnetic resonance; s for singlet; br s for broad singlet; d for duplet or doublet; m for multiplet; t for triplet; q for quartet; LC/MS or LCMS for liquid chromatography-mass spectrometry; min for minute; mL for milliliter; μL for microliter; L for liter; g for gram; mg for milligram; mmol for millimoles; psi for pounds per square inch; HPLC for high pressure liquid chromatography; ppm for parts per million; APCI for atmospheric pressure chemical ionization; DCI for desorption chemical ionization; DSI for droplet spray ionization; ESI for electrospray ionization; RT for retention time; M for molarity (moles/liter); N for normality (equivalent/liter); ee for enantiomeric excess; and de for diastereomeric excess.

### Example 1

### rac-(1r,2s)-1-(2-methoxy-5-methylphenyl)-N-(2-methylquinoline-5-sulfonyl)-2-phenylcyclopropane-1-carboxamide

### Example 1A

### methyl 2-(2-methoxy-5-methylphenyl)-2-oxoacetate

1-Methoxy-4-methylbenzene (6.30 mL, 50 mmol, Aldrich) was added to a suspension of aluminum chloride (8.00 g, 60.0 mmol) in dichloromethane (100 mL) at 0° C. After stirring for 10 minutes, methyl 2-chloro-2-oxoacetate (5.52 mL, 60.0 mmol, Aldrich) was added dropwise, and the reaction was allowed to slowly warm to ambient temperature. After 16 hours, the reaction was quenched with 1 M hydrochloric acid (200 mL) and vigorously stirred for 15 minutes. The layers were separated, and the organic phase was washed with saturated NaHCO₃, brine, dried with MgSO₄, filtered, and concentrated under reduced pressure. The crude residue was purified by flash chromatography (ISCO CombiFlash, 0-30% ethyl acetate/heptanes, 120 g RediSep® gold silica column) to afford the title compound (8.9 g, 42.7 mmol, 85% yield). ¹H NMR (400 MHz, CDCl₃) δ ppm 7.67 (dd, J=2.3, 1.1 Hz, 1H), 7.43-7.34 (m, 1H), 6.89 (d, J=8.5 Hz, 1H), 3.91 (s, 3H), 3.84 (s, 3H), 2.33 (d, J=0.8 Hz, 3H). MS(APCI+) m/z 209.5 (M+H)⁺.

### Example 1B

### methyl 2-diazo-2-(2-methoxy-5-methylphenyl)acetate

A mixture of Example 1A (10.2 g, 49.0 mmol) and 4-methylbenzenesulfonohydrazide (9.12 g, 49.0 mmol, Aldrich) in toluene (100 mL) was heated at reflux with a Dean-Stark trap. After 16 hours, the reaction was concentrated under reduced pressure, and dichloromethane (100 mL) and triethylamine (8.19 mL, 58.8 mmol) were added to the resulting residue. After stirring at ambient temperature for 48 hours, the reaction was washed with saturated NaHCO₃, brine, dried with MgSO₄, and concentrated under reduced pressure. The crude residue was purified by flash chromatography (ISCO CombiFlash, 0-20% ethyl acetate/heptanes, 120 g RediSep® gold silica column) to afford the title compound (7.89 g, 35.8 mmol, 73.1% yield). ¹H NMR (500 MHz, CDCl₃) δ ppm 7.40-7.32 (m, 1H), 7.05 (ddt, J=8.4, 2.3, 0.7 Hz, 1H), 6.79 (d, J=8.4 Hz, 1H), 3.83 (s, 3H), 3.82 (s, 3H), 2.30 (t, J=0.7 Hz, 3H).

### Example 1C

### rac-(1r,2s)-methyl 1-(2-methoxy-5-methylphenyl)-2-phenylcyclopropanecarboxylate

A solution of Example 1B (200 mg, 0.908 mmol) in dichloromethane (3 mL) was added over 4 hours by syringe pump to a solution of styrene (315 μL, 2.72 mmol, Aldrich) and rhodium(II) acetate dimer (2.0 mg, 4.5 μmol, Aldrich) in dichloromethane (6 mL) at ambient temperature. After stirring for an additional 12 hours, the reaction was concentrated under reduced pressure, and the crude residue was purified by flash chromatography (ISCO CombiFlash, 0-30% ethyl acetate/heptanes, 40 g RediSep© gold silica column) to afford the title compound (257 mg, 0.867 mmol, 95% yield). ¹H NMR (500 MHz, CDCl₃) δ ppm 7.06-6.97 (m, 3H), 6.97-6.89 (m, 2H), 6.82-6.71 (m, 2H), 6.42 (d, J=8.2 Hz, 1H), 3.65 (s, 3H), 3.30 (s, 3H), 3.20 (dd, J=9.3, 7.4 Hz, 1H), 2.23 (d, J=0.8 Hz, 3H), 1.96 (dd, J=9.3, 5.0 Hz, 1H), 1.82 (dd, J=7.4, 5.0 Hz, 1H). MS(APCI+) m/z 297.4 (M+H)⁺.

### Example 1D

### rac-(1r,2s)-1-(2-methoxy-5-methylphenyl)-2-phenylcyclopropanecarboxylic acid

Lithium hydroxide (205 mg, 8.57 mmol) was added to a solution of Example 1C (254 mg, 0.857 mmol) in dioxane (4.6 mL) and water (1.1 mL). The reaction mixture was then heated to 80° C. for 16 hours before being acidified with 1 M hydrochloric acid and extracted with ethyl acetate. The organic phase was washed with 1 M hydrochloric acid, brine, dried with MgSO₄, filtered, and concentrated under reduced pressure to afford the title compound (218 mg, 0.772 mmol, 90% yield). ¹H NMR (400 MHz, dimethyl sulfoxide-d₆) δ ppm 12.08 (s, 1H), 7.05-6.90 (m, 4H), 6.87 (dd, J=8.6, 2.2 Hz, 1H), 6.83-6.76 (m, 2H), 6.49 (d, J=8.2 Hz, 1H), 3.27 (s, 3H), 3.03 (dd, J=9.1, 7.2 Hz, 1H), 2.16 (s, 3H), 1.87 (dd, J=7.2, 4.9 Hz, 1H), 1.74 (dd, J=9.2, 4.8 Hz, 1H). MS(APCI+) m/z 283.4 (M+H)⁺.

### Example 1E

### rac-(1r,2s)-1-(2-methoxy-5-methylphenyl)-N-(2-methylquinoline-5-sulfonyl)-2-phenylcyclopropane-1-carboxamide

A mixture of Example 1D (100 mg, 0.354 mmol), 2-methylquinoline-5-sulfonamide (91 mg, 0.407 mmol, prepared as in WO2018154519 A1), 1-(3-dimethylaminopropyl)-3-ethylcarbodiimide hydrochloride (136 mg, 0.708 mmol), and 4-dimethylaminopyridine (56.3 mg, 0.460 mmol) in dichloromethane (3.5 mL) was stirred at ambient temperature. After 4 hours, the reaction was acidified with trifluoroacetic acid (136 μL, 1.77 mmol) and concentrated under reduced pressure. The crude residue was then purified by reverse-phase HPLC (Waters Xbridge Prep C18 column, 42 mL/minute, 5-95% acetonitrile/0.1% trifluoroacetic acid in water) to afford the title compound (137 mg, 0.282 mmol, 79% yield). ¹H NMR (400 MHz, dimethyl sulfoxide-d₆) d 11.44 (s, 1H), 8.85 (d, J=8.9 Hz, 1H), 8.31-8.19 (m, 2H), 7.90 (dd, J=8.5, 7.4 Hz, 1H), 7.58 (d, J=8.9 Hz, 1H), 7.00-6.86 (m, 5H), 6.76-6.66 (m, 2H), 6.45 (d, J=8.2 Hz, 1H), 3.07 (s, 3H), 3.03 (dd, J=9.3, 7.3 Hz, 1H), 2.72 (s, 3H), 2.17 (s, 3H), 1.97 (dd, J=7.3, 5.4 Hz, 1H), 1.37 (dd, J=9.3, 5.4 Hz, 1H). MS(APCI+) m/z 487.2 (M+H)⁺.

### Example 2

### (1R,2S)-1-(2-methoxy-5-methylphenyl)-N-(2-methylquinoline-5-sulfonyl)-2-phenylcyclopropane-1-carboxamide

The enantiomers of Example 1 (100 mg, 0.206 mmol) were separated by preparative chiral supercritical fluid chromatography (ChiralPak IC column, 45% methanol/CO₂, 80 g/minute). The fractions containing the first eluting peak were concentrated under reduced pressure to afford the title compound (45.1 mg, 0.093 mmol, 45% yield). ¹H NMR (400 MHz, dimethyl sulfoxide-d₆) δ ppm 11.45 (s, 1H), 8.85 (d, J=8.9 Hz, 1H), 8.34-8.15 (m, 2H), 7.89 (dd, J=8.5, 7.4 Hz, 1H), 7.57 (d, J=8.9 Hz, 1H), 7.03-6.84 (m, 5H), 6.78-6.67 (m, 2H), 6.44 (d, J=8.3 Hz, 1H), 3.10-2.97 (m, 4H), 2.71 (s, 3H), 2.17 (s, 3H), 1.95 (s, 1H), 1.37 (dd, J=9.2, 5.3 Hz, 1H). MS(APCI+) m/z 487.2 (M+H)⁺.

### Example 3

### (1S,2R)-1-(2-methoxy-5-methylphenyl)-N-(2-methylquinoline-5-sulfonyl)-2-phenylcyclopropane-1-carboxamide

The enantiomers of Example 1 (100 mg, 0.206 mmol) were separated by preparative chiral supercritical fluid chromatography (ChiralPak IC column, 45% methanol/CO₂, 80 mL/minute). The fractions containing the second eluting peak were concentrated under reduced pressure to afford the title compound (45.1 mg, 0.093 mmol, 45% yield). ¹H NMR (500 MHz, dimethyl sulfoxide-d₆) δ ppm 11.48 (s, 1H), 8.85 (d, J=8.9 Hz, 1H), 8.29-8.17 (m, 2H), 7.89 (dd, J=8.5, 7.4 Hz, 1H), 7.57 (d, J=8.9 Hz, 1H), 7.01-6.83 (m, 5H), 6.76 -6.66 (m, 2H), 6.44 (d, J=8.2 Hz, 1H), 3.17 (s, 2H), 3.08-2.96 (m, 4H), 2.72 (s, 3H), 2.17 (s, 3H), 1.95 (s, 1H), 1.37 (dd, J=9.2, 5.3 Hz, 1H). MS(APCI+) m/z 487.2 (M+H)⁺.

### Example 4

### rac-(1r,2s)-1-(2-methoxy-5-methylphenyl)-2-(4-methylphenyl)-N-(2-methylquinoline-5-sulfonyl)cyclopropane-1-carboxamide

### Example 4A

### rac-(1r,2s)-methyl 1-(2-methoxy-5-methylphenyl)-2-(p-tolyl)cyclopropanecarboxylate

A solution of methyl 2-diazo-2-(2-methoxy-5-methylphenyl)acetate (100 mg, 0.454 mmol) in dichloromethane (1514 μL) was added via syringe pump to a solution of 4-methylstyrene (180 μL, 1.362 mmol) and rhodium (II) acetate dimer (1.003 mg, 2.270 μmol) in dichloromethane (3027 μL) at ambient temperature for 4 hours. LC/MS after stirring overnight showed two major peaks corresponding to product and likely excess styrene. The reaction was concentrated under reduced pressure, and the crude residue was purified via flash chromatography (ISCO CombiFlash, 0-30% ethyl acetate/heptanes, 12 g RediSep® gold silica column) to afford the title compound (79.1 mg, 0.255 mmol, 56.1% yield). MS(APCI+) m/z 311.4 (M+H)⁺.

### Example 4B

### rac-(1r,2s)-1-(2-methoxy-5-methylphenyl)-2-(p-tolyl)cyclopropanecarboxylic acid

Lithium hydroxide (61.0 mg, 2.55 mmol) was added to a solution of rac-(1r,2s)-methyl 1-(2-methoxy-5-methylphenyl)-2-(p-tolyl)cyclopropanecarboxylate (79.1 mg, 0.255 mmol) in dioxane (1359 μL) and H₂O (340 μL). The reaction mixture was heated to 80° C. LC/MS after heating overnight showed complete conversion. The reaction was acidified with 1 M HCl and extracted with ethyl acetate. The organic phase was dried with Na₂SO₄, filtered, and concentrated under reduced pressure to afford the title compound (64 mg, 0.216 mmol, 85% yield). ¹H NMR (400 MHz, dimethyl sulfoxide-d₆) δ ppm 12.01 (s, 1H), 6.92 (s, 1H), 6.83 (dd, J=8.3, 1.6 Hz, 1H), 6.79 (d, J=7.9 Hz, 2H), 6.66 (d, J=8.1 Hz, 2H), 6.48 (d, J=8.3 Hz, 1H), 3.27 (s, 3H), 3.01-2.89 (m, 1H), 2.11 (d, J=10.7 Hz, 6H), 1.84-1.72 (m, 1H), 1.69 (s, 1H). MS(APCI+) m/z 297.4 (M+H)⁺.

### Example 4C

### rac-(1r,2s)-1-(2-methoxy-5-methylphenyl)-2-(4-methylphenyl)-N-(2-methylquinoline-5-sulfonyl)cyclopropane-1-carboxamide

To a solution of rac-(1r,2s)-1-(2-methoxy-5-methylphenyl)-2-(p-tolyl)cyclopropanecarboxylic acid (64 mg, 0.216 mmol), 1-(3-dimethylaminopropyl)-3-ethylcarbodiimide hydrochloride (83 mg, 0.432 mmol), and 4-(dimethylamino)pyridine (4-dimethylaminopyridine) (31.7 mg, 0.259 mmol) in dry dichloromethane (1.0 mL) was added 2-methylquinoline-5-sulfonamide (50.4 mg, 0.227 mmol). The reaction mixture was stirred at ambient temperature overnight. LC/MS showed that the reaction was complete. The reaction mixture was quenched with 1.0M citric acid (4 mL), partitioned between ethyl acetate (20 mL) and water (10 mL), and the organic layer was washed with H₂O, and dried over Na₂SO₄. The solution was concentrated, and the residue was triturated with methanol to afford the title compound (43 mg). The supernatant was concentrated and purified via reverse-phase HPLC (5-95% acetonitrile/0.1% trifluoroacetic acid in water) to afford the title compound as second batch (48.5 mg) (total 91.5 mg, 0.183 mmol, 85% yield). ¹H NMR (600 MHz, dimethyl sulfoxide-d₆) δ ppm 11.42 (s, 1H), 8.84 (d, J=8.8 Hz, 1H), 8.24 (d, J=7.8 Hz, 2H), 7.89 (t, J=7.9 Hz, 1H), 7.57 (d, J=8.9 Hz, 1H), 6.94-6.88 (m, 2H), 6.78-6.73 (m, 2H), 6.63-6.58 (m, 2H), 6.47 (d, J=8.2 Hz, 1H), 3.17 (s, 1H), 3.08 (s, 3H), 2.98 (dd, J=9.2, 7.3 Hz, 1H), 2.71 (s, 3H), 2.54 (s, 1H), 2.53 (s, 1H), 2.17 (s, 3H), 2.08 (s, 3H). MS(APCI+) m/z 501.3 (M+H)⁺.

### Example 5

### rac-(1r,2s)-1-(2-methoxy-5-methylphenyl)-N-(2-methylquinoline-5-sulfonyl)-2-[4-(trifluoromethyl)phenyl]cyclopropane-1-carboxamide

The cyclopropane was prepared according to the procedure described in Example 4A by substituting 4-(trifluoromethyl)styrene for 4-methylstyrene, followed by processing as in Examples 4B and 4C to afford the title compound. ¹H NMR (400 MHz, dimethyl sulfoxide-d₆) δ ppm 11.51 (s, 1H), 8.81 (d, J=8.8 Hz, 1H), 8.28-8.16 (m, 2H), 7.87 (dd, J=8.5, 7.4 Hz, 1H), 7.55 (d, J=8.9 Hz, 1H), 7.25 (d, J=8.2 Hz, 2H), 6.97 (d, J=2.2 Hz, 1H), 6.89 (dd, J=8.5, 2.4 Hz, 2H), 6.40 (d, J=8.3 Hz, 1H), 3.10 (dd, J=9.1, 7.2 Hz, 1H), 2.99 (s, 3H), 2.69 (s, 3H), 2.16 (s, 3H), 2.08-2.01 (m, 1H), 1.41 (dd, J=9.2, 5.5 Hz, 1H). MS(APCI+) m/z 555.25 (M+H)⁺.

### Example 6

### (1R,2S)-1-(2-methoxy-5-methylphenyl)-2-(4-methylphenyl)-N-(2-methylquinoline-5-sulfonyl)cyclopropane-1-carboxamide

The racemic mixture of Example 4 (83.5 mg) was purified by preparative chiral supercritical fluid chromatography (ChiralPak IC column, 45% methanol/CO₂, 80 mL/minute). The fractions containing the first eluting peak were concentrated under reduced pressure to afford the title compound (31 mg, 0.062 mmol, 37% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.39 (s, 1H), 8.81 (d, J=8.9 Hz, 1H), 8.22-8.15 (m, 2H), 7.85 (t, J=8.1 Hz, 1H), 7.53 (d, J=8.9 Hz, 1H), 6.86 (d, J=9.0 Hz, 2H), 6.72 (d, J=7.8 Hz, 2H), 6.60-6.53 (m, 2H), 6.43 (d, J=8.2 Hz, 1H), 3.04 (s, 3H), 2.93 (t, J=8.2 Hz, 1H), 2.68 (s, 3H), 2.14 (s, 3H), 2.05 (s, 3H), 1.86 (s, 1H), 1.30 (dd, J=9.3, 5.2 Hz, 1H). MS(APCI+) m/z 501.3 (M+H)⁺.

### Example 7

### (1S,2R)-1-(2-methoxy-5-methylphenyl)-2-(4-methylphenyl)-N-(2-methylquinoline-5-sulfonyl)cyclopropane-1-carboxamide

The racemic mixture of Example 4 (83.5 mg) was purified by preparative chiral supercritical fluid chromatography (ChiralPak IC column, 45% methanol/CO₂, 80 mL/minute). The fractions containing the second eluting peak were concentrated under reduced pressure to afford the title compound (30 mg, 0.060 mmol, 36% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.37 (s, 1H), 8.81 (d, J=8.9 Hz, 1H), 8.20 (d, J=7.9 Hz, 2H), 7.85 (t, J=7.9 Hz, 1H), 7.53 (d, J=8.9 Hz, 1H), 6.88 (s, 2H), 6.86 (d, J=2.1 Hz, OH), 6.72 (d, J=7.9 Hz, 2H), 6.57 (d, J=7.9 Hz, 2H), 6.44 (d, J=8.2 Hz, 1H), 3.05 (s, 3H), 2.94 (t, J=8.3 Hz, 1H), 2.68 (s, 3H), 2.14 (s, 3H), 2.05 (s, 3H), 1.87 (s, 1H), 1.30 (dd, J=9.3, 5.3 Hz, 1H). MS(APCI+) m/z 501.3 (M+H)⁺.

### Example 8

### rac-(1r,2s)-2-(3-fluorophenyl)-1-(2-methoxy-5-methylphenyl)-N-(2-methylquinoline-5-sulfonyl)cyclopropane-1-carboxamide

The cyclopropane was prepared according to the procedure described in Example 4A by substituting 1-fluoro-3-vinylbenzene for 4-methylstyrene, followed by processing as in Examples 4B and 4C to afford the title compound. ¹H NMR (400 MHz, dimethyl sulfoxide-d₆) δ ppm 11.49 (s, 1H), 8.82 (d, J=8.9 Hz, 1H), 8.30-8.15 (m, 2H), 7.88 (dd, J=8.5, 7.4 Hz, 1H), 7.56 (d, J=8.9 Hz, 1H), 7.02-6.84 (m, 2H), 6.71 (td, J=8.5, 2.6 Hz, 1H), 6.58 (d, J=7.8 Hz, 1H), 6.49-6.37 (m, 2H), 3.04 (s, 3H), 2.69 (s, 3H), 2.16 (s, 3H), 2.02 (d, J=13.1 Hz, 1H), 1.33 (dd, J=9.2, 5.6 Hz, 1H). MS(APCI+) m/z 505.25 (M+H)⁺.

### Example 9

### rac-(1r,2s)-1-(2-methoxy-5-methylphenyl)-2-(4-methoxyphenyl)-N-(2-methylquinoline-5-sulfonyl)cyclopropane-1-carboxamide

The cyclopropane was prepared according to the procedure described in Example 4A by substituting 4-methoxystyrene for 4-methylstyrene, followed by processing as in Examples 4B and 4C to afford the title compound. ¹H NMR (500 MHz, dimethyl sulfoxide-d₆) δ ppm 11.45 (s, 1H), 8.91 (d, J=8.9 Hz, 1H), 8.32-8.26 (m, 2H), 7.94 (dd, J=8.5, 7.4 Hz, 1H), 7.64 (d, J=8.9 Hz, 1H), 6.93 (dt, J=7.0, 1.2 Hz, 2H), 6.69-6.65 (m, 2H), 6.55-6.49 (m, 3H), 3.59 (s, 3H), 3.15 (s, 3H), 3.01-2.97 (m, 1H), 2.18 (s, 3H), 1.94-1.41 (m, 1H), 1.33 (dd, J=9.2, 5.6 Hz, 1H). MS(APCI+) m/z 517.28 (M+H)⁺.

### Example 10

### (1R,2S)-1-(2-methoxy-5-methylphenyl)-2-(6-methylpyridin-3-yl)-N-(2-methylquinoline-5-sulfonyl)cyclopropane-1-carboxamide

### Example 10A

### ethyl 2-(2-methoxy-5-methylphenyl)-2-oxoacetate

1-Methoxy-4-methylbenzene (6.30 mL, 50 mmol, Aldrich) was added to a suspension of aluminum chloride (8.00 g, 60.0 mmol) in dichloromethane (100 mL) at 0° C. After stirring for 10 minutes, ethyl 2-chloro-2-oxoacetate (6.70 mL, 60.0 mmol, Aldrich) was added dropwise, and the reaction was allowed to slowly warm to ambient temperature. After 16 hours, the reaction was quenched with 1 M hydrochloric acid (200 mL) and vigorously stirred for 15 minutes. The layers were then separated and the organic phase was washed with saturated NaHCO₃ and brine, dried with MgSO₄, filtered through a pad of diatomaceous earth, and concentrated under reduced pressure to afford the title compound (10.9 g, 49.0 mmol, 98% yield). ¹H NMR (400 MHz, CDCl₃) δ ppm 7.67 (d, J=2.4 Hz, 1H), 7.38 (dd, J=8.4, 2.4 Hz, 1H), 6.88 (d, J=8.4 Hz, 1H), 4.38 (q, J=7.1 Hz, 2H), 3.84 (s, 3H), 2.32 (s, 3H), 1.39 (t, J=7.1 Hz, 3H). MS(APCI+) m/z 223.6 (M+H)⁺.

### Example 10B

### ethyl 2-diazo-2-(2-methoxy-5-methylphenyl)acetate

4-Methylbenzenesulfonohydrazide (8.72 g, 46.8 mmol, Aldrich) was added to a solution of Example 10A (10.4 g, 46.8 mmol) in toluene (100 mL), and the reaction mixture was heated to reflux with a Dean-Stark trap. After 16 hours, the reaction was concentrated under reduced pressure and dichloromethane (100 mL) and triethylamine (7.83 mL, 56.2 mmol) were added to the resulting residue. After stirring at ambient temperature for 72 hours, the reaction was concentrated under reduced pressure and the crude residue was purified by flash chromatography (ISCO CombiFlash, 0-20% ethyl acetate/heptanes, 220 g RediSep® gold silica column) to afford the title compound (7.1 g, 30.3 mmol, 64.8% yield). ¹H NMR (500 MHz, CDCl₃) δ ppm 7.37 (d, J=2.2 Hz, 1H), 7.04 (ddq, J=8.4, 2.2, 0.7 Hz, 1H), 6.79 (d, J=8.4 Hz, 1H), 4.30 (q, J=7.1 Hz, 2H), 3.82 (s, 3H), 2.30 (d, J=0.8 Hz, 3H), 1.32 (t, J=7.1 Hz, 3H).

### Example 10C

### rac-(1r,2s)-ethyl 1-(2-methoxy-5-methylphenyl)-2-(6-methylpyridin-3-yl)cyclopropanecarboxylate

A solution of Example 10B (256 mg, 1.094 mmol) and 2-methyl-5-vinylpyridine (652 mg, 5.47 mmol, CombiBlocks) in dichloromethane (1.0 mL) was irradiated with blue light (Kessil lamp, 34 W) at ambient temperature. After 3 hours, the reaction was concentrated under reduced pressure and the crude residue was purified by flash chromatography (ISCO CombiFlash, 40-80% ethyl acetate/heptanes, 40 g RediSep® gold silica column) to afford the title compound (146 mg, 0.449 mmol, 41.0% yield). ¹H NMR (400 MHz, dimethyl sulfoxide-d₆) δ ppm 8.03 (d, J=2.4 Hz, 1H), 7.01 (d, J=2.3 Hz, 1H), 6.95 (dd, J=8.0, 2.4 Hz, 1H), 6.92-6.87 (m, 1H), 6.84 (d, J=8.1 Hz, 1H), 6.53 (d, J=8.3 Hz, 1H), 4.10 (dq, J=10.8, 7.1 Hz, 1H), 3.95 (dq, J=10.8, 7.1 Hz, 1H), 3.36 (s, 3H), 3.07 (dd, J=9.2, 7.2 Hz, 1H), 2.27 (s, 3H), 2.19 (s, 3H), 2.05 (dd, J=7.2, 5.2 Hz, 1H), 1.73 (dd, J=9.3, 5.2 Hz, 1H), 1.06 (t, J=7.1 Hz, 3H). MS(APCI+) m/z 326.5 (M+H)⁺.

### Example 10D

### (1R,2S)-ethyl 1-(2-methoxy-5-methylphenyl)-2-(6-methylpyridin-3-yl)cyclopropanecarboxylate

The enantiomers of Example 10C (152 mg, 0.47 mmol) were separated by preparative chiral supercritical fluid chromatography (ChiralCel OD-H column, 14% methanol/CO₂, 49 g/minute). The fractions containing the first eluting peak were concentrated under reduced pressure to afford the title compound (52.2 mg, 0.160 mmol, 34% yield). ¹H NMR (400 MHz, dimethyl sulfoxide-d₆) δ ppm 8.03 (d, J=2.4 Hz, 1H), 7.01 (d, J=2.3 Hz, 1H), 6.95 (dd, J=8.0, 2.4 Hz, 1H), 6.92-6.87 (m, 1H), 6.84 (d, J=8.1 Hz, 1H), 6.53 (d, J=8.3 Hz, 1H), 4.10 (dq, J=10.8, 7.1 Hz, 1H), 3.95 (dq, J=10.8, 7.1 Hz, 1H), 3.36 (s, 3H), 3.07 (dd, J=9.2, 7.2 Hz, 1H), 2.27 (s, 3H), 2.19 (s, 3H), 2.05 (dd, J=7.2, 5.2 Hz, 1H), 1.73 (dd, J=9.3, 5.2 Hz, 1H), 1.06 (t, J=7.1 Hz, 3H). MS(APCI+) m/z 326.5 (M+H)⁺.

### Example 10E

### (1R,2S)-1-(2-methoxy-5-methylphenyl)-2-(6-methylpyridin-3-yl)-N-(2-methylquinoline-5-sulfonyl)cyclopropane-1-carboxamide

Sodium hydroxide (42 μL, 0.80 mmol, 50% in water) was added to a solution of Example 10D (52.2 mg, 0.160 mmol) in methanol (0.80 mL) and the reaction was heated to 70° C. After three hours, the reaction was concentrated under reduced pressure. 4 M HCl in dioxane (1 mL, 4 mmol) was added to the resulting residue, and the mixture was again concentrated under reduced pressure. 2-Methylquinoline-5-sulfonamide (44.5 mg, 0.200 mmol), 1-(3-dimethylaminopropyl)-3-ethylcarbodiimide hydrochloride (61.3 mg, 0.320 mmol), 4-dimethylaminopyridine (24.43 mg, 0.200 mmol), N,N-diisopropylethylamine (55.9 μL, 0.320 mmol), and dichloromethane (1.6 mL) were added to the crude acid residue. After stirring at ambient temperature for 16 hours, the reaction was acidified with trifluoroacetic acid (123 μL, 1.600 mmol) and concentrated under reduced pressure. The crude residue was purified by reverse-phase HPLC (Waters Xbridge Prep C18 column, 42 mL/minute, 5-50% acetonitrile/0.1% trifluoroacetic acid in water) to afford the title compound (81 mg, 0.132 mmol, 82% yield). ¹H NMR (500 MHz, dimethyl sulfoxide-d₆) δ ppm 8.87 (d, J=8.9 Hz, 1H), 8.32-8.20 (m, 3H), 7.94 (dd, J=8.5, 7.4 Hz, 1H), 7.73 (dd, J=8.4, 2.1 Hz, 1H), 7.62 (d, J=8.9 Hz, 1H), 7.47 (d, J=8.4 Hz, 1H), 7.11 (d, J=2.2 Hz, 1H), 6.99 (ddd, J=8.2, 2.2, 0.9 Hz, 1H), 6.44 (d, J=8.3 Hz, 1H), 3.27 (dd, J=9.1, 7.1 Hz, 1H), 3.05 (s, 3H), 2.74 (s, 3H), 2.49 (s, 3H), 2.34-2.28 (m, 1H), 2.24 (s, 3H), 1.56 (dd, J=9.1, 5.8 Hz, 1H). MS(APCI+) m/z 502.5 (M+H)⁺.

### Example 11

### (1S,2R)-1-(2-methoxy-5-methylphenyl)-2-(6-methylpyridin-3-yl)-N-(2-methylquinoline-5-sulfonyl)cyclopropane-1-carboxamide

### Example 11A

### (1S,2R)-ethyl 1-(2-methoxy-5-methylphenyl)-2-(6-methylpyridin-3-yl)cyclopropanecarboxylate

The enantiomers of Example 10C (152 mg, 0.47 mmol) were separated by preparative chiral supercritical fluid chromatography (ChiralCel OD-H column, 14% methanol/CO₂, 49 g/minute). The fractions containing the second eluting peak were concentrated under reduced pressure to afford the title compound (59.7 mg, 0.182 mmol, 39% yield). ¹H NMR (400 MHz, dimethyl sulfoxide-d₆) δ ppm 8.03 (d, J=2.4 Hz, 1H), 7.01 (d, J=2.3 Hz, 1H), 6.95 (dd, J=8.0, 2.4 Hz, 1H), 6.92-6.87 (m, 1H), 6.84 (d, J=8.1 Hz, 1H), 6.53 (d, J=8.3 Hz, 1H), 4.10 (dq, J=10.8, 7.1 Hz, 1H), 3.95 (dq, J=10.8, 7.1 Hz, 1H), 3.36 (s, 3H), 3.07 (dd, J=9.2, 7.2 Hz, 1H), 2.27 (s, 3H), 2.19 (s, 3H), 2.05 (dd, J=7.2, 5.2 Hz, 1H), 1.73 (dd, J=9.3, 5.2 Hz, 1H), 1.06 (t, J=7.1 Hz, 3H). MS(APCI+) m/z 326.5 (M+H)⁺.

### Example 11B

### (1S,2R)-1-(2-methoxy-5-methylphenyl)-2-(6-methylpyridin-3-yl)-N-(2-methylquinoline-5-sulfonyl)cyclopropane-1-carboxamide

Sodium hydroxide (48 μL, 0.91 mmol, 50% in water) was added to a solution of Example 11A (59 mg, 0.181 mmol) in methanol (0.91 mL) and the reaction was heated to 70° C. After heating for three hours, the reaction was concentrated under reduced pressure. 4 M HCl in dioxane (1 mL, 4 mmol) was added to the resulting residue, and the mixture was again concentrated under reduced pressure. 2-Methylquinoline-5-sulfonamide (50.0 mg, 0.225 mmol), 1-(3-dimethylaminopropyl)-3-ethylcarbodiimide hydrochloride (69.0 mg, 0.360 mmol), 4-dimethylaminopyridine (27.5 mg, 0.225 mmol), N,N-diisopropylethylamine (62.9 μL, 0.360 mmol) and dichloromethane (1.8 mL) were added to the crude acid residue. After stirring at ambient temperature for 16 hours, the reaction was acidified with trifluoroacetic acid (139 μL, 1.800 mmol) and concentrated under reduced pressure. The crude residue was purified by reverse-phase HPLC (Waters Xbridge Prep C18 column, 42 mL/minute, 5-50% acetonitrile/0.1% trifluoroacetic acid in water) to afford the title compound (47 mg, 0.076 mmol, 42.4% yield). ¹H NMR (400 MHz, dimethyl sulfoxide-d₆) δ ppm 8.89 (d, J=8.9 Hz, 1H), 8.36-8.23 (m, 3H), 7.95 (dd, J=8.5, 7.4 Hz, 1H), 7.74 (dd, J=8.4, 2.2 Hz, 1H), 7.63 (d, J=8.9 Hz, 1H), 7.48 (d, J=8.4 Hz, 1H), 7.11 (d, J=2.4 Hz, 1H), 6.99 (dd, J=8.5, 2.2 Hz, 1H), 6.44 (d, J=8.3 Hz, 1H), 3.28 (dd, J=9.2, 7.1 Hz, 1H), 3.06 (s, 3H), 2.75 (s, 3H), 2.31 (t, J=6.5 Hz, 1H), 2.24 (s, 3H), 1.57 (dd, J=9.1, 5.8 Hz, 1H). MS(APCI+) m/z 502.5 (M+H)⁺.

### Example 12

### rac-(1r,2s)-1-(2-methoxy-5-methylphenyl)-N-(2-methylquinoline-5-sulfonyl)-2-[3-(trifluoromethyl)phenyl]cyclopropane-1-carboxamide

The cyclopropane was prepared according to the procedure described in Example 4A by substituting 3-(trifluoromethyl)styrene for 4-methylstyrene, followed by processing as in Examples 4B and 4C to afford the title compound. ¹H NMR (400 MHz, dimethyl sulfoxide-d₆) δ ppm 11.58 (s, 1H), 8.83 (d, J=8.8 Hz, 1H), 8.23 (dt, J=8.3, 2.2 Hz, 2H), 7.96-7.81 (m, 1H), 7.56 (d, J=8.9 Hz, 1H), 7.23 (d, J=7.8 Hz, 1H), 7.12 (t, J=7.7 Hz, 1H), 7.06-6.90 (m, 3H), 6.86 (dd, J=8.4, 2.2 Hz, 1H), 6.35 (d, J=8.4 Hz, 1H), 3.14 (dd, J=9.2, 7.2 Hz, 1H), 2.96 (s, 3H), 2.69 (s, 3H), 2.15 (m, 4H), 1.38 (dd, J=9.2, 5.7 Hz, 1H). MS(APCI+) m/z 555.25 (M+H)⁺.

### Example 13

### (1R,2R)-1-(2-methoxy-5-methylphenyl)-N-(2-methylquinoline-5-sulfonyl)-2-(pyridin-2-yl)cyclopropane-1-carboxamide

### Example 13A

### rac-(1r,2r)-methyl 1-(2-methoxy-5-methylphenyl)-2-(pyridin-2-yl)cyclopropanecarboxylate

A solution of Example 1B (150 mg, 0.681 mmol) and 2-vinylpyridine (367 μL, 3.41 mmol, Aldrich) in dichloromethane (6.8 mL) was irradiated with blue light (Kessil lamp, 34 W) at ambient temperature. After 4 hours, the reaction was concentrated under reduced pressure and purified by flash chromatography (ISCO CombiFlash, 0-100% ethyl acetate/heptanes, 40 g RediSep® gold silica column) to afford the title compound (122 mg, 0.410 mmol, 60.2% yield). ¹H NMR (400 MHz, dimethyl sulfoxide-d₆) δ ppm 8.10 (ddd, J=4.8, 1.8, 0.9 Hz, 1H), 7.47 (td, J=7.7, 1.8 Hz, 1H), 7.04 (dt, J=7.9, 1.1 Hz, 1H), 6.97 (ddd, J=7.5, 4.9, 1.2 Hz, 1H), 6.94-6.88 (m, 1H), 6.88-6.81 (m, 1H), 6.48 (d, J=8.2 Hz, 1H), 3.28 (s, 3H), 3.24 (dd, J=8.9, 7.1 Hz, 1H), 2.25 (dd, J=7.1, 4.3 Hz, 1H), 2.14 (s, 3H), 1.78 (dd, J=8.9, 4.3 Hz, 1H). MS(APCI+) m/z 298.4 (M+H)⁺.

### Example 13B

### (1R,2R)-methyl 1-(2-methoxy-5-methylphenyl)-2-(pyridin-2-yl)cyclopropanecarboxylate

The enantiomers of Example 13A (119 mg, 0.400 mmol) were separated by preparative chiral supercritical fluid chromatography (ChiralPak IC column, 20% methanol/CO₂, 70 g/minute). The fractions containing the first eluting peak were concentrated under reduced pressure to afford the title compound (44.1 mg, 0.148 mmol, 37% yield). ¹H NMR (400 MHz, dimethyl sulfoxide-d₆) δ ppm 8.10 (ddd, J=4.8, 1.8, 0.9 Hz, 1H), 7.47 (td, J=7.7, 1.8 Hz, 1H), 7.04 (dt, J=7.9, 1.1 Hz, 1H), 6.97 (ddd, J=7.5, 4.9, 1.2 Hz, 1H), 6.94-6.88 (m, 1H), 6.88-6.81 (m, 1H), 6.48 (d, J=8.2 Hz, 1H), 3.28 (s, 3H), 3.24 (dd, J=8.9, 7.1 Hz, 1H), 2.25 (dd, J=7.1, 4.3 Hz, 1H), 2.14 (s, 3H), 1.78 (dd, J=8.9, 4.3 Hz, 1H). MS(APCI+) m/z 298.4 (M+H)⁺.

### Example 13C

### (1R,2R)-1-(2-methoxy-5-methylphenyl)-N-(2-methylquinoline-5-sulfonyl)-2-(pyridin-2-yl)cyclopropane-1-carboxamide

A mixture of Example 13B (44.1 mg, 0.148 mmol) and lithium hydroxide (17.8 mg, 0.742 mmol) in dioxane (0.74 mL) and water (0.25 mL) was heated to 80° C. After 4 hours, the reaction was acidified with 4 M HCl in dioxane (1 mL, 4 mmol) and concentrated under reduced pressure. 2-Methylquinoline-5-sulfonamide (41.7 mg, 0.188 mmol), 1-(3-dimethylaminopropyl)-3-ethylcarbodiimide hydrochloride (57.5 mg, 0.300 mmol), 4-dimethylaminopyridine (22.91 mg, 0.188 mmol), N,N-diisopropylethylamine (52.4 μL, 0.300 mmol), and dichloromethane (1.5 mL) were added to the crude acid residue. After stirring at ambient temperature for 16 hours, the reaction was acidified with trifluoroacetic acid (116 μL, 1.500 mmol) and concentrated under reduced pressure. The crude residue was purified by reverse-phase HPLC (Waters Xbridge Prep C18 column, 42 mL/minute, 5-40% acetonitrile/0.1% trifluoroacetic acid in water). The product containing fractions were concentrated, and the resulting residue was further purified by reverse-phase HPLC (Waters Xbridge Prep C18 column, 42 mL/minute, 5-95% acetonitrile/25 mM NH₄HCO₃) to afford the title compound (17 mg, 0.035 mmol, 23.24% yield). ¹H NMR (500 MHz, dimethyl sulfoxide-d₆) δ ppm 8.85 (d, J=8.8 Hz, 1H), 8.17 (dd, J=7.9, 3.7 Hz, 2H), 8.11-8.06 (m, 1H), 7.84 (dd, J=8.4, 7.4 Hz, 1H), 7.53 (d, J=8.9 Hz, 1H), 7.44 (td, J=7.7, 1.8 Hz, 1H), 6.97 (ddd, J=7.5, 4.9, 1.2 Hz, 1H), 6.90-6.82 (m, 3H), 6.40 (d, J=8.3 Hz, 1H), 3.17 (dd, J=8.8, 6.9 Hz, 1H), 3.04 (s, 3H), 2.70 (s, 3H), 2.16 (s, 3H), 2.12 (s, 1H), 1.47 (dd, J=8.8, 4.4 Hz, 1H). MS(APCI+) m/z 488.3 (M+H)⁺.

### Example 14

### (1S,2S)-1-(2-methoxy-5-methylphenyl)-N-(2-methylquinoline-5-sulfonyl)-2-(pyridin-2-yl)cyclopropane-1-carboxamide Example 14A (1S,2S)-methyl 1-(2-methoxy-5-methylphenyl)-2-(pyridin-2-yl)cyclopropanecarboxylate

The enantiomers of Example 13A (119 mg, 0.400 mmol) were separated by preparative chiral supercritical fluid chromatography (ChiralPak IC column, 20% methanol/CO₂, 70 g/minute). The fractions containing the second eluting peak were concentrated under reduced pressure to afford the title compound (41.6 mg, 0.14 mmol, 35% yield). ¹H NMR (400 MHz, dimethyl sulfoxide-d₆) δ ppm 8.10 (ddd, J=4.8, 1.8, 0.9 Hz, 1H), 7.47 (td, J=7.7, 1.8 Hz, 1H), 7.04 (dt, J=7.9, 1.1 Hz, 1H), 6.97 (ddd, J=7.5, 4.9, 1.2 Hz, 1H), 6.94-6.88 (m, 1H), 6.88-6.81 (m, 1H), 6.48 (d, J=8.2 Hz, 1H), 3.28 (s, 3H), 3.24 (dd, J=8.9, 7.1 Hz, 1H), 2.25 (dd, J=7.1, 4.3 Hz, 1H), 2.14 (s, 3H), 1.78 (dd, J=8.9, 4.3 Hz, 1H). MS(APCI+) m/z 298.4 (M+H)⁺.

### Example 14B

### (1S,2S)-1-(2-methoxy-5-methylphenyl)-N-(2-methylquinoline-5-sulfonyl)-2-(pyridin-2-yl)cyclopropane-1-carboxamide

A mixture of Example 14A (41.6 mg, 0.140 mmol) and lithium hydroxide (16.8 mg, 0.70 mmol) in dioxane (0.70 mL) and water (0.23 mL) was heated to 80° C. After 4 hours, the reaction was acidified with 4 M HCl in dioxane (1 mL, 4 mmol) and concentrated under reduced pressure. 2-Methylquinoline-5-sulfonamide (38.9 mg, 0.175 mmol), 1-(3-dimethylaminopropyl)-3-ethylcarbodiimide hydrochloride (53.7 mg, 0.280 mmol), 4-dimethylaminopyridine (21.4 mg, 0.175 mmol), N,N-diisopropylethylamine (49 μL, 0.28 mmol), and dichloromethane (1.4 mL) were added to the crude acid residue. After stirring at ambient temperature for 16 hours the reaction was acidified with trifluoroacetic acid (116 μL, 1.500 mmol) and concentrated under reduced pressure. The crude residue was purified by reverse-phase HPLC (Waters Xbridge Prep C18 column, 42 mL/minute, 5-40% acetonitrile/0.1% trifluoroacetic acid in water). The product containing fractions were concentrated and the resulting residue was further purified by reverse-phase HPLC (Waters Xbridge Prep C18 column, 42 mL/minute, 5-95% acetonitrile/25 mM NH₄HCO₃) to afford the title compound (40 mg, 0.082 mmol, 58.6% yield). ¹H NMR (400 MHz, dimethyl sulfoxide-d₆) d 8.85 (d, J=8.8 Hz, 1H), 8.17 (dd, J=7.8, 3.3 Hz, 2H), 8.09 (dd, J=5.2, 1.8 Hz, 1H), 7.84 (dd, J=8.5, 7.4 Hz, 1H), 7.53 (d, J=8.9 Hz, 1H), 7.43 (td, J=7.7, 1.8 Hz, 1H), 6.96 (ddd, J=7.6, 4.9, 1.1 Hz, 1H), 6.91-6.78 (m, 3H), 6.40 (d, J=8.3 Hz, 1H), 3.17 (dd, J=8.8, 6.9 Hz, 1H), 3.04 (s, 3H), 2.70 (s, 3H), 2.16 (s, 4H), 1.47 (dd, J=8.9, 4.4 Hz, 1H). MS(APCI+) m/z 488.4 (M+H)⁺.

### Example 15

### rac-(1r,2s)-1-(2-methoxy-5-methylphenyl)-2-(3-methoxyphenyl)-N-(2-methylquinoline-5-sulfonyl)cyclopropane-1-carboxamide

The cyclopropane was prepared according to the procedure described in Example 4A by substituting 3-methoxystyrene for 4-methylstyrene, followed by processing as in Examples 4B and 4C to afford the title compound. ¹H NMR (400 MHz, dimethyl sulfoxide-d₆) δ ppm 11.45 (s, 1H), 8.81 (d, J=8.9 Hz, 1H), 8.21 (d, J=7.8 Hz, 2H), 7.86 (dd, J=8.5, 7.4 Hz, 1H), 7.54 (d, J=8.9 Hz, 1H), 6.94 (d, J=2.0 Hz, 1H), 6.89 (dd, J=8.4, 2.2 Hz, 1H), 6.82 (t, J=7.9 Hz, 1H), 6.49-6.39 (m, 2H), 6.39-6.29 (m, 1H), 6.14 (t, J=2.1 Hz, 1H), 3.43 (s, 2H), 3.13 (s, 2H), 3.03 (s, 3H), 2.98 (dd, J=9.2, 7.2 Hz, 1H), 2.68 (s, 3H), 2.15 (s, 3H), 1.95 (d, J=7.0 Hz, 1H), 1.31 (dd, J=9.3, 5.4 Hz, 1H). MS(APCI+) m/z 517 (M+H)⁺.

### Example 16

### (1R,2S)-1-(2-methoxy-5-methylphenyl)-2-(4-methoxyphenyl)-N-(2-methylquinoline-5-sulfonyl)cyclopropane-1-carboxamide

The enantiomers of Example 9 (81 mg) were separated by preparative chiral supercritical fluid chromatography (ChiralPak IC column, 40% methanol/CO₂, 80 mL/minute). The fractions containing the first eluting peak were concentrated under reduced pressure to afford the title compound (34 mg, 0.065 mmol, 42% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.37 (s, 1H), 8.81 (d, J=8.9 Hz, 1H), 8.19 (d, J=7.5 Hz, 2H), 7.85 (t, J=7.7 Hz, 1H), 7.53 (d, J=8.9 Hz, 1H), 6.87 (d, J=6.5 Hz, 2H), 6.64-6.57 (m, 2H), 6.52-6.41 (m, 3H), 3.53 (s, 3H), 3.07 (s, 3H), 2.93 (t, J=8.2 Hz, 1H), 2.68 (s, 3H), 2.14 (s, 3H), 1.84 (s, 1H), 1.30 (dd, J=9.3, 5.2 Hz, 1H). MS(APCI+) m/z 517.3 (M+H)⁺.

### Example 17

### (1S,2R)-1-(2-methoxy-5-methylphenyl)-2-(4-methoxyphenyl)-N-(2-methylquinoline-5-sulfonyl)cyclopropane-1-carboxamide

The enantiomers of Example 9 (81 mg) were separated by preparative chiral supercritical fluid chromatography (ChiralPak IC column, 40% methanol/CO₂, 80 mL/minute). The fractions containing the second eluting peak were concentrated under reduced pressure to afford the title compound (33 mg, 0.064 mmol, 41% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.36 (s, 1H), 8.82 (d, J=8.9 Hz, 1H), 8.22 (d, J=7.9 Hz, 2H), 7.92-7.83 (m, 1H), 7.56 (d, J=8.9 Hz, 1H), 6.88 (d, J=6.9 Hz, 2H), 6.65-6.57 (m, 2H), 6.52-6.42 (m, 3H), 3.53 (s, 3H), 3.08 (s, 3H), 2.94 (dd, J=9.4, 7.3 Hz, 1H), 2.69 (s, 3H), 2.14 (s, 3H), 1.86 (dd, J=7.4, 5.3 Hz, 1H), 1.29 (dd, J=9.3, 5.3 Hz, 1H). MS(APCI+) m/z 517.3 (M+H)⁺.

### Example 18

### (1S,2R)-1-(2-methoxy-5-methylphenyl)-N-(2-methylquinoline-5-sulfonyl)-2-[4-(trifluoromethyl)phenyl]cyclopropane-1-carboxamide

The enantiomers of Example 5 (101 mg) were separated by preparative chiral supercritical fluid chromatography (ChiralPak IC column, 40% methanol/CO₂, 80 mL/minute). The fractions containing the first eluting peak were concentrated under reduced pressure to afford the title compound (45 mg, 0.081 mmol, 45% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.34 (s, 1H), 8.82 (d, J=8.9 Hz, 1H), 8.22 (d, J=7.9 Hz, 2H), 7.92-7.83 (m, 1H), 7.56 (d, J=8.9 Hz, 1H), 6.88 (d, J=6.9 Hz, 2H), 6.65-6.57 (m, 2H), 6.52-6.42 (m, 3H), 3.53 (s, 3H), 3.08 (s, 3H), 2.94 (dd, J=9.4, 7.3 Hz, 1H), 2.69 (s, 3H), 2.14 (s, 3H), 1.86 (dd, J=7.4, 5.3 Hz, 1H), 1.29 (dd, J=9.3, 5.3 Hz, 1H). MS(APCI+) m/z 555.3 (M+H)⁺.

### Example 19

### (1R,2S)-1-(2-methoxy-5-methylphenyl)-N-(2-methylquinoline-5-sulfonyl)-2-[4-(trifluoromethyl)phenyl]cyclopropane-1-carboxamide

The enantiomers of Example 5 (101 mg) were separated by preparative chiral supercritical fluid chromatography (ChiralPak IC column, 40% methanol/CO₂, 80 mL/minute). The fractions containing the second eluting peak were concentrated under reduced pressure to afford the title compound (34 mg, 0.081 mmol, 34% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.37 (s, 1H), 8.82 (d, J=8.9 Hz, 1H), 8.22 (d, J=7.9 Hz, 2H), 7.92-7.83 (m, 1H), 7.56 (d, J=8.9 Hz, 1H), 6.88 (d, J=6.9 Hz, 2H), 6.65-6.57 (m, 2H), 6.52-6.42 (m, 3H), 3.53 (s, 3H), 3.09 (s, 3H), 2.94 (dd, J=9.4, 7.3 Hz, 1H), 2.69 (s, 3H), 2.14 (s, 3H), 1.86 (dd, J=7.4, 5.3 Hz, 1H), 1.29 (dd, J=9.3, 5.3 Hz, 1H). MS(APCI+) m/z 555.3 (M+H)⁺.

### Example 20

### (1R,2S)-2-(3-fluorophenyl)-1-(2-methoxy-5-methylphenyl)-N-(2-methylquinoline-5-sulfonyl)cyclopropane-1-carboxamide

The cyclopropane was prepared according to the procedure described in Example 4A by substituting 3-fluorostyene for 4-methylstyrene, followed by processing as in Examples 4B and 4C to afford the racemate of the title compound (78 mg). The enantiomers were separated by preparative chiral supercritical fluid chromatography (ChiralPak IC column, 40% methanol/CO₂, 80 mL/minute). The fractions containing the first eluting peak were concentrated under reduced pressure to afford the title compound (28 mg, 0.056 mmol, 36% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.49 (s, 1H), 8.81 (d, J=8.8 Hz, 1H), 8.19 (s, 2H), 7.85 (d, J=8.4 Hz, 1H), 7.53 (d, J=8.9 Hz, 1H), 7.00-6.85 (m, 3H), 6.70 (td, J=8.4, 2.4 Hz, 1H), 6.57 (d, J=7.7 Hz, 1H), 6.43 (d, J=8.3 Hz, 2H), 3.13 (s, 1H), 3.03 (s, 3H), 2.68 (s, 3H), 2.16 (s, 3H), 1.97 (s, 1H), 1.34 (dd, J=9.2, 5.5 Hz, 1H). MS(APCI+) m/z 505.3 (M+H)⁺.

### Example 21

### (1S,2R)-2-(3-fluorophenyl)-1-(2-methoxy-5-methylphenyl)-N-(2-methylquinoline-5-sulfonyl)cyclopropane-1-carboxamide

The cyclopropane was prepared according to the procedure described in Example 4A by substituting 3-fluorostyene for 4-methylstyrene, followed by processing as in Examples 4B and 4C to afford the racemate of the title compound (78 mg). The enantiomers were separated by preparative chiral supercritical fluid chromatography (ChiralPak IC column, 40% methanol/CO₂, 80 mL/minute). The fractions containing the second eluting peak were concentrated under reduced pressure to afford the title compound (26 mg, 0.051 mmol, 33% yield). The material was determined to be >98% ee by analytical supercritical fluid chromatography (ChiralPak IC column, 3 mL/minute, 5-50% methanol/CO₂). ¹H NMR (500 MHz, dimethyl sulfoxide-d₆) δ ppm 11.56 (s, 1H), 8.85 (d, J=8.9 Hz, 1H), 8.26 (d, J=7.9 Hz, 2H), 7.91 (t, J=7.9 Hz, 1H), 7.59 (d, J=8.9 Hz, 1H), 7.04-6.91 (m, 3H), 6.80-6.72 (m, 1H), 6.66-6.60 (m, 1H), 6.51-6.44 (m, 2H), 3.18 (s, 2H), 3.08 (d, J=16.2 Hz, 1H), 2.73 (s, 3H), 2.21 (s, 3H), 2.04 (s, 1H). MS(APCI+) m/z 505.3 (M+H)⁺.

### Example 22

### rac-(1r,2r)-2-(2-fluorophenyl)-1-(2-methoxy-5-methylphenyl)-N-(2-methylquinoline-5-sulfonyl)cyclopropane-1-carboxamide

The cyclopropane was prepared according to the procedure described in Example 4A by substituting 1-fluoro-2-vinylbenzene for 4-methylstyrene, followed by processing as in Examples 4B and 4C to afford the title compound. ¹H NMR (400 MHz, dimethyl sulfoxide-d₆) δ ppm 11.42 (s, 1H), 8.85 (d, J=8.9 Hz, 1H), 8.24 (dd, J=7.7, 2.2 Hz, 2H), 7.90 (dd, J=8.5, 7.4 Hz, 1H), 7.58 (d, J=8.9 Hz, 1H), 7.00-6.81 (m, 3H), 6.61 (td, J=7.4, 1.5 Hz, 1H), 6.41 (d, J=8.2 Hz, 1H), 6.32 (td, J=7.9, 1.7 Hz, 1H), 3.17-3.07 (m, 1H), 3.04 (s, 3H), 2.70 (s, 3H), 2.15 (s, 3H), 2.04 (dd, J=7.4, 5.5 Hz, 1H), 1.40 (dd, J=9.3, 5.5 Hz, 1H). MS(APCI+) m/z 505.3 (M+H)⁺.

### Example 23

### (1S,2R)-1-(2-methoxy-5-methylphenyl)-N-(2-methylquinoline-5-sulfonyl)-2-[3-(trifluoromethyl)phenyl]cyclopropane-1-carboxamide

The enantiomers of Example 12 (49 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (16 mg, 0.03 mmol, 34% yield). ¹H NMR (600 MHz, dimethyl sulfoxide-d₆) δ ppm 11.62 (s, 1H), 8.85 (d, J=8.8 Hz, 1H), 8.23 (s, 2H), 7.89 (s, 1H), 7.56 (d, J=8.9 Hz, 1H), 7.26 (d, J=7.6 Hz, 1H), 7.15 (t, J=7.8 Hz, 1H), 7.01 (s, 2H), 6.97 (s, 1H), 6.88 (d, J=8.4 Hz, 1H), 6.38 (d, J=8.2 Hz, 1H), 3.16 (s, 1H), 2.98 (s, 3H), 2.71 (s, 3H), 2.18 (s, 3H), 1.42 (dd, J=9.1, 5.6 Hz, 1H). MS(APCI+) m/z 555.3 (M+H)⁺.

### Example 24

### (1R,2S)-1-(2-methoxy-5-methylphenyl)-N-(2-methylquinoline-5-sulfonyl)-2-[3-(trifluoromethyl)phenyl]cyclopropane-1-carboxamide

The enantiomers of Example 12 (49 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (10 mg, 0.018 mmol, 21% yield). ¹H NMR (600 MHz, dimethyl sulfoxide-d₆) δ ppm 11.63 (s, 1H), 8.87 (d, J=8.7 Hz, 1H), 8.19 (s, 2H), 7.85 (s, 1H), 7.54 (d, J=8.8 Hz, 1H), 7.27-7.23 (m, 1H), 7.15 (t, J=7.8 Hz, 1H), 7.00 (d, J=8.9 Hz, 2H), 6.95 (s, 1H), 6.86 (d, J=8.0 Hz, 1H), 6.36 (d, J=8.2 Hz, 1H), 3.13 (s, 1H), 2.96 (s, 3H), 2.69 (s, 3H), 2.17 (s, 3H), 2.06 (s, 1H), 1.44 (d, J=8.3 Hz, 1H). MS(APCI+) m/z 555.3 (M+H)⁺.

### Example 25

### (1S,2S)-2-(2-fluorophenyl)-1-(2-methoxy-5-methylphenyl)-N-(2-methylquinoline-5-sulfonyl)cyclopropane-1-carboxamide

The enantiomers of Example 22 (70 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (14 mg, 0.028 mmol, 20% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.41 (s, 1H), 8.81 (d, J=8.9 Hz, 1H), 8.19 (d, J=7.8 Hz, 2H), 7.85 (t, J=7.9 Hz, 1H), 7.52 (d, J=8.9 Hz, 1H), 6.99-6.90 (m, 1H), 6.94-6.83 (m, 3H), 6.60 (ddd, J=8.3, 7.2, 1.6 Hz, 1H), 6.40 (d, J=8.3 Hz, 1H), 6.30 (t, J=7.7 Hz, 1H), 3.13 (m, 1H), 3.03 (s, 3H), 2.68 (s, 3H), 2.15 (s, 3H), 1.97 (d, J=14.7 Hz, 1H), 1.41 (dd, J=9.3, 5.4 Hz, 1H). MS(APCI+) m/z 505.3 (M+H)⁺.

### Example 26

### (1R,2R)-2-(2-fluorophenyl)-1-(2-methoxy-5-methylphenyl)-N-(2-methylquinoline-5-sulfonyl)cyclopropane-1-carboxamide

The enantiomers of Example 22 (70 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (19 mg, 0.037 mmol, 26% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.34 (s, 1H), 8.83 (d, J=8.9 Hz, 1H), 8.26-8.19 (m, 2H), 7.88 (dd, J=8.5, 7.4 Hz, 1H), 7.56 (d, J=8.9 Hz, 1H), 7.00-6.84 (m, 4H), 6.61 (td, J=7.5, 1.5 Hz, 1H), 6.41 (d, J=8.3 Hz, 1H), 6.32 (td, J=7.9, 1.7 Hz, 1H), 3.11 (d, J=8.6 Hz, 1H), 3.03 (s, 3H), 2.69 (s, 3H), 2.15 (s, 3H), 2.03 (dd, J=7.5, 5.5 Hz, 1H), 1.40 (dd, J=9.3, 5.5 Hz, 1H). MS(APCI+) m/z 505.3 (M+H)⁺.

### Example 27

### (1R,2S)-1-(2-methoxy-5-methylphenyl)-2-(3-methoxyphenyl)-N-(2-methylquinoline-5-sulfonyl)cyclopropane-1-carboxamide

The enantiomers of Example 15 (59 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (23 mg, 0.045 mmol, 39% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.43 (s, 1H), 8.81 (d, J=8.9 Hz, 1H), 8.24-8.17 (m, 2H), 7.86 (dd, J=8.5, 7.4 Hz, 1H), 7.53 (d, J=9.0 Hz, 1H), 6.97-6.86 (m, 2H), 6.82 (t, J=7.9 Hz, 1H), 6.45 (dd, J=8.4, 2.3 Hz, 2H), 6.38-6.30 (m, 1H), 3.43 (s, 3H), 3.14 (s, 1H), 3.03 (s, 1H), 2.97 (dd, J=9.2, 7.2 Hz, 1H), 2.68 (s, 3H), 2.15 (s, 3H), 1.93 (s, 1H), 1.32 (dd, J=9.3, 5.4 Hz, 1H). MS(APCI+) m/z 517.3 (M+H)⁺.

### Example 28

### (1S,2R)-1-(2-methoxy-5-methylphenyl)-2-(3-methoxyphenyl)-N-(2-methylquinoline-5-sulfonyl)cyclopropane-1-carboxamide

The enantiomers of Example 15 (59 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (23 mg, 0.045 mmol, 39% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.48 (s, 1H), 8.85 (d, J=8.8 Hz, 1H), 8.23 (d, J=8.0 Hz, 2H), 7.89 (t, J=7.9 Hz, 1H), 7.57 (d, J=8.9 Hz, 1H), 6.99-6.95 (m, 1H), 6.92 (dd, J=8.4, 2.2 Hz, 1H), 6.86 (t, J=7.9 Hz, 1H), 6.48 (ddd, J=8.1, 2.6, 0.9 Hz, 2H), 6.40-6.35 (m, 1H), 6.17 (t, J=2.0 Hz, 1H), 4.09 (s, 1H), 3.47 (s, 3H), 3.17 (s, 3H), 3.00 (t, J=8.2 Hz, 1H), 2.71 (s, 3H), 2.19 (s, 3H), 1.96 (s, 1H), 1.35 (dd, J=9.2, 5.3 Hz, 1H). MS(APCI+) m/z 517.3 (M+H)⁺.

### Example 29

### rac-(1r,2s)-1-(2-methoxy-5-methylphenyl)-N-(2-methylquinoline-5-sulfonyl)-2-[2-(trifluoromethyl)phenyl]cyclopropane-1-carboxamide

The cyclopropane was prepared according to the procedure described in Example 4A by substituting 1-(trifluoromethyl)-2-vinylbenzene for 4-methylstyrene, followed by processing as in Examples 4B and 4C to afford the title compound. ¹H NMR (600 MHz, dimethyl sulfoxide-d₆) δ ppm 11.39 (s, 1H), 8.85 (d, J=8.8 Hz, 1H), 8.33-8.18 (m, 2H), 7.90 (dd, J=8.4, 7.4 Hz, 1H), 7.58 (d, J=8.9 Hz, 1H), 7.53 (dd, J=7.9, 1.3 Hz, 1H), 7.21-7.02 (m, 2H), 6.96 (dd, J=8.5, 2.3 Hz, 1H), 6.86 (s, 1H), 6.49 (d, J=8.2 Hz, 1H), 6.36 (d, J=8.0 Hz, 1H), 3.13 (t, J=8.4 Hz, 1H), 3.04 (s, 3H), 2.72 (s, 3H), 2.19-2.03 (m, 4H), 1.63 (dd, J=9.3, 5.6 Hz, 1H). MS(APCI+) m/z 555.3 (M+H)⁺.

### Example 30

### (1S,2R)-1-(2-methoxy-5-methylphenyl)-N-(2-methylquinoline-5-sulfonyl)-2-[2-(trifluoromethyl)phenyl]cyclopropane-1-carboxamide

The enantiomers of Example 29 (53.3 mg) were separated by preparative chiral supercritical fluid chromatography (ChiralPak IC column, 40% methanol/CO₂, 80 mL/minute). The fractions containing the first eluting peak were concentrated under reduced pressure to afford the title compound (21.6 mg, 0.039 mmol, 40.5% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.35 (s, 1H), 8.82 (d, J=8.9 Hz, 1H), 8.22-8.15 (m, 2H), 7.83 (t, J=8.0 Hz, 1H), 7.55-7.46 (m, 2H), 7.13 (t, J=7.6 Hz, 1H), 7.03 (t, J=7.7 Hz, 1H), 6.91 (d, J=8.3 Hz, 1H), 6.82 (s, 1H), 6.45 (d, J=8.3 Hz, 1H), 6.30 (d, J=8.0 Hz, 1H), 3.10 (s, 1H), 3.01 (s, 3H), 2.67 (s, 3H), 2.11 (s, 3H), 1.98 (s, 1H), 1.60 (dd, J=9.2, 5.4 Hz, 1H). MS(APCI+) m/z 555.3 (M+H)⁺.

### Example 31

### (1R,2S)-1-(2-methoxy-5-methylphenyl)-N-(2-methylquinoline-5-sulfonyl)-2-[2-(trifluoromethyl)phenyl]cyclopropane-1-carboxamide

The enantiomers of Example 29 (53.3 mg) were separated by preparative chiral supercritical fluid chromatography (ChiralPak IC column, 40% methanol/CO₂, 80 mL/minute). The fractions containing the second eluting peak were concentrated under reduced pressure to afford the title compound (24 mg, 0.043 mmol, 45% yield). The material was determined to be >98% ee by analytical supercritical fluid chromatography (ChiralPak IC column, 3 mL/minute, 5-50% methanol/CO₂). ¹H NMR (400 MHz, dimethyl sulfoxide-d₆) δ ppm 11.34 (s, 1H), 8.82 (d, J=8.9 Hz, 1H), 8.26-8.19 (m, 2H), 7.91-7.82 (m, 1H), 7.55 (d, J=8.9 Hz, 1H), 7.50 (dd, J=8.0, 1.4 Hz, 1H), 7.14 (t, J=7.6 Hz, 1H), 7.03 (t, J=7.6 Hz, 1H), 6.93 (dd, J=8.4, 2.2 Hz, 1H), 6.83 (s, 1H), 6.46 (d, J=8.3 Hz, 1H), 3.15-3.05 (m, 1H), 3.01 (s, 3H), 2.69 (s, 3H), 2.12 (s, 3H), 2.03 (t, J=6.5 Hz, 1H), 1.60 (dd, J=9.3, 5.6 Hz, 1H). MS(APCI+) m/z 555.2 (M+H)⁺.

### Example 32

### (1S,2R)-1-(2-methoxy-5-methylphenyl)-2-(6-methoxypyridin-3-yl)-N-(2-methylquinoline-5-sulfonyl)cyclopropane-1-carboxamide

### Example 32A

### rac-(1s,2r)-methyl 2-(6-chloropyridin-3-yl)-1-(2-methoxy-5-methylphenyl)cyclopropanecarboxylate

A solution of Example 1B (337 mg, 1.528 mmol) and 2-chloro-5-vinylpyridine (640 mg, 4.59 mmol, CombiBlocks) in dichloromethane (1.0 mL) was irradiated with blue light (Kessil lamp, 34 W) at ambient temperature. After 3 hours, the reaction was concentrated under reduced pressure and purified by flash chromatography (ISCO CombiFlash, 0-50% ethyl acetate/heptanes, 40 g RediSep® gold silica column) to afford the title compound (364 mg, 1.097 mmol, 71.8% yield). ¹H NMR (500 MHz, dimethyl sulfoxide-d₆) δ ppm 7.98 (dt, J=2.4, 0.7 Hz, 1H), 7.18-7.09 (m, 2H), 7.05 (d, J=2.3 Hz, 1H), 6.94 (ddd, J=8.3, 2.2, 0.8 Hz, 1H), 6.55 (d, J=8.3 Hz, 1H), 3.54 (s, 3H), 3.32 (s, 3H), 3.14 (dd, J=9.2, 7.2 Hz, 1H), 2.20 (d, J=0.7 Hz, 3H), 2.14 (dd, J=7.2, 5.4 Hz, 1H), 1.80 (dd, J=9.2, 5.4 Hz, 1H). MS(APCI+) m/z 332.4 (M+H)⁺.

### Example 32B

### rac-(1s,2r)-methyl 1-(2-methoxy-5-methylphenyl)-2-(6-methoxypyridin-3-yl)cyclopropanecarboxylate

A solution of sodium methoxide (310 μL, 1.356 mmol, 25% in methanol, Aldrich) was added to a solution of Example 32A (150 mg, 0.452 mmol) in N,N-dimethylformamide (1.8 mL), and the reaction was heated to 80° C. After 6 hours, the reaction was quenched with saturated NH₄Cl and extracted with ethyl acetate. The organic phase was washed with water, brine, dried with MgSO₄, filtered, and concentrated under reduced pressure. The crude residue was then purified by flash chromatography (ISCO CombiFlash, 0-50% ethyl acetate/heptanes, 12 g RediSep® gold silica column) to afford the title compound (106 mg, 0.324 mmol, 71.6% yield). ¹H NMR (400 MHz, dimethyl sulfoxide-d₆) δ ppm 7.74 (d, J=2.5 Hz, 1H), 7.06-6.97 (m, 2H), 6.91 (ddd, J=8.3, 2.2, 0.8 Hz, 1H), 6.56 (d, J=8.3 Hz, 1H), 6.43 (d, J=8.6 Hz, 1H), 3.70 (s, 3H), 3.53 (s, 3H), 3.37 (s, 3H), 3.05 (dd, J=9.4, 7.2 Hz, 1H), 2.18 (s, 3H), 2.01 (dd, J=7.2, 5.2 Hz, 1H), 1.74 (dd, J=9.3, 5.2 Hz, 1H). MS(APCI+) m/z 328.4 (M+H)⁺.

### Example 32C

### (1S,2R)-methyl 1-(2-methoxy-5-methylphenyl)-2-(6-methoxypyridin-3-yl)cyclopropanecarboxylate

The enantiomers of Example 32B (103 mg, 0.315 mmol) were separated by preparative chiral supercritical fluid chromatography (ChiralPak IC column, 20% methanol/CO₂, 60 g/minute). The fractions containing the first eluting peak were concentrated under reduced pressure to afford the title compound (49 mg, 0.15 mmol, 48% yield). ¹H NMR (400 MHz, dimethyl sulfoxide-d₆) δ ppm 7.74 (d, J=2.5 Hz, 1H), 7.06-6.97 (m, 2H), 6.91 (ddd, J=8.3, 2.2, 0.8 Hz, 1H), 6.56 (d, J=8.3 Hz, 1H), 6.43 (d, J=8.6 Hz, 1H), 3.70 (s, 3H), 3.53 (s, 3H), 3.37 (s, 3H), 3.05 (dd, J=9.4, 7.2 Hz, 1H), 2.18 (s, 3H), 2.01 (dd, J=7.2, 5.2 Hz, 1H), 1.74 (dd, J=9.3, 5.2 Hz, 1H). MS(APCI+) m/z 328.4 (M+H)⁺.

### Example 32D

### (1S,2R)-1-(2-methoxy-5-methylphenyl)-2-(6-methoxypyridin-3-yl)cyclopropanecarboxylic acid, trifluoroacetic acid

A mixture of Example 32C (49 mg, 0.150 mmol) and lithium hydroxide (10.8 mg, 0.449 mmol) in dioxane (0.75 mL) and water (0.25 mL) was heated at 80° C. for 16 hours. The reaction was then diluted with water and acidified to pH 3 with 1 M hydrochloric acid. The mixture was extracted three times with dichloromethane, and the combined organic layers were dried with MgSO₄, filtered, and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (61.6 mg, 0.144 mmol, 96% yield). ¹H NMR (600 MHz, dimethyl sulfoxide-d₆) δ ppm 7.74 (d, J=2.5 Hz, 1H), 7.03 (dd, J=8.6, 2.5 Hz, 1H), 6.98 (s, 1H), 6.90 (dd, J=8.3, 2.3 Hz, 1H), 6.54 (d, J=8.3 Hz, 1H), 6.44 (d, J=8.6 Hz, 1H), 3.71 (s, 2H), 3.36 (s, 3H), 3.00 (dd, J=9.2, 7.1 Hz, 1H), 2.18 (s, 3H), 2.07 (s, 1H), 1.92 (dd, J=7.1, 5.0 Hz, 1H), 1.71 (dd, J=9.2, 5.0 Hz, 1H). MS(APCI+) m/z 314.5 (M+H)⁺.

### Example 32E

### (1S,2R)-1-(2-methoxy-5-methylphenyl)-2-(6-methoxypyridin-3-yl)-N-(2-methylquinoline-5-sulfonyl)cyclopropane-1-carboxamide

A mixture of Example 32D (58.4 mg, 0.186 mmol), 2-methylquinoline-5-sulfonamide (49.7 mg, 0.224 mmol), 1-(3-dimethylaminopropyl)-3-ethylcarbodiimide hydrochloride (71.5 mg, 0.373 mmol), 4-dimethylaminopyridine (27.3 mg, 0.224 mmol), and N,N-diisopropylethylamine (65.1 μL, 0.373 mmol) in dichloromethane (1.2 mL) was stirred at ambient temperature. After 16 hours, the reaction was acidified with trifluoroacetic acid (72 μL, 0.93 mmol) and concentrated under reduced pressure. The resulting residue was purified by reverse-phase HPLC (Waters Xbridge Prep C18 column, 42 mL/minute, 5-95% acetonitrile/0.1% trifluoroacetic acid in water) to afford the title compound (76.2 mg, 0.147 mmol, 79% yield). ¹H NMR (500 MHz, dimethyl sulfoxide-d₆) δ ppm 11.55 (s, 1H), 8.95 (dd, J=8.9, 0.8 Hz, 1H), 8.30 (ddd, J=8.5, 4.1, 1.1 Hz, 2H), 7.96 (dd, J=8.5, 7.4 Hz, 1H), 7.68 (d, J=9.0 Hz, 1H), 7.62 (d, J=2.5 Hz, 1H), 7.03-6.88 (m, 3H), 6.50 (d, J=8.2 Hz, 1H), 6.38 (dd, J=8.6, 0.7 Hz, 1H), 3.66 (s, 3H), 3.14 (s, 3H), 3.01 (dd, J=9.3, 7.2 Hz, 1H), 2.76 (s, 3H), 2.20 (s, 3H), 2.03 (dd, J=7.2, 5.6 Hz, 1H), 1.35 (dd, J=9.3, 5.5 Hz, 1H). MS(APCI+) m/z 518.3 (M+H)⁺.

### Example 33

### (1R,2S)-1-(2-methoxy-5-methylphenyl)-2-(6-methoxypyridin-3-yl)-N-(2-methylquinoline-5-sulfonyl)cyclopropane-1-carboxamide

### Example 33A

### (1R,2S)-methyl 1-(2-methoxy-5-methylphenyl)-2-(6-methoxypyridin-3-yl)cyclopropanecarboxylate

The enantiomers of Example 32B (103 mg, 0.315 mmol) were separated by preparative chiral supercritical fluid chromatography (ChiralPak IC column, 20% methanol/CO₂, 60 g/minute). The fractions containing the second eluting peak were concentrated under reduced pressure to afford the title compound (52 mg, 0.16 mmol, 50% yield). ¹H NMR (400 MHz, dimethyl sulfoxide-d₆) δ ppm 7.74 (d, J=2.5 Hz, 1H), 7.06-6.97 (m, 2H), 6.91 (ddd, J=8.3, 2.2, 0.8 Hz, 1H), 6.56 (d, J=8.3 Hz, 1H), 6.43 (d, J=8.6 Hz, 1H), 3.70 (s, 3H), 3.53 (s, 3H), 3.37 (s, 3H), 3.05 (dd, J=9.4, 7.2 Hz, 1H), 2.18 (s, 3H), 2.01 (dd, J=7.2, 5.2 Hz, 1H), 1.74 (dd, J=9.3, 5.2 Hz, 1H). MS(APCI+) m/z 328.4 (M+H)⁺.

### Example 33B

### (1R,2S)-1-(2-methoxy-5-methylphenyl)-2-(6-methoxypyridin-3-yl)cyclopropanecarboxylic acid, trifluoroacetic acid

A mixture of Example 33A (52 mg, 0.16 mmol) and lithium hydroxide (12.1 mg, 0.504 mmol) in dioxane (0.84 mL) and water (0.28 mL) was heated at 80° C. After 16 hours, the reaction was diluted with water and acidified to pH 3 with 1 M hydrochloric acid. The mixture was then extracted three times with dichloromethane, and the combined organic layers were dried with MgSO₄, filtered, and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (57.3 mg, 0.134 mmol, 80% yield). ¹H NMR (400 MHz, dimethyl sulfoxide-d₆) δ ppm 7.74 (d, J=2.5 Hz, 1H), 7.06-6.97 (m, 2H), 6.91 (ddd, J=8.3, 2.2, 0.8 Hz, 1H), 6.56 (d, J=8.3 Hz, 1H), 6.43 (d, J=8.6 Hz, 1H), 3.70 (s, 3H), 3.53 (s, 3H), 3.37 (s, 3H), 3.05 (dd, J=9.4, 7.2 Hz, 1H), 2.18 (s, 3H), 2.01 (dd, J=7.2, 5.2 Hz, 1H), 1.74 (dd, J=9.3, 5.2 Hz, 1H). MS(APCI+) m/z 328.4 (M+H)⁺.

### Example 33C

### (1R,2S)-1-(2-methoxy-5-methylphenyl)-2-(6-methoxypyridin-3-yl)-N-(2-methylquinoline-5-sulfonyl)cyclopropane-1-carboxamide

A mixture of Example 33B (59.1 mg, 0.189 mmol), 2-methylquinoline-5-sulfonamide (50.3 mg, 0.226 mmol), 1-(3-dimethylaminopropyl)-3-ethylcarbodiimide hydrochloride (72.3 mg, 0.377 mmol), 4-dimethylaminopyridine (27.7 mg, 0.226 mmol), and N,N-diisopropylethylamine (65.9 μL, 0.377 mmol) in dichloromethane (1.3 mL) was stirred at ambient temperature. After 16 hours, the reaction was acidified with trifluoroacetic acid (15 μL, 0.19 mmol) and concentrated under reduced pressure. The resulting residue was purified by reverse-phase HPLC (Waters Xbridge Prep C18 column, 42 mL/minute, 5-95% acetonitrile/0.1% trifluoroacetic acid in water) to afford the title compound (67.8 mg, 0.131 mmol, 69.5% yield). ¹H NMR (500 MHz, dimethyl sulfoxide-d₆) δ ppm 11.56 (s, 1H), 8.99-8.89 (m, 1H), 8.33-8.26 (m, 2H), 7.95 (dd, J=8.5, 7.4 Hz, 1H), 7.66 (d, J=8.9 Hz, 1H), 7.61 (d, J=2.5 Hz, 1H), 7.00-6.92 (m, 3H), 6.50 (d, J=8.2 Hz, 1H), 6.38 (dd, J=8.6, 0.7 Hz, 1H), 3.66 (s, 3H), 3.14 (s, 3H), 3.01 (dd, J=9.3, 7.2 Hz, 1H), 2.75 (s, 3H), 2.20 (s, 3H), 2.03 (dd, J=7.2, 5.5 Hz, 1H), 1.35 (dd, J=9.4, 5.5 Hz, 1H). MS(APCI+) m/z 518.3 (M+H)⁺.

### Example 34

### (1S,2R)-1-(2-methoxy-5-methylphenyl)-2-(2-methylphenyl)-N-(2-methylquinoline-5-sulfonyl)cyclopropane-1-carboxamide

### Example 34A

### (1S,2R)-methyl 1-(2-methoxy-5-methylphenyl)-2-(o-tolyl)cyclopropanecarboxylate

A solution of methyl 2-diazo-2-(2-methoxy-5-methylphenyl)acetate (85 mg, 0.386 mmol) and pentane (6 mL) was cooled to 0-5° C. while sparging with nitrogen for 5 minutes. A solution of dirhodium tetrakis((R)—N-(dodecylbenzenesulfonyl)prolinate) (Rh₂(R-DOSP)₄) (5.12 mg, 2.70 μmol) in pentane (2 mL) was stirred at ambient temperature, then 1-methyl-2-vinylbenzene (0.249 mL, 1.930 mmol) was added, and this reaction mixture was cooled to −60° C. with N₂ sparging for 5 minutes. The diazo solution was added to the 1-methyl-2-vinylbenzene solution via syringe pump over 30 minutes. After addition, the reaction was stirred for 5 minutes at −60° C., after which LC/MS showed clean and complete conversion to product. The reaction was concentrated under reduced pressure, and the crude residue was purified via flash chromatography (ISCO CombiFlash, 0-30% ethyl acetate/heptanes, 12 g RediSep® gold silica column) to afford title compound (82 mg, 0.264 mmol, 68.4% yield). The material was determined to be >92% ee by analytical supercritical fluid chromatography (ChiralPak IC column, 3 mL/minute, 40-50% methanol/CO₂). ¹H NMR (500 MHz, dimethyl sulfoxide-d₆) δ ppm 7.11-7.05 (m, 1H), 6.97-6.93 (m, 1H), 6.91 (td, J=7.4, 1.3 Hz, 1H), 6.88-6.83 (m, 1H), 6.75-6.69 (m, 1H), 6.50 (d, J=8.2 Hz, 1H), 6.33 (dd, J=7.7, 1.3 Hz, 1H), 3.56 (s, 3H), 3.33 (s, 3H), 3.18 (dd, J=9.3, 7.6 Hz, 1H), 2.44 (s, 3H), 2.15 (t, J=0.7 Hz, 3H), 1.71 (dd, J=9.3, 5.1 Hz, 1H).

### Example 34B

### (1S,2R)-1-(2-methoxy-5-methylphenyl)-2-(o-tolyl)cyclopropanecarboxylic acid

The title compound was prepared from Example 34A according to the procedure described in Example 4B. ¹H NMR (400 MHz, dimethyl sulfoxide-d₆) δ ppm 7.03 (d, J=7.4 Hz, 1H), 6.89-6.82 (m, 2H), 6.79 (dd, J=8.3, 2.2 Hz, 1H), 6.65 (t, J=7.5 Hz, 1H), 6.42 (d, J=8.3 Hz, 1H), 6.24 (d, J=7.7 Hz, 1H), 3.26 (s, 3H), 3.07 (dd, J=9.2, 7.4 Hz, 1H), 2.40 (s, 3H), 2.09 (s, 3H), 1.97 (dd, J=7.5, 4.8 Hz, 1H), 1.65 (dd, J=9.2, 4.9 Hz, 1H). MS(APCI+) m/z 297.2 (M+H)⁺.

### Example 34C

### (1S,2R)-1-(2-methoxy-5-methylphenyl)-2-(2-methylphenyl)-N-(2-methylquinoline-5-sulfonyl)cyclopropane-1-carboxamide

The title compound was prepared according to the procedure described in Example 4C by substituting Example 34B for Example 4B (42.6 mg, 0.085 mmol, 70.1% yield). The material was determined to be >95% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.49 (s, 1H), 8.89 (d, J=8.9 Hz, 1H), 8.30-8.19 (m, 2H), 7.89 (dd, J=8.5, 7.4 Hz, 1H), 7.59 (d, J=8.9 Hz, 1H), 6.90 (d, J=7.3 Hz, 1H), 6.87-6.76 (m, 3H), 6.69-6.60 (m, 1H), 6.42 (d, J=8.2 Hz, 1H), 6.34 (dd, J=7.9, 1.3 Hz, 1H), 3.07 (s, 3H), 2.97 (dd, J=9.3, 7.6 Hz, 1H), 2.70 (s, 3H), 2.17-2.02 (m, 7H), 1.28 (dd, J=9.3, 5.5 Hz, 1H). MS(APCI+) m/z 501.3 (M+H)⁺.

### Example 35

### rac-(1r,2r)-1-(2-methoxy-5-methylphenyl)-2-(6-methylpyrazin-2-yl)-N-(2-methylquinoline-5-sulfonyl)cyclopropane-1-carboxamide

The cyclopropane was prepared according to the procedure described in Example 4A by substituting 2-ethenyl-6-methylpyrazine for 4-methylstyrene, followed by processing as in Examples 4B and 4C to afford the title compound. ¹H NMR (400 MHz, dimethyl sulfoxide-d₆) δ ppm 8.84 (d, J=8.9 Hz, 1H), 8.31-8.18 (m, 2H), 7.96 (d, J=1.9 Hz, 1H), 7.90 (dd, J=8.5, 7.4 Hz, 1H), 7.58 (d, J=8.9 Hz, 1H), 6.98 (d, J=2.2 Hz, 1H), 6.88 (dd, J=8.4, 2.2 Hz, 1H), 6.34 (d, J=8.3 Hz, 1H), 3.20 (dd, J=8.9, 6.9 Hz, 1H), 2.97 (s, 3H), 2.70 (s, 3H), 2.27 (dd, J=7.0, 4.8 Hz, 1H), 2.18 (s, 3H), 2.03 (s, 3H), 1.43 (dd, J=8.9, 4.7 Hz, 1H). MS(APCI+) m/z 503.27 (M+H)⁺.

### Example 36

### rac-(1r,2s)-1-(2-methoxy-5-methylphenyl)-2-(2-methoxypyrimidin-5-yl)-N-(2-methylquinoline-5-sulfonyl)cyclopropane-1-carboxamide

### Example 36A

### rac-(1r,2s)-methyl 2-(2-chloropyrimidin-5-yl)-1-(2-methoxy-5-methylphenyl)cyclopropanecarboxylate

The title compound was prepared according to the procedure described in Example 4A, by substituting 2-chloro-5-vinylpyrimidine for 4-methylstyrene. MS(APCI+) m/z 333.3 (M+H)⁺.

### Example 36B rac-(1r,2s)-1-(2-methoxy-5-methylphenyl)-2-(2-methoxypyrimidin-5-yl)cyclopropanecarboxylic acid

To Example 36A (30 mg, 0.090 mmol) in methanol (601 μL) was added a solution of sodium methoxide (103 μL, 0.451 mmol, 25% weight) in methanol. The reaction mixture was heated at 60° C. for overnight. LC/MS showed about a 1:1 mixture of ester and acid. Additional sodium methoxide (103 μL) was added and heating continued at 80° C. for another 5 hours. LC/MS indicated reaction completion. The reaction mixture was cooled, diluted with ethyl acetate, and acidified with 1 M HCl to pH 4-5. The mixture was extracted with ethyl acetate. The organic layer was washed with water, dried over sodium sulfate, and concentrated to afford the title product (25 mg, 0.080 mmol, 88%). MS(APCI+) m/z 315.26 (M+H)⁺.

### Example 36C

### rac-(1r,2s)-1-(2-methoxy-5-methylphenyl)-2-(2-methoxypyrimidin-5-yl)-N-(2-methylquinoline-5-sulfonyl)cyclopropane-1-carboxamide

The title compound was prepared according to the procedure described in Example 4C by substituting Example 36B for Example 4B. ¹H NMR (500 MHz, dimethyl sulfoxide-d₆) δ ppm 8.91 (d, J=8.9 Hz, 1H), 8.30 (dd, J=7.9, 3.0 Hz, 2H), 7.99-7.90 (m, 3H), 7.66 (d, J=8.9 Hz, 1H), 7.04 (d, J=2.2 Hz, 1H), 6.99 (dd, J=8.3, 2.2 Hz, 1H), 6.51 (d, J=8.3 Hz, 1H), 3.72 (s, 3H), 3.13 (s, 3H), 3.03 (dd, J=9.4, 7.1 Hz, 1H), 2.76 (s, 3H), 2.24 (s, 3H), 2.17 (dd, J=7.1, 5.7 Hz, 1H), 1.41 (dd, J=9.3, 5.6 Hz, 1H). MS(APCI+) m/z 519.21 (M+H)⁺.

### Example 37

### (1R,2S)-1-(2-methoxy-5-methylphenyl)-2-(2-methoxypyrimidin-5-yl)-N-(2-methylquinoline-5-sulfonyl)cyclopropane-1-carboxamide

The enantiomers of Example 36C (136 mg) were separated by preparative chiral supercritical fluid chromatography (ChiralPak IC column, 40% methanol/CO₂, 80 mL/minute). The fractions containing the first eluting peak were concentrated under reduced pressure to afford the title compound (33 mg, 0.064 mmol, 24.6% yield). The material was determined to be >99% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.57 (s, 1H), 8.80 (d, J=8.9 Hz, 1H), 8.25-8.18 (m, 2H), 7.92-7.82 (m, 3H), 7.55 (d, J=8.9 Hz, 1H), 6.99 (d, J=2.2 Hz, 1H), 6.93 (dd, J=8.3, 2.1 Hz, 1H), 6.45 (d, J=8.3 Hz, 1H), 3.67 (s, 3H), 2.98 (dd, J=9.4, 7.1 Hz, 1H), 2.68 (s, 3H), 2.19 (s, 3H), 2.10 (dd, J=7.1, 5.6 Hz, 1H), 1.36 (dd, J=9.3, 5.6 Hz, 1H). MS(APCI+) m/z 519.1 (M+H)⁺.

### Example 38

### (1S,2R)-1-(2-methoxy-5-methylphenyl)-2-(2-methoxypyrimidin-5-yl)-N-(2-methylquinoline-5-sulfonyl)cyclopropane-1-carboxamide

The enantiomers of Example 36C (136 mg) were separated by preparative chiral supercritical fluid chromatography (ChiralPak IC column, 40% methanol/CO₂, 80 mL/minute). The fractions containing the second eluting peak were concentrated under reduced pressure to afford the title compound (34 mg, 0.066 mmol, 25.2% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.57 (s, 1H), 8.81 (d, J=8.9 Hz, 1H), 8.26-8.18 (m, 2H), 7.92-7.83 (m, 3H), 7.55 (d, J=8.9 Hz, 1H), 6.99 (d, J=2.2 Hz, 1H), 6.94 (dd, J=8.3, 2.2 Hz, 1H), 6.46 (d, J=8.3 Hz, 1H), 3.68 (s, 3H), 3.08 (s, 3H), 2.99 (dd, J=9.3, 7.1 Hz, 1H), 2.69 (s, 3H), 2.19 (s, 3H), 2.15-2.07 (m, 1H), 1.36 (dd, J=9.4, 5.7 Hz, 1H). MS(APCI+) m/z 519.2 (M+H)⁺.

### Example 39

### rac-(1r,2r)-1-(2-methoxy-5-methylphenyl)-2-(5-methoxypyrazin-2-yl)-N-(2-methylquinoline-5-sulfonyl)cyclopropane-1-carboxamide

The cyclopropane was prepared according to the procedure described in Example 4A by substituting 2-methoxy-5-vinylpyrazine for 4-methylstyrene, followed by processing as in Examples 4B and 4C to afford the title compound. ¹H NMR (500 MHz, dimethyl sulfoxide-d₆) δ ppm 11.55 (s, 1H), 8.87 (d, J=8.9 Hz, 1H), 8.34-8.23 (m, 2H), 7.94 (dd, J=8.5, 7.4 Hz, 1H), 7.84 (d, J=1.4 Hz, 1H), 7.76 (d, J=1.4 Hz, 1H), 7.62 (d, J=8.9 Hz, 1H), 7.00-6.89 (m, 2H), 6.46 (d, J=8.1 Hz, 1H), 3.74 (s, 3H), 3.22 (dd, J=9.1, 7.0 Hz, 1H), 3.11 (s, 3H), 2.75 (s, 3H), 2.25-2.17 (m, 4H), 1.47 (dd, J=9.1, 4.8 Hz, 1H). MS(APCI+) m/z 520.0 (M+H)⁺.

### Example 40

### rac-(1r,2s)-1-(2-methoxy-5-methylphenyl)-2-(2-methoxypyridin-3-yl)-N-(2-methylquinoline-5-sulfonyl)cyclopropane-1-carboxamide

The cyclopropane was prepared according to the procedure described in Example 4A by substituting 2-methoxy-3-vinylpyridine for 4-methylstyrene, followed by processing as in Examples 4B and 4C to afford the title compound. ¹H NMR (600 MHz, dimethyl sulfoxide-d₆) δ ppm 8.90 (d, J=8.9 Hz, 1H), 8.28 (d, J=7.8 Hz, 2H), 7.93 (t, J=7.9 Hz, 1H), 7.79-7.53 (m, 2H), 6.93 (ddd, J=8.3, 2.3, 0.9 Hz, 1H), 6.88 (s, 1H), 6.53 (dd, J=7.5, 1.9 Hz, 1H), 6.49 (d, J=8.3 Hz, 1H), 6.43 (dd, J=7.4, 4.9 Hz, 1H), 3.74 (s, 3H), 3.14-3.07 (m, 3H), 2.74 (s, 3H), 2.16 (s, 3H), 1.97 (dd, J=7.6, 5.4 Hz, 1H), 1.45 (dd, J=9.3, 5.4 Hz, 1H). MS(APCI+) m/z 518.25 (M+H)⁺.

### Example 41

### rac-(1r,2s)-1-(2-methoxy-5-methylphenyl)-2-(5-methoxypyridin-3-yl)-N-(2-methylquinoline-5-sulfonyl)cyclopropane-1-carboxamide

The cyclopropane was prepared according to the procedure described in Example 4A by substituting 3-methoxy-5-vinylpyridine for 4-methylstyrene, followed by processing as in Examples 4B and 4C to afford the title compound. ¹H NMR (400 MHz, dimethyl sulfoxide-d₆) δ ppm 8.82 (d, J=8.9 Hz, 1H), 8.24 (dt, J=8.3, 2.4 Hz, 2H), 8.08 (d, J=2.6 Hz, 1H), 7.96-7.85 (m, 2H), 7.57 (d, J=8.9 Hz, 1H), 7.09 (d, J=2.2 Hz, 1H), 7.00-6.90 (m, 1H), 6.42 (d, J=8.4 Hz, 1H), 3.57 (s, 3H), 3.19 (dd, J=9.1, 7.0 Hz, 1H), 2.99 (s, 3H), 2.69 (s, 3H), 2.34-2.25 (m, 1H), 2.21 (s, 3H), 1.49 (dd, J=9.1, 5.8 Hz, 1H). MS(APCI+) m/z 518.26 (M+H)⁺.

### Example 42

### rac-(1r,2s)-2-(5-fluoropyridin-3-yl)-1-(2-methoxy-5-methylphenyl)-N-(2-methylquinoline-5-sulfonyl)cyclopropane-1-carboxamide

The cyclopropane was prepared according to the procedure described in Example 4A by substituting 3-fluoro-5-vinylpyridine for 4-methylstyrene, followed by processing as in Examples 4B and 4C to afford the title compound. ¹H NMR (500 MHz, dimethyl sulfoxide-d₆) δ ppm 11.69 (s, 1H), 8.91 (d, J=8.9 Hz, 1H), 8.33-8.25 (m, 2H), 8.13 (d, J=2.7 Hz, 1H), 7.99-7.92 (m, 2H), 7.66 (d, J=9.0 Hz, 1H), 7.06 (d, J=2.2 Hz, 1H), 6.98-6.89 (m, 2H), 6.46 (d, J=8.3 Hz, 1H), 3.15 (dd, J=9.1, 7.1 Hz, 1H), 3.07 (s, 3H), 2.75 (s, 3H), 2.23 (s, 4H), 1.43 (dd, J=9.2, 5.7 Hz, 1H). MS(APCI+) m/z 506.21 (M+H)⁺.

### Example 43

### (1s,2s)-1-(2-methoxy-5-methylphenyl)-2-(6-methoxypyridin-2-yl)-N-(2-methylquinoline-5-sulfonyl)cyclopropane-1-carboxamide

### Example 43A

### 2-chloro-6-vinylpyridine

2-Chloro-6-iodopyridine (1.5 g, 6.26 mmol, CombiBlocks), tris(dibenzylideneacetone)dipalladium(0) (0.029 g, 0.031 mmol, Aldrich), 1,3,5,7-tetramethyl-6-phenyl-2,4,8-trioxa-6-phosphaadamantane (0.027 g, 0.094 mmol, Aldrich), and potassium phosphate (3.32 g, 15.66 mmol) were combined in a reaction vial. The vial was evacuated and backfilled three times with nitrogen before being charged with dioxane (21 mL), 4,4,5,5-tetramethyl-2-vinyl-1,3,2-dioxaborolane (1.12 mL, 6.58 mmol, CombiBlocks), and water (4.2 mL). The resulting mixture was then heated to 80° C. After 16 hours, the reaction was cooled to ambient temperature, and the organic and aqueous layers separated. The organic phase was decanted and concentrated under reduced pressure. The crude material was purified by flash chromatography (ISCO CombiFlash, 0-20% ethyl acetate/heptanes, 40 g RediSep® gold silica column) to afford the title compound (612 mg, 4.38 mmol, 70.0% yield). ¹H NMR (500 MHz, CDCl₃) δ 7.60 (t, J=7.8 Hz, 1H), 7.24 (dd, J=7.7, 0.8 Hz, 1H), 7.19 (dd, J=7.9, 0.8 Hz, 1H), 6.74 (dd, J=17.4, 10.8 Hz, 1H), 6.26 (dd, J=17.4, 1.1 Hz, 1H), 5.53 (dd, J=10.7, 1.1 Hz, 1H).

### Example 43B

### rac-(1s,2s)-methyl 2-(6-chloropyridin-2-yl)-1-(2-methoxy-5-methylphenyl)cyclopropanecarboxylate

A solution of Example 1B (0.6 g, 2.72 mmol) and Example 43A (0.570 g, 4.09 mmol) in dichloromethane (18.2 mL) was irradiated with blue light (Kessil lamp, 34 W) at ambient temperature. After 16 hours, reaction was concentrated under reduced pressure and purified by flash chromatography (ISCO CombiFlash, 0-30% ethyl acetate/heptanes, 80 g RediSep® gold silica column) to afford the title compound (729 mg, 2.197 mmol, 81% yield). ¹H NMR (400 MHz, dimethyl sulfoxide-d₆) d 7.51 (t, J=7.8 Hz, 1H), 7.08 (dd, J=7.8, 0.7 Hz, 1H), 7.02-6.95 (m, 2H), 6.90 (ddd, J=8.2, 2.3, 0.9 Hz, 1H), 6.50 (d, J=8.3 Hz, 1H), 3.55 (s, 3H), 3.30 (s, 3H), 3.24 (dd, J=8.9, 7.0 Hz, 1H), 2.23 (dd, J=7.0, 4.7 Hz, 1H), 2.18 (s, 3H), 1.80 (dd, J=8.9, 4.6 Hz, 1H). MS(APCI+) m/z 332.5 (M+H)⁺.

### Example 43C

### (1S,2S)-methyl 2-(6-chloropyridin-2-yl)-1-(2-methoxy-5-methylphenyl)cyclopropanecarboxylate

The enantiomers of Example 43B (720 mg, 2.17 mmol) were separated by preparative chiral supercritical fluid chromatography (ChiralPak IC column, 15% methanol/CO₂, 80 g/minute). The fractions containing the first eluting peak were concentrated under reduced pressure to afford the title compound (281 mg, 0.847 mmol, 39% yield). ¹H NMR (400 MHz, dimethyl sulfoxide-d₆) d 7.51 (t, J=7.8 Hz, 1H), 7.08 (dd, J=7.8, 0.7 Hz, 1H), 7.02-6.95 (m, 2H), 6.90 (ddd, J=8.2, 2.3, 0.9 Hz, 1H), 6.50 (d, J=8.3 Hz, 1H), 3.55 (s, 3H), 3.30 (s, 3H), 3.24 (dd, J=8.9, 7.0 Hz, 1H), 2.23 (dd, J=7.0, 4.7 Hz, 1H), 2.18 (s, 3H), 1.80 (dd, J=8.9, 4.6 Hz, 1H). MS(APCI+) m/z 332.5 (M+H)⁺.

### Example 43D

### (1S,2S)-1-(2-methoxy-5-methylphenyl)-2-(6-methoxypyridin-2-yl)cyclopropanecarboxylic acid

A sodium methoxide solution (863 μL, 3.78 mmol, 25% in methanol, Aldrich) was added to Example 43C (50 mg, 0.151 mmol), and the reaction mixture was heated to 100° C. After 48 hours, the reaction was diluted with water and 1 M hydrochloric acid was added to acidify the reaction to pH 3. The mixture was then extracted three times with ethyl acetate, and the combined organic layers were dried with MgSO₄, filtered, and concentrated under reduced pressure to afford the title compound (44.4 mg, 0.142 mmol, 94% yield). ¹H NMR (600 MHz, dimethyl sulfoxide-d₆) d 12.16 (s, 1H), 7.42 (dd, J=8.2, 7.3 Hz, 1H), 7.02 (d, J=27.3 Hz, 1H), 6.90-6.82 (m, 2H), 6.47 (d, J=8.2 Hz, 1H), 6.34 (dd, J=8.2, 0.7 Hz, 1H), 3.27 (s, 3H), 3.09 (dd, J=8.7, 6.8 Hz, 1H), 2.23-2.09 (m, 4H), 1.75 (dd, J=8.7, 3.8 Hz, 1H). MS(APCI+) m/z 314.4 (M+H)⁺.

### Example 43E

### (1S,2S)-1-(2-methoxy-5-methylphenyl)-2-(6-methoxypyridin-2-yl)-N-(2-methylquinoline-5-sulfonyl)cyclopropane-1-carboxamide

A mixture Example 43D (50 mg, 0.160 mmol), 2-methylquinoline-5-sulfonamide (39.0 mg, 0.176 mmol), 1-(3-dimethylaminopropyl)-3-ethylcarbodiimide hydrochloride (61.2 mg, 0.319 mmol), and 4-dimethylaminopyridine (29.2 mg, 0.239 mmol) in dichloromethane (2.1 mL) was stirred at ambient temperature. After 16 hours, the reaction was acidified with trifluoroacetic acid (12 μL, 0.160 mmol) and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (79 mg, 0.153 mmol, 96% yield). ¹H NMR (400 MHz, dimethyl sulfoxide-d₆) d 8.91 (d, J=8.9 Hz, 1H), 8.35-8.19 (m, 2H), 7.95 (dd, J=8.5, 7.4 Hz, 1H), 7.65 (d, J=8.9 Hz, 1H), 7.34 (dd, J=8.2, 7.3 Hz, 1H), 7.02 (d, J=2.3 Hz, 1H), 6.93 (dd, J=8.5, 2.2 Hz, 1H), 6.70 (d, J=7.2 Hz, 1H), 6.44 (d, J=8.2 Hz, 1H), 6.29 (d, J=8.0 Hz, 1H), 3.35 (s, 3H), 3.09 (dd, J=8.8, 6.9 Hz, 1H), 3.04 (s, 3H), 2.75 (s, 3H), 2.26 (dd, J=7.0, 4.4 Hz, 1H), 2.19 (s, 3H), 1.43 (dd, J=8.8, 4.3 Hz, 1H). MS(APCI+) m/z 518.3 (M+H)⁺.

### Example 44

### (1R,2R)-1-(2-methoxy-5-methylphenyl)-2-(6-methoxypyridin-2-yl)-N-(2-methylquinoline-5-sulfonyl)cyclopropane-1-carboxamide

### Example 44A

### (1R,2R)-methyl 2-(6-chloropyridin-2-yl)-1-(2-methoxy-5-methylphenyl)cyclopropanecarboxylate

The enantiomers of Example 43B (720 mg, 2.17 mmol) were separated by preparative chiral supercritical fluid chromatography (ChiralPak IC column, 15% methanol/CO₂, 80 g/minute). The fractions containing the second eluting peak were concentrated under reduced pressure to afford the title compound (257 mg, 0.775 mmol, 36% yield). ¹H NMR (400 MHz, dimethyl sulfoxide-d₆) d 7.51 (t, J=7.8 Hz, 1H), 7.08 (dd, J=7.8, 0.7 Hz, 1H), 7.02-6.95 (m, 2H), 6.90 (ddd, J=8.2, 2.3, 0.9 Hz, 1H), 6.50 (d, J=8.3 Hz, 1H), 3.55 (s, 3H), 3.30 (s, 3H), 3.24 (dd, J=8.9, 7.0 Hz, 1H), 2.23 (dd, J=7.0, 4.7 Hz, 1H), 2.18 (s, 3H), 1.80 (dd, J=8.9, 4.6 Hz, 1H). MS(APCI+) m/z 332.5 (M+H)⁺.

### Example 44B

### (1R,2R)-1-(2-methoxy-5-methylphenyl)-2-(6-methoxypyridin-2-yl)cyclopropanecarboxylic acid

A sodium methoxide solution (863 μL, 3.78 mmol, 25% in methanol, Aldrich) was added to Example 44A (50 mg, 0.151 mmol), and the reaction mixture was heated to 100° C. After 48 hours, the reaction was diluted with water and 1 M hydrochloric acid was added to acidify the reaction to pH 3. The mixture was then extracted 3 times with ethyl acetate, and the combined organic layers were dried with MgSO₄, filtered, and concentrated under reduced pressure to afford the title compound (42.5 mg, 0.136 mmol, 90% yield). ¹H NMR (600 MHz, dimethyl sulfoxide-d₆) d 12.17 (s, 1H), 7.42 (dd, J=8.2, 7.3 Hz, 1H), 6.99 (s, 1H), 6.92-6.82 (m, 2H), 6.47 (d, J=8.3 Hz, 1H), 6.34 (dd, J=8.2, 0.8 Hz, 1H), 3.27 (s, 3H), 3.09 (dd, J=8.7, 6.8 Hz, 1H), 2.22 2.08 (m, 4H), 1.75 (dd, J=8.6, 3.7 Hz, 1H). MS(APCI+) m/z 314.4 (M+H)⁺.

### Example 44C

### (1R,2R)-1-(2-methoxy-5-methylphenyl)-2-(6-methoxypyridin-2-yl)-N-(2-methylquinoline-5-sulfonyl)cyclopropane-1-carboxamide

A mixture of Example 44B (50 mg, 0.160 mmol), 2-methylquinoline-5-sulfonamide (39.0 mg, 0.176 mmol), 1-(3-dimethylaminopropyl)-3-ethylcarbodiimide hydrochloride (61.2 mg, 0.319 mmol), and 4-dimethylaminopyridine (29.2 mg, 0.239 mmol) in dichloromethane (2.1 mL) was stirred at ambient temperature. After 16 hours, the reaction was acidified with trifluoroacetic acid (62 μL, 0.80 mmol) and concentrated under reduced pressure. The crude residue was then purified by reverse-phase HPLC (Waters Xbridge Prep C18 column, 42 mL/minute, 5-95% acetonitrile/0.1% trifluoroacetic acid in water) to afford the title compound (81 mg, 0.156 mmol, 98% yield). ¹H NMR (500 MHz, dimethyl sulfoxide-d₆) d 8.93 (dd, J=8.9, 0.8 Hz, 1H), 8.30 (ddd, J=8.4, 4.0, 1.1 Hz, 2H), 7.96 (dd, J=8.5, 7.4 Hz, 1H), 7.67 (d, J=8.9 Hz, 1H), 7.34 (dd, J=8.2, 7.3 Hz, 1H), 7.06-6.97 (m, 1H), 6.94 (ddd, J=8.2, 2.2, 0.8 Hz, 1H), 6.71 (d, J=7.2 Hz, 1H), 6.44 (d, J=8.3 Hz, 1H), 6.35-6.23 (m, 1H), 3.36 (s, 3H), 3.09 (dd, J=8.8, 6.9 Hz, 1H), 3.04 (s, 3H), 2.75 (s, 3H), 2.26 (dd, J=7.0, 4.4 Hz, 1H), 2.20 (s, 3H), 1.43 (dd, J=8.9, 4.4 Hz, 1H). MS(APCI+) m/z 518.3 (M+H)⁺.

### Example 45

### (1R,2R)-1-(2-methoxy-5-methylphenyl)-2-(6-methylpyrazin-2-yl)-N-(2-methylquinoline-5-sulfonyl)cyclopropane-1-carboxamide

The cyclopropane was prepared according to the procedure described in Example 4A by substituting 2-methyl-6-vinylpyrazine for 4-methylstyrene, followed by processing as in Examples 4B and 4C to afford the racemate of the title compound (150 mg). The enantiomers were separated by preparative chiral supercritical fluid chromatography (ChiralPak IC column, 45% methanol/CO₂, 80 mL/minute). The fractions containing the first eluting peak were concentrated under reduced pressure to afford the title compound (47 mg, 0.093 mmol, 31.1% yield). The material was determined to be >97% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.63 (s, 1H), 8.84 (d, J=8.9 Hz, 1H), 8.26 (dd, J=7.6, 3.1 Hz, 2H), 8.01 (s, 2H), 7.92 (dd, J=8.5, 7.3 Hz, 1H), 7.58 (d, J=8.9 Hz, 1H), 7.04-7.00 (m, 1H), 6.95-6.89 (m, 1H), 6.38 (d, J=8.2 Hz, 1H), 3.25 (dd, J=8.8, 6.9 Hz, 1H), 3.00 (s, 3H), 2.72 (s, 1H), 2.30 (s, 1H), 2.23 (s, 3H), 2.08 (s, 3H), 1.48 (dd, J=8.9, 4.7 Hz, 1H). MS(APCI+) m/z 503.3 (M+H)⁺.

### Example 46

### (1S,2S)-1-(2-methoxy-5-methylphenyl)-2-(6-methylpyrazin-2-yl)-N-(2-methylquinoline-5-sulfonyl)cyclopropane-1-carboxamide

The cyclopropane was prepared according to the procedure described in Example 4A by substituting 2-methyl-6-vinylpyrazine for 4-methylstyrene, followed by processing as in Examples 4B and 4C to afford the racemate of the title compound (150 mg). The enantiomers were separated by preparative chiral supercritical fluid chromatography (ChiralPak IC column, 45% methanol/CO₂, 80 mL/minute). The fractions containing the second eluting peak were concentrated under reduced pressure to afford the title compound (41 mg, 0.081 mmol, 27.1% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.61 (s, 1H), 8.83 (d, J=8.8 Hz, 1H), 8.24 (dd, J=7.9, 4.3 Hz, 2H), 7.99 (s, 2H), 7.90 (t, J=7.9 Hz, 1H), 7.56 (d, J=8.9 Hz, 1H), 7.00 (s, 1H), 6.90 (dd, J=8.3, 2.2 Hz, 1H), 6.37 (d, J=8.3 Hz, 1H), 3.23 (dd, J=8.8, 6.9 Hz, 1H), 2.99 (s, 3H), 2.71 (s, 3H), 2.53 (s, 1H), 2.2 (s, 1H), 2.06 (s, 3H), 1.46 (dd, J=8.8, 4.7 Hz, 1H). MS(APCI+) m/z 503.2 (M+H)⁺.

### Example 47

### (1R,2R)-1-(2-methoxy-5-methylphenyl)-2-(5-methoxypyrazin-2-yl)-N-(2-methylquinoline-5-sulfonyl)cyclopropane-1-carboxamide

The cyclopropane was prepared according to the procedure described in Example 4A by substituting 2-methoxy-5-vinylpyrazine for 4-methylstyrene, followed by processing as in Examples 4B and 4C to afford the racemate of the title compound (60 mg). The enantiomers were separated by preparative chiral supercritical fluid chromatography (ChiralPak IC column, 45% methanol/CO₂, 80 mL/minute). The fractions containing the first eluting peak were concentrated under reduced pressure to afford the title compound (20.8 mg, 0.04 mmol, 34.7% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.48 (s, 1H), 8.80 (d, J=8.9 Hz, 1H), 8.18 (d, J=7.9 Hz, 2H), 7.84 (t, J=7.9 Hz, 1H), 7.77 (d, J=1.5 Hz, 1H), 7.71 (d, J=1.4 Hz, 1H), 7.52 (d, J=8.9 Hz, 1H), 6.88 (s, 2H), 6.86 (d, J=2.1 Hz, OH), 6.39 (d, J=8.3 Hz, 1H), 3.69 (s, 3H), 3.14 (dd, J=6.8, 2.2 Hz, 1H), 3.05 (s, 3H), 2.67 (s, 3H), 2.14 (s, 3H), 1.42 (dd, J=9.0, 4.6 Hz, 1H). MS(APCI+) m/z 519.1 (M+H)⁺.

### Example 48

### (1S,2S)-1-(2-methoxy-5-methylphenyl)-2-(5-methoxypyrazin-2-yl)-N-(2-methylquinoline-5-sulfonyl)cyclopropane-1-carboxamide

The cyclopropane was prepared according to the procedure described in Example 4A by substituting 2-methoxy-5-vinylpyrazine for 4-methylstyrene, followed by processing as in Examples 4B and 4C to afford the racemate of the title compound (60 mg). The enantiomers were separated by preparative chiral supercritical fluid chromatography (ChiralPak IC column, 45% methanol/CO₂, 80 mL/minute). The fractions containing the second eluting peak were concentrated under reduced pressure to afford the title compound (24.3 mg, 0.047 mmol, 40.5% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.51 (s, 1H), 8.83 (d, J=8.9 Hz, 1H), 8.28-8.23 (m, 2H), 7.91 (dd, J=8.4, 7.4 Hz, 1H), 7.82 (d, J=1.4 Hz, 1H), 7.74 (d, J=1.4 Hz, 1H), 7.59 (d, J=8.9 Hz, 1H), 6.95-6.89 (m, 2H), 6.44 (d, J=8.2 Hz, 1H), 3.73 (s, 3H), 3.23-3.15 (m, 1H), 3.09 (s, 3H), 2.72 (s, 3H), 2.22-2.18 (m, 1H), 2.18 (s, 3H), 1.45 (dd, J=9.1, 4.8 Hz, 1H). MS(APCI+) m/z 519.1 (M+H)⁺.

### Example 49

### (1R,2S)-1-(2-methoxy-5-methylphenyl)-2-(2-methoxypyridin-3-yl)-N-(2-methylquinoline-5-sulfonyl)cyclopropane-1-carboxamide

The cyclopropane was prepared according to the procedure described in Example 4A by substituting 2-methoxy-3-vinylpyridine for 4-methylstyrene, followed by processing as in Examples 4B and 4C to afford the racemate of the title compound (185 mg). The enantiomers were separated by preparative chiral supercritical fluid chromatography (ChiralPak IC column, 45% methanol/CO₂, 80 mL/minute). The fractions containing the first eluting peak were concentrated under reduced pressure to afford the title compound (48.2 mg, 0.093 mmol, 26.1% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.36 (s, 1H), 8.87 (d, J=8.9 Hz, 1H), 8.30-8.24 (m, 2H), 7.92 (t, J=7.9 Hz, 1H), 7.76 (dd, J=4.9, 1.8 Hz, 1H), 7.61 (d, J=8.9 Hz, 1H), 6.97-6.91 (m, 1H), 6.89 (s, 1H), 6.56-6.47 (m, 2H), 6.45 (dd, J=7.4, 4.9 Hz, 1H), 3.75 (s, 3H), 3.16-3.10 (m, 1H), 3.11 (s, 3H), 2.75 (s, 3H), 2.18 (s, 3H), 1.97 (t, J=6.5 Hz, 1H), 1.47 (dd, J=9.3, 5.4 Hz, 1H). MS(APCI+) m/z 518.3 (M+H)⁺.

### Example 50

### (1S,2R)-1-(2-methoxy-5-methylphenyl)-2-(2-methoxypyridin-3-yl)-N-(2-methylquinoline-5-sulfonyl)cyclopropane-1-carboxamide

The cyclopropane was prepared according to the procedure described in Example 4A by substituting 2-methoxy-3-vinylpyridine for 4-methylstyrene, followed by processing as in Examples 4B and 4C to afford the racemate of the title compound (185 mg). The enantiomers were separated by preparative chiral supercritical fluid chromatography (ChiralPak IC column, 45% methanol/CO₂, 80 mL/minute). The fractions containing the second eluting peak were concentrated under reduced pressure to afford the title compound (47.6 mg, 0.092 mmol, 25.7% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.38 (s, 1H), 8.87 (d, J=8.9 Hz, 1H), 8.26 (d, J=7.7 Hz, 2H), 7.91 (t, J=7.9 Hz, 1H), 7.76 (dd, J=4.9, 1.8 Hz, 1H), 7.60 (d, J=8.9 Hz, 1H), 6.93 (dd, J=8.3, 2.2 Hz, 1H), 6.89 (s, 1H), 6.51 (dd, J=16.8, 7.8 Hz, 2H), 6.45 (dd, J=7.4, 4.9 Hz, 1H), 3.76 (s, 3H), 3.12 (d, J=4.9 Hz, 4H), 2.74 (s, 3H), 2.18 (s, 3H), 1.96 (s, 1H), 1.47 (dd, J=9.4, 5.3 Hz, 1H). MS(APCI+) m/z 518.3 (M+H)⁺.

### Example 51

### (1R,2S)-1-(2-methoxy-5-methylphenyl)-2-(5-methoxypyridin-3-yl)-N-(2-methylquinoline-5-sulfonyl)cyclopropane-1-carboxamide

The cyclopropane was prepared according to the procedure described in Example 4A by substituting 3-methoxy-5-vinylpyridine for 4-methylstyrene, followed by processing as in Examples 4B and 4C to afford the racemate of the title compound (76 mg). The enantiomers were separated by preparative chiral supercritical fluid chromatography (ChiralPak IC column, 45% methanol/CO₂, 80 mL/minute). The fractions containing the first eluting peak were concentrated under reduced pressure to afford the title compound (18.5 mg, 0.036 mmol, 24.3% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.64 (s, 1H), 8.87 (d, J=8.9 Hz, 1H), 8.21 (dd, J=8.4, 3.7 Hz, 2H), 7.87 (t, J=7.9 Hz, 1H), 7.83 (s, 1H), 7.71 (s, 1H), 7.56 (d, J=8.9 Hz, 1H), 7.05 (s, 1H), 6.95 (dd, J=8.3, 2.2 Hz, 1H), 6.49-6.42 (m, 2H), 3.49 (s, 3H), 3.06 (dd, J=9.1, 7.1 Hz, 4H), 2.72 (s, 3H), 2.23 (s, 3H), 2.05 (s, 1H), 1.44 (dd, J=9.2, 5.3 Hz, 1H). MS(APCI+) m/z 518.2 (M+H)⁺.

### Example 52

### (1S,2R)-1-(2-methoxy-5-methylphenyl)-2-(5-methoxypyridin-3-yl)-N-(2-methylquinoline-5-sulfonyl)cyclopropane-1-carboxamide

The cyclopropane was prepared according to the procedure described in Example 4A by substituting 3-methoxy-5-vinylpyridine for 4-methylstyrene, followed by processing as in Examples 4B and 4C to afford the racemate of the title compound (76 mg). The enantiomers were separated by preparative chiral supercritical fluid chromatography (ChiralPak IC column, 45% methanol/CO₂, 80 mL/minute). The fractions containing the second eluting peak were concentrated under reduced pressure to afford the title compound (18 mg, 0.035 mmol, 23.7% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.63 (s, 1H), 8.86 (d, J=8.9 Hz, 1H), 8.24 (dt, J=8.4, 2.5 Hz, 2H), 7.90 (dd, J=8.5, 7.4 Hz, 1H), 7.84 (s, 1H), 7.72 (s, 1H), 7.58 (d, J=8.8 Hz, 1H), 7.07 (d, J=2.3 Hz, 1H), 6.96 (dd, J=8.4, 2.2 Hz, 1H), 6.50-6.44 (m, 2H), 3.50 (s, 3H), 3.08 (dd, J=9.2, 7.1 Hz, 1H), 2.72 (s, 3H), 2.23 (s, 3H), 2.11 (t, J=6.3 Hz, 1H), 1.43 (dd, J=9.2, 5.5 Hz, 1H). MS(APCI+) m/z 518.2 (M+H)⁺.

### Example 53

### (1R,2S)-2-(5-fluoropyridin-3-yl)-1-(2-methoxy-5-methylphenyl)-N-(2-methylquinoline-5-sulfonyl)cyclopropane-1-carboxamide

The enantiomers of Example 42 (90 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (17.8 mg, 0.035 mmol, 19.8% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.65 (s, 1H), 8.83 (d, J=8.9 Hz, 1H), 8.27-8.21 (m, 2H), 8.11 (d, J=2.8 Hz, 1H), 7.94 (t, J=1.8 Hz, 1H), 7.89 (dd, J=8.4, 7.4 Hz, 1H), 7.57 (d, J=8.9 Hz, 1H), 7.06-7.02 (m, 1H), 6.95 (dd, J=8.5, 2.2 Hz, 1H), 6.88 (d, J=10.5 Hz, 1H), 6.44 (d, J=8.3 Hz, 1H), 3.13 (dd, J=9.1, 7.1 Hz, 1H), 3.04 (s, 3H), 2.71 (s, 3H), 2.22 (s, 3H), 2.17 (s, 1H), 1.43 (dd, J=9.1, 5.7 Hz, 1H). MS(APCI+) m/z 506.2 (M+H)⁺.

### Example 54

### (1S,2R)-2-(5-fluoropyridin-3-yl)-1-(2-methoxy-5-methylphenyl)-N-(2-methylquinoline-5-sulfonyl)cyclopropane-1-carboxamide

The enantiomers of Example 42 (90 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (18.1 mg, 0.036 mmol, 20.1% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.64 (s, 1H), 8.83 (d, J=8.9 Hz, 1H), 8.26-8.21 (m, 2H), 8.11 (d, J=2.7 Hz, 1H), 7.94 (d, J=1.9 Hz, 1H), 7.92-7.86 (m, 1H), 7.57 (d, J=8.9 Hz, 1H), 7.04 (s, 1H), 6.94 (dd, J=8.3, 2.4 Hz, 1H), 6.88 (d, J=10.5 Hz, 1H), 6.44 (d, J=8.3 Hz, 1H), 3.17 (s, 2H), 3.04 (s, 3H), 2.71 (s, 3H), 2.22 (s, 3H), 2.17 (s, 1H), 1.43 (dd, J=9.1, 5.7 Hz, 1H). MS(APCI+) m/z 506.2 (M+H)⁺.

### Example 55

### (1S,2S)-2-(6-ethoxypyridin-2-yl)-1-(2-methoxy-5-methylphenyl)-N-(2-methylquinoline-5-sulfonyl)cyclopropane-1-carboxamide

### Example 55A

### (1S,2S)-2-(6-ethoxypyridin-2-yl)-1-(2-methoxy-5-methylphenyl)cyclopropanecarboxylic acid

A sodium ethoxide solution (845 μL, 2.26 mmol, 21% in ethanol, Aldrich) was added to Example 43C (50 mg, 0.151 mmol), and the resulting solution was heated to 100° C. After heating for 4 days, the reaction was diluted with water and 1 M hydrochloric acid was added to adjust the mixture to pH 3. The mixture was then extracted 3 times with ethyl acetate. The combined organic layers were dried with MgSO₄, filtered, and concentrated under reduced pressure to afford the title compound (48.6 mg, 0.148 mmol, 99% yield). ¹H NMR (400 MHz, dimethyl sulfoxide-d₆) δ ppm 12.15 (s, 1H), 7.46-7.35 (m, 1H), 6.98 (s, 1H), 6.91-6.79 (m, 2H), 6.48 (d, J=8.2 Hz, 1H), 6.31 (d, J=8.1 Hz, 1H), 3.77 (dq, J=10.7, 7.1 Hz, 1H), 3.52-3.44 (m, 1H), 3.26 (d, J=9.5 Hz, 3H), 3.08 (dd, J=8.7, 6.7 Hz, 1H), 2.20-2.08 (m, 4H), 1.74 (dd, J=8.8, 3.8 Hz, 1H), 1.05 (t, J=7.1 Hz, 3H). MS(APCI+) m/z 328.4 (M+H)⁺.

### Example 55B

### (1S,2S)-2-(6-ethoxypyridin-2-yl)-1-(2-methoxy-5-methylphenyl)-N-(2-methylquinoline-5-sulfonyl)cyclopropane-1-carboxamide

A mixture of Example 55A (46.3 mg, 0.141 mmol), 2-methylquinoline-5-sulfonamide (34.6 mg, 0.156 mmol), 1-(3-dimethylaminopropyl)-3-ethylcarbodiimide hydrochloride (54.2 mg, 0.283 mmol), and 4-dimethylaminopyridine (25.9 mg, 0.212 mmol) in dichloromethane (1.9 mL) was stirred at ambient temperature. After 16 hours, the reaction was acidified with trifluoroacetic acid (55 μL, 0.71 mmol) and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (48.1 mg, 0.090 mmol, 64.0% yield). ¹H NMR (600 MHz, dimethyl sulfoxide-d₆) d 11.40 (s, 1H), 8.83 (d, J=8.8 Hz, 1H), 8.25 (d, J=7.8 Hz, 2H), 7.90 (dd, J=8.3, 7.5 Hz, 1H), 7.58 (d, J=8.9 Hz, 1H), 7.33 (dd, J=8.1, 7.3 Hz, 1H), 7.00 (s, 1H), 6.96-6.87 (m, 1H), 6.69 (d, J=7.3 Hz, 1H), 6.44 (d, J=8.3 Hz, 1H), 6.25 (dd, J=8.2, 0.7 Hz, 1H), 3.84 (dq, J=10.6, 7.1 Hz, 1H), 3.59 (dq, J=10.7, 7.1 Hz, 1H), 3.07 (dd, J=8.8, 6.9 Hz, 1H), 3.02 (s, 3H), 2.71 (s, 3H), 2.19 (s, 4H), 1.43 (dd, J=8.8, 4.3 Hz, 1H), 1.08 (t, J=7.1 Hz, 3H). MS(APCI+) m/z 532.4 (M+H)⁺.

### Example 56

### (1R,2R)-2-(6-ethoxypyridin-2-yl)-1-(2-methoxy-5-methylphenyl)-N-(2-methylquinoline-5-sulfonyl)cyclopropane-1-carboxamide

### Example 56A

### (1R,2R)-2-(6-ethoxypyridin-2-yl)-1-(2-methoxy-5-methylphenyl)cyclopropanecarboxylic acid

A sodium ethoxide solution (845 μL, 2.26 mmol, 21% in ethanol, Aldrich) was added to Example 44A (50 mg, 0.151 mmol) and the resulting solution was heated to 100° C. After heating for 4 days, the reaction was diluted with water and 1 M hydrochloric acid was added to adjust the reaction to pH 3. The mixture was then extracted 3 times with ethyl acetate. The combined organic layers were then dried with MgSO₄, filtered, and concentrated under reduced pressure to afford the title compound (50.8 mg, 0.155 mmol, 103% yield). ¹H NMR (600 MHz, dimethyl sulfoxide-d₆) δ ppm 12.16 (s, 1H), 7.44-7.37 (m, 1H), 6.96 (d, J=33.9 Hz, 1H), 6.89-6.79 (m, 2H), 6.48 (d, J=8.2 Hz, 1H), 6.31 (dd, J=8.2, 0.7 Hz, 1H), 3.76 (dq, J=10.7, 7.1 Hz, 1H), 3.48 (dq, J=10.7, 7.1 Hz, 1H), 3.27 (s, 3H), 3.08 (dd, J=8.7, 6.8 Hz, 1H), 2.16 (s, 3H), 2.13 (dd, J=6.8, 3.8 Hz, 1H), 1.74 (dd, J=8.7, 3.8 Hz, 1H), 1.05 (t, J=7.1 Hz, 3H). MS(APCI+) m/z 328.4 (M+H)⁺.

### Example 56B

### (1R,2R)-2-(6-ethoxypyridin-2-yl)-1-(2-methoxy-5-methylphenyl)-N-(2-methylquinoline-5-sulfonyl)cyclopropane-1-carboxamide

A mixture of Example 56A (47.4 mg, 0.145 mmol), 2-methylquinoline-5-sulfonamide (35.4 mg, 0.159 mmol), 1-(3-dimethylaminopropyl)-3-ethylcarbodiimide hydrochloride (55.5 mg, 0.290 mmol), and 4-dimethylaminopyridine (26.5 mg, 0.217 mmol) in dichloromethane (1.9 mL) was stirred at ambient temperature. After 16 hours, the reaction was acidified with trifluoroacetic acid (56 μL, 0.72 mmol) and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (49.2 mg, 0.093 mmol, 63.9% yield). ¹H NMR (600 MHz, dimethyl sulfoxide-d₆) d 11.40 (s, 1H), 8.83 (d, J=8.9 Hz, 1H), 8.25 (d, J=7.8 Hz, 2H), 7.94-7.80 (m, 1H), 7.58 (d, J=8.9 Hz, 1H), 7.33 (dd, J=8.2, 7.3 Hz, 1H), 7.00 (s, 1H), 6.93 (ddd, J=8.3, 2.3, 0.9 Hz, 1H), 6.69 (dd, J=7.4, 0.7 Hz, 1H), 6.44 (d, J=8.3 Hz, 1H), 6.25 (dd, J=8.2, 0.7 Hz, 1H), 3.84 (dq, J=10.7, 7.1 Hz, 1H), 3.59 (dq, J=10.6, 7.1 Hz, 1H), 3.07 (dd, J=8.8, 6.9 Hz, 1H), 3.02 (s, 3H), 2.71 (s, 3H), 2.19 (s, 4H), 1.43 (dd, J=8.8, 4.3 Hz, 1H), 1.08 (t, J=7.1 Hz, 3H). MS(APCI+) m/z 532.4 (M+H)⁺.

### Example 57

### (1S,2S)-1-(2-methoxy-5-methylphenyl)-N-(2-methylquinoline-5-sulfonyl)-2-[6-(propan-2-yl)pyridin-2-yl]cyclopropane-1-carboxamide

### Example 57A

### (1S,2S)-methyl 2-(6-isopropylpyridin-2-yl)-1-(2-methoxy-5-methylphenyl)cyclopropanecarboxylate

A solution of isopropylmagnesium chloride (128 μL, 0.256 mmol, 2 M in tetrahydrofuran, Aldrich) was added dropwise to a solution of Example 43C (50 mg, 0.151 mmol) and ferric acetylacetonate (4.0 mg, 0.011 mmol, Aldrich) in tetrahydrofuran (0.60 mL) and 1-methyl-2-pyrrolidinone (0.15 mL) at −20° C. After 30 minutes, the reaction was quenched with water and extracted with ethyl acetate. The organic phase was washed with brine, dried with MgSO₄, filtered, and concentrated under reduced pressure. The crude residue was purified by flash chromatography (ISCO CombiFlash, 0-100% ethyl acetate/heptanes, 12 g RediSep® silica column) to afford the title compound (16 mg, 0.047 mmol, 31.3% yield). ¹H NMR (500 MHz, CDCl₃) δ ppm 7.29 (t, J=7.7 Hz, 1H), 6.98 (d, J=2.2 Hz, 1H), 6.90 (dd, J=7.7, 1.0 Hz, 1H), 6.84 (ddd, J=8.2, 2.3, 0.8 Hz, 1H), 6.70 (dd, J=7.7, 0.9 Hz, 1H), 6.35 (d, J=8.2 Hz, 1H), 3.64 (s, 3H), 3.37-3.26 (m, 4H), 2.67 (hept, J=6.9 Hz, 1H), 2.45 (dd, J=6.9, 4.0 Hz, 1H), 2.19 (s, 3H), 1.89 (dd, J=8.8, 4.0 Hz, 1H), 0.96 (d, J=6.9 Hz, 3H), 0.86 (d, J=6.9 Hz, 3H). MS(APCI+) m/z 340.5 (M+H)⁺.

### Example 57B

### (1S,2S)-1-(2-methoxy-5-methylphenyl)-N-(2-methylquinoline-5-sulfonyl)-2-[6-(propan-2-yl)pyridin-2-yl]cyclopropane-1-carboxamide

A mixture of Example 57A (16 mg, 0.047 mmol) and lithium hydroxide (11.3 mg, 0.471 mmol) in dioxane (0.60 mL) and water (0.20 mL) was heated to 100° C. After 16 hours, the reaction was acidified with 4 M HCl in dioxane (0.5 mL, 2 mmol) and concentrated under reduced pressure. 2-Methylquinoline-5-sulfonamide (15.72 mg, 0.071 mmol), 1-(3-dimethylaminopropyl)-3-ethylcarbodiimide hydrochloride (22.59 mg, 0.118 mmol), 4-dimethylaminopyridine (14.40 mg, 0.118 mmol) and dichloromethane (0.80 mL) were added to the resulting residue. After 16 hours, the reaction was concentrated under reduced pressure and purified by reverse-phase HPLC (Waters Xbridge Prep C18 column, 42 mL/minute, 5-95% acetonitrile/0.1% trifluoroacetic acid in water) to afford the title compound (31.2 mg, 0.048 mmol, 103% yield). ¹H NMR (400 MHz, dimethyl sulfoxide-d₆) δ ppm 8.86 (d, J=8.9 Hz, 1H), 8.28 (ddd, J=8.4, 5.1, 1.1 Hz, 2H), 7.94 (dd, J=8.5, 7.4 Hz, 1H), 7.77 (s, 1H), 7.61 (d, J=9.0 Hz, 1H), 7.29 (d, J=25.8 Hz, 1H), 7.09 (s, 1H), 6.96 (dd, J=8.6, 2.2 Hz, 1H), 6.79 (dd, J=8.1, 1.0 Hz, 1H), 6.43 (d, J=8.3 Hz, 1H), 3.40 (t, J=8.0 Hz, 1H), 3.03 (s, 4H), 2.74 (s, 3H), 2.43 (t, J=6.3 Hz, 1H), 2.23 (s, 3H), 1.66 (dd, J=8.8, 5.4 Hz, 1H), 1.11 (dd, J=9.8, 6.9 Hz, 6H). MS(APCI+) m/z 530.4 (M+H)⁺.

### Example 58

### (1S,2R)-2-(6-ethoxypyridin-3-yl)-1-(2-methoxy-5-methylphenyl)-N-(2-methylquinoline-5-sulfonyl)cyclopropane-1-carboxamide

### Example 58A

### (1S,2R)-methyl 2-(6-chloropyridin-3-yl)-1-(2-methoxy-5-methylphenyl)cyclopropanecarboxylate

The enantiomers of Example 32A (537 mg, 1.62 mmol) were separated by preparative chiral supercritical fluid chromatography (ChiralPak IC column, 80 g/minute, 20% methanol/CO₂). The fractions containing the first eluting peak were concentrated under reduced pressure to afford the title compound (212 mg, 0.639 mmol, 39% yield). ¹H NMR (500 MHz, dimethyl sulfoxide-d₆) δ ppm 7.98 (dt, J=2.4, 0.7 Hz, 1H), 7.18-7.09 (m, 2H), 7.05 (d, J=2.3 Hz, 1H), 6.94 (ddd, J=8.3, 2.2, 0.8 Hz, 1H), 6.55 (d, J=8.3 Hz, 1H), 3.54 (s, 3H), 3.32 (s, 3H), 3.14 (dd, J=9.2, 7.2 Hz, 1H), 2.20 (d, J=0.7 Hz, 3H), 2.14 (dd, J=7.2, 5.4 Hz, 1H), 1.80 (dd, J=9.2, 5.4 Hz, 1H). MS(APCI+) m/z 332.4 (M+H)⁺.

### Example 58B

### (1S,2R)-2-(6-ethoxypyridin-3-yl)-1-(2-methoxy-5-methylphenyl)-N-(2-methylquinoline-5-sulfonyl)cyclopropane-1-carboxamide

A sodium ethoxide solution (1.41 mL, 3.77 mmol, 21% in ethanol, Aldrich) was added to Example 58A (50 mg, 0.151 mmol) and the reaction was heated to 100° C. After 48 hours, the reaction was diluted with water, and 1 M hydrochloric acid was added to adjust the solution to pH 3. The mixture was extracted three times with ethyl acetate, and the combined organic layers were dried with MgSO₄, filtered, and concentrated under reduced pressure. 2-Methylquinoline-5-sulfonamide (39.3 mg, 0.177 mmol), 1-(3-dimethylaminopropyl)-3-ethylcarbodiimide hydrochloride (61.6 mg, 0.321 mmol), 4-dimethylaminopyridine (29.4 mg, 0.241 mmol), and dichloromethane (2.1 mL) were added to the intermediate residue. After stirring at ambient temperature for 16 hours, the reaction was acidified with trifluoroacetic acid (62 μL, 0.80 mmol) and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (75.7 mg, 0.142 mmol, 89% yield). ¹H NMR (500 MHz, dimethyl sulfoxide-d₆) δ ppm 11.55 (s, 1H), 8.92 (dd, J=8.9, 0.8 Hz, 1H), 8.32-8.25 (m, 2H), 7.95 (dd, J=8.5, 7.4 Hz, 1H), 7.65 (d, J=8.9 Hz, 1H), 7.59 (d, J=2.5 Hz, 1H), 6.99-6.91 (m, 3H), 6.50 (d, J=8.2 Hz, 1H), 6.34 (d, J=8.5 Hz, 1H), 4.14-4.04 (m, 3H), 3.13 (s, 3H), 3.00 (dd, J=9.3, 7.2 Hz, 1H), 2.75 (s, 3H), 2.20 (s, 3H), 2.02 (dd, J=7.2, 5.5 Hz, 1H), 1.35 (dd, J=9.3, 5.4 Hz, 1H), 1.18 (t, J=7.0 Hz, 3H). MS(APCI+) m/z 532.4 (M+H)⁺.

### Example 59

### (1R,2S)-2-(6-ethoxypyridin-3-yl)-1-(2-methoxy-5-methylphenyl)-N-(2-methylquinoline-5-sulfonyl)cyclopropane-1-carboxamide

### Example 59A

### (1R,2S)-methyl 2-(6-chloropyridin-3-yl)-1-(2-methoxy-5-methylphenyl)cyclopropanecarboxylate

The enantiomers of Example 32A (537 mg, 1.62 mmol) were separated by preparative chiral supercritical fluid chromatography (ChiralPak IC column, 80 g/minute, 20% methanol/CO₂). The fractions containing the second eluting peak were concentrated under reduced pressure to afford the title compound (221 mg, 0.666 mmol, 41% yield). ¹H NMR (500 MHz, dimethyl sulfoxide-d₆) δ ppm 7.98 (dt, J=2.4, 0.7 Hz, 1H), 7.18-7.09 (m, 2H), 7.05 (d, J=2.3 Hz, 1H), 6.94 (ddd, J=8.3, 2.2, 0.8 Hz, 1H), 6.55 (d, J=8.3 Hz, 1H), 3.54 (s, 3H), 3.32 (s, 3H), 3.14 (dd, J=9.2, 7.2 Hz, 1H), 2.20 (d, J=0.7 Hz, 3H), 2.14 (dd, J=7.2, 5.4 Hz, 1H), 1.80 (dd, J=9.2, 5.4 Hz, 1H). MS(APCI+) m/z 332.4 (M+H)⁺.

### Example 59B

### (1R,2S)-2-(6-ethoxypyridin-3-yl)-1-(2-methoxy-5-methylphenyl)-N-((2-methylquinolin-5-yl)sulfonyl)cyclopropanecarboxamide

A sodium ethoxide solution (1.41 mL, 3.77 mmol, 21% in ethanol, Aldrich) was added to Example 59A (50 mg, 0.151 mmol) and the reaction was heated to 100° C. After 48 hours, the reaction was diluted with water, and 1 M hydrochloric acid was added to adjust the solution to pH 3. The mixture was then extracted three times with ethyl acetate, and the combined organic layers were dried with MgSO₄, filtered, and concentrated under reduced pressure. 2-Methylquinoline-5-sulfonamide (40.3 mg, 0.181 mmol), 1-(3-dimethylaminopropyl)-3-ethylcarbodiimide hydrochloride (63.2 mg, 0.330 mmol), 4-dimethylaminopyridine (30.2 mg, 0.247 mmol), and dichloromethane (2.2 mL) were added to the intermediate residue. After stirring at ambient temperature for 16 hours, the reaction was acidified with trifluoroacetic acid (64 μL, 0.83 mmol) and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (75.7 mg, 0.142 mmol, 89% yield). ¹H NMR (600 MHz, dimethyl sulfoxide-d₆) d 11.54 (s, 1H), 8.92 (dd, J=8.8, 0.8 Hz, 1H), 8.31-8.26 (m, 2H), 7.95 (dd, J=8.5, 7.4 Hz, 1H), 7.65 (d, J=8.9 Hz, 1H), 7.59 (dt, J=2.6, 0.7 Hz, 1H), 6.98-6.92 (m, 3H), 6.50 (d, J=8.2 Hz, 1H), 6.34 (dd, J=8.6, 0.7 Hz, 1H), 4.09 (qd, J=7.1, 1.2 Hz, 2H), 3.13 (s, 3H), 3.00 (dd, J=9.3, 7.2 Hz, 1H), 2.75 (s, 3H), 2.20 (d, J=0.7 Hz, 3H), 2.02 (dd, J=7.2, 5.5 Hz, 1H), 1.35 (dd, J=9.3, 5.5 Hz, 1H), 1.18 (t, J=7.1 Hz, 3H). MS(APCI+) m/z 532.4 (M+H)⁺.

### Example 60

### (1S,2S)-2-[6-(dimethylamino)pyridin-2-yl]-1-(2-methoxy-5-methylphenyl)-N-(2-methylquinoline-5-sulfonyl)cyclopropane-1-carboxamide

### Example 60A

### (1S,2S)-2-(6-(dimethylamino)pyridin-2-yl)-1-(2-methoxy-5-methylphenyl)cyclopropane-1-carboxylic acid

Diethanolamine (39.6 mg, 0.377 mmol, Aldrich) was added to a solution of Example 43C (50 mg, 0.151 mmol) in N,N-dimethylformamide (0.50 mL), and the reaction was heated to 150° C. After 72 hours, the crude reaction was purified by reverse-phase HPLC (Waters Xbridge Prep C18 column, 42 mL/minute, 5-95% acetonitrile/0.1% trifluoroacetic acid in water) to afford the title compound (45 mg, 0.138 mmol, 91% yield). ¹H NMR (600 MHz, CDCl₃) δ ppm 7.38 (dd, J=9.1, 7.5 Hz, 1H), 6.97 (d, J=6.7 Hz, 2H), 6.59 (dd, J=9.1, 0.8 Hz, 1H), 6.54 (d, J=8.7 Hz, 1H), 5.72-5.69 (m, 1H), 3.63-3.57 (m, 1H), 3.53 (s, 3H), 3.25 (s, 6H), 2.21 (s, 3H), 2.17 (dd, J=9.0, 5.5 Hz, 1H), 1.87 (dd, J=7.1, 5.4 Hz, 1H). MS(APCI+) m/z 327.4 (M+H)⁺.

### Example 60B

### (1S,2S)-2-(6-(dimethylamino)pyridin-2-yl)-1-(2-methoxy-5-methylphenyl)-N-((2-methylquinolin-5-yl)sulfonyl)cyclopropanecarboxamide

A mixture of Example 60A (42 mg, 0.129 mmol), 2-methylquinoline-5-sulfonamide (42.9 mg, 0.193 mmol), 1-(3-dimethylaminopropyl)-3-ethylcarbodiimide hydrochloride (61.7 mg, 0.322 mmol), and 4-dimethylaminopyridine (39.3 mg, 0.322 mmol) in dichloromethane (2.6 mL) was stirred at ambient temperature. After 16 hours, the reaction was acidified with trifluoroacetic acid (50 μL, 0.64 mmol) and concentrated under reduced pressure. The crude residue was then purified by reverse-phase HPLC (Waters Xbridge Prep C18 column, 42 mL/minute, 5-95% acetonitrile/0.1% trifluoroacetic acid in water) to afford the title compound (33 mg, 0.059 mmol, 45.9% yield). ¹H NMR (500 MHz, dimethyl sulfoxide-d₆) d 8.85 (d, J=8.9 Hz, 1H), 8.26 (ddd, J=8.4, 3.7, 1.1 Hz, 2H), 7.98-7.78 (m, 1H), 7.60 (d, J=8.9 Hz, 1H), 7.28 (s, 1H), 7.02 6.93 (m, 2H), 6.51 (d, J=8.2 Hz, 1H), 6.42 (s, 1H), 6.12 (s, 1H), 3.12 (s, 3H), 2.85 (s, 6H), 2.72 (s, 3H), 2.20 (s, 5H), 1.45 (s, 1H). MS(APCI+) m/z 531.4 (M+H)⁺.

### Example 61

### rac-(1r,2r)-1-(2-methoxy-5-methylphenyl)-N-(2-methylquinoline-5-sulfonyl)-2-[5-(propan-2-yl)pyrazin-2-yl]cyclopropane-1-carboxamide

### Example 61A

### rac-(1r,2r)-methyl 2-(5-chloropyrazin-2-yl)-1-(2-methoxy-5-methylphenyl)cyclopropanecarboxylate

The title compound was prepared according to the procedure described in Example 4A by substituting 2-chloro-5-vinylpyrazine for 4-methylstyrene. MS(APCI+) m/z 332.94 (M+H)⁺.

### Example 61B

### rac-(1r,2r)-methyl 2-(5-isopropylpyrazin-2-yl)-1-(2-methoxy-5-methylphenyl)cyclopropanecarboxylate

Isopropyl magnesium chloride (307 μL, 0.613 mmol) was added dropwise to a solution of Example 61A (120 mg, 0.361 mmol) and ferric acetylacetonate (9.55 mg, 0.027 mmol) in tetrahydrofuran (1154 μL) and N-methyl-2-pyrrolidinone (288 μL) at −20° C. LC/MS after 30 minutes showed complete conversion to a new major peak with the desired product mass. The reaction was quenched with saturated aqueous NH₄Cl solution and extracted with ethyl acetate. The organic phase was then washed with brine, dried with Na₂SO₄, filtered, and concentrated under reduced pressure. The crude residue was then purified via flash chromatography (ISCO CombiFlash, 0-50% ethyl acetate/heptanes, 12 g RediSep® gold silica column) to afford title product (96 mg, 0.282 mmol, 78%). MS(APCI+) m/z 341.25 (M+H)⁺.

### Example 61C

### rac-(1r,2r)-2-(5-isopropylpyrazin-2-yl)-1-(2-methoxy-5-methylphenyl)cyclopropanecarboxylic acid

The title compound was prepared according to the procedure described in Example 4B by substituting Example 61B for Example 4A. MS(APCI+) m/z 327.25 (M+H)⁺.

### Example 61D

### rac-(1r,2r)-1-(2-methoxy-5-methylphenyl)-N-(2-methylquinoline-5-sulfonyl)-2-[5-(propan-2-yl)pyrazin-2-yl]cyclopropane-1-carboxamide

The title compound was prepared according to the procedure described in Example 4C by substituting Example 61C for Example 4B. ¹H NMR (500 MHz, dimethyl sulfoxide-d₆) δ ppm 8.91 (d, J=8.9 Hz, 1H), 8.35-8.26 (m, 2H), 8.17 (d, J=1.5 Hz, 1H), 8.01-7.91 (m, 2H), 7.66 (d, J=8.9 Hz, 1H), 6.93 (d, J=7.1 Hz, 2H), 6.40 (d, J=8.5 Hz, 1H), 3.24 (dd, J=8.9, 7.0 Hz, 1H), 3.05 (s, 3H), 2.92-2.73 (m, 4H), 2.24 (dd, J=7.0, 4.8 Hz, 1H), 2.20 (s, 3H), 1.53 (dd, J=9.0, 4.7 Hz, 1H), 1.08 (dd, J=11.1, 6.9 Hz, 6H). MS(APCI+) m/z 531.26 (M+H)⁺.

### Example 62

### rac-(1r,2r)-2-(5-ethoxypyrazin-2-yl)-1-(2-methoxy-5-methylphenyl)-N-(2-methylquinoline-5-sulfonyl)cyclopropane-1-carboxamide

### Example 62A

### rac-(1r,2r)-methyl 2-(5-chloropyrazin-2-yl)-1-(2-methoxy-5-methylphenyl)cyclopropanecarboxylate

The title compound was prepared according to the procedure described in Example 4A by substituting 2-chloro-5-vinylpyrazine for 4-methylstyrene. MS(APCI+) m/z 332.94 (M+H)⁺.

### Example 62B

### rac-(1r,2r)-2-(5-ethoxypyrazin-2-yl)-1-(2-methoxy-5-methylphenyl)cyclopropanecarboxylic acid

A sodium ethoxide solution in ethanol (578 μL, 1.548 mmol) was added to Example 62A (51.5 mg, 0.155 mmol), and the reaction was then heated to 85° C. LC/MS after 2 days showed complete conversion.

The reaction was diluted with water and 6 M HCl was added to adjust to pH 3. The mixture was then extracted three times with ethyl acetate. The combined organic layers were dried with Na₂SO₄, filtered, and concentrated under reduced pressure to afford the title compound (55 mg, 0.167 mmol, 108% yield). MS(APCI+) m/z 329.18 (M+H)⁺.

### Example 62C

### rac-(1r,2r)-2-(5-ethoxypyrazin-2-yl)-1-(2-methoxy-5-methylphenyl)-N-(2-methylquinoline-5-sulfonyl)cyclopropane-1-carboxamide

The title compound was prepared according to the procedure described in Example 4C by substituting Example 62B for Example 4B. ¹H NMR (400 MHz, dimethyl sulfoxide-d₆) δ ppm 11.49 (s, 1H), 8.81 (d, J=9.0 Hz, 1H), 8.29-8.17 (m, 2H), 7.88 (dd, J=8.5, 7.4 Hz, 1H), 7.77 (d, J=1.4 Hz, 1H), 7.67 (d, J=1.4 Hz, 1H), 7.56 (d, J=8.9 Hz, 1H), 6.94-6.84 (m, 2H), 6.40 (d, J=8.9 Hz, 1H), 4.12 (q, J=7.0 Hz, 3H), 3.16 (dd, J=9.1, 7.0 Hz, 1H), 3.06 (s, 3H), 2.69 (s, 3H), 2.22-2.09 (m, 3H), 1.42 (dd, J=9.1, 4.8 Hz, 1H), 1.18 (t, J=7.0 Hz, 3H). MS(APCI+) m/z 533.23 (M+H)⁺.

### Example 63

### (1R,2R)-1-(2-methoxy-5-methylphenyl)-N-(2-methylquinoline-5-sulfonyl)-2-[5-(propan-2-yl)pyrazin-2-yl]cyclopropane-1-carboxamide

The cyclopropane was prepared according to the procedure described in Example 4A by substituting 2-isopropyl-5-vinylpyrazine for 4-methylstyrene, followed by processing as in Examples 4B and 4C to afford the racemate of the title compound (124 mg). The enantiomers were separated by preparative chiral supercritical fluid chromatography (ChiralPak IC column, 40% methanol/CO₂, 70 mL/minute). The fractions containing the first eluting peak were concentrated under reduced pressure to afford the title compound (36.1 mg, 0.068 mmol, 29.1% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.46 (s, 1H), 8.79 (d, J=9.0 Hz, 1H), 8.20 (d, J=7.9 Hz, 2H), 8.11 (d, J=1.5 Hz, 1H), 7.91 (d, J=1.5 Hz, 1H), 7.85 (t, J=7.9 Hz, 1H), 7.53 (d, J=9.0 Hz, 1H), 6.87 (d, J=7.4 Hz, 2H), 6.34 (d, J=8.7 Hz, 1H), 3.16 (d, J=17.3 Hz, 1H), 2.98 (s, 3H), 2.81 (p, J=6.9 Hz, 1H), 2.68 (s, 3H), 2.14 (s, 3H), 1.48 (dd, J=9.0, 4.6 Hz, 1H), 1.03 (dd, J=8.7, 6.8 Hz, 7H). MS(APCI+) m/z 531.3 (M+H)⁺.

### Example 64

### (1S,2S)-1-(2-methoxy-5-methylphenyl)-N-(2-methylquinoline-5-sulfonyl)-2-[5-(propan-2-yl)pyrazin-2-yl]cyclopropane-1-carboxamide

The cyclopropane was prepared according to the procedure described in Example 4A by substituting 2-isopropyl-5-vinylpyrazine for 4-methylstyrene, followed by processing as in Examples 4B and 4C to afford the racemate of the title compound (124 mg). The enantiomers were separated by preparative chiral supercritical fluid chromatography (ChiralPak IC column, 40% methanol/CO₂, 70 mL/minute). The fractions containing the second eluting peak were concentrated under reduced pressure to afford the title compound (36.1 mg, 0.068 mmol, 29.1% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.52 (s, 1H), 8.85 (d, J=8.9 Hz, 1H), 8.29-8.24 (m, 2H), 8.15 (d, J=1.5 Hz, 1H), 7.96-7.89 (m, 2H), 7.61 (d, J=8.9 Hz, 1H), 6.94-6.89 (m, 2H), 6.38 (d, J=8.4 Hz, 1H), 3.22 (dd, J=8.9, 7.0 Hz, 1H), 3.03 (s, 3H), 2.85 (p, J=6.9 Hz, 1H), 2.73 (s, 3H), 2.22 (dd, J=7.0, 4.7 Hz, 1H), 2.18 (s, 3H), 1.52 (dd, J=8.9, 4.7 Hz, 1H), 1.06 (dd, J=13.3, 6.8 Hz, 6H). MS(APCI+) m/z 531.3 (M+H)⁺.

### Example 65

### (1R,2R)-2-(5-ethoxypyrazin-2-yl)-1-(2-methoxy-5-methylphenyl)-N-(2-methylquinoline-5-sulfonyl)cyclopropane-1-carboxamide

The cyclopropane was prepared according to the procedure described in Example 4A by substituting 2-ethoxy-5-vinylpyrazine for 4-methylstyrene, followed by processing as in Examples 4B and 4C to afford the racemate of the title compound (31.7 mg). The enantiomers were separated by preparative chiral supercritical fluid chromatography (ChiralPak IC column, 40% methanol/CO₂, 70 mL/minute). The fractions containing the first eluting peak were concentrated under reduced pressure to afford the title compound (7.8 mg, 0.015 mmol, 24.6% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.54 (s, 1H), 8.86 (d, J=8.9 Hz, 1H), 8.22 (s, 2H), 7.88 (s, 1H), 7.80 (s, 1H), 7.72 (d, J=1.4 Hz, 1H), 7.56 (d, J=8.9 Hz, 1H), 6.92 (s, 2H), 6.44 (d, J=8.2 Hz, 1H), 4.17 (q, J=7.0 Hz, 2H), 3.19 (s, 1H), 3.10 (s, 3H), 2.73 (s, 2H), 2.54 (s, 1H), 2.19 (s, 3H), 1.47 (dd, J=9.0, 4.5 Hz, 1H), 1.23 (t, J=7.0 Hz, 3H). MS(APCI+) m/z 533.2 (M+H)⁺.

### Example 66

### (1S,2S)-2-(5-ethoxypyrazin-2-yl)-1-(2-methoxy-5-methylphenyl)-N-(2-methylquinoline-5-sulfonyl)cyclopropane-1-carboxamide

The cyclopropane was prepared according to the procedure described in Example 4A by substituting 2-ethoxy-5-vinylpyrazine for 4-methylstyrene, followed by processing as in Examples 4B and 4C to afford the racemate of the title compound (31.7 mg). The enantiomers were separated by preparative chiral supercritical fluid chromatography (ChiralPak IC column, 40% methanol/CO₂, 70 mL/minute). The fractions containing the second eluting peak were concentrated under reduced pressure to afford the title compound (8.8 mg, 0.017 mmol, 27.8% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.44 (s, 1H), 8.80 (d, J=8.9 Hz, 1H), 8.17 (s, 2H), 7.83 (s, 1H), 7.75 (s, 1H), 7.67 (d, J=1.4 Hz, 1H), 7.52 (d, J=9.0 Hz, 1H), 6.87 (s, 2H), 6.39 (d, J=8.3 Hz, 1H), 4.12 (q, J=7.0 Hz, 2H), 3.14 (s, 2H), 3.05 (s, 3H), 2.67 (s, 3H), 2.14 (s, 3H), 1.42 (dd, J=8.8, 4.6 Hz, 1H), 1.20 (s, OH), 1.18 (t, J=7.0 Hz, 3H). MS(APCI+) m/z 533.3 (M+H)⁺.

### Example 67

### (1S,2R)-1-(2-methoxy-5-methylphenyl)-N-(2-methylquinoline-5-sulfonyl)-2-[6-(propan-2-yl)pyridin-3-yl]cyclopropane-1-carboxamide

### Example 67A

### (1S,2R)-methyl 2-(6-isopropylpyridin-3-yl)-1-(2-methoxy-5-methylphenyl)cyclopropanecarboxylate

A solution of isopropylmagnesium chloride (128 μL, 0.256 mmol, 2 M in tetrahydrofuran, Aldrich) was added dropwise to a solution of Example 58A (50 mg, 0.151 mmol) and ferric acetylacetonate (4.0 mg, 0.011 mmol, Aldrich) in tetrahydrofuran (0.80 mL) and 1-methyl-2-pyrrolidinone (0.20 mL) at −20° C. After 4 hours, the reaction was quenched with saturated NaHCO₃ and extracted with ethyl acetate. The organic phase was washed with brine, dried with MgSO₄, filtered, and concentrated under reduced pressure. The crude residue was then purified by flash chromatography (ISCO CombiFlash, 0-50% ethyl acetate/heptanes, 12 g RediSep® gold silica column) to afford the title compound (21 mg, 0.062 mmol, 41.1% yield). ¹H NMR (500 MHz, dimethyl sulfoxide-d₆) d 8.09 (dd, J=2.5, 0.9 Hz, 1H), 7.05-6.97 (m, 1H), 6.97-6.86 (m, 2H), 6.84 (dd, J=8.2, 0.9 Hz, 1H), 6.51 (d, J=8.2 Hz, 1H), 3.54 (s, 3H), 3.25 (s, 3H), 3.07 (dd, J=9.3, 7.3 Hz, 1H), 2.83 (hept, J=6.9 Hz, 1H), 2.19 (s, 3H), 2.00 (dd, J=7.3, 5.2 Hz, 1H), 1.80 (dd, J=9.3, 5.1 Hz, 1H), 1.09 (d, J=6.9 Hz, 6H). MS(APCI+) m/z 340.6 (M+H)⁺.

### Example 67B

### (1S,2R)-1-(2-methoxy-5-methylphenyl)-N-(2-methylquinoline-5-sulfonyl)-2-[6-(propan-2-yl)pyridin-3-yl]cyclopropane-1-carboxamide

A mixture of Example 67A (19.7 mg, 0.058 mmol) and lithium hydroxide (7.0 mg, 0.29 mmol) in dioxane (0.45 mL) and water (0.15 mL) was heated to 80° C. After 4 hours, the reaction was acidified with excess 4 M HCl in dioxane and concentrated under reduced pressure. 2-Methylquinoline-5-sulfonamide (16.1 mg, 0.073 mmol), 1-(3-dimethylaminopropyl)-3-ethylcarbodiimide hydrochloride (22.3 mg, 0.116 mmol), 4-dimethylaminopyridine (17.7 mg, 0.145 mmol), and dichloromethane (0.6 mL) were added to the intermediate residue. After stirring for 16 hours at ambient temperature, the reaction was concentrated under reduced pressure and purified by reverse-phase HPLC (Waters Xbridge Prep C18 column, 42 mL/minute, 5-95% acetonitrile/0.1% trifluoroacetic acid in water) to afford the title compound (26.4 mg, 0.041 mmol, 70.7% yield). ¹H NMR (400 MHz, dimethyl sulfoxide-d₆) δ ppm 8.85 (d, J=8.9 Hz, 1H), 8.27 (d, J=7.9 Hz, 2H), 8.19 (d, J=2.3 Hz, 1H), 7.98-7.86 (m, 1H), 7.61 (dd, J=16.1, 8.4 Hz, 2H), 7.44 (d, J=8.4 Hz, 1H), 7.09 (d, J=2.3 Hz, 1H), 6.98 (dd, J=8.3, 2.2 Hz, 1H), 6.41 (d, J=8.4 Hz, 1H), 3.26 (dd, J=9.3, 6.9 Hz, 2H), 3.07 (p, J=6.8 Hz, 1H), 3.01 (s, 3H), 2.73 (s, 3H), 2.30-2.19 (m, 4H), 1.58 (dd, J=9.1, 5.8 Hz, 1H), 1.14 (dd, J=7.0, 2.0 Hz, 6H). MS(APCI+) m/z 530.4 (M+H)⁺.

### Example 68

### (1S,2R)-2-[6-(dimethylamino)pyridin-3-yl]-1-(2-methoxy-5-methylphenyl)-N-(2-methylquinoline-5-sulfonyl)cyclopropane-1-carboxamide

### Example 68A

### (1S,2R)-2-(6-(dimethylamino)pyridin-3-yl)-1-(2-methoxy-5-methylphenyl)cyclopropanecarboxylic acid, trifluoroacetic acid

A solution of Example 58A (50 mg, 0.151 mmol) and diethanolamine (79 mg, 0.753 mmol, Aldrich) in N,N-dimethylformamide (1 mL) was heated to 150° C. After 3 days, the reaction was purified by reverse-phase HPLC (Waters Xbridge Prep C18 column, 42 mL/minute, 5-95% acetonitrile/0.1% trifluoroacetic acid in water) to afford the title compound (60 mg, 0.136 mmol, 90% yield). ¹H NMR (600 MHz, CDCl₃) δ ppm 7.54-7.50 (m, 1H), 7.40-7.32 (m, 1H), 7.05-6.93 (m, 2H), 6.63-6.44 (m, 2H), 3.57 (s, 3H), 3.18 (s, 6H), 3.13 (dd, J=9.2, 7.1 Hz, 1H), 2.24 (d, J=2.8 Hz, 3H), 2.03 (dd, J=9.3, 5.5 Hz, 1H), 1.86 (dd, J=7.1, 5.5 Hz, 1H). MS(APCI+) m/z 327.5 (M+H)⁺.

### Example 68B

### (1S,2R)-2-[6-(dimethylamino)pyridin-3-yl]-1-(2-methoxy-5-methylphenyl)-N-(2-methylquinoline-5-sulfonyl)cyclopropane-1-carboxamide

A mixture of Example 68A (58 mg, 0.132 mmol), 2-methylquinoline-5-sulfonamide (43.9 mg, 0.198 mmol), 4-dimethylaminopyridine (40.2 mg, 0.329 mmol), and 1-(3-dimethylaminopropyl)-3-ethylcarbodiimide hydrochloride (50.5 mg, 0.263 mmol) in dichloromethane (1.3 mL) was stirred at ambient temperature. After 16 hours, the reaction was acidified with excess trifluoroacetic acid and concentrated under reduced pressure. The crude residue was then purified by reverse-phase HPLC (Waters Xbridge Prep C18 column, 42 mL/minute, 5-95% acetonitrile/0.1% trifluoroacetic acid in water) to afford the title compound (26.0 mg, 0.040 mmol, 30.6% yield). ¹H NMR (400 MHz, dimethyl sulfoxide-d₆) δ ppm 8.85 (d, J=8.9 Hz, 1H), 8.26 (d, J=7.9 Hz, 2H), 7.91 (t, J=8.0 Hz, 1H), 7.59 (d, J=8.9 Hz, 1H), 7.37 (d, J=2.2 Hz, 1H), 7.31 (dd, J=9.6, 2.3 Hz, 1H), 7.05-6.92 (m, 2H), 6.82 (d, J=9.5 Hz, 1H), 6.55 (d, J=8.2 Hz, 1H), 3.17 (s, 3H), 3.03 (s, 7H), 2.73 (s, 3H), 2.22 (s, 3H), 2.09 (t, J=6.5 Hz, 1H), 1.40 (dd, J=9.4, 5.7 Hz, 1H). MS(APCI+) m/z 531.4 (M+H)⁺.

### Example 69

### (1S,2S)-1-(2-methoxy-5-methylphenyl)-N-(2-methylquinoline-5-sulfonyl)-2-[6-(propan-2-yl)pyridazin-3-yl]cyclopropane-1-carboxamide

### Example 69A

### 3-chloro-6-vinylpyridazine

3,6-Dichloropyridazine (1 g, 6.71 mmol, Aldrich), (1,1′-bis(diphenylphosphino)ferrocenedichloro palladium(II) dichloromethane complex (0.049 g, 0.067 mmol, Aldrich), and potassium carbonate (2.78 g, 20.14 mmol) were combined in a reaction vial. The vial was evacuated and backfilled with nitrogen three times before being charged with dioxane (9.6 mL), vinylboronic acid pinacol ester (1.03 g, 6.71 mmol, CombiBlocks), and water (3.8 mL). After heating at 80° C. for 16 hours, the reaction was diluted with ethyl acetate and washed with water and brine. The organic phase was dried with MgSO₄, filtered, and concentrated under reduced pressure. The crude residue was then purified by flash chromatography (ISCO CombiFlash, 0-40% ethyl acetate/heptanes, 40 g RediSep® gold silica column) to afford the title compound (284 mg, 2.020 mmol, 30.1% yield). ¹H NMR (500 MHz, CDCl₃) δ ppm 7.58 (d, J=8.9 Hz, 1H), 7.48 (d, J=8.9 Hz, 1H), 7.04 (dd, J=17.8, 11.1 Hz, 1H), 6.24 (d, J=17.8 Hz, 1H), 5.74 (d, J=11.1 Hz, 1H).

### Example 69B

### 2-(2-methoxy-5-methylphenyl)-2-oxoacetic acid

Lithium hydroxide (1.06 g, 44.3 mmol) was added to a solution of Example 1A (4.61 g, 22.1 mmol) in tetrahydrofuran (30 mL) and water (15 mL) at ambient temperature. After stirring for 2 hours, the reaction was acidified with 1 M hydrochloric acid and extracted with ethyl acetate. The organic phase was washed with brine, dried with MgSO₄, filtered, and concentrated under reduced pressure to afford the title compound (4.03 g, 20.8 mmol, 94% yield). ¹H NMR (600 MHz, dimethyl sulfoxide-d₆) δ ppm 7.55-7.48 (m, 2H), 7.15 (d, J=8.5 Hz, 1H), 3.81 (s, 3H), 2.29 (t, J=0.7 Hz, 3H). MS(APCI+) m/z 195.6 (M+H)⁺.

### Example 69C

### (R)-4,4-dimethyl-2-oxotetrahydrofuran-3-yl 2-(2-methoxy-5-methylphenyl)-2-oxoacetate

Oxalyl chloride (8.66 mL, 99 mmol) was added dropwise to a mixture of Example 69B (9.6 g, 49.4 mmol) and N,N-dimethylformamide (0.038 mL, 0.494 mmol) in dichloromethane (100 mL) at 0° C. The reaction was slowly warmed to ambient temperature and stirred for 16 hours before being concentrated under reduced pressure. The resulting residue was then taken up in dichloromethane (100 mL) and cooled in an ice bath. Triethylamine (17.23 mL, 124 mmol), 4-dimethylaminopyridine (0.060 g, 0.494 mmol), and (R)-3-hydroxy-4,4-dimethyldihydrofuran-2(3H)-one (9.65 g, 74.2 mmol, CombiBlocks) were added sequentially before warming the mixture to ambient temperature. After stirring for 2 hours, the reaction was washed with 1 M hydrochloric acid, saturated NaHCO₃, and brine. The organic phase was then dried with MgSO₄, filtered and concentrated under reduced pressure. The crude residue was purified by flash chromatography (ISCO CombiFlash, 0-50% ethyl acetate/heptanes, 120 g RediSep® gold silica column) to afford the title compound (13.8 g, 45.1 mmol, 91% yield). ¹H NMR (500 MHz, dimethyl sulfoxide-d₆) δ ppm 7.62-7.55 (m, 2H), 7.19 (d, J=8.5 Hz, 1H), 5.82 (s, 1H), 4.22-4.17 (m, 1H), 4.12 (d, J=8.6 Hz, 1H), 3.84 (s, 3H), 2.31 (d, J=0.8 Hz, 3H), 1.21 (s, 3H), 0.99 (s, 3H). MS(APCI+) m/z 307.3 (M+H)⁺.

### Example 69D

### (R)-4,4-dimethyl-2-oxotetrahydrofuran-3-yl 2-diazo-2-(2-methoxy-5-methylphenyl)acetate

A mixture of Example 69C (8.53 g, 27.8 mmol) and 4-methylbenzenesulfonohydrazide (5.19 g, 27.8 mmol, Aldrich) in toluene (56 mL) was heated to reflux with a Dean-Stark trap. After 16 hours, the reaction was concentrated under reduced pressure and dichloromethane (56 mL) and triethylamine (5.82 mL, 41.8 mmol) were added to the resulting residue. After stirring at ambient temperature for 16 hours, the reaction was washed with saturated NaHCO₃, brine, dried with MgSO₄, filtered, and concentrated under reduced pressure. The crude residue was purified by flash chromatography (ISCO CombiFlash, 0-40% ethyl acetate/heptanes, 120 g RediSep® gold silica column) to afford the title compound (7.3 g, 23 mmol, 82% yield). ¹H NMR (500 MHz, CDCl₃) δ ppm 7.37 (d, J=2.2 Hz, 1H), 7.09-7.04 (m, 1H), 6.80 (d, J=8.4 Hz, 1H), 5.50 (s, 1H), 4.10-4.02 (m, 2H), 3.84 (s, 3H), 2.33-2.29 (m, 3H), 1.25 (s, 3H), 1.12 (s, 3H).

### Example 69E

### (1S,2S)—(R)-4,4-dimethyl-2-oxotetrahydrofuran-3-yl 2-(6-chloropyridazin-3-yl)-1-(2-methoxy-5-methylphenyl)cyclopropanecarboxylate

A solution of Example 69D (80 mg, 0.25 mmol) in dichloromethane (1.25 mL) was added dropwise to a solution of Example 69A (52.7 mg, 0.375 mmol) and rhodium(II) octanoate dimer (1.947 mg, 2.500 μmol, Strem) in dichloromethane (1.25 mL) at 0° C. After 30 minutes, the reaction was concentrated under reduced pressure and purified by flash chromatography (ISCO CombiFlash, 0-100% ethyl acetate/heptanes, 12 g RediSep® gold silica column) to afford the title compound (56.4 mg, 0.131 mmol, 52.4% yield). The material was determined to be 96% de by analytical chiral supercritical fluid chromatography (ChiralPak IC column, 3 mL/minute, 5-50% methanol/CO₂, major diastereomer is the second eluting peak). ¹H NMR (600 MHz, dimethyl sulfoxide-d₆) d 7.61 (d, J=8.9 Hz, 1H), 7.41 (d, J=9.0 Hz, 1H), 7.17-7.08 (m, 1H), 6.94 (ddd, J=8.3, 2.3, 0.8 Hz, 1H), 6.50 (d, J=8.3 Hz, 1H), 5.57 (s, 1H), 4.10 (dd, J=8.6, 0.7 Hz, 1H), 3.94 (d, J=8.5 Hz, 1H), 3.52 (dd, J=8.9, 7.1 Hz, 1H), 3.32 (s, 3H), 2.60 (dd, J=7.1, 5.0 Hz, 1H), 2.21 (s, 3H), 1.96 (dd, J=9.0, 4.9 Hz, 1H), 1.04 (s, 3H), 0.57 (s, 3H). MS(APCI+) m/z 431.4 (M+H)⁺.

### Example 69F

### (1S,2S)—(R)-4,4-dimethyl-2-oxotetrahydrofuran-3-yl 2-(6-isopropylpyridazin-3-yl)-1-(2-methoxy-5-methylphenyl)cyclopropanecarboxylate

A solution of isopropylmagnesium chloride (108 μL, 0.217 mmol, 2 M in tetrahydrofuran, Aldrich) was added dropwise to a solution of Example 69E (55 mg, 0.128 mmol) and ferric acetylacetonate (3.4 mg, 9.57 μmol, Aldrich) in tetrahydrofuran (0.68 mL) and 1-methyl-2-pyrrolidinone (0.17 mL) at −20° C. After 30 minutes, the reaction was quenched with saturated NH₄Cl and extracted with ethyl acetate. The organic phase was washed with water, brine, dried with MgSO₄, filtered, and concentrated under reduced pressure. The crude residue was then purified by flash chromatography (ISCO CombiFlash, 0-100% ethyl acetate/heptanes, 12 g RediSep® gold silica column) to afford the title compound (29.6 mg, 0.068 mmol, 52.9% yield). ¹H NMR (500 MHz, dimethyl sulfoxide-d₆) δ ppm 7.26 (d, J=8.7 Hz, 1H), 7.07 (d, J=8.8 Hz, 2H), 6.91 (ddd, J=8.3, 2.3, 0.9 Hz, 1H), 6.46 (d, J=8.4 Hz, 1H), 5.56 (s, 1H), 4.10 (d, J=8.6 Hz, 1H), 3.94 (d, J=8.6 Hz, 1H), 3.46 (dd, J=9.1, 7.3 Hz, 1H), 3.24 (s, 3H), 3.05 (hept, J=7.0 Hz, 1H), 2.19 (s, 3H), 1.94 (dd, J=9.1, 4.9 Hz, 1H), 1.14 (dd, J=6.9, 1.0 Hz, 6H), 1.04 (s, 3H), 0.58 (s, 3H). MS(APCI+) m/z 439.4 (M+H)⁺.

### Example 69G

### (1S,2S)-2-(6-isopropylpyridazin-3-yl)-1-(2-methoxy-5-methylphenyl)cyclopropanecarboxylic acid

A mixture of Example 69F (28.5 mg, 0.065 mmol), lithium hydroxide (7.8 mg, 0.33 mmol) in dioxane (0.45 mL), and water (0.15 mL) was heated to 80° C. After 4 hours, the reaction was acidified with 4 M HCl in dioxane and concentrated under reduced pressure. The crude reaction was purified by reverse-phase HPLC (Waters Xbridge Prep C18 column, 42 mL/minute/minute, 5-95% acetonitrile/0.1% trifluoroacetic acid in water) to afford the title compound (14.9 mg, 0.046 mmol, 70.2% yield). ¹H NMR (600 MHz, CDCl₃) δ ppm 7.47 (d, J=8.9 Hz, 1H), 7.22 (d, J=9.0 Hz, 1H), 7.10 (s, 1H), 7.00 (ddd, J=8.2, 2.3, 0.8 Hz, 1H), 6.40 (d, J=8.3 Hz, 1H), 3.74 (t, J=7.9 Hz, 1H), 3.44 (p, J=6.9 Hz, 1H), 3.37 (s, 3H), 2.35-2.20 (m, 5H), 1.29 (dd, J=12.2, 6.8 Hz, 6H). MS(APCI+) m/z 327.4 (M+H)⁺.

### Example 69H

### (1S,2S)-1-(2-methoxy-5-methylphenyl)-N-(2-methylquinoline-5-sulfonyl)-2-[6-(propan-2-yl)pyridazin-3-yl]cyclopropane-1-carboxamide

A mixture of Example 69G (13.9 mg, 0.043 mmol), 2-methylquinoline-5-sulfonamide (11.83 mg, 0.053 mmol), 1-(3-dimethylaminopropyl)-3-ethylcarbodiimide hydrochloride (16.33 mg, 0.085 mmol), and 4-dimethylaminopyridine (13.01 mg, 0.106 mmol) in dichloromethane (0.57 mL) was stirred at ambient temperature. After 16 hours, the reaction was acidified with excess trifluoroacetic acid and concentrated under reduced pressure. The crude residue was then purified by reverse-phase HPLC (Waters Xbridge Prep C18 column, 42 mL/minute, 5-95% acetonitrile/0.1% trifluoroacetic acid in water) to afford the title compound (25.9 mg, 0.040 mmol, 94% yield). ¹H NMR (500 MHz, dimethyl sulfoxide-d₆) δ ppm 8.88 (d, J=8.9 Hz, 1H), 8.32-8.20 (m, 2H), 7.94 (dd, J=8.5, 7.4 Hz, 1H), 7.63 (d, J=8.9 Hz, 1H), 7.46 (d, J=8.8 Hz, 1H), 7.21 (d, J=8.9 Hz, 1H), 7.00 (d, J=2.3 Hz, 1H), 6.95 (dd, J=8.6, 2.2 Hz, 1H), 6.41 (d, J=8.3 Hz, 1H), 3.38 (dd, J=8.9, 7.1 Hz, 1H), 3.05 (dq, J=13.9, 6.9 Hz, 1H), 3.00 (s, 3H), 2.75 (s, 3H), 2.36 (dd, J=7.1, 5.2 Hz, 1H), 2.20 (s, 3H), 1.64 (dd, J=9.0, 5.1 Hz, 1H), 1.12 (dd, J=7.0, 5.2 Hz, 6H). MS(APCI+) m/z 531.4 (M+H)⁺.

### Example 70

### (1S,2R)-2-[6-(difluoromethyl)pyridin-3-yl]-1-(2-methoxy-5-methylphenyl)-N-(2-methylquinoline-5-sulfonyl)cyclopropane-1-carboxamide

### Example 70A

### 2-(difluoromethyl)-5-vinylpyridine

To 5-bromo-2-(difluoromethyl)pyridine (0.5 g, 2.404 mmol), 4,4,5,5-tetramethyl-2-vinyl-1,3,2-dioxaborolane (0.370 g, 2.404 mmol), potassium carbonate (0.831 g, 6.01 mmol) in ethanol (3 mL), and tetrahydrofuran (6 mL) was added bis(triphenylphosphine)palladium(II) dichloride (0.169 g, 0.240 mmol). The reaction mixture was bubbled with nitrogen before being stirred overnight at 60° C. in a sealed vessel. The mixture was cooled to ambient temperature before adding water (5 mL) and extracting with ethyl acetate (3×10 mL). The combined organic layers were dried over MgSO₄, filtered, concentrated, and then purified on a silica column (ISCO CombiFlash, 0-30% ethyl acetate/heptanes) to afford the title compound (187.7 mg, 1.210 mmol, 50.3% yield). ¹H NMR (500 MHz, CDCl₃) δ 8.64 (dt, J=2.2, 0.7 Hz, 1H), 7.86 (dd, J=8.2, 2.2 Hz, 1H), 7.60 (d, J=8.2 Hz, 1H), 6.75 (dd, J=17.7, 11.0 Hz, 1H), 6.63 (t, J=55.5 Hz, 1H), 5.91 (d, J=17.9 Hz, 1H), 5.48 (d, J=11.0 Hz, 1H). MS(ESI+) m/z 156.1 (M+H)⁺.

### Example 70B

### (1S,2R)-2-(6-(difluoromethyl)pyridin-3-yl)-1-(2-methoxy-5-methylphenyl)-N-((2-methylquinolin-5-yl)sulfonyl)cyclopropanecarboxamide

The cyclopropane was prepared according to the procedure described in Example 69E by substituting 2-(difluoromethyl)-5-vinylpyridine for Example 69A, followed by processing as in Examples 69G and 69H to afford the title compound. ¹H NMR (400 MHz, CDCl₃) δ 9.01 (d, J=8.9 Hz, 1H), 8.58 (d, J=8.5 Hz, 1H), 8.49 (d, J=7.4 Hz, 1H), 8.26 (s, 1H), 8.09 (s, 1H), 7.99-7.90 (m, 1H), 7.52 (d, J=8.9 Hz, 1H), 7.21 (d, J=8.1 Hz, 1H), 7.10 (s, 1H), 6.90 (d, J=7.9 Hz, 1H), 6.63-6.33 (m, 2H), 3.14 (s, 3H), 3.08-2.99 (m, 1H), 2.92 (s, 3H), 2.31 (s, 3H), 2.00 (dd, J=9.3, 5.3 Hz, 1H). MS(APCI+) m/z 538.4 (M+H)⁺.

### Example 71

### (1S,2R)-2-(2-ethoxypyridin-3-yl)-1-(2-methoxy-5-methylphenyl)-N-(2-methylquinoline-5-sulfonyl)cyclopropane-1-carboxamide

The cyclopropane was prepared according to the procedure described in Example 4A by substituting 2-ethoxy-3-vinylpyridine for 4-methylstyrene, followed by processing as in Examples 4B and 4C to afford the racemate of the title compound (206 mg). The enantiomers were separated by preparative chiral supercritical fluid chromatography (ChiralPak IC column, 40% methanol/CO₂, 70 mL/minute). The fractions containing the second eluting peak were concentrated under reduced pressure to afford the title compound (52.5 mg, 0.099 mmol, 25.5% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.34 (s, 1H), 8.90 (d, J=8.8 Hz, 1H), 8.22 (s, 2H), 7.88 (s, 1H), 7.73 (dd, J=4.8, 1.9 Hz, 1H), 7.57 (d, J=8.9 Hz, 1H), 6.92 (d, J=8.2 Hz, 1H), 6.86 (s, 1H), 6.50 (d, J=8.4 Hz, 2H), 6.42 (dd, J=7.3, 4.9 Hz, 1H), 4.26-4.11 (m, 1H), 3.15-3.11 (m, 3H), 3.07 (s, 1H), 2.72 (s, 1H), 2.16 (s, 3H), 1.89 (s, 1H), 1.11 (t, J=7.0 Hz, 3H). MS(APCI+) m/z 532.2 (M+H)⁺.

### Example 72

### (1S,2R)-1-(2-methoxy-5-methylphenyl)-2-(6-methoxy-2-methylpyridin-3-yl)-N-(2-methylquinoline-5-sulfonyl)cyclopropane-1-carboxamide

### Example 72A

### 6-methoxy-2-methyl-3-vinylpyridine

Tris(dibenzylideneacetone)dipalladium(0) (22.9 mg, 0.025 mmol, Aldrich), 1,3,5,7-tetramethyl-6-phenyl-2,4,8-trioxa-6-phosphaadamantane (21.9 mg, 0.075 mmol, Aldrich), and potassium phosphate tribasic (2653 mg, 12.50 mmol) were combined in a reaction vial. The vial was evacuated and backfilled with nitrogen three times before being charged with dioxane (14 mL), 3-bromo-6-methoxy-2-methylpyridine (688 μL, 5 mmol, ArkPharm), vinylboronic acid pinacol ester (847 mg, 5.50 mmol, CombiBlocks), and water (2.8 mL). The reaction mixture was heated at 80° C. for 16 hours before cooling to ambient temperature. The organic phase was decanted off, concentrated under reduced pressure, and purified by flash chromatography (ISCO CombiFlash, 0-20% ethyl acetate/heptanes, 40 g RediSep® gold silica column) to afford the title compound (348 mg, 2.33 mmol, 47% yield). ¹H NMR (600 MHz, CDCl₃) δ ppm 7.66 (d, J=8.5 Hz, 1H), 6.84 (ddt, J=17.5, 11.0, 0.6 Hz, 1H), 6.56 (dp, J=8.5, 0.6 Hz, 1H), 5.52 (dd, J=17.5, 1.2 Hz, 1H), 5.23 (dd, J=11.0, 1.1 Hz, 1H), 3.92 (s, 3H), 2.50-2.45 (m, 3H).

### Example 72B

### (1S,2R)—(R)-4,4-dimethyl-2-oxotetrahydrofuran-3-yl 2-(6-methoxy-2-methylpyridin-3-yl)-1-(2-methoxy-5-methylphenyl)cyclopropanecarboxylate

A solution of Example 69D (200 mg, 0.628 mmol) in dichloromethane (1.4 mL) was added dropwise to a solution of Example 72A (141 mg, 0.942 mmol) and rhodium(II) octanoate dimer (4.9 mg, 6.3 μmol, Strem) in dichloromethane (2.8 mL) at 0° C. After 1 hour, the reaction was concentrated under reduced pressure, and the crude residue was purified by flash chromatography (ISCO CombiFlash, 0-100% ethyl acetate/heptanes, 40 g RediSep® gold silica column) to afford the title compound (207 mg, 0.471 mmol, 75.0% yield).

The material was determined to be 93% de by analytical chiral supercritical fluid chromatography (ChiralPak IC column, 3 mL/minute, 5-50% methanol/CO₂, major diastereomer is the second peak). ¹H NMR (500 MHz, CDCl₃) δ ppm 7.02 (d, J=2.3 Hz, 1H), 6.89 (ddd, J=8.2, 2.3, 0.8 Hz, 1H), 6.38 (dd, J=8.4, 4.2 Hz, 2H), 6.12-6.03 (m, 1H), 5.37 (s, 1H), 3.98-3.92 (m, 2H), 3.82 (s, 3H), 3.37 (s, 3H), 3.34 (dd, J=9.5, 7.6 Hz, 1H), 2.65 (d, J=0.6 Hz, 3H), 2.23 (s, 3H), 2.01 (dd, J=9.5, 5.2 Hz, 1H), 1.95 (dd, J=7.6, 5.2 Hz, 1H), 1.15 (s, 3H), 0.81 (s, 3H). MS(APCI+) m/z 440.4 (M+H)⁺.

### Example 72C

### (1S,2R)-2-(6-methoxy-2-methylpyridin-3-yl)-1-(2-methoxy-5-methylphenyl)cyclopropanecarboxylic acid

A mixture of Example 72B (205 mg, 0.466 mmol) and lithium hydroxide (112 mg, 4.66 mmol) in dioxane (1.75 mL) and water (0.58 mL) was heated to 80° C. After 16 hours, the reaction was diluted with water, acidified with 1 M hydrochloric acid to pH 3, and extracted with ethyl acetate. The organic phase was dried with MgSO₄, filtered, and concentrated under reduced pressure to afford the title compound (119 mg, 0.363 mmol, 78% yield). ¹H NMR (600 MHz, dimethyl sulfoxide-d₆) δ ppm 12.11 (s, 1H), 6.96 (d, J=2.3 Hz, 1H), 6.86 (ddd, J=8.2, 2.3, 0.9 Hz, 1H), 6.57 (d, J=8.5 Hz, 1H), 6.51 (d, J=8.3 Hz, 1H), 6.13 (d, J=8.5 Hz, 1H), 3.72 (s, 3H), 3.36 (s, 3H), 3.06 (dd, J=9.3, 7.3 Hz, 1H), 2.55 (s, 3H), 2.16 (s, 3H), 2.02 (dd, J=7.3, 5.0 Hz, 1H), 1.66 (dd, J=9.3, 5.0 Hz, 1H). MS(APCI+) m/z 328.3 (M+H)⁺.

### Example 72D

### (1S,2R)-1-(2-methoxy-5-methylphenyl)-2-(6-methoxy-2-methylpyridin-3-yl)-N-(2-methylquinoline-5-sulfonyl)cyclopropane-1-carboxamide

A mixture of Example 72C (50 mg, 0.153 mmol), 2-methylquinoline-5-sulfonamide (40.7 mg, 0.183 mmol), 1-(3-dimethylaminopropyl)-3-ethylcarbodiimide hydrochloride (58.6 mg, 0.305 mmol), and 4-dimethylaminopyridine (28.0 mg, 0.229 mmol) in dichloromethane (1.5 mL) was stirred at ambient temperature. After 16 hours, the reaction was acidified with trifluoroacetic acid and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (98 mg, 0.152 mmol, 99% yield). ¹H NMR (500 MHz, dimethyl sulfoxide-d₆) δ ppm 11.65 (s, 1H), 8.96 (dd, J=8.9, 0.8 Hz, 1H), 8.34-8.26 (m, 2H), 7.96 (dd, J=8.5, 7.4 Hz, 1H), 7.66 (d, J=8.9 Hz, 1H), 6.95-6.88 (m, 2H), 6.75 (d, J=8.6 Hz, 1H), 6.50 (d, J=8.2 Hz, 1H), 6.18 (d, J=8.5 Hz, 1H), 3.69 (s, 3H), 3.14 (s, 3H), 3.00 (dd, J=9.3, 7.3 Hz, 1H), 2.76 (s, 3H), 2.29 (s, 3H), 2.18 (s, 3H), 2.15 (dd, J=7.3, 5.6 Hz, 1H), 1.27 (dd, J=9.4, 5.7 Hz, 1H). MS(APCI+) m/z 532.1 (M+H)⁺.

### Example 73

### (1R,2S)-2-(2-ethoxypyridin-3-yl)-1-(2-methoxy-5-methylphenyl)-N-(2-methylquinoline-5-sulfonyl)cyclopropane-1-carboxamide

The cyclopropane was prepared according to the procedure described in Example 4A by substituting 2-ethoxy-3-vinylpyridine for 4-methylstyrene, followed by processing as in Examples 4B and 4C to afford the racemate of the title compound (206 mg). The enantiomers were separated by preparative chiral supercritical fluid chromatography (ChiralPak IC column, 40% methanol/CO₂, 70 mL/minute). The fractions containing the first eluting peak were concentrated under reduced pressure to afford the title compound (52.5 mg, 0.099 mmol, 25.5% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.92 (s, 1H), 8.99 (d, J=8.8 Hz, 1H), 7.92 (t, J=8.5 Hz, 2H), 7.69 (s, 1H), 7.65 (dd, J=8.4, 7.2 Hz, 1H), 7.38 (d, J=8.8 Hz, 1H), 7.07 (s, 4H), 6.78 (s, 1H), 6.37 (s, 2H), 6.16 (s, OH), 4.31 (s, 1H), 4.28-4.20 (m, 1H), 2.97 (s, 3H), 2.66 (s, 2H), 2.18 (s, 2H), 2.05 (s, 1H), 1.48 (s, OH), 1.35 (s, 1H), 1.18 (s, 3H). MS(APCI+) m/z 532.2 (M+H)⁺.

### Example 74

### (1R,2R)-1-(2-methoxy-5-methylphenyl)-2-(6-methoxypyrazin-2-yl)-N-(2-methylquinoline-5-sulfonyl)cyclopropane-1-carboxamide

The cyclopropane was prepared according to the procedure described in Example 4A by substituting 2-methoxy-6-vinylpyrazine for 4-methylstyrene, followed by processing as in Examples 4B and 4C to afford the racemate of the title compound (26.8 mg). The enantiomers were separated by preparative chiral supercritical fluid chromatography (ChiralPak IC column, 40% methanol/CO₂, 70 mL/minute). The fractions containing the first eluting peak were concentrated under reduced pressure to afford the title compound (7.4 mg, 0.014 mmol, 27.6% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.58 (s, 1H), 8.85 (d, J=8.8 Hz, 1H), 8.23 (s, 2H), 7.99 (s, 1H), 7.90 (d, J=8.8 Hz, 1H), 7.77 (s, 1H), 7.57 (d, J=8.9 Hz, 1H), 7.08 (s, 1H), 6.95 (d, J=8.2 Hz, 1H), 6.41 (d, J=8.2 Hz, 1H), 4.11 (s, 1H), 3.39 (s, 3H), 3.18 (s, 2H), 3.00 (s, 3H), 2.72 (s, 3H), 2.23 (s, 3H), 1.53 (dd, J=8.7, 4.3 Hz, 1H). MS(APCI+) m/z 519.1 (M+H)⁺.

### Example 75

### (1S,2S)-1-(2-methoxy-5-methylphenyl)-2-(6-methoxypyrazin-2-yl)-N-(2-methylquinoline-5-sulfonyl)cyclopropane-1-carboxamide

The cyclopropane was prepared according to the procedure described in Example 4A by substituting 2-methoxy-6-vinylpyrazine for 4-methylstyrene, followed by processing as in Examples 4B and 4C to afford the racemate of the title compound (26.8 mg). The enantiomers were separated by preparative chiral supercritical fluid chromatography (ChiralPak IC column, 40% methanol/CO₂, 70 mL/minute). The fractions containing the second eluting peak were concentrated under reduced pressure to afford the title compound (7.6 mg, 0.015 mmol, 28.4% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.67 (s, 1H), 8.87 (d, J=8.8 Hz, 1H), 8.18 (s, 2H), 7.97 (s, 1H), 7.85 (s, 1H), 7.76 (s, 1H), 7.54 (d, J=8.9 Hz, 1H), 7.07 (s, 1H), 6.92 (d, J=8.3 Hz, 1H), 6.40 (d, J=8.2 Hz, 1H), 3.18 (s, 1H), 3.17 (s, 1H), 3.00 (s, 3H), 2.73 (s, 3H), 2.22 (s, 3H), 1.53 (dd, J=8.8, 4.1 Hz, 1H). MS(APCI+) m/z 519.0 (M+H)⁺.

### Example 76

### (1S,2R)-2-(5-fluoro-6-methoxypyridin-3-yl)-1-(2-methoxy-5-methylphenyl)-N-(2-methylquinoline-5-sulfonyl)cyclopropane-1-carboxamide

### Example 76A

### 3-fluoro-2-methoxy-5-vinylpyridine

5-Bromo-3-fluoro-2-methoxypyridine (250 mg, 1.214 mmol, CombiBlocks), tris(dibenzylideneacetone)dipalladium(0) (5.6 mg, 6.1 μmol, Aldrich), 1,3,5,7-tetramethyl-6-phenyl-2,4,8-trioxa-6-phosphaadamantane (5.3 mg, 0.018 mmol, Aldrich), and potassium phosphate tribasic (644 mg, 3.03 mmol) were combined in a reaction vial. The vial was evacuated and backfilled with nitrogen three times before being charged with dioxane (4.0 mL), vinylboronic acid pinacol ester (224 mg, 1.456 mmol, CombiBlocks), and water (0.8 mL). After heating for 16 hours at 80° C., the reaction was cooled to ambient temperature and the layers separated. The organic phase was decanted and concentrated under reduced pressure. The crude material was purified by flash chromatography (ISCO CombiFlash, 0-20% ethyl acetate/heptanes, 12 g RediSep® gold silica column) to afford the title compound (78 mg, 0.509 mmol, 42.0% yield). ¹H NMR (400 MHz, CDCl₃) δ ppm 7.88 (d, J=2.0 Hz, 1H), 7.44 (dd, J=11.2, 2.0 Hz, 1H), 6.64 (ddd, J=17.7, 11.0, 1.7 Hz, 1H), 5.63 (d, J=17.6 Hz, 1H), 5.27 (d, J=11.0 Hz, 1H), 4.03 (s, 3H).

### Example 76B

### (1S,2R)—(R)-4,4-dimethyl-2-oxotetrahydrofuran-3-yl 2-(5-fluoro-6-methoxypyridin-3-yl)-1-(2-methoxy-5-methylphenyl)cyclopropanecarboxylate

A solution of Example 69D (130 mg, 0.408 mmol) in dichloromethane (0.90 mL) was added dropwise to a solution of Example 76A (75 mg, 0.490 mmol) and rhodium(II) octanoate dimer (3.2 mg, 4.1 μmol, Strem) in dichloromethane (1.8 mL) at 0° C. After 2 hours the reaction was concentrated under reduced pressure and purified by flash chromatography (ISCO CombiFlash, 0-40% ethyl acetate/heptanes, 12 g RediSep® gold silica column) to afford the title compound (133 mg, 0.300 mmol, 73.5% yield). ¹H NMR (500 MHz, dimethyl sulfoxide-d₆) δ ppm 7.64 (d, J=2.0 Hz, 1H), 7.10 (d, J=2.2 Hz, 1H), 7.04 (dd, J=12.1, 2.0 Hz, 1H), 6.95 (ddd, J=8.2, 2.3, 0.8 Hz, 1H), 6.56 (d, J=8.3 Hz, 1H), 5.55 (s, 1H), 4.f10 (d, J=8.6 Hz, 1H), 3.94 (d, J=8.5 Hz, 1H), 3.80 (s, 3H), 3.42 (s, 3H), 3.17 (dd, J=9.3, 7.3 Hz, 1H), 2.29 (dd, J=7.3, 5.5 Hz, 1H), 2.21 (s, 3H), 1.79 (dd, J=9.3, 5.5 Hz, 1H), 1.05 (s, 3H), 0.58 (s, 3H). MS(APCI+) m/z 444.2 (M+H)⁺.

### Example 76C

### (1S,2R)-2-(5-fluoro-6-methoxypyridin-3-yl)-1-(2-methoxy-5-methylphenyl)-N-(2-methylquinoline-5-sulfonyl)cyclopropane-1-carboxamide

A mixture of Example 76B (130 mg, 0.293 mmol) and lithium hydroxide (70.2 mg, 2.93 mmol) in dioxane (1.5 mL) and water (0.5 mL) was heated to 80° C. After 2 hours, the reaction was diluted with water and acidified to pH 3 with 1 M hydrochloric acid. The mixture was extracted with ethyl acetate, and the organic phase was dried with MgSO₄, filtered, and concentrated under reduced pressure. 2-Methylquinoline-5-sulfonamide (82 mg, 0.369 mmol), 1-(3-dimethylaminopropyl)-3-ethylcarbodiimide hydrochloride (118 mg, 0.616 mmol), 4-dimethylaminopyridine (56.4 mg, 0.462 mmol) and dichloromethane (1.5 mL) were added to the crude acid residue. After stirring at ambient temperature for 16 hours, the reaction was acidified with trifluoroacetic acid and concentrated under reduced pressure. The crude residue was then purified by reverse-phase HPLC (Waters Xbridge Prep C18 column, 42 mL/minute, 5-95% acetonitrile/0.1% trifluoroacetic acid in water) to afford the title compound (144.5 mg, 0.270 mmol, 88% yield). ¹H NMR (600 MHz, dimethyl sulfoxide-d₆) δ ppm 11.62 (s, 1H), 8.92 (dd, J=8.8, 0.9 Hz, 1H), 8.34-8.23 (m, 2H), 7.95 (dd, J=8.5, 7.4 Hz, 1H), 7.65 (d, J=8.9 Hz, 1H), 7.51-7.44 (m, 1H), 7.01 (d, J=2.4 Hz, 1H), 6.97 (ddd, J=8.2, 2.2, 0.8 Hz, 1H), 6.89 (dd, J=12.2, 2.0 Hz, 1H), 6.52 (d, J=8.3 Hz, 1H), 3.76 (s, 3H), 3.13 (s, 3H), 3.05 (dd, J=9.2, 7.1 Hz, 1H), 2.75 (s, 3H), 2.22 (t, J=0.7 Hz, 3H), 2.09 (dd, J=7.1, 5.7 Hz, 1H), 1.37 (dd, J=9.3, 5.6 Hz, 1H). MS(APCI+) m/z 536.3 (M+H)⁺.

### Example 77

### (1S,2S)-2-[6-(difluoromethyl)pyridin-2-yl]-1-(2-methoxy-5-methylphenyl)-N-(2-methylquinoline-5-sulfonyl)cyclopropane-1-carboxamide

### Example 77A

### 2-(difluoromethyl)-6-vinylpyridine

To 2-bromo-6-(difluoromethyl)pyridine (0.5 g, 2.404 mmol), 4,4,5,5-tetramethyl-2-vinyl-1,3,2-dioxaborolane (0.370 g, 2.404 mmol), and potassium carbonate (0.831 g, 6.01 mmol) in ethanol (3 mL) and tetrahydrofuran (6 mL) was added bis(triphenylphosphine)palladium(II) dichloride (0.169 g, 0.240 mmol). The reaction mixture was bubbled with nitrogen before being stirred overnight at 60° C. in a sealed vessel. The mixture was cooled to ambient temperature before adding water (5 mL) and extracting with ethyl acetate (3×10 mL). The combined organic layers were dried over MgSO₄, filtered, concentrated, and then purified via flash chromatography (ISCO CombiFlash, 0-30% ethyl acetate/heptanes, 12 g RediSep® gold silica column) to afford 2-(difluoromethyl)-6-vinylpyridine (201 mg, 1.296 mmol, 26.9% yield). ¹H NMR (600 MHz, CDCl₃) δ 7.80 (t, J=7.8 Hz, 1H), 7.51 (dd, J=7.7, 0.9 Hz, 1H), 7.45 (dd, J=7.8, 1.1 Hz, 1H), 6.85 (dd, J=17.5, 10.8 Hz, 1H), 6.63 (t, J=55.5 Hz, 1H), 6.29 (dd, J=17.5, 1.1 Hz, 1H), 5.57 (dd, J=10.8, 1.2 Hz, 1H). MS(ESI+) m/z 156.2 (M+H)⁺.

### Example 77B

### (1S,2S)-2-(6-(difluoromethyl)pyridin-2-yl)-1-(2-methoxy-5-methylphenyl)-N-((2-methylquinolin-5-yl)sulfonyl)cyclopropanecarboxamide

The cyclopropane was prepared according to the procedure described in Example 69E by substituting 2-(difluoromethyl)-6-vinylpyridine for Example 69A, followed by processing as in Examples 69G, and 69H to afford the title compound. ¹H NMR (600 MHz, CDCl₃) δ 9.53 (d, J=9.0 Hz, 1H), 8.81 (d, J=8.5 Hz, 1H), 8.57 (dd, J=7.5, 0.9 Hz, 1H), 8.41 (s, 1H), 8.10 (dd, J=8.5, 7.5 Hz, 1H), 7.73 (d, J=9.0 Hz, 1H), 7.51 (t, J=7.7 Hz, 1H), 7.20 (d, J=7.6 Hz, 1H), 7.09-7.06 (m, 2H), 7.05 (ddd, J=8.3, 2.2, 0.8 Hz, 1H), 6.42 (d, J=8.3 Hz, 1H), 6.02 (t, J=55.7 Hz, 1H), 3.20-3.17 (m, 1H), 3.17 (s, 3H), 3.09 (s, 3H), 2.32 (dd, J=7.0, 4.5 Hz, 1H), 2.30 (s, 3H), 1.91 (dd, J=8.9, 4.5 Hz, 1H). MS(ESI+) m/z 537.9 (M+H)⁺.

### Example 78

### rac-(1r,2s)-1-(2-methoxy-5-methylphenyl)-2-[2-methoxy-6-(propan-2-yl)pyridin-3-yl]-N-(2-methylquinoline-5-sulfonyl)cyclopropane-1-carboxamide

The title compound was prepared according to the procedures described in Example 61 by substituting 6-chloro-2-methoxy-3-vinylpyridine for 2-chloro-5-vinylpyrazine in Example 61A. ¹H NMR (400 MHz, dimethyl sulfoxide-d₆) δ ppm 8.84 (d, J=8.9 Hz, 1H), 8.23 (dd, J=8.0, 3.3 Hz, 2H), 7.89 (t, J=7.9 Hz, 1H), 7.59 (d, J=9.0 Hz, 1H), 6.90 (dd, J=8.3, 2.2 Hz, 1H), 6.75 (s, 1H), 6.47 (d, J=8.4 Hz, 1H), 6.35 (d, J=7.6 Hz, 1H), 6.24 (d, J=7.6 Hz, 1H), 3.70 (s, 3H), 3.09 (s, 3H), 2.98 (t, J=8.6 Hz, 1H), 2.70 (s, 3H), 2.10 (s, 3H), 1.81 (dd, J=7.8, 5.3 Hz, 1H), 1.48 (dd, J=9.4, 5.3 Hz, 1H), 1.04 (dd, J=6.8, 3.9 Hz, 6H). MS(APCI+) m/z 560.3 (M+H)⁺.

### Example 79

### rac-(1r,2s)-2-(2,6-dimethoxypyridin-3-yl)-1-(2-methoxy-5-methylphenyl)-N-(2-methylquinoline-5-sulfonyl)cyclopropane-1-carboxamide

### Example 79A

### 6-chloro-2-methoxy-3-vinylpyridine

n-Butyllithium (1340 μL, 3.35 mmol, 2.5 M in hexanes) was added dropwise to a suspension of methyl triphenylphosphonium bromide (1197 mg, 3.35 mmol) in tetrahydrofuran (9714 μL) at −78° C. The reaction was stirred at −78° C. for 30 minutes, and then a solution of 6-chloro-2-methoxynicotinaldehyde (500 mg, 2.91 mmol) in tetrahydrofuran (4857 μL) was added dropwise. The reaction mixture was warmed to ambient temperature and stirred overnight. The reaction was quenched with saturated aqueous NH₄Cl and extracted with ethyl acetate. The organic phase was washed with brine, dried with Na₂SO₄, concentrated, purified by flash chromatography (ISCO CombiFlash, 0-50% ethyl acetate/heptanes, 12 g RediSep® gold silica column) to afford the title compound (148 mg, 0.873 mmol, 29.9% yield).

### Example 79B

### rac-(1r,2s)-methyl 2-(6-chloro-2-methoxypyridin-3-yl)-1-(2-methoxy-5-methylphenyl)cyclopropanecarboxylate

The title compound was prepared according to the procedure described in Example 61A by substituting Example 79A for 2-chloro-5-vinylpyrazine. MS(APCI+) m/z 362.15 (M+H)⁺.

### Example 79C

### rac-(1r,2s)-2-(2,6-dimethoxypyridin-3-yl)-1-(2-methoxy-5-methylphenyl)cyclopropanecarboxylic acid

The title compound was prepared according to the procedures described in Example 62B by substituting Example 79B for Example 62A and by substituting sodium methoxide for sodium ethoxide. MS(APCI+) m/z 344.19 (M+H)⁺.

### Example 79D

rac-(1r,2s)-2-(2,6-dimethoxypyridin-3-yl)-1-(2-methoxy-5-methylphenyl)-N-(2-methylquinoline-5-sulfonyl)cyclopropane-1-carboxamide

The title compound was prepared according to the procedures described in Example 62C by substituting Example 79C for Example 62B. ¹H NMR (600 MHz, dimethyl sulfoxide-d₆) δ ppm 11.32 (s, 1H), 8.90 (d, J=8.9 Hz, 1H), 8.32-8.21 (m, 2H), 7.93 (dd, J=8.3, 7.6 Hz, 1H), 7.63 (d, J=8.9 Hz, 1H), 6.94 (ddd, J=8.2, 2.3, 0.9 Hz, 1H), 6.85 (s, 1H), 6.54 (d, J=8.3 Hz, 1H), 6.50 (d, J=8.2 Hz, 1H), 5.83 (d, J=8.1 Hz, 1H), 3.73 (d, J=23.8 Hz, 6H), 3.17 (s, 3H), 3.00 (dd, J=9.4, 7.7 Hz, 1H), 2.74 (s, 3H), 2.16 (s, 3H), 1.90 (dd, J=7.6, 5.4 Hz, 1H), 1.41 (dd, J=9.5, 5.4 Hz, 1H). MS(APCI+) m/z 548.25 (M+H)⁺.

### Example 80

### (1S,2S)-2-(5,6-dimethoxypyrazin-2-yl)-1-(2-methoxy-5-methylphenyl)-N-(2-methylquinoline-5-sulfonyl)cyclopropane-1-carboxamide

### Example 80A

### 2,3-dimethoxy-5-vinylpyrazine

To 5-bromo-2,3-dimethoxypyrazine (0.5 g, 2.283 mmol), 4,4,5,5-tetramethyl-2-vinyl-1,3,2-dioxaborolane (0.352 g, 2.283 mmol), and potassium carbonate (0.789 g, 5.71 mmol) in ethanol (3 mL) and tetrahydrofuran (6 mL) was added bis(triphenylphosphine)palladium(II) dichloride (0.160 g, 0.228 mmol). The reaction mixture was bubbled with nitrogen before heated to 60° C. in a microwave reactor. The mixture was cooled to ambient temperature before adding water (5 mL) and extracting with ethyl acetate (3×10 mL). The combined organic layers were dried over MgSO₄, filtered, concentrated, and then purified via flash chromatography (ISCO CombiFlash, 0-30% ethyl acetate/heptanes) to afford the title compound (86 mg, 0.518 mmol, 22.67% yield). MS(ESI+) m/z 167.1 (M+H)⁺.

### Example 80B

### (1S,2S)-2-(5,6-dimethoxypyrazin-2-yl)-1-(2-methoxy-5-methylphenyl)-N-((2-methylquinolin-5-yl)sulfonyl)cyclopropanecarboxamide

The cyclopropane was prepared according to the procedure described in Example 69E by substituting 2,3-dimethoxy-5-vinylpyrazine for Example 69A, followed by processing as in Examples 69G and 69H to afford the title compound. ¹H NMR (500 MHz, CDCl₃) δ 9.24 (d, J=9.0 Hz, 1H), 8.70 (d, J=8.5 Hz, 1H), 8.52 (d, J=7.5 Hz, 1H), 8.33 (s, 1H), 8.00 (dd, J=8.4, 7.5 Hz, 1H), 7.60 (d, J=8.9 Hz, 1H), 7.49 (s, 1H), 7.06 (ddd, J=8.4, 2.3, 0.9 Hz, 1H), 6.49 (d, J=8.3 Hz, 1H), 3.88 (s, 3H), 3.35 (s, 3H), 3.24 (s, 3H), 3.02-2.98 (m, 4H), 2.26 (s, 3H), 2.12 (dd, J=7.0, 4.3 Hz, 1H), 1.85 (dd, J=9.1, 4.2 Hz, 1H). MS(APCI+) m/z 549.4 (M+H)⁺.

### Example 81

### (1S,2R)-2-(2,6-dimethylpyridin-3-yl)-1-(2-methoxy-5-methylphenyl)-N-(2-methylquinoline-5-sulfonyl)cyclopropane-1-carboxamide

### Example 81A

### 2,6-dimethyl-3-vinylpyridine

Tris(dibenzylideneacetone)dipalladium(0)) (Pd₂(dba)₃) (12.30 mg, 0.013 mmol), 1,3,5,7-tetramethyl-6-phenyl-2,4,8-trioxa-6-phosphaadamantane (11.78 mg, 0.040 mmol), and potassium phosphate tribasic (1426 mg, 6.72 mmol) were combined in a reaction vial with septum cap and stir bar. The vial was degassed and backfilled with nitrogen three times before being charged with dioxane (7465 μL), 3-bromo-2,6-dimethylpyridine (500 mg, 2.69 mmol), vinylboronic acid pinacol ester (455 mg, 2.96 mmol) and water (1493 μL). The reaction mixture was then heated overnight at 80° C. After cooling to ambient temperature, the reaction mixture was diluted with ethyl acetate, and the organic and aqueous layers were separated. The organic phase was concentrated under reduced pressure. The crude residue was then purified via flash chromatography (ISCO CombiFlash, 0-30% ethyl acetate/heptanes, 12 g RediSep® gold silica column) to afford the title compound (339 mg, 2.55 mmol, 95% yield). MS (APCI+) m/z 134.2 (M+H)⁺.

### Example 81B

### (1S,2R)—(R)-4,4-dimethyl-2-oxotetrahydrofuran-3-yl 2-(2,6-dimethylpyridin-3-yl)-1-(2-methoxy-5-methylphenyl)cyclopropanecarboxylate

A solution of Example 81A (126 mg, 0.942 mmol) in dichloromethane (2792 μL) was added to a reaction vial containing rhodium(II) octanoate dimer (4.89 mg, 6.28 μmol) to afford a solution that was cooled in an ice bath. A solution of Example 69D (200 mg, 0.628 mmol) in dichloromethane (1396 μL) was then added dropwise over 2 minutes. After stirring overnight, LC/MS showed complete consumption of the diazo compound and a new major peak with the product mass. The reaction was concentrated under reduced pressure, and the crude residue was purified via flash chromatography (ISCO CombiFlash, 0-100% ethyl acetate/heptanes, 12 g RediSep® gold silica column) to afford the title compound (155 mg, 0.366 mmol, 58.3% yield). The material was determined to be >90% de by supercritical fluid chromatography (ChiralPak IC, column, 40% methanol/CO₂, 70 mL/minute). MS(APCI+) m/z 423.97 (M+H)⁺.

### Example 81C

### (1S,2R)-2-(2,6-dimethylpyridin-3-yl)-1-(2-methoxy-5-methylphenyl)cyclopropanecarboxylic acid

The title compound was prepared according to the procedure described in Example 69G by substituting Example 81B for Example 69F. MS(APCI+) m/z 312.20 (M+H)⁺.

### Example 81D

### (1S,2R)-2-(2,6-dimethylpyridin-3-yl)-1-(2-methoxy-5-methylphenyl)-N-(2-methylquinoline-5-sulfonyl)cyclopropane-1-carboxamide

The title compound was prepared according to the procedure described in Example 69H by substituting Example 81C for Example 69G. ¹H NMR (500 MHz, dimethyl sulfoxide-d₆) δ ppm 11.97 (s, 1H), 9.01 (d, J=8.8 Hz, 1H), 8.02 (s, 2H), 7.72 (t, J=7.6 Hz, 1H), 7.47 (d, J=8.8 Hz, 1H), 7.19 (s, 1H), 7.09 (s, 1H), 6.99 (s, 1H), 6.83 (s, 1H), 6.60 (s, 1H), 6.50 (s, 1H), 6.37 (d, J=8.8 Hz, 1H), 2.99 (s, 1H), 2.70 (s, 3H), 2.42 (s, 3H), 2.25 (s, 3H), 2.15 (s, 3H), 1.74 (s, 1H), 1.42 (s, 1H). MS(APCI+) m/z 516.2 (M+H)⁺.

### Example 82

### (1S,2S)-2-(3,5-dimethoxypyrazin-2-yl)-1-(2-methoxy-5-methylphenyl)-N-(2-methylquinoline-5-sulfonyl)cyclopropane-1-carboxamide

### Example 82A

### 3,5-dimethoxy-2-vinylpyrazine

To 2-bromo-3,5-dimethoxypyrazine (0.5 g, 2.283 mmol), 4,4,5,5-tetramethyl-2-vinyl-1,3,2-dioxaborolane (0.352 g, 2.283 mmol), and potassium carbonate (0.789 g, 5.71 mmol) in ethanol (3 mL) and tetrahydrofuran (6 mL) was added bis(triphenylphosphine)palladium(II) dichloride (0.160 g, 0.228 mmol). The reaction mixture was bubbled with nitrogen before heated to 60° C. in a microwave reactor. The mixture was cooled to ambient temperature before adding water (5 mL) and extracting with ethyl acetate (3×10 mL). The combined organic layers were dried over MgSO₄, filtered, concentrated, and then purified on a silica column (ISCO CombiFlash, 0-30% ethyl acetate/heptanes) to afford the title compound (272.3 mg, 1.639 mmol, 71.8% yield). ¹H NMR (600 MHz, CDCl₃) δ 7.76 (s, 1H), 6.94 (ddd, J=17.6, 11.1, 0.4 Hz, 1H), 6.18 (dd, J=17.5, 1.9 Hz, 1H), 5.37 (dd, J=11.1, 1.9 Hz, 1H), 3.98 (s, 3H), 3.95 (s, 3H). MS(ESI+) m/z 167.1 (M+H)⁺.

### Example 82B

### (1S,2S)-2-(3,5-dimethoxypyrazin-2-yl)-1-(2-methoxy-5-methylphenyl)-N-((2-methylquinolin-5-yl)sulfonyl)cyclopropanecarboxamide

The cyclopropane was prepared according to the procedure described in Example 69E by substituting 3,5-dimethoxy-2-vinylpyrazine for Example 69A, followed by processing as in Examples 69G and 69H to afford the title compound. ¹H NMR (600 MHz, CDCl₃) δ 9.43 (dd, J=9.0, 0.8 Hz, 1H), 8.77 (d, J=8.5 Hz, 1H), 8.54 (dd, J=7.5, 1.1 Hz, 1H), 8.06 (dd, J=8.6, 7.5 Hz, 1H), 7.66 (d, J=9.0 Hz, 1H), 7.16 (s, 1H), 7.02 (ddd, J=8.3, 2.3, 0.8 Hz, 1H), 6.95 (d, J=2.3 Hz, 1H), 6.47 (d, J=8.3 Hz, 1H), 3.93 (s, 3H), 3.84 (s, 3H), 3.31 (s, 3H), 3.28 (dd, J=9.2, 7.4 Hz, 1H), 3.05 (s, 3H), 2.26-2.23 (m, 1H), 2.23 (q, J=0.9 Hz, 3H), 1.82 (dd, J=9.2, 4.4 Hz, 1H). MS(APCI+) m/z 549.4 (M+H)⁺.

### Example 83

### (1S,2S)-1-(2-methoxy-5-methylphenyl)-2-(5-methoxy-6-methylpyrazin-2-yl)-N-(2-methylquinoline-5-sulfonyl)cyclopropane-1-carboxamide

### Example 83A

### (1S,2S)—(R)-4,4-dimethyl-2-oxotetrahydrofuran-3-yl 2-(5-chloro-6-methylpyrazin-2-yl)-1-(2-methoxy-5-methylphenyl)cyclopropanecarboxylate

The title compound was prepared according to the procedure described in Example 81B by substituting 2-chloro-3-methyl-5-vinylpyrazine for Example 81A. MS(APCI+) m/z 444.91 (M+H)⁺.

### Example 83B

### (1S,2S)-1-(2-methoxy-5-methylphenyl)-2-(5-methoxy-6-methylpyrazin-2-yl)cyclopropanecarboxylic acid

The title compound was prepared according to the procedure described in Example 81C by substituting Example 83A for Example 81B. MS(APCI+) m/z 329.07 (M+H)⁺.

### Example 83C

### (1S,2S)-1-(2-methoxy-5-methylphenyl)-2-(5-methoxy-6-methylpyrazin-2-yl)-N-(2-methylquinoline-5-sulfonyl)cyclopropane-1-carboxamide

The title compound was prepared according to the procedure described in Example 81D by substituting Example 83B for Example 81C. ¹H NMR (500 MHz, dimethyl sulfoxide-d₆) δ ppm 11.57 (s, 1H), 8.90 (d, J=8.9 Hz, 1H), 8.29 (ddd, J=8.5, 3.9, 1.1 Hz, 2H), 7.95 (dd, J=8.5, 7.4 Hz, 1H), 7.64 (d, J=9.0 Hz, 1H), 7.57 (s, 1H), 6.99 (d, J=2.2 Hz, 1H), 6.93 (dd, J=8.5, 2.2 Hz, 1H), 6.45 (d, J=8.3 Hz, 1H), 3.73 (s, 3H), 3.16 (dd, J=9.1, 7.0 Hz, 1H), 3.07 (s, 3H), 2.75 (s, 3H), 2.22 (d, J=11.9 Hz, 1H), 2.02 (s, 3H), 1.43 (dd, J=9.1, 4.8 Hz, 1H). MS(APCI+) m/z 533.2 (M+H)⁺.

### Example 84

### (1S,2R)-2-(5-fluoro-2-methoxypyridin-3-yl)-1-(2-methoxy-5-methylphenyl)-N-(2-methylquinoline-5-sulfonyl)cyclopropane-1-carboxamide

### Example 84A

### 5-fluoro-2-methoxy-3-vinylpyridine

The title compound was prepared according to the procedure described in Example 81A by substituting 3-bromo-5-fluoro-2-methoxypyridine for 3-bromo-2,6-dimethylpyridine. MS(APCI+) m/z 154.07 (M+H)⁺.

### Example 84B

### (1S,2R)—(R)-4,4-dimethyl-2-oxotetrahydrofuran-3-yl 2-(5-fluoro-2-methoxypyridin-3-yl)-1-(2-methoxy-5-methylphenyl)cyclopropanecarboxylate

The title compound was prepared according to the procedure described in Example 81B by substituting Example 84A for Example 81A. MS(APCI+) m/z 444.25 (M+H)⁺.

### Example 84C

### (1S,2R)-2-(5-fluoro-2-methoxypyridin-3-yl)-1-(2-methoxy-5-methylphenyl)cyclopropanecarboxylic acid

The title compound was prepared according to the procedure described in Example 81C by substituting Example 84B for Example 81B. MS(APCI+) m/z 332.25 (M+H)⁺.

### Example 84D

### (1S,2R)-2-(5-fluoro-2-methoxypyridin-3-yl)-1-(2-methoxy-5-methylphenyl)-N-(2-methylquinoline-5-sulfonyl)cyclopropane-1-carboxamide

The title compound was prepared according to the procedure described in Example 81D by substituting Example 84C for Example 81C. ¹H NMR (500 MHz, dimethyl sulfoxide-d₆) δ ppm 11.49 (s, 1H), 8.88 (d, J=8.8 Hz, 1H), 8.28 (d, J=7.8 Hz, 2H), 7.93 (t, J=7.9 Hz, 1H), 7.72 (d, J=2.9 Hz, 1H), 7.62 (d, J=8.9 Hz, 1H), 6.99 (d, J=2.2 Hz, OH), 6.98 (s, 2H), 6.51 (dd, J=9.0, 3.4 Hz, 2H), 3.75 (s, 3H), 3.15 (t, J=8.3 Hz, 1H), 3.09 (s, 3H), 2.74 (s, 3H), 2.22 (s, 3H), 2.10 (dd, J=7.5, 5.7 Hz, 1H), 2.09 (s, 1H), 1.45 (dd, J=9.2, 5.6 Hz, 1H). MS(APCI+) m/z 536.2 (M+H)⁺.

### Example 85

### (1R,2S)-1-(2-methoxy-5-methylphenyl)-2-(2-methoxy-6-methylpyridin-3-yl)-N-(2-methylquinoline-5-sulfonyl)cyclopropane-1-carboxamide

### Example 85A

### 2-methoxy-6-methyl-3-vinylpyridine

The title compound was prepared according to the procedure described in Example 81A by substituting 3-bromo-2-methoxy-6-methylpyridine for 3-bromo-2,6-dimethylpyridine.

### Example 85B

### rac-(1s,2r)-(R)-4,4-dimethyl-2-oxotetrahydrofuran-3-yl 1-(2-methoxy-5-methylphenyl)-2-(2-methoxy-6-methylpyridin-3-yl)cyclopropanecarboxylate

The title compound was prepared according to the procedure described in Example 81B by substituting Example 85A for Example 81A. MS(APCI+) m/z 440.28 (M+H)⁺.

### Example 85C

### rac (1s,2r)-1-(2-methoxy-5-methylphenyl)-2-(2-methoxy-6-methylpyridin-3-yl)cyclopropanecarboxylic acid

The title compound was prepared according to the procedure described in Example 81C by substituting Example 85B for Example 81B. MS(APCI+) m/z 327.94 (M+H)⁺.

### Example 85D

### rac-(1s,2r)-1-(2-methoxy-5-methylphenyl)-2-(2-methoxy-6-methylpyridin-3-yl)-N-((2-methylquinolin-5-yl)sulfonyl)cyclopropanecarboxamide

The title compound was prepared according to the procedure described in Example 81D by substituting Example 85C for Example 81C. ¹H NMR (600 MHz, dimethyl sulfoxide-d₆) δ ppm 11.30 (s, 1H), 8.86 (s, 1H), 8.21 (s, 3H), 7.86 (s, 1H), 7.55 (s, 1H), 6.89 (s, 2H), 6.49 (s, 1H), 6.37 (s, 1H), 6.28 (s, 1H), 3.71 (s, 3H), 3.03-3.13 (m, 3H), 2.71 (s, 3H), 2.18 (s, 3H), 2.15 (s, 4H), 1.88 (s, 1H), 1.43 (s, 1H). MS(APCI+) m/z 532.24 (M+H)⁺.

### Example 85E

### (1R,2S)-1-(2-methoxy-5-methylphenyl)-2-(2-methoxy-6-methylpyridin-3-yl)-N-(2-methylquinoline-5-sulfonyl)cyclopropane-1-carboxamide

The enantiomers of Example 85D (97 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (5.8 mg, 0.001 mmol, 5.99% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.32 (s, 1H), 8.88 (s, 1H), 8.21 (s, 3H), 7.86 (s, 1H), 7.55 (s, 1H), 6.89 (s, 2H), 6.49 (s, 1H), 6.37 (s, 1H), 6.28 (s, 1H), 3.73 (s, 3H), 3.03-3.13 (m, 3H), 2.71 (s, 3H), 2.18 (s, 3H), 2.15 (s, 4H), 1.88 (s, 1H), 1.43 (s, 1H). MS(APCI+) m/z 532.2 (M+H)⁺.

### Example 86

### (1S,2R)-1-(2-methoxy-5-methylphenyl)-2-(2-methoxy-6-methylpyridin-3-yl)-N-(2-methylquinoline-5-sulfonyl)cyclopropane-1-carboxamide

The enantiomers of Example 85D (97 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (50.5 mg, 0.095 mmol, 52.1% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.26 (s, 1H), 8.83 (d, J=8.9 Hz, 1H), 8.23 (d, J=8.0 Hz, 2H), 7.88 (t, J=7.9 Hz, 1H), 7.57 (d, J=8.9 Hz, 1H), 6.90 (dd, J=8.4, 2.2 Hz, 1H), 6.82 (d, J=2.3 Hz, 1H), 6.48 (d, J=8.3 Hz, 1H), 6.37 (d, J=7.5 Hz, 1H), 6.25 (d, J=8.9 Hz, 1H), 3.68 (s, 3H), 3.09 (s, 3H), 3.02 (dd, J=9.3, 7.7 Hz, 1H), 2.69 (s, 3H), 2.13 (d, J=6.9 Hz, 6H), 1.87 (dd, J=7.7, 5.4 Hz, 1H), 1.40 (dd, J=9.4, 5.3 Hz, 1H). MS(APCI+) m/z 532.2 (M+H)⁺.

### Example 87

### (1R,2S)-1-(2-methoxy-5-methylphenyl)-2-[2-methoxy-6-(propan-2-yl)pyridin-3-yl]-N-(2-methylquinoline-5-sulfonyl)cyclopropane-1-carboxamide

### Example 87A

### rac-(1r,2s)-methyl 2-(6-isopropyl-2-methoxypyridin-3-yl)-1-(2-methoxy-5-methylphenyl)cyclopropanecarboxylate

The title compound was prepared according to the procedure described in Example 61B by substituting Example 79B for Example 61A. MS(APCI+) m/z 370.23 (M+H)⁺.

### Example 87B

### rac-(1r,2s)-2-(6-isopropyl-2-methoxypyridin-3-yl)-1-(2-methoxy-5-methylphenyl)cyclopropanecarboxylic acid

The title compound was prepared according to the procedure described in Example 4B by substituting Example 87A for Example 4A. MS(APCI+) m/z 356.21 (M+H)⁺.

### Example 87C

### rac-(1r,2s)-2-(6-isopropyl-2-methoxypyridin-3-yl)-1-(2-methoxy-5-methylphenyl)-N-((2-methylquinolin-5-yl)sulfonyl)cyclopropanecarboxamide

The title compound was prepared according to the procedure described in Example 4C by substituting Example 87B for Example 4B. ¹H NMR (400 MHz, dimethyl sulfoxide-d₆) δ ppm 11.22 (s, 1H), 8.81 (d, J=8.9 Hz, 1H), 8.17 (s, 2H), 7.83 (s, 1H), 7.53 (s, 1H), 6.87 (s, 1H), 6.75 (s, 1H), 6.45 (d, J=8.2 Hz, 1H), 6.31 (s, 1H), 6.23 (d, J=7.5 Hz, 1H), 3.71 (s, 3H), 3.08 (s, 3H), 2.74-2.62 (m, 1H), 2.68 (s, 4H), 2.09 (s, 3H), 1.48 (d, J=9.0 Hz, 1H), 1.21 (s, OH), 1.04 (dd, J=6.9, 3.9 Hz, 7H). MS(APCI+) m/z 560.3 (M+H)⁺.

### Example 87D

### (1R,2S)-1-(2-methoxy-5-methylphenyl)-2-[2-methoxy-6-(propan-2-yl)pyridin-3-yl]-N-(2-methylquinoline-5-sulfonyl)cyclopropane-1-carboxamide

The enantiomers of Example 87C (25 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (8 mg, 0.014 mmol, 32% yield). The material was determined to be >98% ee by analytical supercritical fluid chromatography (ChiralPak IC column, 3 mL/minute, 40-50% methanol/CO₂). ¹H NMR (400 MHz, dimethyl sulfoxide-d₆) δ ppm 11.22 (s, 1H), 8.83 (d, J=8.9 Hz, 1H), 8.17 (s, 2H), 7.83 (s, 1H), 7.53 (s, 1H), 6.87 (s, 1H), 6.75 (s, 1H), 6.45 (d, J=8.2 Hz, 1H), 6.32 (s, 1H), 6.23 (d, J=7.5 Hz, 1H), 3.71 (s, 3H), 3.08 (s, 3H), 2.74-2.62 (m, 1H), 2.68 (s, 4H), 2.09 (s, 3H), 1.48 (d, J=9.0 Hz, 1H), 1.20 (s, OH), 1.04 (dd, J=6.9, 3.9 Hz, 7H). MS(APCI+) m/z 560.3 (M+H)⁺.

### Example 88

### (1S,2R)-1-(2-methoxy-5-methylphenyl)-2-[2-methoxy-6-(propan-2-yl)pyridin-3-yl]-N-(2-methylquinoline-5-sulfonyl)cyclopropane-1-carboxamide

The enantiomers of Example 87C (25 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (8 mg, 0.014 mmol, 32% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 11.31 (s, 1H), 8.89 (d, J=8.7 Hz, 1H), 8.20 (s, 2H), 7.86 (s, 1H), 7.57 (s, 1H), 6.91 (s, 1H), 6.80 (s, 1H), 6.50 (s, 1H), 6.36 (s, 1H), 6.28 (d, J=7.5 Hz, 1H), 3.77 (s, 3H), 3.12 (s, 2H), 3.03 (s, 1H), 2.73 (p, J=6.8 Hz, 1H), 2.73 (s, 3H), 2.14 (s, 3H), 1.78 (s, 1H), 1.53 (s, 1H), 1.10 (dd, J=6.8, 4.8 Hz, 6H). MS(APCI+) m/z 560.2 (M+H)⁺.

### Example 89

### rac-(1r,2r)-1-(2-methoxy-5-methylphenyl)-2-(5-methylpyrazin-2-yl)-N-(2-methylquinoline-5-sulfonyl)cyclopropane-1-carboxamide

### Example 89A

### 2-methyl-5-vinylpyrazine

The title compound was prepared according to the procedure described in Example 81A by substituting 2-bromo-5-methylpyrazine (Combi-Blocks) for 3-bromo-2,6-dimethylpyridine.

### Example 89B

### rac-(1r,2r)-1-(2-methoxy-5-methylphenyl)-2-(5-methylpyrazin-2-yl)-N-(2-methylquinoline-5-sulfonyl)cyclopropane-1-carboxamide

The cyclopropane was prepared according to the procedure described in Example 4A by substituting Example 89A for 4-methylstyrene, followed by processing as in Examples 4B and 4C to afford the title compound. ¹H NMR (500 MHz, dimethyl sulfoxide-d₆) δ ppm 8.95 (d, J=9.0 Hz, 1H), 8.35-8.28 (m, 2H), 8.12 (d, J=1.5 Hz, 1H), 8.03-7.94 (m, 2H), 7.69 (d, J=8.9 Hz, 1H), 6.99 (d, J=2.2 Hz, 1H), 6.94 (ddd, J=8.2, 2.2, 0.9 Hz, 1H), 6.44 (d, J=8.2 Hz, 1H), 3.30-3.21 (m, 1H), 3.08 (s, 3H), 2.77 (s, 3H), 2.30-2.23 (m, 4H), 2.21 (s, 3H), 1.48 (dd, J=9.0, 4.8 Hz, 1H). MS(APCI+) m/z 503.2 (M+H)⁺.

### Example 90

### (1S,2S)-1-(2-methoxy-5-methylphenyl)-2-[6-(methoxymethyl)pyridin-2-yl]-N-(2-methylquinoline-5-sulfonyl)cyclopropane-1-carboxamide

### Example 90A

### 2-bromo-6-(methoxymethyl)pyridine

To a solution of (6-bromopyridin-2-yl)methanol (590 mg, 3.14 mmol; Aldrich) in tetrahydrofuran (25 mL) was added sodium hydride (138 mg, 3.45 mmol; Aldrich) at 0° C. and the mixture was stirred for 30 minutes. Methyl iodide (0.216 mL, 3.45 mmol; Aldrich) was added, and the reaction allowed to stir for 24 hours. The reaction was quenched by addition of saturated aqueous NH₄Cl solution, and then diluted with ethyl acetate. The layers were separated, and the aqueous phase was extracted with ethyl acetate (2×30 mL) and dichloromethane. The combined organic extracts were dried over Na₂SO₄, filtered and concentrated to afford the title compound (564 mg, 2.79 mmol, 89% yield).

### Example 90B

### (1S,2S)-1-(2-methoxy-5-methylphenyl)-2-[6-(methoxymethyl)pyridin-2-yl]-N-(2-methylquinoline-5-sulfonyl)cyclopropane-1-carboxamide

The title compound was prepared according to the procedure described in Example 81 by substituting Example 90A for 3-bromo-2,6-dimethylpyridine in Example 81A, followed by processing as in Examples 81B, 81C, and 81D. ¹H NMR (500 MHz, dimethyl sulfoxide-d₆) δ ppm 8.87 (d, J=8.9 Hz, 1H), 8.32-8.25 (m, 2H), 7.95 (dd, J=8.5, 7.4 Hz, 1H), 7.62 (d, J=8.9 Hz, 1H), 7.62 (s, 1H), 7.15 (s, 1H), 7.03 (s, 1H), 6.93 (dd, J=8.4, 2.2 Hz, 1H), 6.83 (d, J=7.9 Hz, 1H), 6.42 (d, J=8.3 Hz, 1H), 4.35 (d, J=13.5 Hz, 1H), 4.17 (d, J=13.7 Hz, 1H), 3.32 (s, 1H), 3.18 (s, 3H), 3.02 (s, 3H), 2.75 (s, 3H), 2.56 (s, 1H), 2.35 (t, J=6.0 Hz, 1H), 2.23 (s, 3H), 1.55 (dd, J=8.9, 5.0 Hz, 1H). MS(APCI+) m/z 532.2 (M+H)⁺.

### Example 91

### (1R,2R)-1-(2-methoxy-5-methylphenyl)-2-(5-methylpyrazin-2-yl)-N-(2-methylquinoline-5-sulfonyl)cyclopropane-1-carboxamide

The enantiomers of Example 89 (231 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (61 mg, 0.121 mmol, 26.4% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.55 (s, 1H), 8.83 (d, J=8.9 Hz, 1H), 8.22 (s, 2H), 8.09 (s, 1H), 7.97-7.93 (m, 1H), 7.88 (s, 1H), 7.56 (d, J=8.9 Hz, 1H), 6.95 (s, 1H), 6.90 (d, J=8.3 Hz, 1H), 6.40 (d, J=8.2 Hz, 1H), 3.21 (m, 1H), 3.04 (s, 3H), 2.71 (s, 3H), 2.54 (s, 1H), 2.24 (s, 3H), 2.20 (s, 1H), 2.18 (s, 3H), 1.47 (dd, J=8.8, 4.6 Hz, 1H). MS(APCI+) m/z 503.2 (M+H)⁺.

### Example 92

### (1S,2S)-1-(2-methoxy-5-methylphenyl)-2-(5-methylpyrazin-2-yl)-N-(2-methylquinoline-5-sulfonyl)cyclopropane-1-carboxamide

The enantiomers of Example 89 (231 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (61.4 mg, 0.122 mmol, 26.6% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.54 (s, 1H), 8.83 (d, J=8.8 Hz, 1H), 8.24-8.20 (m, 2H), 8.11-8.07 (m, 1H), 7.95 (d, J=1.5 Hz, 1H), 7.88 (t, J=8.0 Hz, 1H), 7.56 (d, J=8.9 Hz, 1H), 6.95 (s, 1H), 6.93-6.87 (m, 1H), 6.40 (d, J=8.2 Hz, 1H), 3.21 (s, 1H), 3.04 (s, 3H), 2.71 (s, 3H), 2.24 (s, 3H), 2.22 (s, 1H), 2.18 (s, 3H), 1.47 (dd, J=8.9, 4.6 Hz, 1H). MS(APCI+) m/z 503.2 (M+H)⁺.

### Example 93

### (1R,2S)-2-(2,6-dimethoxypyridin-3-yl)-1-(2-methoxy-5-methylphenyl)-N-(2-methylquinoline-5-sulfonyl)cyclopropane-1-carboxamide

The enantiomers of Example 79 (13 mg) were separated by preparative chiral supercritical fluid chromatography (ChiralPak IC column, 45% methanol/CO₂, 80 mL/minute). The fractions containing the first eluting peak were concentrated under reduced pressure to afford the title compound (4.6 mg, 8.4 μmol, 35.4% yield). The material was determined to be >98% ee by analytical supercritical fluid chromatography (ChiralPak IC column, 3 mL/minute, 40-50% methanol/CO₂). ¹H NMR (400 MHz, Methanol-d₄) δ ppm 8.91 (d, J=8.9 Hz, 1H), 8.30 (d, J=7.4 Hz, 1H), 8.15 (d, J=8.4 Hz, 1H), 7.80 (t, J=7.8 Hz, 1H), 7.47 (d, J=8.9 Hz, 1H), 6.89 (d, J=8.5 Hz, 1H), 6.84 (s, 1H), 6.45 (d, J=8.3 Hz, 1H), 6.35 (d, J=8.1 Hz, 1H), 5.75 (d, J=8.1 Hz, 1H), 4.54 (s, 1H), 3.76 (d, J=10.9 Hz, 5H), 3.08 (d, J=8.3 Hz, 1H), 2.15 (s, 3H), 1.67 (d, J=8.4 Hz, 2H), 1.30-1.21 (m, 1H). MS(APCI+) m/z 548.2 (M+H)⁺.

### Example 94

### (1S,2R)-2-(2,6-dimethoxypyridin-3-yl)-1-(2-methoxy-5-methylphenyl)-N-(2-methylquinoline-5-sulfonyl)cyclopropane-1-carboxamide

The enantiomers of Example 79 (13 mg) were separated by preparative chiral supercritical fluid chromatography (ChiralPak IC column, 45% methanol/CO₂, 80 mL/minute). The fractions containing the second eluting peak were concentrated under reduced pressure to afford the title compound (3.2 mg, 5.85 μmol, 24.6% yield). The material was determined to be >98% ee by analytical supercritical fluid chromatography (ChiralPak IC column, 3 mL/minute, 40-50% methanol/CO₂). ¹H NMR (600 MHz, Methanol-d₄) δ ppm 8.92 (d, J=8.6 Hz, 1H), 8.34 (d, J=5.2 Hz, 1H), 8.20 (d, J=8.5 Hz, 1H), 7.86 (t, J=7.6 Hz, 1H), 7.52 (d, J=8.7 Hz, 1H), 6.95 (d, J=8.3 Hz, 1H), 6.89 (s, 1H), 6.49 (d, J=8.3 Hz, 1H), 6.39 (d, J=8.1 Hz, 1H), 5.77 (d, J=8.1 Hz, 1H), 3.79 (d, J=14.9 Hz, 4H), 3.36 (s, 1H), 3.33 (s, OH), 3.11 (s, 1H), 2.78 (s, 2H), 2.20 (s, 2H), 1.75-1.67 (m, 1H). MS(APCI+) m/z 548.2 (M+H)⁺.

### Example 95

### rac-(1r,2r)-1-(2-methoxy-5-methylphenyl)-2-(2-methoxypyrimidin-4-yl)-N-(2-methylquinoline-5-sulfonyl)cyclopropane-1-carboxamide

### Example 95A

### 2-methoxy-4-vinylpyrimidine

The title compound was prepared according to the procedure described in Example 81A by substituting 4-bromo-2-methoxypyrimidine (Arkpharm) for 2-bromo-5-methylpyrazine. MS(APCI+) m/z 137.15 (M+H)⁺.

### Example 95B

### rac-(1r,2r)-1-(2-methoxy-5-methylphenyl)-2-(2-methoxypyrimidin-4-yl)-N-(2-methylquinoline-5-sulfonyl)cyclopropane-1-carboxamide

The cyclopropane was prepared according to the procedure described in Example 4A by substituting Example 95A for 4-methylstyrene, followed by processing as in Examples 4B and 4C to afford the title compound. ¹H NMR (500 MHz, dimethyl sulfoxide-d₆) δ ppm 8.89 (d, J=8.9 Hz, 1H), 8.35-8.27 (m, 2H), 8.24 (d, J=6.0 Hz, 1H), 7.96 (dd, J=8.5, 7.4 Hz, 1H), 7.64 (d, J=8.9 Hz, 1H), 7.09 (s, 1H), 7.01 (dd, J=8.5, 2.5 Hz, 1H), 6.58 (d, J=6.0 Hz, 1H), 6.50 (d, J=8.3 Hz, 1H), 3.43 (s, 3H), 3.21 (dd, J=8.6, 7.0 Hz, 1H), 3.04 (s, 3H), 2.76 (s, 3H), 2.26-2.20 (m, 4H), 1.64 (dd, J=8.7, 4.5 Hz, 1H). MS(APCI+) m/z 519.12 (M+H)⁺.

### Example 96

### (1S,2S)-1-(2-methoxy-5-methylphenyl)-2-(6-methylpyridin-2-yl)-N-(2-methylquinoline-5-sulfonyl)cyclopropane-1-carboxamide

The cyclopropane was prepared according to the procedure described in Example 69E by substituting 2-methyl-6-vinylpyridine for Example 69A, followed by processing as in Examples 69G and 69H to afford the title compound. RT 0.64 min. ¹H NMR (500 MHz, CDCl₃) δ 9.75 (d, J=8.9 Hz, 1H), 8.75 (d, J=8.4 Hz, 1H), 8.60 (d, J=7.4 Hz, 1H), 8.05 (t, J=7.9 Hz, 1H), 7.79 (d, J=8.9 Hz, 1H), 7.70 (t, J=7.9 Hz, 1H), 7.29 (d, J=4.0 Hz, 1H), 7.14 (d, J=2.2 Hz, 1H), 7.05 (dd, J=8.5, 2.1 Hz, 1H), 6.43 (dd, J=8.2, 4.4 Hz, 2H), 3.80 (t, J=7.8 Hz, 1H), 3.29 (s, 3H), 3.07 (s, 3H), 2.75 (s, 3H), 2.28 (s, 3H), 2.17-2.04 (m, 2H). MS(APCI+) m/z 502.5 (M+H)⁺.

### Example 97

### (1S,2S)-1-(2-methoxy-5-methylphenyl)-N-(2-methylquinoline-5-sulfonyl)-2-{6-[(propan-2-yl)oxy]pyridin-2-yl}cyclopropane-1-carboxamide

The title compound was prepared according to the procedure described in Example 81 by substituting 2-bromo-6-isopropoxypyridine (Combi-Blocks) for 3-bromo-2,6-dimethylpyridine in Example 81A, followed by processing as in Examples 81B to Example 81D to afford the title compound. ¹H NMR (400 MHz, dimethyl sulfoxide-d₆) δ ppm 8.88 (d, J=8.9 Hz, 1H), 8.31-8.19 (m, 2H), 7.97-7.84 (m, 1H), 7.62 (d, J=8.9 Hz, 1H), 7.34-7.24 (m, 1H), 6.96-6.85 (m, 2H), 6.65 (d, J=7.3 Hz, 1H), 6.43 (d, J=8.3 Hz, 1H), 6.18 (d, J=8.1 Hz, 1H), 4.45 (p, J=6.2 Hz, 1H), 3.03 (s, 3H), 2.72 (s, 3H), 2.20-2.07 (m, 3H), 1.41 (dd, J=8.8, 4.3 Hz, 1H), 1.06 (d, J=6.1 Hz, 3H), 0.91 (d, J=6.2 Hz, 3H). MS(APCI+) m/z 546.23 (M+H)⁺.

### Example 98

### (1S,2S)-1-(2-methoxy-5-methylphenyl)-2-(2-methoxypyrimidin-4-yl)-N-(2-methylquinoline-5-sulfonyl)cyclopropane-1-carboxamide

The enantiomers of Example 95 (165 mg) were separated by preparative chiral supercritical fluid chromatography (ChiralPak IC column, 40% methanol/CO₂, 80 mL/minute). The fractions containing the second eluting peak were concentrated under reduced pressure to afford the title compound (50.6 mg, 0.098 mmol, 30.7% yield). The material was determined to be >98% ee by analytical supercritical fluid chromatography (ChiralPak IC column, 3 mL/minute, 40-50% methanol/CO₂). ¹H NMR (500 MHz, dimethyl sulfoxide-d₆) δ ppm 8.84 (d, J=8.9 Hz, 1H), 8.28-8.22 (m, 2H), 8.15 (d, J=5.8 Hz, 1H), 7.91 (dd, J=8.4, 7.4 Hz, 1H), 7.58 (d, J=8.9 Hz, 1H), 7.03 (s, 1H), 6.98 (ddd, J=8.2, 2.3, 0.9 Hz, 1H), 6.47 (dd, J=13.9, 7.0 Hz, 2H), 3.41 (s, 3H), 3.21-3.11 (m, 1H), 3.03 (s, 3H), 2.73 (s, 3H), 2.22 (s, 3H), 2.18 (dd, J=7.0, 4.4 Hz, 1H), 1.58 (dd, J=8.7, 4.3 Hz, 1H). MS(APCI+) m/z 519.0 (M+H)⁺.

### Example 99

### (1R,2R)-1-(2-methoxy-5-methylphenyl)-2-(2-methoxypyrimidin-4-yl)-N-(2-methylquinoline-5-sulfonyl)cyclopropane-1-carboxamide

The enantiomers of Example 95 (165 mg) were separated by preparative chiral supercritical fluid chromatography (ChiralPak IC column, 40% methanol/CO₂, 80 mL/minute). The fractions containing the first eluting peak were concentrated under reduced pressure to afford the title compound (45 mg, 0.087 mmol, 27.3% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 8.82 (d, J=8.8 Hz, 1H), 8.23 (d, J=7.8 Hz, 2H), 8.13 (d, J=5.8 Hz, 1H), 7.89 (dd, J=8.4, 7.4 Hz, 1H), 7.56 (d, J=8.9 Hz, 1H), 7.02 (s, 1H), 6.99-6.94 (m, 1H), 6.46 (dd, J=17.0, 7.0 Hz, 2H), 3.40 (s, 3H), 3.17 (s, 1H), 3.12 (dd, J=8.6, 6.9 Hz, 1H), 3.02 (s, 4H), 2.71 (s, 3H), 2.20 (s, 4H), 2.17 (s, 1H), 1.56 (dd, J=8.7, 4.3 Hz, 1H). MS(APCI+) m/z 519.0 (M+H)⁺.

### Example 100

### (1S,2R)-2-(5-fluoro-6-methylpyridin-3-yl)-1-(2-methoxy-5-methylphenyl)-N-(2-methylquinoline-5-sulfonyl)cyclopropane-1-carboxamide

### Example 100A

### 3-fluoro-2-methyl-5-vinylpyridine

The title compound was prepared according to the procedure described in Example 81A by substituting 5-bromo-3-fluoro-2-methylpyridine (Matrix) for 2-bromo-5-methylpyrazine.

### Example 100B

### rac-(1s,2r)-2-(5-fluoro-6-methylpyridin-3-yl)-1-(2-methoxy-5-methylphenyl)-N-(2-methylquinoline-5-sulfonyl)cyclopropane-1-carboxamide

The cyclopropane was prepared according to the procedure described in Example 4A by substituting Example 100A for 4-methylstyrene, followed by processing as in Examples 4B and 4C to afford the title compound. MS(APCI+) m/z 519.88 (M+H)⁺.

### Example 100C

### (1S,2R)-2-(5-fluoro-6-methylpyridin-3-yl)-1-(2-methoxy-5-methylphenyl)-N-(2-methylquinoline-5-sulfonyl)cyclopropane-1-carboxamide

The enantiomers of Example 100B (67 mg) were separated by preparative chiral supercritical fluid chromatography (ChiralPak IC column, 40% methanol/CO₂, 80 mL/minute). The fractions containing the second eluting peak were concentrated under reduced pressure to afford the title compound (17.5 mg, 0.034 mmol, 26.1% yield). The material was determined to be >98% ee by analytical supercritical fluid chromatography (ChirualPakIC column, 3 mL/minute, 40-50% methanol/CO₂). ¹H NMR (600 MHz, dimethyl sulfoxide-d₆) δ ppm 11.66 (s, 1H), 8.91-8.86 (m, 1H), 8.20 (s, 1H), 8.09 (s, 2H), 7.78 (d, J=1.8 Hz, 1H), 7.50 (d, J=8.6 Hz, 1H), 6.98 (s, 1H), 6.89 (d, J=8.1 Hz, 1H), 6.66 (s, 1H), 6.44 (d, J=7.9 Hz, 1H), 3.17 (s, 1H), 3.02 (s, 3H), 2.92 (q, J=6.7 Hz, 1H), 2.69 (s, 3H), 2.21 (d, J=2.8 Hz, 3H), 2.20 (s, 3H), 1.43 (s, 1H). MS(APCI+) m/z 519.9 (M+H)⁺.

### Example 101

### (1R,2S)-2-(5-fluoro-6-methylpyridin-3-yl)-1-(2-methoxy-5-methylphenyl)-N-(2-methylquinoline-5-sulfonyl)cyclopropane-1-carboxamide

The enantiomers of Example 100B (67 mg) were separated by preparative chiral supercritical fluid chromatography (ChiralPak IC column, 40% methanol/CO₂, 80 mL/minute). The fractions containing the first eluting peak were concentrated under reduced pressure to afford the title compound (17.2 mg, 0.033 mmol, 25.7% yield). The material was determined to be >98% ee by analytical supercritical fluid chromatography (ChiralPak IC column, 3 mL/minute, 40-50% methanol/CO₂). ¹H NMR (600 MHz, dimethyl sulfoxide-d₆) δ ppm 11.66 (s, 1H), 8.91 (s, 1H), 8.17 (s, 1H), 8.09 (s, 2H), 7.96 (s, 2H), 7.78 (s, 1H), 7.71 (s, 1H), 6.97 (s, 1H), 6.87 (s, 1H), 6.56 (s, 1H), 6.43 (s, 1H), 3.01 (s, 4H), 2.95-2.89 (m, 1H), 2.68 (s, 3H), 2.21 (d, J=2.8 Hz, 3H), 2.20 (s, 3H), 1.43 (s, 1H). MS(APCI+) m/z 519.9 (M+H)⁺.

### Example 102

### (1S,2R)-2-(5-chloro-6-methoxypyridin-3-yl)-1-(2-methoxy-5-methylphenyl)-N-(2-methylquinoline-5-sulfonyl)cyclopropane-1-carboxamide

### Example 102A

### (R)-4,4-dimethyl-2-oxotetrahydrofuran-3-yl (1S,2R)-2-(5-chloro-6-methoxypyridin-3-yl)-1-(2-methoxy-5-methylphenyl)cyclopropane-1-carboxylate

To a −20° C. solution of rhodium(II) octanoate dimer (11.51 mg, 0.015 mmol) and 3-chloro-2-methoxy-5-vinylpyridine (275.9 mg, 1.627 mmol) in dichloromethane (16 mL) was slowly added, dropwise, a solution of Example 62D (471 mg, 1.479 mmol) dichloromethane (8 mL). The reaction was stirred at ambient temperature overnight before concentrating and purifying by flash chromatography (0-30% ethyl acetate/heptanes, 12 g RediSep®) to afford the title compound (435.5 mg, 0.947 mmol, 64.0% yield). MS(ESI+) m/z 460.0 (M+H)⁺.

### Example 102B

### (1S,2R)-2-(5-chloro-6-methoxypyridin-3-yl)-1-(2-methoxy-5-methylphenyl)cyclopropane-1-carboxylic acid

To a solution of Example 102A (435.5 mg, 0.947 mmol) in tetrahydrofuran (5 mL), methanol (1 mL), and water (1 mL) was added sodium hydroxide (379 mg, 9.47 mmol), and the reaction was stirred at 60° C. overnight. The reaction was acidified by addition of 3 N HCl solution (5 mL) and extracted with ethyl acetate (3×20 mL). The combined organic layers were dried over MgSO₄ and concentrated under reduced pressure. The crude residue was purified by reverse-phase HPLC (Phenomenex Luna C8(2) 5 μm 100 Å AXIA column (30 mm×75 mm), 50 mL/minute, 5-100% acetonitrile/0.1% trifluoroacetic acid in water) to afford the title compound (185.1 mg, 0.532 mmol, 56.2% yield). MS(ESI+) m/z 348.1 (M+H)⁺.

### Example 102C

### (1S,2R)-2-(5-chloro-6-methoxypyridin-3-yl)-1-(2-methoxy-5-methylphenyl)-N-(2-methylquinoline-5-sulfonyl)cyclopropane-1-carboxamide

To Example 102B (34.8 mg, 0.10 mmol) in a 4 mL vial was added 1-ethyl-3-[3-(dimethylamino)propyl]-carbodiimide hydrochloride (21.1 mg, 0.11 mmol) and 4-(dimethylamino)pyridine (24.4 mg, 0.20 mmol) in dichloromethane (0.3 mL). The reaction was stirred for 5 minutes at ambient temperature, and 2-methylquinoline-5-sulfonamide (26.6 mg, 0.12 mmol) in dichloromethane (0.3 mL) was added. The reaction was stirred overnight at ambient temperature, and then the solvent was removed under a stream of nitrogen. The residue was reconstituted in 1:1 dimethyl sulfoxide/methanol and purified via reverse-phase HPLC (Phenomenex® Luna® C8(2) 5 μm 100 Å AXIA column (50 mm×30 mm), gradient of acetonitrile (A) and 0.1% trifluoroacetic acid in water (B), 40 mL/minute, (0-0.5 minutes 25% A, 0.5-8.0 minutes linear gradient 25-100% A, 8.0-9.0 minutes 100% A, 7.0-8.9 minutes 100% A, 9.0-9.1 minutes linear gradient 100-25% A, 9.1-10 minutes 25% A) to afford the title compound (25.4 mg, 38% yield). ¹H NMR (400 MHz, d₆-dimethyl sulfoxide) δ ppm 11.55 (s, 1H), 8.87 (d, J=9.0 Hz, 1H), 8.28-8.20 (m, 2H), 7.90 (dd, J=8.5, 7.4 Hz, 1H), 7.61 (d, J=8.9 Hz, 1H), 7.55 (d, J=2.1 Hz, 1H), 7.09 (d, J=2.1 Hz, 1H), 6.97 (d, J=2.3 Hz, 1H), 6.92 (ddd, J=8.1, 2.2, 0.8 Hz, 1H), 6.47 (d, J=8.3 Hz, 1H), 3.71 (s, 3H), 3.10 (s, 3H), 3.00 (dd, J=9.3, 7.1 Hz, 1H), 2.71 (s, 3H), 2.18 (s, 3H), 2.08 (dd, J=7.1, 5.7 Hz, 1H), 1.32 (dd, J=9.3, 5.6 Hz, 1H). MS(APCI+) m/z 552.2 (M+H)⁺.

### Example 103

### (1S,2R)-1-(2-methoxy-5-methylphenyl)-2-(6-methoxy-5-methylpyridin-3-yl)-N-(2-methylquinoline-5-sulfonyl)cyclopropane-1-carboxamide

### Example 103A

### (R)-4,4-dimethyl-2-oxotetrahydrofuran-3-yl (1S,2R)-1-(2-methoxy-5-methylphenyl)-2-(6-methoxy-5-methylpyridin-3-yl)cyclopropane-1-carboxylate

To a −20° C. solution of rhodium(II) octanoate dimer (20.46 mg, 0.026 mmol) and 2-methoxy-3-methyl-5-vinylpyridine (588 mg, 3.94 mmol) in dichloromethane (16 mL) was slowly added, dropwise, a solution of (R)-4,4-dimethyl-2-oxotetrahydrofuran-3-yl 2-diazo-2-(2-methoxy-5-methylphenyl)acetate (836 mg, 2.63 mmol) dichloromethane (8 mL). The reaction was stirred at ambient temperature overnight, concentrated under reduced pressure, and purified by flash chromatography (0-30% ethyl acetate/heptanes, 12 g RediSep® column) to afford the title compound (796.6 mg, 1.813 mmol, 69.0% yield). MS(ESI+) m/z 440.1 (M+H)⁺.

### Example 103B

### (1S,2R)-1-(2-methoxy-5-methylphenyl)-2-(6-methoxy-5-methylpyridin-3-yl)cyclopropane-1-carboxylic acid

To a solution of Example 103A (796.6 mg, 1.813 mmol) in tetrahydrofuran (10 mL), methanol (2 mL), and water (2 mL) was added sodium hydroxide (3625 mg, 91 mmol). The reaction was stirred at 60° C. overnight, then acidified with 3 N HCl solution (5 mL) and extracted with ethyl acetate (3×20 mL). The combined organic layers were dried over MgSO₄, concentrated under reduced pressure, and purified via flash chromatography (0-10%, methanol/dichloromethane, 12 g RediSep® column) to afford the title compound (626.7 mg, 1.914 mmol, 106% yield). MS(ESI+) m/z 327.5 (M+H)⁺.

### Example 103C

### (1S,2R)-1-(2-methoxy-5-methylphenyl)-2-(6-methoxy-5-methylpyridin-3-yl)-N-(2-methylquinoline-5-sulfonyl)cyclopropane-1-carboxamid

To Example 103B (32.7 mg, 0.10 mmol) in a 4 mL vial was added 1-ethyl-3-[3-(dimethylamino)propyl]-carbodiimide hydrochloride (21.1 mg, 0.11 mmol) and 4-(dimethylamino)pyridine (24.4 mg, 0.20 mmol) in dichloromethane (0.3 mL), and the reaction was stirred for 5 minutes at ambient temperature. 2-Methylquinoline-5-sulfonamide (26.6 mg, 0.12 mmol) in dichloromethane (0.3 mL) was added, and the reaction was stirred overnight at ambient temperature and then the solvent was removed under a stream of nitrogen. The residue was reconstituted in 1:1 dimethyl sulfoxide/methanol and purified via reverse-phase HPLC (Phenomenex® Luna® C8(2) 5 μm 100 AXIA column (50 mm×30 mm), gradient of acetonitrile (A) and 0.1% trifluoroacetic acid in water (B), 40 mL/minute, (0-0.5 minutes 25% A, 0.5-8.0 minutes linear gradient 25-100% A, 8.0-9.0 minutes 100% A, 7.0-8.9 minutes 100% A, 9.0-9.1 minutes linear gradient 100-25% A, 9.1-10 minutes 25% A)) to afford the title compound as an impure mixture. The material dissolved in methanol and purified via reverse-phase HPLC (Phenomenex® Luna® C8(2) 5 μm 100 AXIA column (50 mm×30 mm), gradient of acetonitrile (A) and 0.1% ammonium acetate in water (B), 40 mL/minute, (0-0.5 minutes 5% A, 0.5-8.0 minutes linear gradient 5-100% A, 8.0-9.0 minutes 100% A, 7.0-8.9 minutes 100% A, 9.0-9.1 minutes linear gradient 100-5% A, 9.1-10 minutes 5% A) to afford the title compound (5.4 mg). ¹H NMR (400 MHz, d₆-dimethyl sulfoxide) δ ppm 11.46 (s, 1H), 8.82 (d, J=8.9 Hz, 1H), 8.14 (s, 2H), 7.83-7.78 (m, 1H), 7.50 (d, J=8.9 Hz, 1H), 7.33 (d, J=2.3 Hz, 1H), 6.94-6.82 (m, 2H), 6.77 (s, 1H), 6.43 (d, J=8.2 Hz, 1H), 3.64 (s, 3H), 3.06 (s, 3H), 2.88 (s, 1H), 2.67 (s, 3H), 2.15 (s, 3H), 1.84-1.80 (m, 4H), 1.31 (dd, J=9.4, 5.1 Hz, 1H). MS(APCI+) m/z 532.2 (M+H)⁺.

### Example 104

### (1S,2R)-2-(5,6-dimethoxypyridin-3-yl)-1-(2-methoxy-5-methylphenyl)-N-(2-methylquinoline-5-sulfonyl)cyclopropane-1-carboxamide

### Example 104A

### (R)-4,4-dimethyl-2-oxotetrahydrofuran-3-yl (1S,2R)-2-(5,6-dimethoxypyridin-3-yl)-1-(2-methoxy-5-methylphenyl)cyclopropane-1-carboxylate

To a −20° C. solution of rhodium(II) octanoate dimer (5.83 mg, 7.49 μmol) and 2,3-dimethoxy-5-vinylpyridine (136.1 mg, 0.824 mmol) in dichloromethane (8 mL) was slowly added, dropwise, a solution of Example 69D (238 mg, 0.749 mmol) in dichloromethane (4 mL). The reaction was stirred at ambient temperature overnight, concentrated under reduced pressure, and purified by flash chromatography (0-30% ethyl acetate/heptanes, 12 g RediSep® silica column) to afford the title compound (315.2 mg, 0.692 mmol, 92% yield). MS(ESI+) m/z 455.8 (M+H)⁺.

### Example 104B

### (1S,2R)-2-(5,6-dimethoxypyridin-3-yl)-1-(2-methoxy-5-methylphenyl)cyclopropane-1-carboxylic acid

To a solution of Example 104A (315.2 mg, 0.692 mmol) in tetrahydrofuran (5 mL), methanol (1 mL), and water (1 mL) was added sodium hydroxide (277 mg, 6.92 mmol). The reaction was stirred at 60° C. overnight, acidified with 3 N HCl solution (5 mL), and extracted with ethyl acetate (3×20 mL). The combined organic layers were dried over MgSO₄, concentrated under reduced pressure, and purified via reverse-phase HPLC (Phenomenex Luna C8(2) 5 μm 100 AXIA column (30 mm×75 mm), 50 mL/minute, 5-100% acetonitrile/0.1% trifluoroacetic acid in water) to afford the title compound (116.1 mg, 0.338 mmol, 48.9% yield).

### Example 104C

### (1S,2R)-2-(5,6-dimethoxypyridin-3-yl)-1-(2-methoxy-5-methylphenyl)cyclopropane-1-carboxylic acid

To Example 104B (34.3 mg, 0.10 mmol) in a 4 mL vial was added 1-ethyl-3-[3-(dimethylamino)propyl]-carbodiimide hydrochloride (21.1 mg, 0.11 mmol) and 4-(dimethylamino)pyridine (24.4 mg, 0.20 mmol) in dichloromethane (0.3 mL). The reaction was stirred for 5 minutes at ambient temperature, and then 2-methylquinoline-5-sulfonamide (26.6 mg, 0.12 mmol) in dichloromethane (0.3 mL) was added. The reaction was stirred overnight at ambient temperature, and the solvent was removed under a stream of nitrogen. The residue reconstituted in 1:1 dimethyl sulfoxide/methanol, and purified via reverse-phase HPLC (Phenomenex® Luna® C8(2) 5 μm 100 Å AXIA column (50 mm×30 mm), gradient of acetonitrile (A) and 0.1% trifluoroacetic acid in water (B), 40 mL/minute, (0-0.5 minutes 25% A, 0.5-8.0 minutes linear gradient 25-100% A, 8.0-9.0 minutes 100% A, 7.0-8.9 minutes 100% A, 9.0-9.1 minutes linear gradient 100-25% A, 9.1-10 minutes 25% A)) to afford the title compound as an impure mixture. The material dissolved in methanol and purified via reverse-phase HPLC (Phenomenex® Luna® C8(2) 5 μm 100 Å AXIA column (50 mm×30 mm), gradient of acetonitrile (A) and 0.1% ammonium acetate in water (B), 40 mL/minute, (0-0.5 minutes 5% A, 0.5-8.0 minutes linear gradient 5-100% A, 8.0-9.0 minutes 100% A, 7.0-8.9 minutes 100% A, 9.0-9.1 minutes linear gradient 100-5% A, 9.1-10 minutes 5% A)) to afford the title compound (1.0 mg). ¹H NMR (400 MHz, d₆-dimethyl sulfoxide) δ ppm 11.48 (s, 1H), 8.83 (d, J=8.9 Hz, 1H), 8.14 (s, 2H), 7.83-7.78 (m, 1H), 7.50 (d, J=8.9 Hz, 1H), 7.19 (d, J=1.9 Hz, 1H), 6.99-6.95 (m, 1H), 6.94-6.86 (m, 1H), 6.45 (d, J=8.2 Hz, 1H), 6.26 (s, 1H), 3.64 (s, 3H), 3.36 (s, 3H), 3.03 (s, 3H), 2.96-2.91 (m, 1H), 2.66 (s, 3H), 2.17 (s, 3H), 1.92-1.85 (m, 1H), 1.34 (dd, J=9.4, 5.1 Hz, 1H). MS(APCI+) m/z 548.3 (M+H)⁺.

### Example 105

### (1S,2S)-2-(6-chloropyridin-2-yl)-1-(2-methoxy-5-methylphenyl)-N-(2-methylquinoline-5-sulfonyl)cyclopropane-1-carboxamide

### Example 105A

### (1S,2S)-2-(6-chloropyridin-2-yl)-1-(2-methoxy-5-methylphenyl)cyclopropanecarboxylic acid

NaH (60 weight %, 36.2 mg, 0.904 mmol) was added to 2,2,2-trifluoroethanol (1 mL, 0.301 mmol) at 0° C., and the mixture was warmed to ambient temperature and stirred for 5 minutes. Example 43C (100 mg, 0.301 mmol) was added, and the reaction mixture was heated to 85° C. overnight. The mixture was concentrated under reduced pressure and purified by flash chromatography (ISCO CombiFlash, 0-100% ethyl acetate/heptanes, 12 g RediSep® Rf Gold® Normal-Phase Silica) to afford the title compound (63 mg, 0.198 mmol, 65.8% yield). ¹H NMR (600 MHz, CDCl₃) δ ppm 7.28 (t, J=7.8 Hz, 1H), 7.03 (s, 1H), 6.96-6.89 (m, 2H), 6.73 (dd, J=7.7, 0.8 Hz, 1H), 6.42 (d, J=8.3 Hz, 1H), 3.40 (s, 3H), 3.37 (dd, J=8.9, 7.1 Hz, 1H), 2.26 (dd, J=7.1, 4.7 Hz, 1H), 2.24 (s, 3H), 2.07-2.01 (m, 1H). MS (ESI) m/z 318.2 (M+H)⁺.

### Example 105B

### (1S,2S)-2-(6-chloropyridin-2-yl)-1-(2-methoxy-5-methylphenyl)-N-(2-methylquinoline-5-sulfonyl)cyclopropane-1-carboxamide

A mixture of 1-(3-dimethylaminopropyl)-3-ethylcarbodiimide hydrochloride (24.13 mg, 0.126 mmol), 4-(dimethylamino)pyridine (9.23 mg, 0.076 mmol), 2-methylquinoline-5-sulfonamide (15.39 mg, 0.069 mmol) and Example 105A (20 mg, 0.063 mmol) in dichloromethane (1 mL) was stirred at ambient temperature, then concentrated under reduced pressure and partially purified via reverse-phase HPLC (Biotage® Sfar C18 D Duo 100 Å 30 μm 30 g, 10-100% acetonitrile/pH 7 buffer). This material was re-purified via reverse-phase HPLC (Biotage® Sfar C18 D Duo 100 Å 30 μm 30 g, 10-100% acetonitrile/0.1% aqueous trifluoroacetic acid solution) to afford the title compound (28 mg, 0.044 mmol, 69.9% yield) as a trifluoroacetic acid salt: ¹H NMR (600 MHz, CDCl₃) δ ppm 9.23 (d, J=8.9 Hz, 1H), 8.69 (dt, J=8.6, 1.0 Hz, 1H), 8.51 (dd, J=7.5, 1.1 Hz, 1H), 8.35 (s, 1H), 8.00 (dd, J=8.5, 7.5 Hz, 1H), 7.61 (d, J=9.0 Hz, 1H), 7.27 (t, J=7.8 Hz, 1H), 7.09-7.04 (m, 2H), 6.88 (dd, J=7.9, 0.8 Hz, 1H), 6.77 (dd, J=7.7, 0.9 Hz, 1H), 6.46 (d, J=8.3 Hz, 1H), 3.19 (s, 3H), 3.11 (dd, J=9.0, 7.1 Hz, 1H), 2.99 (s, 3H), 2.29 (s, 3H), 2.22 (dd, J=7.1, 4.7 Hz, 1H), 1.87 (dd, J=9.0, 4.7 Hz, 1H). MS (ESI) m/z 521.9 (M+H)⁺.

### Example 106

### (1S,2S)-2-(6-ethoxy-5-fluoropyridin-2-yl)-1-(2-methoxy-5-methylphenyl)-N-(2-methylquinoline-5-sulfonyl)cyclopropane-1-carboxamide

### Example 106A

### (1S,2S)-methyl 2-(6-chloro-5-fluoropyridin-2-yl)-1-(2-methoxy-5-methylphenyl)cyclopropanecarboxylate

To a mixture of 1,1,1,3,3,3-hexafluoropropan-2-ol (644 μL, 6.13 mmol), 2-chloropyridine (203 μL, 2.147 mmol), 2-chloro-3-fluoro-6-vinylpyridine (145 mg, 0.920 mmol), and Rh₂(S-TPPTTL)₄ (3.78 mg, 1.534 μmol) at 0° C. was added Example 1B (135 mg, 0.613 mmol). The reaction mixture was stirred overnight and allowed to return to ambient temperature as the ice bath melted. The reaction mixture was concentrated under a stream of nitrogen and purified via flash chromatography (ISCO CombiFlash, 0-50% ethyl acetate/heptanes, RediSep® Rf Gold® Normal-Phase Silica) to afford the title compound (198 mg, 0.566 mmol, 92% yield). The material was determined to be 94% ee by analytical chiral supercritical fluid chromatography (ChiralPak IC, 3 mL/minute, 5-50% methanol/CO₂, 220 and 254 nm). ¹H NMR (500 MHz, CDCl₃) δ ppm 7.14-7.10 (m, 1H), 7.02 (d, J=2.3 Hz, 1H), 6.94-6.90 (m, 1H), 6.83 (dd, J=8.3, 3.2 Hz, 1H), 6.42 (d, J=8.3 Hz, 1H), 3.64 (s, 3H), 3.43 (s, 3H), 3.36 (dd, J=8.9, 6.8 Hz, 1H), 2.25 (t, J=0.7 Hz, 3H), 2.23 (dd, J=6.9, 4.7 Hz, 1H), 1.93 (dd, J=8.9, 4.7 Hz, 1H). MS (ESI) m/z 350.1 (M+H)⁺.

### Example 106B

### (1S,2S)-2-(6-ethoxy-5-fluoropyridin-2-yl)-1-(2-methoxy-5-methylphenyl)cyclopropanecarboxylic acid

A sodium tert-butoxide solution (2 M in tetrahydrofuran, 368 μL, 0.736 mmol) was added to a degassed mixture of ethanol (198 μL, 3.40 mmol), (2-di-tert-butylphosphino-3,6-dimethoxy-2′,4′,6′-triisopropyl-1,1′-biphenyl)-2-(2′-amino-1,1′-biphenyl)]palladium(II) methanesulfonate (tBuBrettPhos Pd G3) (14.51 mg, 0.017 mmol), 2-(di-tert-butylphosphino)-2′,4′,6′-triisopropyl-3,6-dimethoxy-1,1′-biphenyl (tBuBrettPhos) (8.23 mg, 0.017 mmol), and Example 106A (198 mg, 0.566 mmol) in 1,4-dioxane (1.89 mL). The reaction was stirred at ambient temperature for 2 hours, then diluted with ethyl acetate, and concentrated under reduced pressure. The crude residue was mixed with potassium hydroxide (318 mg, 5.66 mmol) in a 1:1:1 methanol/H₂O/tetrahydrofuran solution at ambient temperature and stirred rapidly overnight. The mixture was concentrated under reduced pressure to remove organic solvents, and the resulting aqueous layer was washed twice using tert-butyl methyl ether. The aqueous layer was then acidified using 1 M citric acid and extracted three times using dichloromethane. The combined dichloromethane washes were dried with MgSO₄, and concentrated. The crude material was purified by flash chromatography (ISCO CombiFlash, 0-100% ethyl acetate/heptanes, 24 g RediSep® Rf Gold® Normal-Phase Silica) to afford the title compound (153 mg, 0.443 mmol, 78% yield). ¹H NMR (500 MHz, CDCl₃) δ ppm 7.10 (dd, J=10.2, 8.0 Hz, 1H), 7.05 (s, 1H), 6.92 (ddt, J=8.2, 2.3, 0.8 Hz, 1H), 6.75 (dd, J=8.0, 2.7 Hz, 1H), 6.43 (d, J=8.3 Hz, 1H), 3.89 (dq, J=10.6, 7.1 Hz, 1H), 3.58 (dq, J=10.6, 7.1 Hz, 1H), 3.36 (s, 3H), 3.23-3.15 (m, 1H), 2.27-2.13 (m, 4H), 1.98 (dd, J=8.9, 4.0 Hz, 1H), 1.16 (t, J=7.1 Hz, 3H). MS (ESI) m/z 346.2 (M+H)⁺.

### Example 106C

### (1S,2S)-2-(6-ethoxy-5-fluoropyridin-2-yl)-1-(2-methoxy-5-methylphenyl)-N-(2-methylquinoline-5-sulfonyl)cyclopropane-1-carboxamide

4-(Dimethylamino)pyridine (12.73 mg, 0.104 mmol), 1-ethyl-3-[3-(dimethylamino)propyl]-carbodiimide hydrochloride (33.3 mg, 0.174 mmol), 2-methylquinoline-5-sulfonamide (21.24 mg, 0.096 mmol), and Example 106B (30 mg, 0.087 mmol) were mixed in dichloromethane (1.5 mL) at ambient temperature and stirred overnight. The mixture was acidified using 1 M citric acid, extracted three times using dichloromethane, dried with MgSO₄, and concentrated. The resulting residue was dissolved in a minimal amount of methanol. After precipitation was finished, the supernatant was removed, and the precipitate was washed twice with a minimal amount of methanol and dried under reduced pressure to afford the title compound (33 mg, 0.060 mmol, 69.1% yield). ¹H NMR (500 MHz, CDCl₃) δ ppm 8.60 (s, 1H), 8.44 (dd, J=7.5, 1.2 Hz, 1H), 8.33 (s, 1H), 8.28 (s, 1H), 7.83 (dd, J=8.4, 7.5 Hz, 1H), 7.09-7.00 (m, 3H), 6.62 (dd, J=8.0, 2.7 Hz, 1H), 6.51-6.44 (m, 1H), 3.80 (dq, J=10.5, 7.1 Hz, 1H), 3.47 (dq, J=10.5, 7.1 Hz, 1H), 3.10 (s, 3H), 2.97 (dd, J=9.0, 7.0 Hz, 1H), 2.79 (s, 3H), 2.26 (s, 3H), 2.07 (dd, J=7.0, 4.1 Hz, 1H), 1.83 (dd, J=9.0, 4.1 Hz, 1H), 1.13 (t, J=7.1 Hz, 3H). MS (ESI) m/z 550.0 (M+H)⁺.

### Determination of Biological Activity

### Trans-Epithelial Current Clamp (TECC) Assay Using Primary Human Bronchial Epithelial Cells

A cell-based assay was developed using the primary human bronchial epithelial cells (hBE cells) with F508del/F508del CFTR and other mutations.

### Human Bronchial Epithelial Cells

Primary human bronchial epithelial (hBE) cells from CFTR patients with homozygous F508del/F508del mutation were expanded from 1×10⁶ to 250×10⁶ cells (Neuberger, T et al., 2011, Methods Mol Biol 741:39-54). For this purpose, cells isolated from CF patients with the homozygous mutation, procured from the CF center tissue procurement and cell culture core at the Marsico Lung Institute at UNC (Randell), the Cystic Fibrosis translational research center at McGill University (University), and Rosalind Franklin University Medical School (RFUMS) were seeded onto 24 well Corning (Cat #3378) filter plates that were coated with 3T3 conditioned media and grown at an air-liquid interface for 35 days using an Ultroser® G supplemented differentiation media. All the primary human bronchial epithelial cells were collected in accordance with institutional review board approval protocols. Apical surface mucus was removed 72 hours before the experiment by incubating the apical surface of the cells for 30 minutes with 3 mM dithiothreitol (DTT) prepared in Dulbecco's phosphate buffered saline (DPBS) with Ca²⁺ and Mg²⁺. This was followed with aspiration of the mucus from the apical surface along with DPBS. The apical surface was re-washed with phosphate buffered saline (PBS) incubated for 30 minutes followed with aspiration. These hBE cells were then incubated with the desired concentrations of the test C2 corrector compounds along with co-corrector, 4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid and potentiator, (5-{3-amino-5-[4-(trifluoromethoxy)benzene-1-sulfonyl]pyridin-2-yl}-1,3,4-oxadiazol-2-yl)methanol, for 18-24 hours at 37° C., 5% CO₂. The desired concentrations of the correctors and potentiator compounds were prepared from the 10 mM stocks in differentiation media and were always applied on the basolateral side of the epithelial cells. We also used an assay format where a fixed concentration of potentiator was added chronically along with the corrector compounds. This chronic treatment with the potentiator helped eliminate any interaction that might happen with CFTR modulators (correctors and potentiators) and thereby determined the true efficacy of the modulator combinations reflective of clinical relevance.

### TECC (Transepithelial Current Clamp) Assay

The assay uses a Transepithelial Current Clamp (TECC) (Vu, C B et al., 2017; J Med Chem 60:458-473) instrument that can measure the functionality of the mutated channel by measuring the equivalent CFTR current (I_{EQ}) generated by the polarized primary epithelial cells. We used a TECC-24 with a 24-channel electrode manifold allowing for the simultaneous measurement of the transepithelial voltage, V_{T}, and transepithelial resistance, R_{T}, under current clamp conditions, from 24 filters using a 24 well Costar filter plate. The design of the filters in the 24 well filter plates was exactly the same as the design of an individual Transwell filter used in the classical Ussing Chamber with a surface area of 0.33 cm². Each measured V_{T} values were corrected for the electrode offset potential measured using buffer alone in a separate plate, and each measured R_{T} values were then corrected for the combined solution series and empty filter resistances. The corrected V_{T} and R_{T} values were then used to calculate the equivalent current, I_{EQ} using Ohm's law (I_{EQ}=V_{T}/R_{T}). The area under the curve (AUC) for the time period between the forskolin peak I_{EQ} response and at the time of bumetanide addition was also calculated using a one-third trapezoid method, in addition to calculating the I_{EQ}. The assay was run in a 24-well format and all 24-wells were measured at the same time point giving a higher throughput for this assay. On the day of measuring the corrector activity on the TECC, the cells were switched into a bicarbonate and serum free F-12 Coon's medium and allowed to equilibrate for 30 minutes for hBE cells in a CO₂ free incubator. At the time of measurement, the apical and basolateral sides of the filter were bathed with the F-12 Coon's modification media (with 20 mM 4-(2-hydroxyethyl)-1-piperazineethanesulfonic acid (HEPES), pH 7.4 (using 1 M tris(hydroxymethyl)aminomethane (Tris)), and the measurements were made at 36.5° C. Current responses before and after the sequential addition of benzamil (apical 6 μM addition; for inhibiting epithelial ENaC channel), forskolin (apical and basolateral 10 μM addition; for activating the CFTR channel), and bumetanide (basolateral 20 μM addition; for inhibiting the Na:2Cl:K co-transporter, an indirect measure of inhibiting the chloride secretion driven by CFTR channel) were measured.

All plates contained negative controls (dimethyl sulfoxide, DMSO) that sets the null response; and positive controls 4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid (0.15 μM) coupled with the control potentiator (5-{3-amino-5-[4-(trifluoromethoxy)benzene-1-sulfonyl]pyridin-2-yl}-1,3,4-oxadiazol-2-yl)methanol (0.45 μM) sets the 100% response to measure the correction of the mutated CFTR channel. The maximum percent activity (Emax) was reported relative to the positive control value.

The % activity measured at each of the 6 test concentrations of the test compound was normalized to the on-plate positive control using the following formula:

% activity=[(test compound response−DMSO response)/(positive control response−DMSO response)]*100

The I_{EQ} and AUC at different test concentrations were fit and an EC₅₀ was calculated using the general sigmoidal curve with variable Hill slope equation included in the Prism v5 software.

TABLE 2
Human Bronchial Epithelial
Cell TECC Assay Data

| Ex | EC₅₀ (μM) | Emax (% Dual) |
|---|---|---|
| 1 | 0.005 | 389 |
| 2 | 0.0129 | 250 |
| 3 | 0.000992 | 360 |
| 4 | 0.0015 | 417 |
| 5 | 0.0035 | 253 |
| 6 | 0.0053 | 378 |
| 7 | 0.0008 | 487 |
| 8 | 0.0014 | 429 |
| 9 | 0.0011 | 381 |
| 10 | 0.804 | 194 |
| 11 | 0.049 | 214 |
| 12 | 0.00051 | 407 |
| 13 | 0.0577 | 232 |
| 14 | 0.799 | 165 |
| 15 | 0.00084 | 308 |
| 16 | 0.0084 | 308 |
| 17 | 0.00066 | 296 |
| 18 | 0.0013 | 270 |
| 19 | 0.0096 | 215 |
| 20 | 0.001 | 250 |
| 21 | 0.002 | 213 |
| 22 | 0.009 | 268 |
| 23 | 0.0062 | 294 |
| 24 | 0.002 | 286 |
| 25 | 0.0043 | 305 |
| 26 | 0.0028 | 293 |
| 27 | 0.0157 | 275 |
| 28 | 0.0026 | 304 |
| 29 | 0.0077 | 241 |
| 30 | 0.0466 | 313 |
| 31 | 0.0062 | 307 |
| 32 | 0.0091 | 259 |
| 33 | 0.0488 | 215 |
| 34 | 0.0029 | 459 |
| 35 | 0.0479 | 331 |
| 36 | >0.3 | 173 |
| 37 | 0.042 | 152 |
| 38 | 0.219 | 202 |
| 39 | 0.018 | 317 |
| 40 | 0.0196 | 328 |
| 41 | 0.11 | 244 |
| 42 | 0.182 | 127 |
| 43 | 0.0029 | 272 |
| 44 | 0.0166 | 193 |
| 45 | >0.3 | 179 |
| 46 | 0.0301 | 364 |
| 47 | 0.0852 | 247 |
| 48 | 0.0471 | 401 |
| 49 | 0.0358 | 321 |
| 50 | 0.0052 | 359 |
| 51 | >0.3 | 135 |
| 52 | 0.0266 | 283 |
| 53 | >0.3 | 169 |
| 54 | 0.0508 | 355 |
| 55 | 0.00586 | 341 |
| 56 | 0.0085 | 290 |
| 57 | 0.0192 | 253 |
| 58 | 0.0314 | 246 |
| 59 | 0.0638 | 161 |
| 60 | 0.0067 | 413 |
| 61 | 0.0745 | 281 |
| 62 | 0.261 | 329 |
| 63 | 0.097 | 315 |
| 64 | 0.0337 | 342 |
| 65 | 0.0193 | 208 |
| 66 | 0.0152 | 253 |
| 67 | 0.0207 | 174 |
| 68 | 0.0211 | 217 |
| 69 | >0.3 | 111 |
| 70 | 0.0118 | 291 |
| 71 | 0.0113 | 288 |
| 72 | 0.0136 | 359 |
| 73 | 0.0306 | 230 |
| 74 | 0.222 | 222 |
| 75 | 0.0362 | 402 |
| 76 | 0.0024 | 269 |
| 77 | 0.0004 | 289 |
| 78 | 0.0303 | 282 |
| 79 | 0.0216 | 205 |
| 80 | 0.0158 | 275 |
| 81 | 0.0944 | 173 |
| 82 | 0.0522 | 210 |
| 83 | 0.0332 | 246 |
| 84 | 0.0175 | 215 |
| 85 | 0.0247 | 206 |
| 86 | 0.0241 | 176 |
| 87 | 0.0069 | 271 |
| 88 | 0.0126 | 275 |
| 89 | 0.142 | 322 |
| 90 | 0.0404 | 348 |
| 91 | >0.8 | 138 |
| 92 | 0.0876 | 187 |
| 93 | 0.0426 | 271 |
| 94 | 0.007 | 246 |
| 95 | 0.0126 | 209 |
| 96 | 0.0078 | 256 |
| 97 | 0.0009 | 198 |
| 98 | 0.021 | 201 |
| 99 | >1 | 126 |
| 100 | 0.114 | 207 |
| 101 | 0.147 | 199 |
| 102 | 0.0327 | 397 |
| 103 | 0.0018 | 266 |
| 104 | 0.0038 | 350 |
| 105 | 0.0031 | 296 |
| 106 | 0.0116 | 188 |

### Cell Surface Expression-Horse Radish Peroxidase (CSE-TIRP) Assay

A cellular assay for measuring the F508delCFTR cell surface expression after correction with test compounds either without or with a co-corrector (2 μM of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid), was developed in human lung derived epithelial cell line (CFBE4lo-) (Veit G et al, (2012) Mol Biol Cell. 23(21): 4188-4202). The development was achieved by expressing the F508delCFTR mutation along with a horseradish peroxidase (HRP) in the fourth exofacial loop, and then measuring the HRP activity using luminescence readout from these cells, CFBE4lo-F508delCFTR-HRP, that were incubated overnight with the test corrector compounds, either without or with the co-corrector. For this primary assay, the CFBE4lo-F508delCFTR-HRP cells were plated in 384-well plates (Greiner Bio-one; Cat 781080) at 4,000 cells/well along with 0.5 μg/mL doxycycline to induce the F508delCFTR-RP expression and further incubated at 37° C., 5% CO₂ for 68-72 hours. The test compounds were then added either without or with a co-corrector at the required concentrations and further incubated for 18-24 hours at 33° C. The highest concentration tested was 20 μM or 30 μM (GI-1 to GIII-36) with an 8-point concentration response curve using a 3-fold dilution in both the test compound without or with the co-corrector. Three replicate plates were run to determine one EC₅₀. All plates contained negative controls (dimethyl sulfoxide, DMSO) and positive control (2 μM or 3 μM (GI-1 to GIII-36) of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid) as well as on-plate concentration response of the positive control. Post incubation, the plates were washed 5× times with Dulbecco's phosphate buffered saline (DPBS), followed by the addition of the HRP substrate, luminol (50 μL), and measuring the HRP activity using luminescence readout on EnVision® Multilabel Plate Reader (Perkin Elmer; product number 2104-0010). The raw counts from the experiment were analyzed using Accelrys® Assay Explorer v3.3.

Z′ greater than 0.5 was used as passing quality control criteria for the plates. The Z′ is defined as:

1−[3*SD_{Positive Control}+3*SD_{Negative Control}/Absolute (Mean_{Positive Control}−Mean_{Negative Control})]

wherein “SD” is standard deviation.

The % activity measured at each of the 8 test concentrations of the test compound added either without or with a co-corrector (2 μM or 3 μM (GI-1 to GIII-36) of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid) was normalized to the on-plate positive control using the following formulae:

% activity (Test compound without co-corrector)=[(test compound without co-corrector response−DMSO response)/(positive control response−DMSO response)]*100

% activity (Test compound with co-corrector)=[(test compound with co-corrector response−DMSO response)/(positive control response−DMSO response)]*100

The maximum % activity achieved for the test compound either without or with a co-corrector at any tested concentration is presented in Table 3 along with the respective EC₅₀'s calculated using the following general sigmoidal curve with variable Hill slope equation (described as Model 42 in the Accelrys® Assay Explorer v3.3 software):

y=(a−d)/(1+(x/c)\hat{ }b)+d

General sigmoidal curve with concentration, response, top, bottom, EC₅₀ and Hill slope. This model describes a sigmoidal curve with an adjustable baseline, a. The equation can be used to fit curves where response is either increasing or decreasing with respect to the independent variable, “x”.

“x” is a concentration of drug under test.

“y” is the response.

“a” is the maximum response, and “d” is the minimum response

“c” is the inflection point (EC₅₀) for the curve. That is, “y” is halfway between the lower and upper asymptotes when x=c.

“b” is the slope-factor or Hill coefficient. The sign of b is positive when the response increases with increasing dose and is negative when the response decreases with increasing dose (inhibition).

TABLE 3
Cell Surface Expression-Horse Radish Peroxidase Assay Data

| | | Maximum | | Maximum |
|---|---|---|---|---|
| | EC₅₀ | % activity | EC₅₀ | % activity |
| | (without | (without | (with co- | (with co- |
| | co-corrector) | co-corrector) | corrector) | corrector) |
| Ex | (μM) | (%) | (μM) | (%) |
| 1 | 2.84 | 1240 | 1.43 | 2160 |
| 2 | 0.626 | 523 | 0.497 | 1190 |
| 3 | 2.45 | 2750 | 0.994 | 3360 |
| 4 | 0.489 | 944 | 0.421 | 2030 |
| 5 | 0.306 | 741 | 0.187 | 1750 |
| 6 | 0.187 | 982 | 0.202 | 2890 |
| 7 | 1.45 | 5940 | 0.926 | 8700 |
| 8 | 0.844 | 3180 | 0.504 | 6530 |
| 9 | 1.67 | 2790 | — | — |
| 11 | 4.6 | 679 | 2.82 | 1310 |
| 12 | 0.479 | 2740 | 0.327 | 6300 |
| 13 | 2.48 | 2100 | 1.49 | 3500 |
| 14 | 3.81 | 520 | 2.8 | 1190 |
| 15 | 1.01 | 3440 | 0.698 | 6730 |
| 16 | 0.328 | 784 | 0.393 | 2360 |
| 17 | 2.38 | 4780 | — | — |
| 18 | 0.198 | 1040 | — | — |
| 19 | 1.01 | 3300 | 0.588 | 5950 |
| 20 | 0.244 | 716 | 0.203 | 2090 |
| 21 | 1.09 | 5140 | 0.506 | 7440 |
| 22 | 0.929 | 2910 | 0.844 | 6250 |
| 23 | 0.327 | 1680 | 0.259 | 4570 |
| 24 | 0.583 | 4040 | 0.201 | 6870 |
| 25 | 1.01 | 2550 | 0.388 | 3940 |
| 26 | 0.368 | 515 | 0.337 | 1530 |
| 27 | 0.405 | 465 | 0.429 | 1430 |
| 28 | 0.951 | 2610 | 0.417 | 4210 |
| 29 | 0.273 | 666 | 0.164 | 1780 |
| 30 | 0.263 | 589 | 0.26 | 1570 |
| 31 | 0.239 | 776 | 0.163 | 1690 |
| 32 | 2.96 | 1350 | 1.44 | 2290 |
| 33 | 1.27 | 470 | 0.906 | 1090 |
| 34 | 0.783 | 1730 | 0.385 | 2960 |
| 35 | 1.71 | 582 | 1.22 | 1200 |
| 36 | 4.32 | 248 | 2.48 | 666 |
| 37 | 7.26 | 113 | 3.68 | 460 |
| 38 | 3.02 | 419 | 1.81 | 995 |
| 39 | 1.23 | 563 | 0.639 | 1101 |
| 40 | 1.55 | 448 | 0.749 | 934 |
| 41 | 2.98 | 497 | 1.77 | 1110 |
| 42 | 1.85 | 690 | 1.15 | 1410 |
| 43 | 2.42 | 1920 | 0.806 | 2990 |
| 44 | 1.41 | 384 | 0.865 | 982 |
| 45 | 6.15 | 128 | 4.62 | 427 |
| 46 | 2.35 | 783 | 1.52 | 1620 |
| 47 | 2.21 | 322 | 1.28 | 775 |
| 48 | 1.78 | 790 | 1.08 | 1650 |
| 49 | 1.99 | 453 | 1.17 | 1060 |
| 50 | 2.34 | 951 | 0.954 | 1770 |
| 51 | 5.44 | 108 | 4.38 | 406 |
| 52 | 3.32 | 608 | 2.14 | 1350 |
| 53 | 3.98 | 229 | 2.61 | 625 |
| 54 | 2.93 | 890 | 2 | 1890 |
| 55 | 1 | 1930 | 0.339 | 3120 |
| 56 | 0.89 | 370 | 0.386 | 917 |
| 57 | 1.13 | 1610 | 0.51 | 2560 |
| 58 | 3.91 | 1040 | 2.12 | 1950 |
| 59 | 1.52 | 438 | 0.779 | 1020 |
| 60 | 2.14 | 2130 | 1.03 | 3190 |
| 61 | 2.44 | 700 | 1.11 | 1180 |
| 62 | 0.99 | 483 | 0.474 | 1015 |
| 63 | 1.74 | 399 | 0.89 | 847 |
| 65 | 1.34 | 394 | 0.586 | 828 |
| 67 | 3.32 | 492 | 1.61 | 989 |
| 68 | 2.76 | 657 | 1.12 | 1217 |
| 69 | 6.01 | 170 | 3.43 | 495 |
| 70 | 4.23 | 482 | 2.6 | 1090 |
| 72 | 3.33 | 651 | 1.54 | 1240 |
| 73 | 1.44 | 539 | 0.785 | 1220 |
| 74 | 1.29 | 188 | 0.743 | 545 |
| 75 | 1.65 | 939 | 0.635 | 1625 |
| 76 | 2.14 | 1119 | 0.989 | 1900 |
| 77 | 1.66 | 1133 | 0.327 | 1600 |
| 78 | 0.825 | 825 | 0.364 | 1650 |
| 79 | 0.465 | 388 | 0.171 | 860 |
| 80 | 1.32 | 925 | 0.461 | 1493 |
| 81 | 5.78 | 476 | 3.98 | 876 |
| 82 | 0.73 | 937 | 0.351 | 1680 |
| 84 | 1.36 | 1310 | 0.945 | 2240 |
| 85 | 1.01 | 786 | 0.81 | 1660 |
| 86 | 2.21 | 1210 | 1.44 | 2020 |
| 87 | 0.452 | 881 | 0.165 | 1810 |
| 88 | 1.18 | 910 | 0.534 | 1740 |
| 89 | 2.24 | 447 | 1.26 | 935 |
| 90 | 1.72 | 812 | 0.9 | 1580 |
| 91 | 5.24 | 226 | 4.34 | 579 |
| 92 | 2.13 | 653 | 1.15 | 1180 |
| 93 | 0.268 | 491 | 0.132 | 1110 |
| 95 | 1.81 | 749 | 0.855 | 1350 |
| 96 | 1.98 | 1150 | 0.99 | 1990 |
| 97 | 0.988 | 2010 | 0.359 | 3320 |
| 98 | 1.44 | 1050 | 0.804 | 1940 |
| 99 | 3.98 | 180 | 2.76 | 509 |
| 100 | 4.49 | 1179 | 2.78 | 2285 |
| 101 | 1 | 243 | 0.884 | 842 |
| 102 | 2.3 | 1226 | 0.825 | 2057 |
| 103 | 0.714 | 800 | 0.587 | 2348 |
| 105 | 1.45 | 1067 | 0.277 | 2229 |
| 106 | — | — | 0.222 | 2407 |

The data provided in the present application demonstrate that the compounds of the invention demonstrate activity in vitro and may be useful in vivo in the treatment of cystic fibrosis.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, Formulations and/or methods of use of the invention, may be made without departing from the spirit and scope thereof. All publications, patents, and patent applications cited herein are hereby incorporated by reference in their entirety for all purposes.

We claim:

1. A compound, or pharmaceutically acceptable salt thereof, of formula (I) 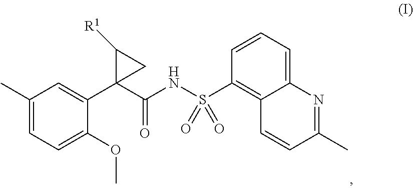
wherein
R¹ is selected from the group consisting of phenyl and 6-membered heteroaryl; wherein R¹ is optionally substituted with one or more R²;
R² is selected from the group consisting of fluoro, chloro, bromo, C₁-C₄ alkyl, C₁-C₄ haloalkyl, C₂-C₆ alkoxyalkyl, —OR^{2a}, and —NR^{2b}R^{2c};
R^{2a} is C₁-C₄ alkyl; and
R^{2b} and R^{2c} are each independently selected from the group consisting of hydrogen and C₁-C₄ alkyl.

2. The compound of claim 1, or pharmaceutically acceptable salt thereof, where R¹ is selected from the group consisting of phenyl, pyridyl, pyrazinyl, pyridazinyl, and pyrimidinyl; wherein R¹ is optionally substituted with one or more R².

3. The compound of claim 2, or pharmaceutically acceptable salt thereof, where R¹ is selected from the group consisting of 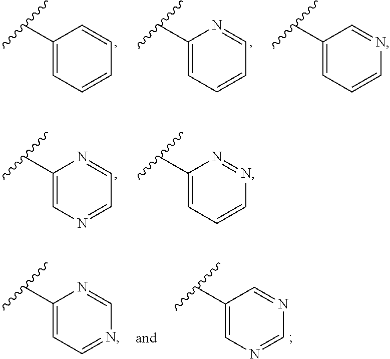
wherein R¹ is optionally substituted with one or more R².

4. The compound of claim 1, or pharmaceutically acceptable salt thereof, where R¹ is pyridyl; wherein R¹ is optionally substituted with one or more R².

5. The compound of claim 4, or pharmaceutically acceptable salt thereof, where R² is selected from the group consisting of C₁-C₄ alkyl, C₁-C₄ haloalkyl, C₂-C₆ alkoxyalkyl, and —OR^{2a}.

6. The compound of claim 5, or pharmaceutically acceptable salt thereof, where R² is selected from the group consisting of C₁-C₄ alkyl and —OR^{2a}.

7. The compound of claim 6, or pharmaceutically acceptable salt thereof, where R² is —OR^{2a}.

8. The compound of claim 7, or pharmaceutically acceptable salt thereof, which is

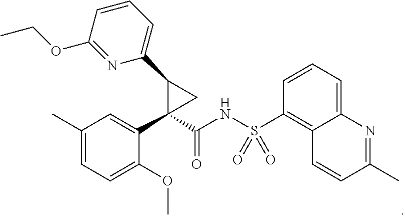 [SMILES: CCOc1cccc([C@H]2C[C@@]2(C(=O)NS(=O)(=O)c2cccc3nc(C)ccc23)c2cc(C)ccc2OC)n1]

9. The compound of claim 8 which is

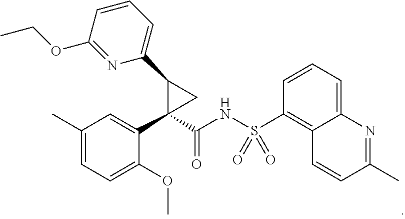 [SMILES: CCOc1cccc([C@H]2C[C@@]2(C(=O)NS(=O)(=O)c2cccc3nc(C)ccc23)c2cc(C)ccc2OC)n1]

10. The compound of claim 1, or pharmaceutically acceptable salt thereof, where R¹ is 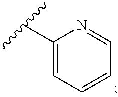
wherein R¹ is optionally substituted with one or more R².

11. The compound of claim 10, or pharmaceutically acceptable salt thereof, where R² is selected from the group consisting of C₁-C₄ alkyl and —OR^{2a}.

12. The compound of claim 1, or pharmaceutically acceptable salt thereof, where R¹ is phenyl; wherein R¹ is optionally substituted with one or more R²; and R² is selected from the group consisting of fluoro, C₁-C₄ alkyl, C₁-C₄ haloalkyl, and —OR^{2a}.

13. The compound of claim 1, or pharmaceutically acceptable salt thereof, where R¹ is pyrazinyl; wherein R¹ is optionally substituted with one or more R²; and R² is selected from the group consisting of C₁-C₄ alkyl and —OR^{2a}.

14. The compound of claim 1, or pharmaceutically acceptable salt thereof, where R¹ is pyrimidinyl; wherein R¹ is optionally substituted with one or more R².

15. The compound of claim 1, or a pharmaceutically acceptable salt thereof, selected from the group consisting of
rac-(1r,2s)-1-(2-methoxy-5-methylphenyl)-N-(2-methylquinoline-5-sulfonyl)-2-phenylcyclopropane-1-carboxamide;
(1R,2S)-1-(2-methoxy-5-methylphenyl)-N-(2-methylquinoline-5-sulfonyl)-2-phenylcyclopropane-1-carboxamide;
(1S,2R)-1-(2-methoxy-5-methylphenyl)-N-(2-methylquinoline-5-sulfonyl)-2-phenylcyclopropane-1-carboxamide;
rac-(1r,2s)-1-(2-methoxy-5-methylphenyl)-2-(4-methylphenyl)-N-(2-methylquinoline-5-sulfonyl)cyclopropane-1-carboxamide;
rac-(1r,2s)-1-(2-methoxy-5-methylphenyl)-N-(2-methylquinoline-5-sulfonyl)-2-[4-(trifluoromethyl)phenyl]cyclopropane-1-carboxamide;
(1R,2S)-1-(2-methoxy-5-methylphenyl)-2-(4-methylphenyl)-N-(2-methylquinoline-5-sulfonyl)cyclopropane-1-carboxamide;
(1S,2R)-1-(2-methoxy-5-methylphenyl)-2-(4-methylphenyl)-N-(2-methylquinoline-5-sulfonyl)cyclopropane-1-carboxamide;
rac-(1r,2s)-2-(3-fluorophenyl)-1-(2-methoxy-5-methylphenyl)-N-(2-methylquinoline-5-sulfonyl)cyclopropane-1-carboxamide;
rac-(1r,2s)-1-(2-methoxy-5-methylphenyl)-2-(4-methoxyphenyl)-N-(2-methylquinoline-5-sulfonyl)cyclopropane-1-carboxamide;
(1R,2S)-1-(2-methoxy-5-methylphenyl)-2-(6-methylpyridin-3-yl)-N-(2-methylquinoline-5-sulfonyl)cyclopropane-1-carboxamide;
(1S,2R)-1-(2-methoxy-5-methylphenyl)-2-(6-methylpyridin-3-yl)-N-(2-methylquinoline-5-sulfonyl)cyclopropane-1-carboxamide;
rac-(1r,2s)-1-(2-methoxy-5-methylphenyl)-N-(2-methylquinoline-5-sulfonyl)-2-[3-(trifluoromethyl)phenyl]cyclopropane-1-carboxamide;
(1R,2R)-1-(2-methoxy-5-methylphenyl)-N-(2-methylquinoline-5-sulfonyl)-2-(pyridin-2-yl)cyclopropane-1-carboxamide;
(1S,2S)-1-(2-methoxy-5-methylphenyl)-N-(2-methylquinoline-5-sulfonyl)-2-(pyridin-2-yl)cyclopropane-1-carboxamide;
rac-(1r,2s)-1-(2-methoxy-5-methylphenyl)-2-(3-methoxyphenyl)-N-(2-methylquinoline-5-sulfonyl)cyclopropane-1-carboxamide;
(1R,2S)-1-(2-methoxy-5-methylphenyl)-2-(4-methoxyphenyl)-N-(2-methylquinoline-5-sulfonyl)cyclopropane-1-carboxamide;
(1S,2R)-1-(2-methoxy-5-methylphenyl)-2-(4-methoxyphenyl)-N-(2-methylquinoline-5-sulfonyl)cyclopropane-1-carboxamide;
(1S,2R)-1-(2-methoxy-5-methylphenyl)-N-(2-methylquinoline-5-sulfonyl)-2-[4-(trifluoromethyl)phenyl]cyclopropane-1-carboxamide;
(1R,2S)-1-(2-methoxy-5-methylphenyl)-N-(2-methylquinoline-5-sulfonyl)-2-[4-(trifluoromethyl)phenyl]cyclopropane-1-carboxamide;
(1R,2S)-2-(3-fluorophenyl)-1-(2-methoxy-5-methylphenyl)-N-(2-methylquinoline-5-sulfonyl)cyclopropane-1-carboxamide;
(1S,2R)-2-(3-fluorophenyl)-1-(2-methoxy-5-methylphenyl)-N-(2-methylquinoline-5-sulfonyl)cyclopropane-1-carboxamide;
rac-(1r,2r)-2-(2-fluorophenyl)-1-(2-methoxy-5-methylphenyl)-N-(2-methylquinoline-5-sulfonyl)cyclopropane-1-carboxamide;
(1S,2R)-1-(2-methoxy-5-methylphenyl)-N-(2-methylquinoline-5-sulfonyl)-2-[3-(trifluoromethyl)phenyl]cyclopropane-1-carboxamide;
(1R,2S)-1-(2-methoxy-5-methylphenyl)-N-(2-methylquinoline-5-sulfonyl)-2-[3-(trifluoromethyl)phenyl]cyclopropane-1-carboxamide;
(1S,2S)-2-(2-fluorophenyl)-1-(2-methoxy-5-methylphenyl)-N-(2-methylquinoline-5-sulfonyl)cyclopropane-1-carboxamide;
(1R,2R)-2-(2-fluorophenyl)-1-(2-methoxy-5-methylphenyl)-N-(2-methylquinoline-5-sulfonyl)cyclopropane-1-carboxamide;
(1R,2S)-1-(2-methoxy-5-methylphenyl)-2-(3-methoxyphenyl)-N-(2-methylquinoline-5-sulfonyl)cyclopropane-1-carboxamide;
(1S,2R)-1-(2-methoxy-5-methylphenyl)-2-(3-methoxyphenyl)-N-(2-methylquinoline-5-sulfonyl)cyclopropane-1-carboxamide;
rac-(1r,2s)-1-(2-methoxy-5-methylphenyl)-N-(2-methylquinoline-5-sulfonyl)-2-[2-(trifluoromethyl)phenyl]cyclopropane-1-carboxamide;
(1S,2R)-1-(2-methoxy-5-methylphenyl)-N-(2-methylquinoline-5-sulfonyl)-2-[2-(trifluoromethyl)phenyl]cyclopropane-1-carboxamide;
(1R,2S)-1-(2-methoxy-5-methylphenyl)-N-(2-methylquinoline-5-sulfonyl)-2-[2-(trifluoromethyl)phenyl]cyclopropane-1-carboxamide;
(1S,2R)-1-(2-methoxy-5-methylphenyl)-2-(6-methoxypyridin-3-yl)-N-(2-methylquinoline-5-sulfonyl)cyclopropane-1-carboxamide;
(1R,2S)-1-(2-methoxy-5-methylphenyl)-2-(6-methoxypyridin-3-yl)-N-(2-methylquinoline-5-sulfonyl)cyclopropane-1-carboxamide;
(1S,2R)-1-(2-methoxy-5-methylphenyl)-2-(2-methylphenyl)-N-(2-methylquinoline-5-sulfonyl)cyclopropane-1-carboxamide;
rac-(1r,2r)-1-(2-methoxy-5-methylphenyl)-2-(6-methylpyrazin-2-yl)-N-(2-methylquinoline-5-sulfonyl)cyclopropane-1-carboxamide;
rac-(1r,2s)-1-(2-methoxy-5-methylphenyl)-2-(2-methoxypyrimidin-5-yl)-N-(2-methylquinoline-5-sulfonyl)cyclopropane-1-carboxamide;
(1R,2S)-1-(2-methoxy-5-methylphenyl)-2-(2-methoxypyrimidin-5-yl)-N-(2-methylquinoline-5-sulfonyl)cyclopropane-1-carboxamide;
(1S,2R)-1-(2-methoxy-5-methylphenyl)-2-(2-methoxypyrimidin-5-yl)-N-(2-methylquinoline-5-sulfonyl)cyclopropane-1-carboxamide;
rac-(1r,2r)-1-(2-methoxy-5-methylphenyl)-2-(5-methoxypyrazin-2-yl)-N-(2-methylquinoline-5-sulfonyl)cyclopropane-1-carboxamide;
rac-(1r,2s)-1-(2-methoxy-5-methylphenyl)-2-(2-methoxypyridin-3-yl)-N-(2-methylquinoline-5-sulfonyl)cyclopropane-1-carboxamide;
rac-(1r,2s)-1-(2-methoxy-5-methylphenyl)-2-(5-methoxypyridin-3-yl)-N-(2-methylquinoline-5-sulfonyl)cyclopropane-1-carboxamide;
rac-(1r,2s)-2-(5-fluoropyridin-3-yl)-1-(2-methoxy-5-methylphenyl)-N-(2-methylquinoline-5-sulfonyl)cyclopropane-1-carboxamide;
(1s,2s)-1-(2-methoxy-5-methylphenyl)-2-(6-methoxypyridin-2-yl)-N-(2-methylquinoline-5-sulfonyl)cyclopropane-1-carboxamide;
(1R,2R)-1-(2-methoxy-5-methylphenyl)-2-(6-methoxypyridin-2-yl)-N-(2-methylquinoline-5-sulfonyl)cyclopropane-1-carboxamide;
(1R,2R)-1-(2-methoxy-5-methylphenyl)-2-(6-methylpyrazin-2-yl)-N-(2-methylquinoline-5-sulfonyl)cyclopropane-1-carboxamide;
(1S,2S)-1-(2-methoxy-5-methylphenyl)-2-(6-methylpyrazin-2-yl)-N-(2-methylquinoline-5-sulfonyl)cyclopropane-1-carboxamide;
(1R,2R)-1-(2-methoxy-5-methylphenyl)-2-(5-methoxypyrazin-2-yl)-N-(2-methylquinoline-5-sulfonyl)cyclopropane-1-carboxamide;
(1S,2S)-1-(2-methoxy-5-methylphenyl)-2-(5-methoxypyrazin-2-yl)-N-(2-methylquinoline-5-sulfonyl)cyclopropane-1-carboxamide;
(1R,2S)-1-(2-methoxy-5-methylphenyl)-2-(2-methoxypyridin-3-yl)-N-(2-methylquinoline-5-sulfonyl)cyclopropane-1-carboxamide;
(1S,2R)-1-(2-methoxy-5-methylphenyl)-2-(2-methoxypyridin-3-yl)-N-(2-methylquinoline-5-sulfonyl)cyclopropane-1-carboxamide;
(1R,2S)-1-(2-methoxy-5-methylphenyl)-2-(5-methoxypyridin-3-yl)-N-(2-methylquinoline-5-sulfonyl)cyclopropane-1-carboxamide;
(1S,2R)-1-(2-methoxy-5-methylphenyl)-2-(5-methoxypyridin-3-yl)-N-(2-methylquinoline-5-sulfonyl)cyclopropane-1-carboxamide;
(1R,2S)-2-(5-fluoropyridin-3-yl)-1-(2-methoxy-5-methylphenyl)-N-(2-methylquinoline-5-sulfonyl)cyclopropane-1-carboxamide;
(1S,2R)-2-(5-fluoropyridin-3-yl)-1-(2-methoxy-5-methylphenyl)-N-(2-methylquinoline-5-sulfonyl)cyclopropane-1-carboxamide;
(1S,2S)-2-(6-ethoxypyridin-2-yl)-1-(2-methoxy-5-methylphenyl)-N-(2-methylquinoline-5-sulfonyl)cyclopropane-1-carboxamide;
(1R,2R)-2-(6-ethoxypyridin-2-yl)-1-(2-methoxy-5-methylphenyl)-N-(2-methylquinoline-5-sulfonyl)cyclopropane-1-carboxamide;
(1S,2S)-1-(2-methoxy-5-methylphenyl)-N-(2-methylquinoline-5-sulfonyl)-2-[6-(propan-2-yl)pyridin-2-yl]cyclopropane-1-carboxamide;
(1S,2R)-2-(6-ethoxypyridin-3-yl)-1-(2-methoxy-5-methylphenyl)-N-(2-methylquinoline-5-sulfonyl)cyclopropane-1-carboxamide;
(1R,2S)-2-(6-ethoxypyridin-3-yl)-1-(2-methoxy-5-methylphenyl)-N-(2-methylquinoline-5-sulfonyl)cyclopropane-1-carboxamide;
(1S,2S)-2-[6-(dimethylamino)pyridin-2-yl]-1-(2-methoxy-5-methylphenyl)-N-(2-methylquinoline-5-sulfonyl)cyclopropane-1-carboxamide;
rac-(1r,2r)-1-(2-methoxy-5-methylphenyl)-N-(2-methylquinoline-5-sulfonyl)-2-[5-(propan-2-yl)pyrazin-2-yl]cyclopropane-1-carboxamide;
rac-(1r,2r)-2-(5-ethoxypyrazin-2-yl)-1-(2-methoxy-5-methylphenyl)-N-(2-methylquinoline-5-sulfonyl)cyclopropane-1-carboxamide;
(1R,2R)-1-(2-methoxy-5-methylphenyl)-N-(2-methylquinoline-5-sulfonyl)-2-[5-(propan-2-yl)pyrazin-2-yl]cyclopropane-1-carboxamide;
(1S,2S)-1-(2-methoxy-5-methylphenyl)-N-(2-methylquinoline-5-sulfonyl)-2-[5-(propan-2-yl)pyrazin-2-yl]cyclopropane-1-carboxamide;
(1R,2R)-2-(5-ethoxypyrazin-2-yl)-1-(2-methoxy-5-methylphenyl)-N-(2-methylquinoline-5-sulfonyl)cyclopropane-1-carboxamide;
(1S,2S)-2-(5-ethoxypyrazin-2-yl)-1-(2-methoxy-5-methylphenyl)-N-(2-methylquinoline-5-sulfonyl)cyclopropane-1-carboxamide;
(1S,2R)-1-(2-methoxy-5-methylphenyl)-N-(2-methylquinoline-5-sulfonyl)-2-[6-(propan-2-yl)pyridin-3-yl]cyclopropane-1-carboxamide;
(1S,2R)-2-[6-(dimethylamino)pyridin-3-yl]-1-(2-methoxy-5-methylphenyl)-N-(2-methylquinoline-5-sulfonyl)cyclopropane-1-carboxamide;
(1S,2S)-1-(2-methoxy-5-methylphenyl)-N-(2-methylquinoline-5-sulfonyl)-2-[6-(propan-2-yl)pyridazin-3-yl]cyclopropane-1-carboxamide;
(1S,2R)-2-[6-(difluoromethyl)pyridin-3-yl]-1-(2-methoxy-5-methylphenyl)-N-(2-methylquinoline-5-sulfonyl)cyclopropane-1-carboxamide;
(1S,2R)-2-(2-ethoxypyridin-3-yl)-1-(2-methoxy-5-methylphenyl)-N-(2-methylquinoline-5-sulfonyl)cyclopropane-1-carboxamide;
(1S,2R)-1-(2-methoxy-5-methylphenyl)-2-(6-methoxy-2-methylpyridin-3-yl)-N-(2-methylquinoline-5-sulfonyl)cyclopropane-1-carboxamide;
(1R,2S)-2-(2-ethoxypyridin-3-yl)-1-(2-methoxy-5-methylphenyl)-N-(2-methylquinoline-5-sulfonyl)cyclopropane-1-carboxamide;
(1R,2R)-1-(2-methoxy-5-methylphenyl)-2-(6-methoxypyrazin-2-yl)-N-(2-methylquinoline-5-sulfonyl)cyclopropane-1-carboxamide;
(1S,2S)-1-(2-methoxy-5-methylphenyl)-2-(6-methoxypyrazin-2-yl)-N-(2-methylquinoline-5-sulfonyl)cyclopropane-1-carboxamide;
(1S,2R)-2-(5-fluoro-6-methoxypyridin-3-yl)-1-(2-methoxy-5-methylphenyl)-N-(2-methylquinoline-5-sulfonyl)cyclopropane-1-carboxamide;
(1S,2S)-2-[6-(difluoromethyl)pyridin-2-yl]-1-(2-methoxy-5-methylphenyl)-N-(2-methylquinoline-5-sulfonyl)cyclopropane-1-carboxamide;
rac-(1r,2s)-1-(2-methoxy-5-methylphenyl)-2-[2-methoxy-6-(propan-2-yl)pyridin-3-yl]-N-(2-methylquinoline-5-sulfonyl)cyclopropane-1-carboxamide;
rac-(1r,2s)-2-(2,6-dimethoxypyridin-3-yl)-1-(2-methoxy-5-methylphenyl)-N-(2-methylquinoline-5-sulfonyl)cyclopropane-1-carboxamide;
(1S,2S)-2-(5,6-dimethoxypyrazin-2-yl)-1-(2-methoxy-5-methylphenyl)-N-(2-methylquinoline-5-sulfonyl)cyclopropane-1-carboxamide;
(1S,2R)-2-(2,6-dimethylpyridin-3-yl)-1-(2-methoxy-5-methylphenyl)-N-(2-methylquinoline-5-sulfonyl)cyclopropane-1-carboxamide;
(1S,2S)-2-(3,5-dimethoxypyrazin-2-yl)-1-(2-methoxy-5-methylphenyl)-N-(2-methylquinoline-5-sulfonyl)cyclopropane-1-carboxamide;
(1S,2S)-1-(2-methoxy-5-methylphenyl)-2-(5-methoxy-6-methylpyrazin-2-yl)-N-(2-methylquinoline-5-sulfonyl)cyclopropane-1-carboxamide;
(1S,2R)-2-(5-fluoro-2-methoxypyridin-3-yl)-1-(2-methoxy-5-methylphenyl)-N-(2-methylquinoline-5-sulfonyl)cyclopropane-1-carboxamide;
(1R,2S)-1-(2-methoxy-5-methylphenyl)-2-(2-methoxy-6-methylpyridin-3-yl)-N-(2-methylquinoline-5-sulfonyl)cyclopropane-1-carboxamide;
(1S,2R)-1-(2-methoxy-5-methylphenyl)-2-(2-methoxy-6-methylpyridin-3-yl)-N-(2-methylquinoline-5-sulfonyl)cyclopropane-1-carboxamide;
(1R,2S)-1-(2-methoxy-5-methylphenyl)-2-[2-methoxy-6-(propan-2-yl)pyridin-3-yl]-N-(2-methylquinoline-5-sulfonyl)cyclopropane-1-carboxamide;
(1S,2R)-1-(2-methoxy-5-methylphenyl)-2-[2-methoxy-6-(propan-2-yl)pyridin-3-yl]-N-(2-methylquinoline-5-sulfonyl)cyclopropane-1-carboxamide;
rac-(1r,2r)-1-(2-methoxy-5-methylphenyl)-2-(5-methylpyrazin-2-yl)-N-(2-methylquinoline-5-sulfonyl)cyclopropane-1-carboxamide;
(1S,2S)-1-(2-methoxy-5-methylphenyl)-2-[6-(methoxymethyl)pyridin-2-yl]-N-(2-methylquinoline-5-sulfonyl)cyclopropane-1-carboxamide;
(1R,2R)-1-(2-methoxy-5-methylphenyl)-2-(5-methylpyrazin-2-yl)-N-(2-methylquinoline-5-sulfonyl)cyclopropane-1-carboxamide;
(1S,2S)-1-(2-methoxy-5-methylphenyl)-2-(5-methylpyrazin-2-yl)-N-(2-methylquinoline-5-sulfonyl)cyclopropane-1-carboxamide;
(1R,2S)-2-(2,6-dimethoxypyridin-3-yl)-1-(2-methoxy-5-methylphenyl)-N-(2-methylquinoline-5-sulfonyl)cyclopropane-1-carboxamide;
(1S,2R)-2-(2,6-dimethoxypyridin-3-yl)-1-(2-methoxy-5-methylphenyl)-N-(2-methylquinoline-5-sulfonyl)cyclopropane-1-carboxamide;
rac-(1r,2r)-1-(2-methoxy-5-methylphenyl)-2-(2-methoxypyrimidin-4-yl)-N-(2-methylquinoline-5-sulfonyl)cyclopropane-1-carboxamide;
(1S,2S)-1-(2-methoxy-5-methylphenyl)-2-(6-methylpyridin-2-yl)-N-(2-methylquinoline-5-sulfonyl)cyclopropane-1-carboxamide;
(1S,2S)-1-(2-methoxy-5-methylphenyl)-N-(2-methylquinoline-5-sulfonyl)-2-{6-[(propan-2-yl)oxy]pyridin-2-yl}cyclopropane-1-carboxamide;
(1S,2S)-1-(2-methoxy-5-methylphenyl)-2-(2-methoxypyrimidin-4-yl)-N-(2-methylquinoline-5-sulfonyl)cyclopropane-1-carboxamide;
(1R,2R)-1-(2-methoxy-5-methylphenyl)-2-(2-methoxypyrimidin-4-yl)-N-(2-methylquinoline-5-sulfonyl)cyclopropane-1-carboxamide;
(1S,2R)-2-(5-fluoro-6-methylpyridin-3-yl)-1-(2-methoxy-5-methylphenyl)-N-(2-methylquinoline-5-sulfonyl)cyclopropane-1-carboxamide;
(1R,2S)-2-(5-fluoro-6-methylpyridin-3-yl)-1-(2-methoxy-5-methylphenyl)-N-(2-methylquinoline-5-sulfonyl)cyclopropane-1-carboxamide;
(1S,2R)-2-(5-chloro-6-methoxypyridin-3-yl)-1-(2-methoxy-5-methylphenyl)-N-(2-methylquinoline-5-sulfonyl)cyclopropane-1-carboxamide;
(1S,2R)-1-(2-methoxy-5-methylphenyl)-2-(6-methoxy-5-methylpyridin-3-yl)-N-(2-methylquinoline-5-sulfonyl)cyclopropane-1-carboxamide;
(1S,2R)-2-(5,6-dimethoxypyridin-3-yl)-1-(2-methoxy-5-methylphenyl)-N-(2-methylquinoline-5-sulfonyl)cyclopropane-1-carboxamide;
(1S,2S)-2-(6-chloropyridin-2-yl)-1-(2-methoxy-5-methylphenyl)-N-(2-methylquinoline-5-sulfonyl)cyclopropane-1-carboxamide; and
(1S,2S)-2-(6-ethoxy-5-fluoropyridin-2-yl)-1-(2-methoxy-5-methylphenyl)-N-(2-methylquinoline-5-sulfonyl)cyclopropane-1-carboxamide.

16. The compound of claim 15, or a pharmaceutically acceptable salt thereof which is (1S,2S)-2-(6-ethoxypyridin-2-yl)-1-(2-methoxy-5-methylphenyl)-N-(2-methylquinoline-5-sulfonyl)cyclopropane-1-carboxamide.

17. The compound of claim 16 which is the pharmaceutically acceptable salt of (1S,2S)-2-(6-ethoxypyridin-2-yl)-1-(2-methoxy-5-methylphenyl)-N-(2-methylquinoline-5-sulfonyl)cyclopropane-1-carboxamide.

18. The compound of claim 16 which is (1S,2S)-2-(6-ethoxypyridin-2-yl)-1-(2-methoxy-5-methylphenyl)-N-(2-methylquinoline-5-sulfonyl)cyclopropane-1-carboxamide.

19. A pharmaceutical composition comprising a therapeutically effective amount of a compound of Formula (I) according to claim 1, or a pharmaceutically acceptable salt thereof, in combination with a pharmaceutically acceptable carrier.

20. A pharmaceutical composition comprising a compound of claim 1, or a pharmaceutically acceptable salt thereof, one or more potentiator, and one or more additional correctors.

21. A method for treating cystic fibrosis in a subject comprising administering a therapeutically effective amount of a compound of claim 1, or a pharmaceutically acceptable salt thereof, to a subject in need thereof.

22. The method of claim 21, further comprising administering a therapeutically effective amount of one or more potentiator; and administering a therapeutically effective amount of one or more correctors.
